(19) United States
(12) Patent Application Publication
(10) Pub. No.: US 2024/0277776 A1
(43) Pub. Date: Aug. 22, 2024

(54) HYPOIMMUNE CELLS

(71) Applicant: VERTEX PHARMACEUTICALS INCORPORATED, Boston, MA (US)

(72) Inventors: Michael CONWAY, Boston, MA (US); Tudor FULGA, Boston, MA (US)

(73) Assignee: VERTEX PHARMACEUTICALS INCORPORATED, Boston, MA (US)

(21) Appl. No.: 18/640,564

(22) Filed: Apr. 19, 2024

Related U.S. Application Data
(63) Continuation of application No. PCT/US2022/078411, filed on Oct. 20, 2022.
(60) Provisional application No. 63/270,277, filed on Oct. 21, 2021.

(51) Int. Cl. A61K 35/545; C07K 14/705; C12N 5/0735; C12N 9/22; C12N 15/113; C12N 15/90

(52) U.S. Cl. CPC A61K 35/545; C07K 14/70532; C12N 5/0606; C12N 9/22; C12N 15/1138; C12N 15/907; C12N 2310/20; C12N 2800/80

(57) ABSTRACT

Disclosed herein are compositions and methods related to isolated cells (e.g., isolated stem cells) comprising a disruption in the 3′-UTR of an immunosuppressor, cells differentiated from such stem cells (e.g., pancreatic islet cells or immune cells) and methods of using the cells to treat diseases (e.g., diabetes or cancer). Methods of producing (i.e., genetically modifying) the isolated cells (e.g., isolated stem cells) are also provided.

### RELATED APPLICATION

This application claims the benefit under 35 U.S.C. § 119(e) to U.S. Provisional Application No. 63/270,277, filed on Oct. 21, 2021, entitled “HYPOIMMUNE CELLS,” the entire contents of which are incorporated herein by reference.

### INCORPORATION BY REFERENCE OF SEQUENCE LISTING

The Sequence Listing in an XML file, named 41582Z_Sequence_Listing.xml of 220 KB, created on Apr. 16, 2024, and submitted to the United States Patent and Trademark Office via Patent Center, is incorporated herein by reference.

### BACKGROUND

Generation of stem cell (e.g., human embryonic stem cells or human pluripotent stem cells) derived human adult cells for administration to subjects as cell-based therapies provide potential to treat most if not all degenerative diseases. However, the success of such therapy may be limited by the subject's immune response. Strategies that have been considered to overcome the immune rejection include reducing or eliminating the expression of MHC-1 and/or MHC-II human leukocyte antigens and/or increasing the expression of tolerogenic factors in the cells for transplantation.

### INCORPORATION BY REFERENCE

All publications, patents, and patent applications mentioned in this specification are herein incorporated by reference to the same extent as if each individual publication, patent, or patent application was specifically and individually indicated to be incorporated by reference. Absent any indication otherwise, publications, patents, and patent applications mentioned in this specification are incorporated herein by reference in their entireties.

### SUMMARY

The present disclosure relates, at least in part, to hypoimmune cells that can be used for cell-based therapies and methods of producing such hypoimmune cells. In some aspects, the hypoimmune cells are produced from stem cells (e.g., human embryonic stem cells or human pluripotent stem cells). As described herein, such stem cells are manipulated to have one or more genetic disruptions, for example in the 3′-UTR of a gene encoding an immunosuppressor to generate hypoimmune cells that, if stem cells, may in turn be further differentiated to a cell type of choice for subsequent use in cell-based therapies. As such, also provided herein are the genetic modifications made to the cells (e.g., stem cells) and methods and compositions for making such genetic modifications. Methods of using the hypoimmune cells for treating diseases (e.g., diabetes, cancer) are also provided.

Some aspects of the present disclosure provide isolated cells (e.g., isolated stem cells) comprising a disruption in the 3′-untranslated region (3′-UTR) of an allele encoding an immunosuppressor. In some embodiments, the disruption comprises a deletion, an insertion, a translocation, an inversion, or a substitution in the 3′-UTR. In some embodiments, the disruption reduces binding of the 3′-UTR to endogenous RNA-binding proteins and/or microRNAs.

In some embodiments, the immunosuppressor is selected from the group consisting of: PDL1, CD47, HLA-G, and combinations thereof. In some embodiments, the deletion in the 3′-UTR results in increased expression of the immunosuppressor. In some embodiments, the increased expression of the immunosuppressor is induced or increased by a cytokine, optionally wherein the cytokine is interferon gamma. In some embodiments, the immunosuppressor is PDL1. In some embodiments, the disruption results in a deletion of the PDL1 3′-UTR. In some embodiments, the disruption results in an inversion of the PDL1 3′-UTR. In some embodiments, the disruption results in one or more substitutions of nucleotide in the PD-L1 3′-UTR. In some embodiments, the disruption reduces binding of one or more of endogenous microRNAs to PDL1 3′-UTR, optionally wherein the one or more of endogenous microRNA are selected from the group consisting of: miR-34a, miR-140, miR-200a, miR-200b/c, miR-142, miR-340, miR-383, miR-424(322), miR-338-5p, miR-324-5p, miR-152, miR-200b, miR-138-5p, miR-195, miR-16, miR-15a, miR15b miR-193a-3p, miR-497-5p, miR-33a, miR17-5p, miR-155, and miR-513. In some embodiments, the disruption results in deletion of 1-7 nucleotides in one or more of PDL1 3′-UTR sequences as set forth in any one of SEQ ID NOs: 32, 34, 36, 38, 40, 42, 45, 48, 36, 58, 59, 61, 63, 65, 67, 69, 71, and 73. In some embodiments, the disruption results in deletion of 1-24 nucleotides in one or more of PDL1 3′-UTR sequences as set forth in any one of SEQ ID NOs: 31, 33, 35, 37, 39, 41, 44, 47, 57, 60, 62, 64, 66, 68, 70, and 72.

In some embodiments, the immunosuppressor is HLA-G. In some embodiments, the disruption reduces binding of one or more of endogenous microRNAs to HLA-G 3′-UTR, optionally wherein the one or more of endogenous microRNA are selected from the group consisting of: miR-133A, miR-148A, miR-148B, miR-152, miR-548q and/or miR-628-5p. In some embodiments, the disruption results in deletion of at least 5 consecutive nucleotides beginning at and inclusive of position +2961 of the HLA-G 3′-UTR, and/or insertion of at least 5 nucleotides at position +2961. In some embodiments, the disruption is in an HLA-G 3′-UTR sequence as set forth in SEQ ID NO: 74. In some embodiments, the disruption results in a deletion of at least 1 nucleotide of an HLA-G 3′-UTR sequence as set forth in SEQ ID NO: 75. In some embodiments, the disruption results in one or more mutations selected from C120G, G252C, A297G, and/or C306G in an HLA-G 3′-UTR sequence as set forth in SEQ ID NO: 74.

In some embodiments, the isolated cells (e.g., isolated stem cells) further comprise an insertion of a sequence encoding CD47, CTLA-4, PDL1, PDL2, HLA-C, HLA-E, HLA-G, C1-inhibitor, IL-35, DUX4, IDO1, IL10, CCL21, CCL22, CD16, CD52, H2-M3, CD200, FASLG, MFGE8, and/or SERPINB9 into the disrupted 3′-UTR locus. In some embodiments, insertion of the sequence encoding CD47 into the PDL1 3′-UTR locus results in an RNA comprising coding sequences for the immunosuppressor and CD47.

In some embodiments, the isolated cells (e.g., isolated stem cells) further comprise an insertion of a sequence encoding CD47, CTLA-4, PDL1, PDL2, HLA-C, HLA-E, HLA-G, C1-inhibitor, IL-35, DUX4, IDO1, IL10, CCL21, CCL22, CD16, CD52, H2-M3, CD200, FASLG, MFGE8, and/or SERPINB9 into a safe harbor locus.

In some embodiments, the isolated cell (e.g., isolated stem cell) does not contain an insertion of an exogenous coding sequence in its genome.

In some embodiments, the isolated cell (e.g., isolated stem cell) has reduced expression of MHC-I and MHC-II human leukocyte antigens (HLA) relative to a wild-type stem cell of the same cell type. In some embodiments, the reduced expression of MHC-I HLA results from a disruption in an allele encoding 3-2 microglobulin (B2M). In some embodiments, the reduced expression of MHC-II HLA results from a disruption in an allele encoding class II major histocompatibility complex transactivator (CIITA).

In some embodiments, the stem cell is an embryonic stem cell. In some embodiments, the stem cell is a pluripotent stem cell. In some embodiments, the stem cell is a human stem cell. In some embodiments, the stem cell is negative for A antigen and negative for B antigen. In some embodiments, the stem cell is negative for A antigen. In some embodiments, the stem cell is negative for B antigen. In some embodiments, the stem cell is negative for A antigen and positive for B antigen. In some embodiments, the stem cell is positive for A antigen and negative for B antigen. In some embodiments, the stem cell is negative for Rh antigen.

Other aspects of the present disclosure provide cells differentiated from any of the isolated stem cells described herein. In some embodiments, the cell is selected from the group consisting of: a fibroblast cell, an endothelial cell, a definitive endoderm cell, a primitive gut tube cell, a pancreatic progenitor cell, a pancreatic endocrine cell, a pancreatic islet cell, a stem cell-derived β cell, a stem cell-derived α cell, a stem cell-derived δ cell, a stem cell-derived enterochromaffin (EC) cell, an insulin producing cell, an insulin-positive β-like cell, a hematopoietic stem cell, a hematopoietic progenitor cell, a muscle cell, a satellite stem cell, a liver cell, a neuron, or an immune cell. In some embodiments, the cell is an immune cell, optionally wherein the immune cell expresses a chimeric antigen receptor (CAR) or an engineered T-cell receptor (TCR). In some embodiments, the cell is less immunogenic relative to a cell of the same cell type.

Compositions comprising the isolated cells (e.g., isolated stem cells) or the cell differentiated from the isolated stem cells described herein are also provided. In some embodiments, the composition comprises NKX6.1-positive, ISL-positive cells and NKX6.1-negative, ISL-positive cells; wherein the population comprises more NKX6.1-positive, ISL-positive cells than NKX6.1-negative, ISL-positive cells; wherein at least 15% of the cells in the population are NKX6.1-negative, ISL-positive cells; and wherein less than 12% of the cells in the population are NKX6.1-negative, ISL-negative cells.

Further provided herein are methods comprising administering to a subject in need thereof the isolated cells (e.g., isolated stem cells) or the cell differentiated from the isolated stem cells described herein. In some embodiments, the method is a method of treating diabetes and comprises administering to a subject in need thereof pancreatic islet cells differentiated from the isolated stem cells described herein, or the composition comprising such cells. In some embodiments, the method is a method of treating cancer and comprises administering to a subject in need thereof immune cells differentiated from the isolated stem cell described herein or the composition comprising such cells. In some embodiments, the cancer is a hematologic cancer.

Other aspects of the present disclosure provide methods of producing the isolated cells (e.g., isolated stem cells) described herein, the method comprising delivering to a stem cell a CRISPR system comprising an RNA-targeted endonuclease and one or more guide RNAs (gRNA) comprising a nucleotide sequence that targets the 3′-UTR of an allele encoding the immunosuppressor.

In some embodiments, the RNA-targeted endonuclease is a Cas protein. In some embodiments, the Cas protein is a Cas9 protein, a Cas12i protein or a Casϕ protein. In some embodiments, the immunosuppressor is PDL1, CD47, or HLA-G. In some embodiments, the immunosuppressor is PDL1. In some embodiments, the gRNA targets a target sequence corresponding to positions 1003-1022 or positions 1021-1040 of a PDL1 sequence as set forth in SEQ ID NO: 1, or targets a target sequence downstream of the 3′-UTR of PDL1 on opposite strand. In some embodiments, the composition comprises a first gRNA that targets a target sequence corresponding to positions 1003-1022 or positions 1021-1040 of a PDL1 sequence as set forth in SEQ ID NO: 1 and a second gRNA that targets a target sequence downstream of the 3′-UTR of PDL1 on opposite strand. In some embodiments, the gRNA is modified. In some embodiments, the gRNA is delivered in a lipid nanoparticle (LNP). In some embodiments, the gRNA is delivered via a nucleic acid comprising a nucleotide sequence encoding the gRNAs, optionally wherein the nucleic acid is a viral vector. In some embodiments, RNA-targeted endonuclease is delivered via a nucleic acid comprising a nucleotide sequence encoding the RNA-targeted endonuclease, optionally wherein the nucleic acid is a viral vector.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1B is a series of graphs that show disrupting the 3′-UTR of PDL1 reduces CD69 expression, which indicates T-cell activation. FIG. 1A is a graph that shows the percent of CD8+ T-cells with surface expression of CD69 when mixed with media alone, media containing CD3/CD28, endothelial cells differentiated from wild-type human embryonic stem cells (hESCs), from hESCs having B2M/CIITA double knockout (DKO), or from three clones (#12, #19, #25) hESCs having deletion of the 3′-UTR of PDL1. FIG. 1B is a graph that shows HLA-I expression measured by flow cytometry and reported as mean fluorescence intensity (MFI)) for the indicated cells. Cells in which the 3′-UTR of PDL1 is disrupted express HLA-I at a comparable level as wild type cells.

FIG. 2 is a graph that shows the percent of CD8+ T-cells with surface expression of CD69 when mixed with media alone, a positive control, GMP-WCB, three clones of endothelial cells differentiated from wild-type human embryonic stem cells (hESCs), or from three clones of hESCs having deletion of the 3′-UTR of PDL1. *** indicates p<0.001. Consistent with what was observed in FIG. 1A, deletion of PDL1's 3′-UTR reduced T-cell activation to a similar extent observed with the removal of HLA class I and II.

FIG. 3 is a graph that shows surface expression of PDL1 (measured by flow cytometry and reported as MFI) on endothelial cells differentiated from wild-type human embryonic stem cells (hESCs) or from hESCs having deletion of the 3′-UTR of PDL1 without stimulation, or from hESCs having deletion of the 3′-UTR of PDL1 in the presence of CD8+ T-cells or IFNγ.

FIG. 4 is a graph that shows the percent of endothelial cells remaining (% Survival) following co-culture with purified human CD8+ T-cells. The cells tested were differentiated from wild-type human embryonic stem cells (hESCs), from four different clones of hESCs having B2M/CIITA double knockout (DKO), or from four different clones of hESCs having deletion of the 3′-UTR of PDL1. The results indicate that deletion of the 3′-UTR of PDL1 makes cells resistant to T-cell mediated killing.

### DETAILED DESCRIPTION

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “diabetes” and its grammatical equivalents as used herein can refer to is a disease characterized by high blood sugar levels over a prolonged period. For example, the term “diabetes” and its grammatical equivalents as used herein can refer to all or any type of diabetes, including, but not limited to, type 1, type 2, cystic fibrosis-related, surgical, gestational diabetes, and mitochondrial diabetes. In some cases, diabetes can be a form of hereditary diabetes. In some embodiments, diabetes can be an autoimmune form of diabetes.

The term “endocrine cell(s),” if not particularly specified, can refer to hormone-producing cells present in the pancreas of an organism, such as “islet”, “islet cells”, “islet equivalent”, “islet-like cells”, “pancreatic islets” and their grammatical equivalents. In an embodiment, the endocrine cells can be differentiated from pancreatic progenitor cells or precursors. Islet cells can comprise different types of cells, including, but not limited to, pancreatic α cells, pancreatic β cells, pancreatic δ cells, pancreatic F cells, and/or pancreatic ε cells. Islet cells can also refer to a group of cells, cell clusters, or the like.

The terms “progenitor” and “precursor” cell are used interchangeably herein and refer to cells that have a cellular phenotype that is more primitive (e.g., is at an earlier step along a developmental pathway or progression than is a fully differentiated cell) relative to a cell which it can give rise to by differentiation. Often, progenitor cells can also have significant or very high proliferative potential. Progenitor cells can give rise to multiple distinct differentiated cell types or to a single differentiated cell type, depending on the developmental pathway and on the environment in which the cells develop and differentiate.

A “precursor thereof” as the term related to an insulin-positive endocrine cell can refer to any cell that is capable of differentiating into an insulin-positive endocrine cell, including for example, a pluripotent stem cell, a definitive endoderm cell, a primitive gut tube cell, a pancreatic progenitor cell, or endocrine progenitor cell, that if cultured under suitable conditions will differentiate the precursor cell into the insulin-positive endocrine cell.

The terms “stem cell-derived β cell,” “SC-β cell,” “functional β cell,” “functional pancreatic β cell,” “mature SC-β cell,” and their grammatical equivalents can refer to cells (e.g., non-native pancreatic β cells) that display at least one marker indicative of a pancreatic β cell (e.g., PDX-1 or NKX6.1), expresses insulin, and display a glucose stimulated insulin secretion (GSIS) response similar or superior to that of an endogenous mature β cell. In some embodiments, the terms “SC-β cell” and “non-native β cell” as used herein are interchangeable. In some embodiments, the “SC-β cell” expresses lower levels of MAFA than a pancreatic β cell from a healthy adult human patient. In some embodiments, the “SC-β cell” expresses higher levels of MAFB than a pancreatic β cell from a healthy adult human patient. In some embodiments, the “SC-β cell” expresses higher levels of SIX2, HOPX, IAPP and/or UCN3 than a pancreatic β cell from a healthy adult human patient. In some embodiments, the “SC-β cell” comprises a mature pancreatic cell. It is to be understood that the SC-β cells need not be derived (e.g., directly) from stem cells, as the methods of the disclosure are capable of deriving SC-β cells from any insulin-positive endocrine cell or precursor thereof using any cell as a starting point (e.g., one can use embryonic stem cells, induced-pluripotent stem cells, progenitor cells such as definitive endoderm cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc., as the invention is not intended to be limited in this manner). In some embodiments, the SC-β cells exhibit a response to multiple glucose challenges (e.g., at least one, at least two, or at least three or more sequential glucose challenges). In some embodiments, the response resembles the response of endogenous islets (e.g., human islets) to multiple glucose challenges. In some embodiments, the morphology of the SC-β cell resembles the morphology of an endogenous β cell. In some embodiments, the SC-β cell exhibits an in vitro GSIS response that resembles the GSIS response of an endogenous β cell. In some embodiments, the SC-β cell exhibits an in vivo GSIS response that resembles the GSIS response of an endogenous β cell. In some embodiments, the SC-β cell exhibits both an in vitro and in vivo GSIS response that resembles the GSIS response of an endogenous β cell. In some embodiments, the GSIS response of the SC-β cell can be observed within two weeks of transplantation of the SC-β cell into a host (e.g., a human or animal). In some embodiments, the GSIS response of the SC-β cell can be observed within three weeks of transplantation of the SC-cell into a host (e.g., a human or animal). In some embodiments, the GSIS response of the SC-cell can be observed within four weeks of transplantation of the SC-β cell into a host (e.g., a human or animal). In some embodiments, the GSIS response of the SC-β cell can be observed within one to three months of transplantation of the SC-β cell into a host (e.g., a human or animal). In some embodiments, the SC-β cells package insulin into secretory granules. In some embodiments, the SC-β cells exhibit encapsulated crystalline insulin granules. In some embodiments, the SC-β cells exhibit a stimulation index of greater than 1. In some embodiments, the SC-β cells exhibit a stimulation index of greater than 1.1. In some embodiments, the SC-β cells exhibit a stimulation index of greater than 2. In some embodiments, the SC-β cells exhibit cytokine-induced apoptosis in response to cytokines. In some embodiments, insulin secretion from the SC-β cells is enhanced in response to known antidiabetic drugs (e.g., secretagogues). In some embodiments, the SC-β cells are monohormonal. In some embodiments, the SC-β cells do not abnormally co-express other hormones, such as glucagon, somatostatin or pancreatic polypeptide. In some embodiments, the SC-β cells exhibit a low rate of replication. In some embodiments, the SC-β cells increase intracellular Ca2+ in response to glucose. In some embodiments, the stimulation index of the cell is characterized by the ratio of insulin secreted in response to high glucose concentrations (e.g. 15 mM) compared to low glucose concentrations (e.g., 2.5 mM).

The terms “stem cell-derived α cell,” “SC-α cell,” “functional α cell,” “functional pancreatic α cell,” “mature SC-α cell,” and their grammatical equivalents can refer to cells (e.g., non-native pancreatic α cells) that display at least one marker indicative of a pancreatic α cell (e.g., glucagon, expressing ISL1 but not NKX6.1), expresses glucagon, and secretes functional glucagon. In some embodiments, the “SC-α cell” does not express somatostatin. In some embodiments, the “SC-α cell” does not express insulin. In some embodiments, the terms “SC-α cell” and “non-native α cell” as used herein are interchangeable. In some embodiments, the “SC-α cell” comprises a mature pancreatic cell.

The terms “stem cell-derived δ cell,” “SC-δ cell,” “functional δ cell,” “functional pancreatic δ cell,” “mature SC-δ cell,” and their grammatical equivalents can refer to cells (e.g., non-native pancreatic δ cells) that display at least one marker indicative of a pancreatic δ cell (e.g., somatostatin), expresses and secretes somatostatin. In some embodiments, “SC-δ cell” does not express glucagon. In some embodiments, “SC-δ cell” does not express insulin. In some embodiments, the terms “SC-δ cell” and “non-native δ cell” as used herein are interchangeable. In some embodiments, the “SC-δ cell” comprises a mature pancreatic cell.

The terms “stem cell-derived enterochromaffin (EC) cell,” “SC-EC cell,” and their grammatical equivalents can refer to cells (e.g., non-native pancreatic EC cells) that display at least one marker indicative of a pancreatic EC cell (e.g., VMAT1 (vesicular monoamine transporter 1), expressing NKX6.1 but not ISL1). In some embodiments, the terms “SC-EC cell” and “non-native EC cell” as used herein are interchangeable.

Similar to SC-β cells, it is to be understood that the SC-α, SC-δ cells, and SC-EC cells need not be derived (e.g., directly) from stem cells, as the methods of the disclosure are capable of deriving SC-α cells from other precursor cells generated during in vitro differentiation of SC-β cells as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc., as the invention is not intended to be limited in this manner).

As used herein, the term “insulin producing cell” and its grammatical equivalent refer to a cell differentiated from a pancreatic progenitor, or precursor thereof, which secretes insulin. An insulin-producing cell can include pancreatic β cell as that term is described herein, as well as pancreatic β-like cells (e.g., insulin-positive, endocrine cells) that synthesize (e.g., transcribe the insulin gene, translate the proinsulin mRNA, and modify the proinsulin mRNA into the insulin protein), express (e.g., manifest the phenotypic trait carried by the insulin gene), or secrete (release insulin into the extracellular space) insulin in a constitutive or inducible manner. A population of insulin producing cells e.g., produced by differentiating insulin-positive endocrine cells or a precursor thereof into SC-β cells according to the methods of the present disclosure can be pancreatic β cell or (β-like cells, e.g., cells that have at least one, or at least two characteristics of an endogenous β cell and exhibit a glucose stimulated insulin secretion (GSIS) response that resembles an endogenous adult β cell. The population of insulin-producing cells, e.g., produced by the methods as disclosed herein can comprise mature pancreatic β cell or SC-β cells, and can also contain non-insulin-producing cells (e.g., cells of cell like phenotype with the exception they do not produce or secrete insulin).

The terms “insulin-positive β-like cell,” “insulin-positive endocrine cell,” and their grammatical equivalents can refer to cells (e.g., pancreatic endocrine cells) that display at least one marker indicative of a pancreatic β cell and also expresses insulin but lack a glucose stimulated insulin secretion (GSIS) response characteristic of an endogenous β cell. Exemplary markers of “insulin-positive endocrine cell” include, but are not limited to, NKX6.1 (NK6 homeobox 1), ISL1 (Islet1), and insulin.

The term “β cell marker” refers to, without limitation, proteins, peptides, nucleic acids, polymorphism of proteins and nucleic acids, splice variants, fragments of proteins or nucleic acids, elements, and other analyte which are expressed or present in pancreatic β cells. Exemplary β cell markers include, but are not limited to, pancreatic and duodenal homeobox 1 (PDX1) polypeptide, insulin, c-peptide, amylin, E-cadherin, Hnf3β, PCI/3, B2, Nkx2.2, GLUT2, PC2, ZnT-8, ISL1, Pax6, Pax4, NeuroD, 1 Inf1b, Hnf-6, Hnf-3beta, VMAT2, NKX6.1, and MafA, and those described in Zhang et al., Diabetes. 50(10):2231-6 (2001). In some embodiments, the β cell marker is a nuclear β-cell marker. In some embodiments, the β cell marker is PDX1 or PH3.

The term “pancreatic endocrine marker” can refer to without limitation, proteins, peptides, nucleic acids, polymorphism of proteins and nucleic acids, splice variants, fragments of proteins or nucleic acids, elements, and other analytes which are expressed or present in pancreatic endocrine cells. Exemplary pancreatic endocrine cell markers include, but are not limited to, Ngn-3, NeuroD and Islet-1.

The term “pancreatic progenitor,” “pancreatic endocrine progenitor,” “pancreatic precursor,” “pancreatic endocrine precursor” and their grammatical equivalents are used interchangeably herein and can refer to a stem cell which is capable of becoming a pancreatic hormone expressing cell capable of forming pancreatic endocrine cells, pancreatic exocrine cells or pancreatic duct cells. These cells are committed to differentiating towards at least one type of pancreatic cell, e.g., β cells that produce insulin; α cells that produce glucagon; δ cells (or D cells) that produce somatostatin; and/or F cells that produce pancreatic polypeptide. Such cells can express at least one of the following markers: NGN3, NKX2.2, NeuroD, ISL-1, Pax4, Pax6, or ARX.

The term “PDX1-positive pancreatic progenitor” as used herein can refer to a cell which is a pancreatic endoderm (PE) cell which has the capacity to differentiate into SC-β cells, such as pancreatic β cells. A PDX1-positive pancreatic progenitor expresses the marker PDX1. Other markers include, but are not limited to Cdcp1, or Ptf1a, or HNF6 or NRx2.2. The expression of PDX1 may be assessed by any method known by the skilled person such as immunochemistry using an anti-PDX1 antibody or quantitative RT-PCR. In some cases, a PDX1-positive pancreatic progenitor cell lacks expression of NKX6.1. In some cases, a PDX1-positive pancreatic progenitor cell can also be referred to as PDX1-positive, NKX6.1-negative pancreatic progenitor cell due to its lack of expression of NKX6.1. In some cases, the PDX1-positive pancreatic progenitor cells can also be termed as “pancreatic foregut endoderm cells.”

The terms “PDX1-positive, NKX6.1-positive pancreatic progenitor,” and “NKX6.1-positive pancreatic progenitor” are used interchangeably herein and can refer to a cell which is a pancreatic endoderm (PE) cell which has the capacity to differentiate into insulin-producing cells, such as pancreatic β cells. A PDX1-positive, NKX6.1-positive pancreatic progenitor expresses the markers PDX1 and NKX6-1. Other markers may include, but are not limited to Cdcp1, or Ptf1a, or HNF6 or NRx2.2. The expression of NKX6-1 may be assessed by any method known by the skilled person such as immunochemistry using an anti-NKX6-1 antibody or quantitative RT-PCR. As used herein, the terms “NKX6.1” and “NKX6-1” are equivalent and interchangeable. In some cases, the PDX1-positive, NKX6.1-positive pancreatic progenitor cells can also be termed as “pancreatic foregut precursor cells.”

The terms “NeuroD” and “NeuroD1” are used interchangeably and identify a protein expressed in pancreatic endocrine progenitor cells and the gene encoding it.

The term “differentiated cell” or its grammatical equivalents means any primary cell that is not, in its native form, pluripotent as that term is defined herein. Stated another way, the term “differentiated cell” can refer to a cell of a more specialized cell type derived from a cell of a less specialized cell type (e.g., a stem cell such as an induced pluripotent stem cell) in a cellular differentiation process. Without wishing to be limited to theory, a pluripotent stem cell in the course of normal ontogeny can differentiate first to an endoderm cell that is capable of forming pancreas cells and other endoderm cell types. Further differentiation of an endoderm cell may lead to the pancreatic pathway, where about 98% of the cells become exocrine, ductular, or matrix cells, and about 2% become endocrine cells. Early endocrine cells are islet progenitors, which can then differentiate further into insulin-producing cells (e.g., functional endocrine cells) which secrete insulin, glucagon, somatostatin, or pancreatic polypeptide. Endoderm cells can also be differentiated into other cells of endodermal origin, e.g., lung, liver, intestine, thymus etc.

As used herein, the term “somatic cell” can refer to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods for converting at least one insulin-positive endocrine cell or precursor thereof to an insulin-producing, glucose responsive cell can be performed either in vivo and in vitro, or both in vivo and in vitro (where in vivo is practiced when at least one insulin-positive endocrine cell or precursor thereof are present within a subject, and where in vitro is practiced using an isolated at least one insulin-positive endocrine cell or precursor thereof maintained in culture).

As used herein, the term “adult cell” can refer to a cell found throughout the body after embryonic development.

The term “endoderm cell” as used herein can refer to a cell which is from one of the three primary germ cell layers in the very early embryo (the other two germ cell layers are the mesoderm and ectoderm). The endoderm is the innermost of the three layers. An endoderm cell differentiates to give rise first to the embryonic gut and then to the linings of the respiratory and digestive tracts (e.g., the intestine), the liver and the pancreas.

The term “a cell of endoderm origin” as used herein can refer to any cell which has developed or differentiated from an endoderm cell. For example, a cell of endoderm origin includes cells of the liver, lung, pancreas, thymus, intestine, stomach and thyroid. Without wishing to be bound by theory, liver and pancreas progenitors (also referred to as pancreatic progenitors) are developed from endoderm cells in the embryonic foregut. Shortly after their specification, liver and pancreas progenitors rapidly acquire markedly different cellular functions and regenerative capacities. These changes are elicited by inductive signals and genetic regulatory factors that are highly conserved among vertebrates. Interest in the development and regeneration of the organs has been fueled by the intense need for hepatocytes and pancreatic β cells in the therapeutic treatment of liver failure and type I diabetes. Studies in diverse model organisms and humans have revealed evolutionarily conserved inductive signals and transcription factor networks that elicit the differentiation of liver and pancreatic cells and provide guidance for how to promote hepatocyte and β cell differentiation from diverse stem and progenitor cell types.

The term “definitive endoderm” as used herein can refer to a cell differentiated from an endoderm cell and which can be differentiated into a SC-β cell (e.g., a pancreatic β cell). A definitive endoderm cell expresses the marker Sox17. Other markers characteristic of definitive endoderm cells may include, but are not limited to MIXL2, GATA4, HNF3b, GSC, FGF17, VWF, CALCR, FOXQ1, CXCR4, Cerberus, OTX2, goosecoid, C-Kit, CD99, CMKOR1 and CRIP1. In particular, definitive endoderm cells herein express Sox17 and in some embodiments Sox17 and HNF3B, and do not express significant levels of GATA4, SPARC, APF or DAB. Definitive endoderm cells are not positive for the marker PDX1 (e.g., they are PDX1-negative). Definitive endoderm cells have the capacity to differentiate into cells including those of the liver, lung, pancreas, thymus, intestine, stomach and thyroid. The expression of Sox17 and other markers of definitive endoderm may be assessed by any method known by the skilled person such as immunochemistry, e.g., using an anti-Sox17 antibody, or quantitative RT-PCR.

The term “pancreatic endoderm” can refer to a cell of endoderm origin which is capable of differentiating into multiple pancreatic lineages, including pancreatic β cells, but no longer has the capacity to differentiate into non-pancreatic lineages.

The term “pancreatic islet cells” refers to a population of cells that include different types of pancreatic endocrine cells (β-cells, α-cells, δ-cells, ε-cells) and enterochromaffin (EC) cells, e.g., as described in Xavier et al. (J Clin Med. 2018 March; 7(3): 54), incorporated herein by reference.

The term “primitive gut tube cell” or “gut tube cell” as used herein can refer to a cell differentiated from an endoderm cell and which can be differentiated into a SC-β cell (e.g., a pancreatic β cell). A primitive gut tube cell expresses at least one of the following markers: HNP1-β, HNF3-β or HNF4-α. In some cases, a primitive gut tube cell is FOXA2-positive and SOX2-positive, i.e., expresses both FOXA2 (also known as HNF3-β) and SOX2. In some cases, a primitive gut tube cell is FOXA2-positive and PDX1-negative, i.e., expresses FOXA2 but not PDX1. Primitive gut tube cells have the capacity to differentiate into cells including those of the lung, liver, pancreas, stomach, and intestine. The expression of HNF1-β and other markers of primitive gut tube may be assessed by any method known by the skilled person such as immunochemistry, e.g., using an anti-HNF1-β antibody.

The term “stem cell” as used herein, can refer to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” can refer to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retro-differentiation” by persons of ordinary skill in the art. As used herein, the term “pluripotent stem cell” includes embryonic stem cells, induced pluripotent stem cells, placental stem cells, etc.

The term “pluripotent” as used herein can refer to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and can refer to a pluripotent stem cell artificially derived (e.g., induced or by complete reversal) from a non-pluripotent cell, typically an adult somatic cell, for example, by inducing a forced expression of one or more genes.

The term “phenotype” can refer to one or a number of total biological characteristics that define the cell or organism under a particular set of environmental conditions and factors, regardless of the actual genotype.

The terms “patient,” “subject,” and “individual” may be used interchangeably and refer to either a human or a non-human animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g., dog, cat, horse, and the like, or production mammal, e.g., cow, sheep, pig, and the like. “Patient in need thereof” or “subject in need thereof” is referred to herein as a patient diagnosed with or suspected of having a disease or disorder, for instance, but not restricted to diabetes.

“Administering” used herein can refer to providing one or more compositions described herein to a patient or a subject. By way of example and not limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route. Additionally, administration can also be by surgical deposition of a bolus or pellet of cells, or positioning of a medical device. In an embodiment, a composition of the present disclosure can comprise engineered cells or host cells expressing nucleic acid sequences described herein, or a vector comprising at least one nucleic acid sequence described herein, in an amount that is effective to treat or prevent proliferative disorders. A pharmaceutical composition can comprise the cell population as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Some numerical values disclosed throughout are referred to as, for example, “X is at least or at least about 100; or 200 [or any numerical number].” This numerical value includes the number itself and all of the following:
i) X is at least 100;
ii) X is at least 200;
iii) X is at least about 100; and
iv) X is at least about 200.

All these different combinations are contemplated by the numerical values disclosed throughout. All disclosed numerical values should be interpreted in this manner, whether it refers to an administration of a therapeutic agent or referring to days, months, years, weight, dosage amounts, etc., unless otherwise specifically indicated to the contrary.

The ranges disclosed throughout are sometimes referred to as, for example, “X is administered on or on about day 1 to 2; or 2 to 3 [or any numerical range].” This range includes the numbers themselves (e.g., the endpoints of the range) and all of the following:
i) X being administered on between day 1 and day 2;
ii) X being administered on between day 2 and day 3;
iii) X being administered on between about day 1 and day 2;
iv) X being administered on between about day 2 and day 3;
v) X being administered on between day 1 and about day 2;
vi) X being administered on between day 2 and about day 3;
vii) X being administered on between about day 1 and about day 2; and
viii) X being administered on between about day 2 and about day 3.

All these different combinations are contemplated by the ranges disclosed throughout. All disclosed ranges should be interpreted in this manner, whether it refers to an administration of a therapeutic agent or referring to days, months, years, weight, dosage amounts, etc., unless otherwise specifically indicated to the contrary.

The disclosure contemplates complements (e.g., reverse complements) and/or RNA equivalents of any of the DNA sequences disclosed herein. For example, any of the DNA sequences disclosed herein may alternatively be presented with “U” replacing each “T” in the sequence to generate an RNA equivalent.

### Hypoimmune Cells

The present disclosure, in some aspects, provides cells that are hypoimmune. In some embodiments, the disclosure provides isolated cells (e.g., somatic cells) that are hypoimmune. In some embodiments, the disclosure provides stem cells that can be differentiated into cells (e.g., somatic cells) that are hypoimmune. Such differentiated cells (e.g., somatic cells) can be used, in some embodiments, for administration to a subject to treat a disease (e.g., diabetes or cancer). In some embodiments, the cells (e.g., isolated cells, or cells differentiated from the isolated stem cells) described herein are less immunogenic (e.g., at least 10%, at least 20%, at least 30%, at least 40%, at least 50%, at least 60%, at least 70%, at least 80%, at least 90%, at least 95%, or at least 99% less immunogenic) when administered to a subject relative to a wild type cell of the same type. In some embodiments, the cells (e.g., isolated cells, or cells differentiated from the isolated stem cells) described herein exhibit a reduced level (e.g., at least 10%, at least 20%, at least 30%, at least 40%, at least 50%, at least 60%, at least 70%, at least 80%, at least 90%, at least 95%, or at least 99% reduction) of CD8+ T-cell activation when administered to a subject relative to a wild type cell of the same type. In some embodiments, the cells (e.g., isolated cells, or cells differentiated from the isolated stem cells) described herein exhibit an increased resistance (e.g., increased by at least 10%, at least 20%, at least 30%, at least 40%, at least 50%, at least 60%, at least 70%, at least 80%, at least 90%, at least 100%, at least 2-fold, at least 5-fold, at least 10-fold, at least 50-fold, at least 100-fold or higher) against CD8+ T-cell-mediated killing when administered to a subject relative to a wild type cell of the same type.

In some embodiments, a cell of the present disclosure (e.g., an isolated cell, or a cell differentiated from an isolated stem cell) comprises a disruption in the 3′-untranslated region (3′-UTR) of an allele encoding an immunosuppressor. In some embodiments, a disruption in the 3′-UTR comprises a deletion (e.g., deletion of a fragment of the 3′-UTR or the entire 3′-UTR), an insertion, a translocation, an inversion (e.g., inversion of the entire 3′-UTR or a sequence within the 3′-UTR), or a substitution (e.g., substitution of one or more nucleotides in the 3′-UTR), or combinations thereof. Accordingly, in some embodiments, a disruption of the 3′-UTR comprises an insertion. In some embodiments, a disruption of the 3′-UTR comprises an insertion. In some embodiments, a disruption of the 3′-UTR comprises a translocation. In some embodiments, a disruption of the 3′-UTR comprises an inversion. In some embodiments, a disruption of the 3′-UTR comprises a substitution. In some embodiments, any genetic modification described herein is a homozygous modification. In some embodiments, genetic modification described herein is a heterozygous modification.

In some embodiments, the 3′-UTR comprises a nucleotide sequence that is at least 75%, 80%, 85%, 90%, 91%, 92%, 93%, 94%, 95%, 96%, 97%, 98%, 99%, or 100% identical to any of the 3′-UTR sequences disclosed herein. For example, in some embodiments, the 3′-UTR comprises a nucleotide sequence that is at least 75%, 80%, 85%, 90%, 91%, 92%, 93%, 94%, 95%, 96%, 97%, 98%, 99%, or 100% identical to any of SEQ ID Nos: 3, 74, or 76-89.

In some embodiments, the disclosure contemplates a cell in which a sequence within the 3′-UTR of an immunosuppressor has been disrupted. In some embodiments, the disruption comprises the deletion of 1, 2, 3, 4, 5, 1-25, 1-20, 1-15, 1-10, 1-5, 1-3, 5-25, 5-20, 5-15, 5-10, 10-25, 10-20, 10-15, 15-25, 15-20, 20-25, 25-100, 100-200, 200-300, 300-400, 400-500, 500-1000, or 1000-5000 nucleotides from the 3′-UTR of the immunosuppressor. In some embodiments, the disruption comprises the insertion of 1, 2, 3, 4, 5, 1-25, 1-20, 1-15, 1-10, 1-5, 1-3, 5-25, 5-20, 5-15, 5-10, 10-25, 10-20, 10-15, 15-25, 15-20, or 20-25 nucleotides into the 3′-UTR of the immunosuppressor. In some embodiments, the disruption comprises the substitution of 1, 2, 3, 4, 5, 1-25, 1-20, 1-15, 1-10, 1-5, 1-3, 5-25, 5-20, 5-15, 5-10, 10-25, 10-20, 10-15, 15-25, 15-20, or 20-25 nucleotides in the 3′-UTR of the immunosuppressor. In particular embodiments, the disruption of the 3′-UTR results in reduced or ablated binding of a miRNA to the 3′-UTR. In particular embodiments, the disruption of the 3′-UTR results in at least 10%, 20%, 30%, 40%, 50%, 60%, 70%, 80%, 90%, 95%, or 100% reduced binding of a miRNA to the 3′-UTR. In particular embodiments, the disruption of the 3′-UTR results in at least 10%, 30%, 50%, 75%, 100%, 150%, 200%, or 250% increased expression of the immunosuppressor as compared to a cell in which the 3′-UTR has not been disrupted. In some embodiments, the sequence that has been disrupted comprises the sequence ATTTA, ATTTTA, or ATTTTTA. In some embodiments, the sequence that has been disrupted is within 1-25, 1-20, 1-15, 1-10, 1-5, 1-3, 5-25, 5-20, 5-15, 5-10, 10-25, 10-20, 10-15, 15-25, 15-20, or 20-25 nucleotides from the sequence ATTTA, ATTTTA, or ATTTTTA in the 3′-UTR of the immunosuppressor.

In some embodiments, the disclosure contemplates a cell in which the 3′-UTR of an immunosuppressor gene in the cell has been disrupted such that an endogenous RNA-binding protein and/or microRNA in the cell is unable to bind to, or has significantly reduced binding to, the 3′-UTR of an RNA encoded by the immunosuppressor gene. In some embodiments, the RNA-binding protein and/or microRNA is unable to bind to the 3′-UTR because one or more nucleotides in the binding site in the 3′-UTR for the microRNA have been deleted. In some embodiments, the RNA-binding protein and/or microRNA is unable to bind to the 3′-UTR because one or more nucleotides in the binding site in the 3′-UTR for the microRNA have been inserted (e.g., if several nucleotides are inserted or if an entire transgene is inserted into the binding site for the microRNA in the immunosuppressor gene). In some embodiments, the RNA-binding protein and/or microRNA is unable to bind to the 3′-UTR because one or more nucleotides in the binding site in the 3′-UTR for the microRNA have been substituted such that the microRNA no longer is capable of binding to the 3′-UTR (e.g., to disrupt complementarity/base pairing). In some embodiments, the immunosuppressor gene is PDL1 and the microRNA is any one or more of miR-34a, miR-140, miR-200a, miR-200b/c, miR-142, miR-340, miR-383, miR-424(322), miR-338-5p, miR-324-5p, miR-152, miR-200b, miR-138-5p, miR-195, miR-16, miR-15, miR-193a-3p, miR-497-5p, miR-33a, miR17-5p, miR-155 and/or miR-513. See, e.g., Xie et al., 2017 PLOS One, DOI:10.1371/journal.pone.0168822; Zhao et al., 2016, Oncotarget, 7(29):45370-84; He et al., 2018 Biomedicine and Pharmacology, 98:95-101; Tao et al., 2018, Cell Physiol Biochem., 48:801-814; Kao et al., 2017, J. Thoracic Oncology, 12(9):1421-1433; Audrito et al., 2017, Oncotarget, 8(9):15894-15911; Holla et al., 2016, Scientific Reports, 6(24193); Danbaran et al., 2020, International Immunopharmacology, 84:106594; Gong et al., 2009, J Immunol., 182(3):1325-1333; Chen et al., 2014, Nat. Commun., 5:5241; Wang et al., 2015, Cellular Signaling, 27(3):443-452; Xu et al., 2016, Nat. Comm., 7:11406; and Dong et al., 2018, Oncogene, 37:5257-5268. In some embodiments, the immunosuppressor gene is HLA-G and the microRNA is any one or more of miR-133A, miR-148A, miR-148B, miR-152, miR-548q and/or miR-628-5p. See, e.g., Schwich et al., 2019, Scientific Reports, 9:5407. In some embodiments, the disclosure contemplates a cell comprising the disruption (e.g., deletion of all of or a portion) of the gene encoding a microRNA that binds to the 3′-UTR of an immunosuppression gene and reduces its expression. In some embodiments, the disclosure contemplates a cell comprising the disruption (e.g., deletion) of the gene encoding any one or more of miR-34a, miR-140, miR-200a, miR-200b/c, miR-142, miR-340, miR-383, miR-424(322), miR-338-5p, miR-324-5p, miR-152, miR-200b, miR-138-5p, miR-195, miR-16, miR-15, miR-193a-3p, miR-497-5p, miR-33a, miR17-5p, miR-155, miR-513, miR-133A, miR-148A, miR-148B, miR-152, miR-548q and/or miR-628-5p.

In some embodiments, a disruption in the 3′-UTR of an allele encoding an immunosuppressor leads to increased expression (e.g., increased by at least 10%, at least 20%, at least 30%, at least 40%, at least 50%, at least 60%, at least 70%, at least 80%, at least 90%, at least 100%, at least 2-fold, at least 5-fold, at least 10-fold, at least 50-fold, at least 100-fold or higher) of the immunosuppressor in the isolated cell, stem cell, and/or cells differentiated from the isolated stem cell. In some embodiments, the increased expression of the immunosuppressor is induced or increased by a cytokine, such as interferon gamma. In some embodiments, the increased expression of the immunosuppressor is induced or increased by interferon gamma. In some embodiments, it may be advantageous to have the immunosuppressor responsive to a cytokine such as interferon-gamma as contemplated herein, rather than have constitutive expression of an inserted transgene of the immunosuppressor in a cell. In some embodiments, any of the cells disclosed herein is exposed to a cytokine (e.g., interferon gamma) upon implantation into a subject. In particular embodiments, it is not necessary to expose any of the cells disclosed herein to a cytokine (e.g., interferon gamma) prior to implantation into a subject.

An “immunosuppressor,” as used herein, refers to a gene or molecule that attenuates an immune response. In some embodiments, an immunosuppressor inhibits the adaptive arm of the immune system. In some embodiments, an immunosuppressor inhibits the innate arm of the immune system. In some embodiments, an immunosuppressor attenuates a normal immune response during development. In some embodiments, an immunosuppressor attenuates a normal immune response during a disease state. In some embodiments, an immunosuppressor is used to attenuate an immune response during tissue or cell transplant. Non-limiting examples of immunosuppressors that can be used in accordance with the present disclosure include: PDL1, PDL2, CD47, HLA-G, CTLA-4, HLA-C, HLA-E, C1-inhibitor, IL-10, IL-35, HLA-C, HLA-E, HLA-G, C1-inhibitor, IL-35, DUX4, IDO1, IL10, CCL21, CCL22, CD16, CD52, H2-M3, CD200, FASLG, MFGE8, and/or SERPINB9. In some embodiments, the hypoimmune cells described herein are produced by disrupting the 3′ UTR of one or more immunosuppressors.

In some embodiments, a cell (e.g., an isolated stem cell) described herein comprises a disruption in the 3′-UTR of an allele encoding Programmed death-ligand 1 (PDL1). In some embodiments, the disruption is a homozygous modification. In some embodiments, the disruption is a heterozygous modification. “Programmed death-ligand 1 (PDL1)” is an immune inhibitory receptor ligand that is expressed by hematopoietic and non-hematopoietic cells, such as T cells and B cells and various types of tumor cells. The PDL1 protein is a type I transmembrane protein that has immunoglobulin V-like and C-like domains. Interaction of this ligand with its receptor inhibits T-cell activation and cytokine production. During infection of inflammation of normal tissue, this interaction is important for preventing autoimmunity by maintaining homeostasis of the immune response.

An example of a Homo sapiens PDL1 gene sequence is provided in NCBI Gene ID: 29126 (SEQ ID NO: 28; corresponding to positions 5450542-5470554 of Homo Sapiens chromosome 9 sequence as provided in NCBI Accession No.: NC_000009.12). In addition, examples of human PDL1 transcript variants that encode different isoforms of PDL1 proteins are provided in NCBI Accession Nos.: NM_014143.4 (SEQ ID NO: 1), NM_001267706.2 (SEQ ID NO: 2), or NM_001314029.2 (SEQ ID NO: 3).

Human PDL1 gene—NCBI Gene ID: 29126 (SEQ ID NO: 28); 3′-UTR underlined (SEQ ID No: 87)

AGTTCTGCGCAGCTTCCCGAGGCTCCGCACCAGCCGCGCTTCTGTCCGCCTGCAGGTAGGGAGCG
TTGTTCCTCCGCGGGTGCCCACGGCCCAGTATCTCTGGCTAGCTCGCTGGGCACTTTAGGACGGA
GGGTCTCTACACCCTTTCTTTGGGATGGAGAGAGGAGAAGGGAAAGGGAACGCGATGGTCTAGGG
GGCAGTAGAGCCAATTACCTGTTGGGGTTAATAAGAACAGGCAATGCATCTGGCCTTCCTCCAGG
CGCGATTCAGTTTTGCTCTAAAAATAATTTATACCTCTAAAAATAAATAAGATAGGTAGTATAGG
ATAGGTAGTCATTCTTATGCGACTGTGTGTTCAGAATATAGCTCTGATGCTAGGCTGGAGGTCTG
GACACGGGTCCAAGTCCACCGCCAGCTGCTTGCTAGTAACATGACTTGTGTAAGTTATCCCAGCT
GCAGCATCTAAGTAAGTCTCTTCCTGCGCTAAGCAGGTCCAGGATCCCTGAACGGAATTTATTTG
CTCTGTCCATTCTGAGAACCCAAAGGAGTCCTAAAAGAGGAATGGAGGAGCCTAAGAATAAAAAT
AGTATAATAAAACATTTCTTAGACACATTGACCTTGGCCTATGTCAAAGTTCAGTCTGGGTTTGT
CTTATAACACAAGGAGTAAAAGTACCATTGTTCTACCTCTTTTTTTAATACTTGAAAAAAATTTA
CTGTGGATGCTTTTCTATGAATTAAATAACCTTCTAAAAAATGTTTTCATTGCTGCATTCGATTA
GATTGGGTAACTAAATGAAATTAATTCCTCACTGTTGGGTATAAAGGTTATTTACAGTGGTTCTG
TCTTAGCCATTCACTGAACTCATTGCATATATATCTCTGGAATATTGCTGATTGTTTCCTTCAAG
TAAACTTAGAAGTGTAACTACTTAGTCAAAGAGCCTGAATATTTTAAAGGCCTTTTGAAGAAAAC
TGAAAATGCTTTCCAGAAAGGATGTATCAGTTGACAATGACAGTCGTCAACAGTATTTAAGGAGA
ACTATGATACTCTGAAGAAAAACTTAGCCTTTCTCAGTAAAAGTAGGTAGGCAGAGGCCACATGA
CAGCAGTTAGAGTGTGGTCTTCAAGGAAGTCACAGAAATACTGTGGGGAATTGAAACCCCATGTG
GAAAATGTACAAGAGTGTCTCAGTGTGACTGAGAAGGAGGTTGGGCATGGGGTTTCATGGAGTTT
AATAAAGTTTGGTCACTTAGTAGAGGTTTAATAAATCAACTGTCTTAATCTTTGATCCTACTTAA
GAATTTTTTTTTTGTTTTTGTAGAGATGGGGCTCTTGTTATGTTGCCCAGGCTGTTCTCGAACTC
CTAGCCTCAGGCGATCCTCCCTCCTCAGGCTCCAGAAGTCCTGGGATTACTGGCGGGAGCCACCA
TGCAGGCCTCTTGCTCCTACTTTTGAGAAAGGAAGTTTAACCGGTTTTTTTTGTCTTTTTTTTTT
TTTTTTTGAGACAGAGTCTCACTCTGTTGCCCATGCTGGAGTGCAGTGGTGCAATCTCAGCTCAC
TGCCTCCCGGGTTCAAGTGATTCTCCTGCCTCAGCCTCCCGAGTAGCTGGGACTACAGGCACCTG
CCACCACGCCCAGCTAATTTTTGTATTTTTAGTAGAAATGGGGTTTCACCATATTGGCCAGGCTG
ATCTCGAACTCCTGACCTCAGGTGATCCGCCTGCCTCGGCCTCCCAAAGTGCTGGGATTACAGGC
ATGAGCCACTGCTCCTGGCTGCTTAACTTTTTCTCTATCTCATCCTCCTACCCATCCTACCCTTG
GAAGATAGAGAAGTAGTATTAGTTCCATAGTGTTATACTGGGCTTCCCCCAGGGACAAACCCACT
TCCCCAACCTGAATGAGCCATCACTTCTTCCCCAGTTTACATTTCATTGCTCTTTAAATGTCTCC
ATTCGGATATGGGAATTCACATATGGTCATAATTCTTACCTGAAGAAGATGTCAGTCTTCTTCTC
TTAGACCAACTGCCCTGATATGAGGTTTAGAGGTTAAAGAACATGTGTGTATTTACATGATCTTT
GTATTCTGCCTTTTCGTCCCTCACTAATGACAGCTGCACCCCAAGGAAATGGAGCTGTGGAAGAG
AGGGTTTGATAAGAAATTAAGTAAATATTGGATCTAATCCATCACCCTCCAGGAAGCCTTTATTA
CTCCTAAAAATTTCAACCAAATTCATTAAAGGACAAGAACTCCACCAGAGTAGGCCATAAACATT
GGCAAAATTAGTTGTAATCCATGACTAGATTTAATGTCCCTTTGTTTTATTCCCATATGGTTATA
ATGCTTTGCTTGGCATTAGGGGTATTTTAAGTTTTCTTCTGCCTAGTAAGTGAATTTGTGTTTAT
AATACAATAATCATAAAATATCACATTAATATTTTATAACTGTACAGTTATAAAATATTTTATAA
GTAATATTTATATTTTATAAGTAATATTTTATAACTGTACAGTTAACTCTGGCCCAAGGAAAAGA
TAGTCTGATAGATGCTGCAGCCCCATTTTAGCAAATGTGACCTCACAGGCCTGAATGCCATCGCT
ATTCCACATCTACAGGATAGACGGAAAGGAAAGAAATAAAAAAATAGGTACCTAACACTGGCAAG
AGGATGATGACTCATGTTATTTCACTTAACCTTTTTATCTTTTAACATGAAGGACTCATACAGGT
TGATAAGAAACCAGTGACATAAACAGACCAAAAAATGATCAGATCTTTCAAATTAGCAAAAAAAT
AATATTTTTTAAACAATGGGTGAAAATACAGTGTAACAGTACCAATTATCAACATGTGTTGAGAA
CCAGAAAAATGTTCTTTTTCTTTGATCAGCAACACTATTTGGGAAAATCTATCCTCAGGGCCTAG
CCTGGGGCCCTGGCACACAGTAGGCACTCAACGAATATTTGCTGAACACACAAATACTTATGATA
TTTTAAAAAATTGGCAACAATCTGATACCTAACAATAGAGGGATTAAATATTATGGAACTGTTAA
ATAAGATGCTTATGAATACCATGCAGTAAGATGGGCAATATTTATGCCATAAGCTTTAATGAAAC
AAATGGGTATTAAATGTATGATAAGGTTATAAATTACTTTTTAAAAGATTACAGGGAAAAAAATT
GAAAGATATACACTGAAATGTTTTTTGCTCACAGTGGTGACAAGGTTTCTCAGCACTGGCACTGT
TGACGTTTTAGGCTGTATGTCTTTGCTGTGGGAGGCTGGCCTGTGCACTGCAGGGTGTTTGGCAG
CACTCTTGGCCTCTGCCCCTAGATAGCAATAGCAGTCCTCCCTCAACCAGCCCAATTTTGACAAC
CAAAAATGTTTCCAGGCATCACCAGATGCTCCCTGGGTGAGAGTGATGAAATAGTAGGGGATTTT
CCCCTTCTTTTCTTATTTTCTGTAATTCCATTATATTACTTTAATAATAAAGAAAAAAACATAAA
AAATAAACGAATGTTATTATTCTACGTCAGTTTGGATGTTTGGACTCCATTTTGGGGTTCTTTCC
ATTATATCACTTGGTCTGCTAAACATTCTACGGTTTGGTAAGGTGAAGTGATTCATGAAATTTTG
GTTTTATTTTTTTCCTGATACTAAAAATAAAACATTCTTTCACTTGGAAATTTGGACACAGAACA
CCAAAAAAAATCCATAATCTCATCTCTCTTTTTCTGTCTTTTCCTTCCTTTTTTCCCTTTAAAAA
CAATAAAGAGTGAAACCTACCTGTTCTCCCTCTAATTTAATTCCTAAATATAATCACTGTCAATA
TCTTGGACATTTCCTGTGTCTAAACACACACACACACTTTTTTTTTTCAGCAAAAGTGGATTTCT
GCTACATGTAGTGTTCTGCAACTTACTTTCTATGTGTTTACAAAATCAGTACATGTACATATGCT
GAATTCAGTCCTTAATGGTATTATATTTTGTGAATATACCAAAATTTGTTTAACCACTTAGACAA
TCTAGGATATTCTCAGTTTGCTGTTATGAGCAATGCTCTTCCTTTACATATACAGACATATATAT
ATATATGTGTGTGTGTGTGTTTTTGTTTTAGTAGGATAGATTTCTAGGAGAGGGTGAAAGGTCTT
ATGACATCCGCATTTACGATTGTAATAGGAAGTATCAAAGTGCCCCCTAAAGAAAAAAATCCTCC
CATTAGTGGGTAAGAAAGCCTATTTGTTCATATCTTCACAAACACTAAATATTAGAAATATTTAC
AATTGTGGTCAAGCTCATAAGTGAAAATGGTATTTCATATCTTATATTTTTTATTGTGAGATTGA
ACATCTTTCATATGTTTACATGTCACCTGTATTTCTTATTCTCTGAACTATATGTTATGACCTTT
CACTTTTTTTCCTCATGGGTTATGTGTAGTTTGTATAGTTGTCTTATTGATTGTTAGGAGCTATT
TATATATTAGGAACATTAATCTCCTGTCTTATATATACGTGGCATCGATTAGTTGATCATTTGTG
AGTTCATGTCTGTATACAAAGATTGGAGAGGCACTAAGAGGGAAAACTTACCTCTTTCTTATCAA
AGTTTGTAAATATATGTATAACAGAAGAGGGAGAAAATATTAATAAATGCACAGATTGGCTGAAA
TAGAGTATAAATCTTTTACTCCCCTACTTCAACATAAACTGCAAAAGGAGAGTGACTTTTCTTTC
ACTCTGACTTCCGTATTCCTCATGCTTAAAATAGTGCCTAGCACAGAAGAGGTGCTCAATCAGTG
TTTGCTAAACGAAATAATTAGTCACATTTCAAGCAGGATGACTAAATGAAGAATAGAATCTAGGC
AGATACTCTGGAAGAGTGGCTGTGAGTCATTCATATCTTAGTATGAATTAGTCAAATCCAACTCT
CTCCCCTTCCCACTCCCCACTGTTAGTAGAAGAATCTGTTTATTGAGAGAATAGATTTATAATTT
AGAATAAGTGAGAGGGGCAGAAGAGGAGATTTTGAAGGATGGCACCTGAAGGAGGACTAGCATGG
CTGAGACAGTGAAGTGGAAGCCTTGAATAGCTAAAGGGTAAGATGAAAGTATTTAGCTGTAGGGG
GAAAAAGCATTGACAGGTTGGAAAAGTAAAAGTCAGATTCTCCTTGCTCTGAAATTTTGTACAGG
GCAGGTTCTACTAGGTATGTTACAATGCAGAAAAAACATGAAATAATTGAGAGGAATTTGGTGCA
ATATTATCTTCTTGGCTTCTTTTGAGTGGGCAGATTTTTTTCACGGCCTGTAACTATAATAAATT
TGAAACTTCTCATCTTTTAGTAACTTTTTTCACTTAAGTTTATGTGGCTGTGGGCAATGGAATGA
AGATATTGAACTTCCAATTCCCTGTTGGGTTTCCACAATTACAAGTCAATCATGACTGGTTATTA
GAAGACTATTTCAGTTAGAACCACCAAGTCCCATATTGTCATATTGTATGTTTAATTATTAAGTG
AAGCAGTCTTCTTTTCGTGTTTTCCATAATTAGGGCATTCCAGAAAGATGAGGATATTTGCTGTC
TTTATATTCATGACCTACTGGCATTTGCTGAACGGTAAGACACCAAATCCTTCCATTAGGTTCTA
TATTTTAAATATTTTAACCATGAGTTTAAAACTAAAATGATCATTTAAAATGCATGCAATTTTCT
TATAGAGAGAACATTCTATTCTTTCTTCTACTTTACACAATGGCAAAGTCTTCTTTCTACTTTAC
GCAATGATAAAGTTACCTGTGTCATTTTGTAAAAATATAGAGAATATAGACAAATTGAAAGACAC
AAAATAATCTATTACCCATTTCCCAGGGTTAACTACTGAAAATATCTGGGGAAATGGCCTGTATG
TATACATTTATTTGTTTGCTTTCAACAAGGCCAAGATCCTTTGATCTTTCAGTCTTGGTTGCTCT
GTGACATGCCTTTCCTGATGAGGATACTTTAAGGAAGAATTGTAAGATACATGGAAAATGTCAGG
CTAACACAGTACTGGCATCACCCTGTGCTCTTTCCTGAACTCCATACCAATGTACTTCTTGCCAG
AAAACTGATCAAAAGTTTAGGGAAGTAAAAAGAGATGACTGTTAGAATCTACCATTCCCTCTATG
TAGGAAGCAAATAGGTGTCCTGTCAAAGGACATTCTGGGGATGTCTACATGAAACCAAGTCTCCC
TGGTTGTAAGGACTCCATCTCCATATAATATTTATACAGTAATATATGTTTATAAATTGTGGGGG
CAACTTGTTTAGCTAATTTTATTATTCTGCTATTGGGACACTGTGTCTCAGCATGAGATATAGTG
TCCCAAAACATATTTCAAGCCCATTGGATAAAATATGTGTTTAGCAAGTTCTTAAATATAATGAT
AACATAACCGACCAGATAAAGTGATTTATAAACGCTGTGCCAATTTTGTAAATGTTTCGAGGAAT
TTTCCCTTTTCTGAAGATTGTCCTTCTTTCTTTTTAGCATTTACTGTCACGGTTCCCAAGGACCT
ATATGTGGTAGAGTATGGTAGCAATATGACAATTGAATGCAAATTCCCAGTAGAAAAACAATTAG
ACCTGGCTGCACTAATTGTCTATTGGGAAATGGAGGATAAGAACATTATTCAATTTGTGCATGGA
GAGGAAGACCTGAAGGTTCAGCATAGTAGCTACAGACAGAGGGCCCGGCTGTTGAAGGACCAGCT
CTCCCTGGGAAATGCTGCACTTCAGATCACAGATGTGAAATTGCAGGATGCAGGGGTGTACCGCT
GCATGATCAGCTATGGTGGTGCCGACTACAAGCGAATTACTGTGAAAGTCAATGGTAAGAATTAT
TATAGATGAGAGGCCTGATCTTTATTGAAAACATATTCCAAGTGTTGAAGACTTTTCATTCTTGT
AAGTCCATACTTATTTTCAAACAGAACAGCATAGTCTGTTCATTCATTCATTCAATTCATGAATT
CATTCACATAATTATCCAATTTCTTGAGCACCTATTTGATAGTCACTGGAAATCCAGAGACAAAC
AACACAGAGCCATGTTCTACAGTATGTACAGTTTTCCAAAAAGAATTTCTAGTCTTTACTTTTTT
ATTACAAATGGAATACGTATACTTGCAAATAATTCAGATACTGTGGAAGAGATCAAATGAATTGC
AAAAGTGTCCCTCCTCCCTTCACCACTATCTCCCATGGCATGCAGAGAGAGTAACCATTATTTGT
GTGTCCCTCCAGAAATTTTTTTATTCAACTACTATTTTTTTATTTTATTAGGTCCGTCAGTTTTC
CTTTTTTGAGCCTCTCTATATCAAATGCAAATAAATATATTCAGAACAAACCCCACTGTAAGGTT
CACATTAAAAAAGACTTGAAGTCACCCTATGAAGACAAAAAATAATCACATTAAGTGTGAAAGAA
CCTATTCTTCCAGTACAGGATAAGCCATACTTACTGGGCATATATTCATCTTGAAAATCTATACT
GATGTTGTCTTGGGGAATTGAAAAGGAACTAGGAGTGTTAGTTCCTCGGTATTGACCCACAGTTA
TGTTATCAGGTCACTTGAGTTCAAAGTTTTGTGTTGGCACTAGCTAAGTAAAGGAAAACACCTCT
GCTTTCATTGTTGAGTTTCACAGAATTGAGAGCTGAAAGGATCCCAGGCAGGAGCAGCTAATCCA
AACTCCCACAAAGAACAAAAATCCCCCAGAGGATCTTCTGTTCTTATATTTCCTGCAATGGCGTC
CCTGTCATATCCCACAATGGCCTCCCTGCCATTTGGATATCCCTTCCATATCCTGTTGAAATTAC
TCCCTAATAGTAAGCTGAAATCTGCCCCTCTAGTTGTAGTCTTGGGATTATTTCATTTACATGAT
GACCTTTTAATATTTGACTAGAATTAAATCATCTCCCCTTGGTCTTTCCATTCCTGGGCTAACTA
CCATCAATCTGAGGGCTAACAATACAAGTAGAAAAAGTATACATTTGTCACTGATCACTGATCAA
TTATTAATCAATGATCACTGATAACTATAAACTCAAAAACAAAATCATGTGGGGATTAAGAGAAA
TGTATCAGTTTTATGTTGTATTTCTGGTCCCTGATACTGGCTCAGGTAATGCCACTATTGTCAAG
AAGATACCACTTGTAAAGTAGATTTAATTTTCATTATATTTTACCATATGCTTCTCCATTCATGA
CATCTCTTGAGATGTTGTGGTTTATACTTTCAGTTTTTCTCCAGTCCATCCGCAAATATCAGGCA
TCTACTGTGTTCCAAGATATTAAAGAAATCATCATGACTTAGCCTCATCAACAGCATTGCTAGAT
CTGGGATGGAAAGGAAGAGTATAATCCTGGCAGTCAGGAAGAAGGCAGCATAAAGTATAAGTTTC
TGCTTCCAAAAAAGGTCTCTCATCAGCCTGTAGGGAGTGTGTAGGGAAGGGACAGCTGTCCTTGT
AGTAGGGAAGGGTTTTATTCAGGTCGTCTGGGCTCCATAATATCCCTTGTGTATCTGCAGTCTCC
TTTGCCATGGATCAACACAATAGGAAATCTTCCGGCACTGATGGTTTTTCCAAGGGGGAGTTCTT
CCTGGAGCAAAGCAAATGACCAACCAGGTTTGAGGACCTGATTTGTTTGACAATTCCATTTTGTA
TTGTAAATTACTTAATTGGCATTCTACTCCCAATCCATCTTGTCATTTGCATACAGTGGTTTTGG
GATTGAGTTCAGCTATACCAAAAGTCTGAACCTTCTGCACTTAGAACAAGGCAACCACCAAGCTT
CACTTGCACTGAGGCCGTGTCTCCAATGGAAATGAGGCAGCTGGCTTGCAGGAGCTTCCCAACTC
AGGGAAGTAGAACTCCTGAGTCACCTCCATATGCAAATGATTTCACAGTAATGCTGTTGAACTTC
ACTTCCCATCACAGCAAATGTGTGGTAACATAGCTTCCCCACAGGAGTTTACTCACCATGGTATT
TTAAAGGTGAAACATTTCAAAACTGAAATTTGAAAGAATTTAGTTTTGGATTCACTCAATTATCA
CTATCACTTCGGGTGTTATTGCACCTTTCTTGTTTGTGAGTTTAAATGCCAGACTCTCAGGCCAC
TAACTTTCAATTAAAAGTGTTTTTCTTTAATCGCTGAACCTAACAGCAGGGAAAACGAAATGTTC
ATTCAGACTTTCAGAACCTTCAATGAGATTAGGCAGCTGAAAGATCAAAGTGTTGCATAGTTGTC
CCGATAAAGCTATTTGGATCATATGGACCAAATCGACTGCTGTCATTCCCCACCAACCCCATCTC
TCCCCAAAATTCCCAGCCCTGTTTAAGTGTTCTCTGTAGCATTTATCTCTATCTAGTATATTGTG
TAGCATATCATATCATACTTTTCTGTTTTGTTTATTGTCTCTCTCCTCCTAGAATATAAACTCCA
CAAGCACAAAGATTTGGGCCTGTTTTATAATATTGTTGCATCCCCAGGGCCTGATATACAGCAGA
GTGGTGGTACGAAAAGAGCACACAAAAAAATATTTGTTGAGTCAATGAATGAATGATTTCCTCAA
ATAGGATTAGCCTAAAATTTTGGAAACATGAACAGATTTGGATATGTGAAAATTTATTTCCAGAC
TGTTCATCAGGAACTGTTAGCAGCTTCTAAAGGGTACACTGGAGCAGCAGTAGTAAAAGGAGGAA
GAGGAGCAGCTCTGCTACTGCTACTATCGAGTACTACTACAATTAGCACTTGCTTATTCTGTGTG
TTAGGCCCTGTACTGAACACTCTGTCTAAATTAGTTCATTTCCTCCTGGAAATGACTCTAGGGGG
TAAGTGCTTCATCATGTAAGATGAGTATTTTTCACATTTTGTTGTGTCTGAAATCTGAGTGTGTC
TTTCAATGATGGAATCTTTGATTCCATGATAAGTGGTATTATTCCCATTTTAAGGATGAGGAAAC
TGAGGTCCAAAGAAATTAAGTAATTTGCCCAAATTCACCCAGCCTAGAAAATGATAAAGCTAGTT
CTAAACCCAAGCAGATTAGCTCTGAAGTCTGGGCCCTTAATAACCACTTTTTATTGCCTATATTT
GTACCTCTGGTGTACGTATCAAGTTATATGTTGACTTCAAAACTATCATGACCTTTTCTTGGTTT
TGATTGTCCAACATTAGTATAGTGTTCTGGGTCTGCAAAAATTTTGATTACTCATCTCATCTGTA
AAACATTTTGAACTCGTGTGTTTGTGCATGCACATTTGTGTGTAATTATAAAAATTTTACTTTCT
GTTAATATATAAGTTGTATCATAAGAAACTGCCGTTTTTGAAGAGCAAAAAAAGGTTGAATGTTA
CCAGTTACATCTGGTTCAACCTAATAGACATTTGTACAAAAACAGACATTTTAAGAGGTTGAAAT
AAAAATTTAATAAACAATATTTTCAGTTTTTACTAATTGTGATGCTTCACTATCATTAGCTAATA
TGTCAAGGCATAATATACCTTAGGGTGAACTTTATCATTAACAAAGGTGGATGGTGTCAATAATC
TTGAGGTTTGTGTTTTTTTATATAACACTGCGAGGTCTAATTAAGTACTTACTGTTTACCACCTC
ATACAGTGGCCGATAAAAAGTGTCACTTCTGCTGTTTCCTCTGGGTTGTGCTTGAATTATTAGTA
TTATCTTCAGTCCTCAGTTTCTTTGTGGGAAACTTTTTAATTAGTTGTTTAATTTTGTAAGATGG
TTAGTTTAGTCAAAATTAGATAAGAGAATTTGAAAATCCGTAGCTACCCCAAAGCAACCTACACA
TAAGAACTATTATTTTTGTGTTTTGAAATCATAATTTTATTGATTTCCAGTGTTTCCACTGGTAG
TGGTTTCATTGATATAGGAGTATCAAAACATCACTCATTATTTATTTCAGTTTCATTTGATCCTA
GCCGTTTTGTATTAACTCTCTGTGAAGAAATTACCTCACAAATCTATTGCTGTCCTTGGTAAAGG
AATGGAGAATTAAGGCTCTAGATCATTAGTGGTTACACTATAGTATTAGAAGTAAAAAAAAGATT
ATACCAACAAAATAAGAACATGTTAATGTACTTGTAATGAATAAACATGAATAAAGCTCTTATGC
TATATAGGTGCACTAAACAATCTACTAGAATTGTCAGCAAACTACGTATCTTAATCCTGAAAGGG
TCCCAAACCAATGATCTAAAATTGAATCAAACTTTCTTCCTTGAGCATAATTACTTAAATGATTT
ATTAAAATAGCCAGCATTTAAAAGCTTAAAATGTAAATATCATAATGTGGTATCCTAGATAGCAT
CCCAGAACAGAAAAAGGATATTAGGGAAAAACTGGAGGAATGGAATAAATTATGCAGTTTAGTTA
TTAATAATGTACTAACGTCCTTAGTTATGACGATTGTACCATGGTAATGTAAGATACTAACAATA
GAGGAAACCGGGTAAGGAGTATACAGTAACTCTATACTATCTTTGCAACTTTTTTGTAAATTTAA
AACTTCTAAAATAAAGAACAAATTTAAACATTAAAAAGTATCACCAGGAACATATATCACTGTTT
ACAGATGAAATACTATGTATTTTCATATCTAATTTCTGATCATTGACTTCAAATCAGAAAAGTGA
ATGACACCTCAAAATCAGGTTTTCTGTTTACTGAAGTCTAAGAAAAGAAAGCATACCAGCTGGAG
AGATTCATGTTTATAAAGACAGATTTATAACAACAAAAATAAAATATCCAAGAATAAATTTAAGA
AGAAGCACTTTACTGAGAAACATATGAAAACCTGAACAAATGGAGAGGGATATTTTGTATTTGAA
TAGAAAGACTTCTGGTTTAAAGATAATTCTCTTTAAATTATTTTTTGTAGAAATTTAAGGGGTAC
AAGAGCAGTGTTGTCACATGGATATATTACATAGTGGTGAAGTCTGGGGTTTTAGTGTAAATTAA
TCTTTACATTTTGTTTGAGCCCAATAAATGTACCAACATGATTTTTATAGAAAGATAGTCATTCC
TATTAATCCAAACTTGTCCCAACTTTGAATTGAATTGAGGCAGAGCTAGCAGGTGTTCCCCACGG
CTGAGGCATCTGAACATTAAGCATATCCCTCTGAGAACCAGCCTGCATTGATACTCTTTCTAATG
TGGACAGCATCAAGCTATGTACGTAGTTCTGTGCTCAGCAAAAGCCCTGACTTCTTTTTGTTTAT
GTCCTAGCCCCATACAACAAAATCAACCAAAGAATTTTGGTTGTGGATCCAGTCACCTCTGAACA
TGAACTGACATGTCAGGCTGAGGGCTACCCCAAGGCCGAAGTCATCTGGACAAGCAGTGACCATC
AAGTCCTGAGTGGTAAGACCACCACCACCAATTCCAAGAGAGAGGAGAAGCTTTTCAATGTGACC
AGCACACTGAGAATCAACACAACAACTAATGAGATTTTCTACTGCACTTTTAGGAGATTAGATCC
TGAGGAAAACCATACAGCTGAATTGGTCATCCCAGGTAATATTCTGAATGTGTCCATTAAAATAT
GTCTAACACTGTCCCCTAGCACCTAGCATGATGTCTGCCTATCATAGTCATTCAGTGATTGTTGA
ATAAATGAATGAATGAATAACACTATGTTTACAAAATATATCCTAATTCCTCACCTCCATTCATC
CAAACCATATTGTTACTTAATAAACATTCAGCAGATATTTATGGAATATACCTTTTGTTCCATGC
ATTGTAGTACTCATTGGATACACATAGAATAATAAGACTCAGTTCACACTCTTCAGGAAACAGAT
AAAAAACTAAGAAACAAACAAAAAACAGGCAATCCAACACCATGTGGGAAATGCTTTCATAGCCG
GGAAACCTGGGGAATACCTGAGAGGAATACTCAATTCAGGCCTTGTTTCAGGAATCCAAATCCTG
GCACATCAGAGCTGCTTCCCTCTTTCCAGGGTGGCAGGAAATAAATGGAACATATTTTTCTATCT
TATGCCAAACATGAGGGACCCTTTCTCCCCGGTGCCTCTCCCAAGGTAGTCTACAATATTTCAAC
TCTAGCAGTCTGCTTAGTGCATAGAACATGAGGCTGTGTGTCCCTGGGCAAATTACTAGACTTCT
GTGTGCTTCACTTTCCCTGTAGGATTATAATCTACTGAGCAAGCTTATTGTAAGGGTCAGATTAG
CAACAGTGTATGAAAATGATTTGAGACCATTGCCTGCACAAATTCAACTATTTTTTTTTATCTCA
CTACTCTACAGAAGTAGGTAGGGTGGGAGACAGAGTCTGATGAGAGGCTCAGAATGTGAAAGAAA
GTGAGGCGAGTGAGCATGATATTTAATATAAACACAAAGATATTCTGAGAAGAGCTGCTCACTGC
CCCCTCCCCCAATACATGTTGATAGGAAAATGCCACGTACTTCAGCAAAAACAACTGAAAAATTA
GATAGAAAAGTCAATCAATAGGAAAAGATAATCCAGGACGGTGTTGTGAACAGAAAGAGGGGGAA
AAAACTTTAGAAAATGATGGGGATGCTCTTACTGGGGTACGAGTCCTCAGGTATTGAACTGGCTT
TCAGTAAAAGCTAGATTAGTGGGTTCCTGCCATTTACAAGCTGTTTTATGACAACTTACTTGTTG
GGTGGCCTACAGTAACTCACCTAACTGCACTGAGTCTGTTTCCTCATCTGTAAATTGGGGATTTT
TTTTTAAATACCTGGCATGCCTAACTCATAAAGTTGTTCTGAAACTGAAATAAAACATACGTGAA
CAGGCATTGTAAACTGTAAGTTACGGAAAAAGCTGGCTGTTGTTGTGTCTTTAAAGTTTCACCTG
GGTAGTCAAAGATGGATCATGGGTCTCAGTGGAGAGCTGAGCCAGGCAGGAGCTGACTAAGGGTG
AGAGGTGGGAGTTAGCAGCCTCTGAACATCTGTGTACCATGGGACCCCCTTTCCTCCTGCATGGT
ACCCCAGACAAGGAGCCTAGTAAGAGATACTAATGGCTTGTTGTCCAGAGATGTTCAAACTGCAG
AGAAAGATAAGACAACAAGCATTGGCCTCCAATCATGATGACAGATAGGAGGAGGTGGGAGCTCC
TTAGCAGTGCTGGTTGGCCTTCCATGTTCTACTGTGGGCCATCTCTGCCATGTACTGTAGGCTAC
TAGCTTCTATATTAAAGAATGCAAGAGGGGCCAGGAGCGGAGGCTCATGCCTGTAATCTCAGCAC
TTTGGGAGGCCAAGGTGGGCAGATCACTTGAGGTCAGGAGTTTGTGACCAGCCTGGCCAACATGG
TGAAACTCTGCCTTTACTAAAAATATAAAAATTAGCTGGGTGTGGTGGTGTGCACCTGTAATCCC
AGCTACTCGGGAGACTGAGGCACAAGAATTGCTTGAACCTGGGAGGCGGAAGTTGCAGTGAGCCC
AGATTGCGCCACTGCACTCCACCCTGGGCAACAGAGAAAGACTCTGCCTCAAAAAAAAAAAAAAA
AAGCAAGAGGAAGTGAAATAATCAAGGCCGCCATTTAATAGTGAGCAGCCACTCCATGTGGTACT
GTGCAAGCACATTATAAATATTAGCCTCACAAGAAATGTATTAGCATTTGTATTTTGTACACTGG
TTAAGTATCTTGCCCAAGACCTCAAAACTGGTTAAGGGCAGCAGAATTTAGCCCCAGCACCACCT
TTTCAAAGCCTGGGCTTCTCACACTTCTCCATGCTGTTCCCATTTTAACACAGGTATCTCGCCAT
TCCAGCCACTCAAACTTTGGCATTTAAGAAAATTATCCTAAAGCTAAACTAAACTTCAAGGATGA
CCATTCTCCTGACCCCTTCCCATCAAAATTTTATCTTTAGTCAGTTTGTTTTCGTTTTGTTTTGT
TTTTCAGAACTACCTCTGGCACATCCTCCAAATGAAAGGACTCACTTGGTAATTCTGGGAGCCAT
CTTATTATGCCTTGGTGTAGCACTGACATTCATCTTCCGTTTAAGAAAAGGTAGTATTTCCTTAA
TTGCAGTGGTCTCCACTGGGGGTGAGGAAGGGGTGAGAATTGGATCATGGCTGCAAGGAAACCCG
ACTTAACCTCTGCAAGGTGGTGCAAAGGCATTCCACTGTTCAACAGCAATTATATTGAAGCTGAG
TGGGATCACTGGGTGAAGATGAAGCGTAAGGGGTGAGGGGCAGGAGAATGGGTATGGATGGAGGT
AGAAGATGCAGTGTCATACAGTTTTTTTCTATCATGAAAATAACCACAGACTTACAGAAGAGAAA
GAGCTAAAATGCCCGTCATTTTCAGTTGCATTTTAGTCTTGCATTAGTTGCAACCAGCTGGTTTC
TGGGTACCCTAAGTAATAAAAATAGTTCCTCTGTAGAACTGTAGTATGTTTACCATAGAGTATTT
TGCAAAATTTTTGGTAGAGGATGTTACATAATTTGCATGTGTTCATTTCTCCATTTACCTGTGGG
AACAATTAAAATCCAGGAAAATGAGTATATTCAAATAATTTCCTCCCATTTAAGATGAGTCAGAG
TAAATAATTCCTCCAATACTTAGAGAAGTATACCAAGAGATCCAGTGATGGTATAGAGTTGTCTG
ATGTTAAATAGGGAAGTAGAATATGGAAGGGGATTCCAATAGTCGTTGAAAAATTCCCCATAACC
CCTTACATGGGGGAAAGTAGTGTTAACTGAGAGAGTAGAGATAAGCTGTTTCCAAAAATTATATT
CTTAACAGGACTGAGATAGCCAGAATATAAGGATCAAGTTTCAATGACAGTAAGATCCTGAGATG
GAGTTGATTTGCACAAAGAAATAATTGTTGCCAGCATGCATTTTGAATATTTCTCTGGAAAAAAA
GATTAGTTGGCAGTAGAAATGGATAGAAATCAATAGATATTAAAATACCTCAGAATTTGGTTCAT
CTCTGGGAAAAGATGAAAAATAAAAGTGTATACTCCTCAAGAACATCTAGGATCAAAAGCATGTG
CCCTACACTATTGAATTAATTAACCTCATAAGTTGGGACCTGTGGAATAAGGATGTCCACCAGAC
TTCCTAGGGATTACAAATGTTTCACAGAACTTGAAATTTAAACTTGGGTCACTGTATGGGATGTA
GAGCTGTGCTATATGGAAATAAAAATGATTTCTTTTTCTCAAGGGAGAATGATGGATGTGAAAAA
ATGTGGCATCCAAGATACAAACTCAAAGAAGCAAAGTGGTAAGAATATCAGAAGGAATTGGGAAG
TAAAAGTCAAAGGAAACAAAAAGCTAAAGCAATAACAAAGAGAAATCCATCAGTCATAATCTCCT
CTCCTTTTAAAGAATGCTGGTTCCCCTTTGCCTCACAGCTAACACAAGAACTCCTCCACCGTCTG
AGGAGGTTTAGGAGCAGGGAAGGGGAAGGAGTCAGCTTCATTTGCTAATCTTCTGTTGCCCTGCA
CCCTAGCAGCTCCTTGCAGCAGGGGACAAGGATGACTTAGGTGGATGGATAATTAATTGATTCTA
AAATATTGTGTGTCAGTATTGTAATACTATGTTAATTGCACCATGCACGGTATCTCATTTAATCC
CCCACCCCTTGCCATTACCAAAGAGAGAGAGAGAGAGAGAGAGAGAAATACTAGAATTTATCCTC
ATTTTACAGTAGAGAAAACAGAGGGTCAAGAAGATAATGTAAAGTGCCCAAGAACACACAGCTGA
TCACAAAAATCAAGCTTGGGGGCCATTAGCCTAACCACAGACCCTTACTCTTAACCCATCTGCTT
CAATCCATTTTGCTACAAATGTTTACATTTATAAGCAGGGCAGAAAAACCTCATCCAGGTTATTG
AACTAAGAAGAAAGTTATATTAAGGTTTCTAATTTTTTTAATGTAGTTAGAAACCAAACTTAACA
ATGAGCCCAAGTTTAAAGCAGTCTAATTAACCTGGACAAGCTCAGGCAAGTTTCATTCTGTGGCC
CATAGCATCATCTGTGTTGTAAAGCTAAGTAGCAAATGTTGTTTGGGTCATGCTGGGGGACAAGC
CATCCCAATTTGCTCAGGACTGAGGGGTTTTCCAGGATATCATGTAAGGATAATTGGGTACAAAT
ATAACCTGCTGCTTTCTCTCATTTCAAATTTATCATTTATCATATCAGCAACTATGAGTTATGTT
TTTTATTAGATTTCTTGTTACTTTTTCCCCAGACCACTTCCCATGAAATTAATATACTATTATCA
CTCTCCAGATACACATTTGGAGGAGACGTAATCCAGCATTGGAACTTCTGATCTTCAAGCAGGGA
TTCTCAACCTGTGGTTTAGGGGTTCATCGGGGCTGAGCGTGACAAGAGGAAGGAATGGGCCCGTG
GGATGCAGGCAATGTGGGACTTAAAAGGCCCAAGCACTGAAAATGGAACCTGGCGAAAGCAGAGG
AGGAGAATGAAGAAAGATGGAGTCAAACAGGGAGCCTGGAGGGAGACCTTGATACTTTCAAATGC
CTGAGGGGCTCATCGACGCCTGTGACAGGGAGAAAGGATACTTCTGAACAAGGAGCCTCCAAGCA
AATCATCCATTGCTCATCCTAGGAAGACGGGTTGAGAATCCCTAATTTGAGGGTCAGTTCCTGCA
GAAGTGCCCTTTGCCTCCACTCAATGCCTCAATTTGTTTTCTGCATGACTGAGAGTCTCAGTGTT
GGAACGGGACAGTATTTATGTATGAGTTTTTCCTATTTATTTTGAGTCTGTGAGGTCTTCTTGTC
ATGTGAGTGTGGTTGTGAATGATTTCTTTTGAAGATATATTGTAGTAGATGTTACAATTTTGTCG
CCAAACTAAACTTGCTGCTTAATGATTTGCTCACATCTAGTAAAACATGGAGTATTTGTAAGGTG
CTTGGTCTCCTCTATAACTACAAGTATACATTGGAAGCATAAAGATCAAACCGTTGGTTGCATAG
GATGTCACCTTTATTTAACCCATTAATACTCTGGTTGACCTAATCTTATTCTCAGACCTCAAGTG
TCTGTGCAGTATCTGTTCCATTTAAATATCAGCTTTACAATTATGTGGTAGCCTACACACATAAT
CTCATTTCATCGCTGTAACCACCCTGTTGTGATAACCACTATTATTTTACCCATCGTACAGCTGA
GGAAGCAAACAGATTAAGTAACTTGCCCAAACCAGTAAATAGCAGACCTCAGACTGCCACCCACT
GTCCTTTTATAATACAATTTACAGCTATATTTTACTTTAAGCAATTCTTTTATTCAAAAACCATT
TATTAAGTGCCCTTGCAATATCAATCGCTGTGCCAGGCATTGAATCTACAGATGTGAGCAAGACA
AAGTACCTGTCCTCAAGGAGCTCATAGTATAATGAGGAGATTAACAAGAAAATGTATTATTACAA
TTTAGTCCAGTGTCATAGCATAAGGATGATGCGAGGGGAAAACCCGAGCAGTGTTGCCAAGAGGA
GGAAATAGGCCAATGTGGTCTGGGACGGTTGGATATACTTAAACATCTTAATAATCAGAGTAATT
TTCATTTACAAAGAGAGGTCGGTACTTAAAATAACCCTGAAAAATAACACTGGAATTCCTTTTCT
AGCATTATATTTATTCCTGATTTGCCTTTGCCATATAATCTAATGCTTGTTTATATAGTGTCTGG
TATTGTTTAACAGTTCTGTCTTTTCTATTTAAATGCCACTAAATTTTAAATTCATACCTTTCCAT
GATTCAAAATTCAAAAGATCCCATGGGAGATGGTTGGAAAATCTCCACTTCATCCTCCAAGCCAT
TCAAGTTTCCTTTCCAGAAGCAACTGCTACTGCCTTTCATTCATATGTTCTTCTAAAGATAGTCT
ACATTTGGAAATGTATGTTAAAAGCACGTATTTTTAAAATTTTTTTCCTAAATAGTAACACATTG
TATGTCTGCTGTGTACTTTGCTATTTTTATTTATTTTAGTGTTTCTTATATAGCAGATGGAATGA
ATTTGAAGTTCCCAGGGCTGAGGATCCATGCCTTCTTTGTTTCTAAGTTATCTTTCCCATAGCTT
TTCATTATCTTTCATATGATCCAGTATATGTTAAATATGTCCTACATATACATTTAGACAACCAC
CATTTGTTAAGTATTTGCTCTAGGACAGAGTTTGGATTTGTTTATGTTTGCTCAAAAGGAGACCC
ATGGGCTCTCCAGGGTGCACTGAGTCAATCTAGTCCTAAAAAGCAATCTTATTATTAACTCTGTA
TGACAGAATCATGTCTGGAACTTTTGTTTTCTGCTTTCTGTCAAGTATAAACTTCACTTTGATGC
TGTACTTGCAAAATCACATTTTCTTTCTGGAAATTCCGGCAGTGTACCTTGACTGCTAGCTACCC
TGTGCCAGAAAAGCCTCATTCGTTGTGCTTGAACCCTTGAATGCCACCAGCTGTCATCACTACAC
AGCCCTCCTAAGAGGCTTCCTGGAGGTTTCGAGATTCAGATGCCCTGGGAGATCCCAGAGTTTCC
TTTCCCTCTTGGCCATATTCTGGTGTCAATGACAAGGAGTACCTTGGCTTTGCCACATGTCAAGG
CTGAAGAAACAGTGTCTCCAACAGAGCTCCTTGTGTTATCTGTTTGTACATGTGCATTTGTACAG
TAATTGGTGTGACAGTGTTCTTTGTGTGAATTACAGGCAAGAATTGTGGCTGAGCAAGGCACATA
GTCTACTCAGTCTATTCCTAAGTCCTAACTCCTCCTTGTGGTGTTGGATTTGTAAGGCACTTTAT
CCCTTTTGTCTCATGTTTCATCGTAAATGGCATAGGCAGAGATGATACCTAATTCTGCATTTGAT
TGTCACTTTTTGTACCTGCATTAATTTAATAAAATATTCTTATTTATTTTGTTACTTGGTACACC
AGCATGTCCATTTTCTTGTTTATTTTGTGTTTAATAAAATGTTCAGTTTAACATCCCA

### Human PDL1 Transcript Variant 1—NM_014143.4 (SEQ ID NO: 1); 3′-UTR Underlined (SEQ ID NO: 78)

AGTTCTGCGCAGCTTCCCGAGGCTCCGCACCAGCCGCGCTTCTGTCCGCCTGCAGGGCATTCCAG
AAAGATGAGGATATTTGCTGTCTTTATATTCATGACCTACTGGCATTTGCTGAACGCATTTACTG
TCACGGTTCCCAAGGACCTATATGTGGTAGAGTATGGTAGCAATATGACAATTGAATGCAAATTC
CCAGTAGAAAAACAATTAGACCTGGCTGCACTAATTGTCTATTGGGAAATGGAGGATAAGAACAT
TATTCAATTTGTGCATGGAGAGGAAGACCTGAAGGTTCAGCATAGTAGCTACAGACAGAGGGCCC
GGCTGTTGAAGGACCAGCTCTCCCTGGGAAATGCTGCACTTCAGATCACAGATGTGAAATTGCAG
GATGCAGGGGTGTACCGCTGCATGATCAGCTATGGTGGTGCCGACTACAAGCGAATTACTGTGAA
AGTCAATGCCCCATACAACAAAATCAACCAAAGAATTTTGGTTGTGGATCCAGTCACCTCTGAAC
ATGAACTGACATGTCAGGCTGAGGGCTACCCCAAGGCCGAAGTCATCTGGACAAGCAGTGACCAT
CAAGTCCTGAGTGGTAAGACCACCACCACCAATTCCAAGAGAGAGGAGAAGCTTTTCAATGTGAC
CAGCACACTGAGAATCAACACAACAACTAATGAGATTTTCTACTGCACTTTTAGGAGATTAGATC
CTGAGGAAAACCATACAGCTGAATTGGTCATCCCAGAACTACCTCTGGCACATCCTCCAAATGAA
AGGACTCACTTGGTAATTCTGGGAGCCATCTTATTATGCCTTGGTGTAGCACTGACATTCATCTT
CCGTTTAAGAAAAGGGAGAATGATGGATGTGAAAAAATGTGGCATCCAAGATACAAACTCAAAGA
AGCAAAGTGATACACATTTGGAGGAGACGTAATCCAGCATTGGAACTTCTGATCTTCAAGCAGGG
ATTCTCAACCTGTGGTTTAGGGGTTCATCGGGGCTGAGCGTGACAAGAGGAAGGAATGGGCCCGT
GGGATGCAGGCAATGTGGGACTTAAAAGGCCCAAGCACTGAAAATGGAACCTGGCGAAAGCAGAG
GAGGAGAATGAAGAAAGATGGAGTCAAACAGGGAGCCTGGAGGGAGACCTTGATACTTTCAAATG
CCTGAGGGGCTCATCGACGCCTGTGACAGGGAGAAAGGATACTTCTGAACAAGGAGCCTCCAAGC
AAATCATCCATTGCTCATCCTAGGAAGACGGGTTGAGAATCCCTAATTTGAGGGTCAGTTCCTGC
AGAAGTGCCCTTTGCCTCCACTCAATGCCTCAATTTGTTTTCTGCATGACTGAGAGTCTCAGTGT
TGGAACGGGACAGTATTTATGTATGAGTTTTTCCTATTTATTTTGAGTCTGTGAGGTCTTCTTGT
CATGTGAGTGTGGTTGTGAATGATTTCTTTTGAAGATATATTGTAGTAGATGTTACAATTTTGTC
GCCAAACTAAACTTGCTGCTTAATGATTTGCTCACATCTAGTAAAACATGGAGTATTTGTAAGGT
GCTTGGTCTCCTCTATAACTACAAGTATACATTGGAAGCATAAAGATCAAACCGTTGGTTGCATA
GGATGTCACCTTTATTTAACCCATTAATACTCTGGTTGACCTAATCTTATTCTCAGACCTCAAGT
GTCTGTGCAGTATCTGTTCCATTTAAATATCAGCTTTACAATTATGTGGTAGCCTACACACATAA
TCTCATTTCATCGCTGTAACCACCCTGTTGTGATAACCACTATTATTTTACCCATCGTACAGCTG
AGGAAGCAAACAGATTAAGTAACTTGCCCAAACCAGTAAATAGCAGACCTCAGACTGCCACCCAC
TGTCCTTTTATAATACAATTTACAGCTATATTTTACTTTAAGCAATTCTTTTATTCAAAAACCAT
TTATTAAGTGCCCTTGCAATATCAATCGCTGTGCCAGGCATTGAATCTACAGATGTGAGCAAGAC
AAAGTACCTGTCCTCAAGGAGCTCATAGTATAATGAGGAGATTAACAAGAAAATGTATTATTACA
ATTTAGTCCAGTGTCATAGCATAAGGATGATGCGAGGGGAAAACCCGAGCAGTGTTGCCAAGAGG
AGGAAATAGGCCAATGTGGTCTGGGACGGTTGGATATACTTAAACATCTTAATAATCAGAGTAAT
TTTCATTTACAAAGAGAGGTCGGTACTTAAAATAACCCTGAAAAATAACACTGGAATTCCTTTTC
TAGCATTATATTTATTCCTGATTTGCCTTTGCCATATAATCTAATGCTTGTTTATATAGTGTCTG
GTATTGTTTAACAGTTCTGTCTTTTCTATTTAAATGCCACTAAATTTTAAATTCATACCTTTCCA
TGATTCAAAATTCAAAAGATCCCATGGGAGATGGTTGGAAAATCTCCACTTCATCCTCCAAGCCA
TTCAAGTTTCCTTTCCAGAAGCAACTGCTACTGCCTTTCATTCATATGTTCTTCTAAAGATAGTC
TACATTTGGAAATGTATGTTAAAAGCACGTATTTTTAAAATTTTTTTCCTAAATAGTAACACATT
GTATGTCTGCTGTGTACTTTGCTATTTTTATTTATTTTAGTGTTTCTTATATAGCAGATGGAATG
AATTTGAAGTTCCCAGGGCTGAGGATCCATGCCTTCTTTGTTTCTAAGTTATCTTTCCCATAGCT
TTTCATTATCTTTCATATGATCCAGTATATGTTAAATATGTCCTACATATACATTTAGACAACCA
CCATTTGTTAAGTATTTGCTCTAGGACAGAGTTTGGATTTGTTTATGTTTGCTCAAAAGGAGACC
CATGGGCTCTCCAGGGTGCACTGAGTCAATCTAGTCCTAAAAAGCAATCTTATTATTAACTCTGT
ATGACAGAATCATGTCTGGAACTTTTGTTTTCTGCTTTCTGTCAAGTATAAACTTCACTTTGATG
CTGTACTTGCAAAATCACATTTTCTTTCTGGAAATTCCGGCAGTGTACCTTGACTGCTAGCTACC
CTGTGCCAGAAAAGCCTCATTCGTTGTGCTTGAACCCTTGAATGCCACCAGCTGTCATCACTACA
CAGCCCTCCTAAGAGGCTTCCTGGAGGTTTCGAGATTCAGATGCCCTGGGAGATCCCAGAGTTTC
CTTTCCCTCTTGGCCATATTCTGGTGTCAATGACAAGGAGTACCTTGGCTTTGCCACATGTCAAG
GCTGAAGAAACAGTGTCTCCAACAGAGCTCCTTGTGTTATCTGTTTGTACATGTGCATTTGTACA
GTAATTGGTGTGACAGTGTTCTTTGTGTGAATTACAGGCAAGAATTGTGGCTGAGCAAGGCACAT
AGTCTACTCAGTCTATTCCTAAGTCCTAACTCCTCCTTGTGGTGTTGGATTTGTAAGGCACTTTA
TCCCTTTTGTCTCATGTTTCATCGTAAATGGCATAGGCAGAGATGATACCTAATTCTGCATTTGA
TTGTCACTTTTTGTACCTGCATTAATTTAATAAAATATTCTTATTTATTTTGTTACTTGGTACAC
CAGCATGTCCATTTTCTTGTTTATTTTGTGTTTAATAAAATGTTCAGTTTAACATCCCA

### Human PDL1 Transcript Variant 2—NM_001267706.2 (SEQ ID NO: 2); 3′-UTR Underlined (SEQ ID NO: 79)

AGTTCTGCGCAGCTTCCCGAGGCTCCGCACCAGCCGCGCTTCTGTCCGCCTGCAGGGCATTCCAG
AAAGATGAGGATATTTGCTGTCTTTATATTCATGACCTACTGGCATTTGCTGAACGCCCCATACA
ACAAAATCAACCAAAGAATTTTGGTTGTGGATCCAGTCACCTCTGAACATGAACTGACATGTCAG
GCTGAGGGCTACCCCAAGGCCGAAGTCATCTGGACAAGCAGTGACCATCAAGTCCTGAGTGGTAA
GACCACCACCACCAATTCCAAGAGAGAGGAGAAGCTTTTCAATGTGACCAGCACACTGAGAATCA
ACACAACAACTAATGAGATTTTCTACTGCACTTTTAGGAGATTAGATCCTGAGGAAAACCATACA
GCTGAATTGGTCATCCCAGAACTACCTCTGGCACATCCTCCAAATGAAAGGACTCACTTGGTAAT
TCTGGGAGCCATCTTATTATGCCTTGGTGTAGCACTGACATTCATCTTCCGTTTAAGAAAAGGGA
GAATGATGGATGTGAAAAAATGTGGCATCCAAGATACAAACTCAAAGAAGCAAAGTGATACACAT
TTGGAGGAGACGTAATCCAGCATTGGAACTTCTGATCTTCAAGCAGGGATTCTCAACCTGTGGTT
TAGGGGTTCATCGGGGCTGAGCGTGACAAGAGGAAGGAATGGGCCCGTGGGATGCAGGCAATGTG
GGACTTAAAAGGCCCAAGCACTGAAAATGGAACCTGGCGAAAGCAGAGGAGGAGAATGAAGAAAG
ATGGAGTCAAACAGGGAGCCTGGAGGGAGACCTTGATACTTTCAAATGCCTGAGGGGCTCATCGA
CGCCTGTGACAGGGAGAAAGGATACTTCTGAACAAGGAGCCTCCAAGCAAATCATCCATTGCTCA
TCCTAGGAAGACGGGTTGAGAATCCCTAATTTGAGGGTCAGTTCCTGCAGAAGTGCCCTTTGCCT
CCACTCAATGCCTCAATTTGTTTTCTGCATGACTGAGAGTCTCAGTGTTGGAACGGGACAGTATT
TATGTATGAGTTTTTCCTATTTATTTTGAGTCTGTGAGGTCTTCTTGTCATGTGAGTGTGGTTGT
GAATGATTTCTTTTGAAGATATATTGTAGTAGATGTTACAATTTTGTCGCCAAACTAAACTTGCT
GCTTAATGATTTGCTCACATCTAGTAAAACATGGAGTATTTGTAAGGTGCTTGGTCTCCTCTATA
ACTACAAGTATACATTGGAAGCATAAAGATCAAACCGTTGGTTGCATAGGATGTCACCTTTATTT
AACCCATTAATACTCTGGTTGACCTAATCTTATTCTCAGACCTCAAGTGTCTGTGCAGTATCTGT
TCCATTTAAATATCAGCTTTACAATTATGTGGTAGCCTACACACATAATCTCATTTCATCGCTGT
AACCACCCTGTTGTGATAACCACTATTATTTTACCCATCGTACAGCTGAGGAAGCAAACAGATTA
AGTAACTTGCCCAAACCAGTAAATAGCAGACCTCAGACTGCCACCCACTGTCCTTTTATAATACA
ATTTACAGCTATATTTTACTTTAAGCAATTCTTTTATTCAAAAACCATTTATTAAGTGCCCTTGC
AATATCAATCGCTGTGCCAGGCATTGAATCTACAGATGTGAGCAAGACAAAGTACCTGTCCTCAA
GGAGCTCATAGTATAATGAGGAGATTAACAAGAAAATGTATTATTACAATTTAGTCCAGTGTCAT
AGCATAAGGATGATGCGAGGGGAAAACCCGAGCAGTGTTGCCAAGAGGAGGAAATAGGCCAATGT
GGTCTGGGACGGTTGGATATACTTAAACATCTTAATAATCAGAGTAATTTTCATTTACAAAGAGA
GGTCGGTACTTAAAATAACCCTGAAAAATAACACTGGAATTCCTTTTCTAGCATTATATTTATTC
CTGATTTGCCTTTGCCATATAATCTAATGCTTGTTTATATAGTGTCTGGTATTGTTTAACAGTTC
TGTCTTTTCTATTTAAATGCCACTAAATTTTAAATTCATACCTTTCCATGATTCAAAATTCAAAA
GATCCCATGGGAGATGGTTGGAAAATCTCCACTTCATCCTCCAAGCCATTCAAGTTTCCTTTCCA
GAAGCAACTGCTACTGCCTTTCATTCATATGTTCTTCTAAAGATAGTCTACATTTGGAAATGTAT
GTTAAAAGCACGTATTTTTAAAATTTTTTTCCTAAATAGTAACACATTGTATGTCTGCTGTGTAC
TTTGCTATTTTTATTTATTTTAGTGTTTCTTATATAGCAGATGGAATGAATTTGAAGTTCCCAGG
GCTGAGGATCCATGCCTTCTTTGTTTCTAAGTTATCTTTCCCATAGCTTTTCATTATCTTTCATA
TGATCCAGTATATGTTAAATATGTCCTACATATACATTTAGACAACCACCATTTGTTAAGTATTT
GCTCTAGGACAGAGTTTGGATTTGTTTATGTTTGCTCAAAAGGAGACCCATGGGCTCTCCAGGGT
GCACTGAGTCAATCTAGTCCTAAAAAGCAATCTTATTATTAACTCTGTATGACAGAATCATGTCT
GGAACTTTTGTTTTCTGCTTTCTGTCAAGTATAAACTTCACTTTGATGCTGTACTTGCAAAATCA
CATTTTCTTTCTGGAAATTCCGGCAGTGTACCTTGACTGCTAGCTACCCTGTGCCAGAAAAGCCT
CATTCGTTGTGCTTGAACCCTTGAATGCCACCAGCTGTCATCACTACACAGCCCTCCTAAGAGGC
TTCCTGGAGGTTTCGAGATTCAGATGCCCTGGGAGATCCCAGAGTTTCCTTTCCCTCTTGGCCAT
ATTCTGGTGTCAATGACAAGGAGTACCTTGGCTTTGCCACATGTCAAGGCTGAAGAAACAGTGTC
TCCAACAGAGCTCCTTGTGTTATCTGTTTGTACATGTGCATTTGTACAGTAATTGGTGTGACAGT
GTTCTTTGTGTGAATTACAGGCAAGAATTGTGGCTGAGCAAGGCACATAGTCTACTCAGTCTATT
CCTAAGTCCTAACTCCTCCTTGTGGTGTTGGATTTGTAAGGCACTTTATCCCTTTTGTCTCATGT
TTCATCGTAAATGGCATAGGCAGAGATGATACCTAATTCTGCATTTGATTGTCACTTTTTGTACC
TGCATTAATTTAATAAAATATTCTTATTTATTTTGTTACTTGGTACACCAGCATGTCCATTTTCT
TGTTTATTTTGTGTTTAATAAAATGTTCAGTTTAACATCCCA

### Human PDL1 Transcript Variant 4—NM_001314029.2 (SEQ ID NO: 3); 3′-UTR Underlined

AGTTCTGCGCAGCTTCCCGAGGCTCCGCACCAGCCGCGCTTCTGTCCGCC
TGCAGGGCATTCCAGAAAGATGAGGATATTTGCTGTCTTTATATTCATGA
CCTACTGGCATTTGCTGAACGCATTTACTGTCACGGTTCCCAAGGACCTA
TATGTGGTAGAGTATGGTAGCAATATGACAATTGAATGCAAATTCCCAGT
AGAAAAACAATTAGACCTGGCTGCACTAATTGTCTATTGGGAAATGGAGG
ATAAGAACATTATTCAATTTGTGCATGGAGAGGAAGACCTGAAGGTTCAG
CATAGTAGCTACAGACAGAGGGCCCGGCTGTTGAAGGACCAGCTCTCCCT
GGGAAATGCTGCACTTCAGATCACAGATGTGAAATTGCAGGATGCAGGGG
TGTACCGCTGCATGATCAGCTATGGTGGTGCCGACTACAAGCGAATTACT
GTGAAAGTCAATGCCCCATACAACAAAATCAACCAAAGAATTTTGGTTGT
GGATCCAGTCACCTCTGAACATGAACTGACATGTCAGGCTGAGGGCTACC
CCAAGGCCGAAGTCATCTGGACAAGCAGTGACCATCAAGTCCTGAGTGGT
AAGACCACCACCACCAATTCCAAGAGAGAGGAGAAGCTTTTCAATGTGAC
CAGCACACTGAGAATCAACACAACAACTAATGAGATTTTCTACTGCACTT
TTAGGAGATTAGATCCTGAGGAAAACCATACAGCTGAATTGGTCATCCCA
GGTAATATTCTGAATGTGTCCATTAAAATATGTCTAACACTGTCCCCTAG
CACCTAGCATGATGTCTGCCTATCATAGTCATTCAGTGATTGTTGAATAA
ATGAATGAATGAATAACA

In some embodiments, a disruption in the 3′-UTR of an allele encoding PDL1 comprises a deletion of the 3′-UTR. In some embodiments, a deletion comprises deletion of one or more fragments of the 3′-UTR. In some embodiments, a deletion is a complete deletion of the 3′-UTR. In some embodiments, a deletion is a partial deletion of the 3′-UTR. In some embodiments, a disruption in the 3′-UTR of an allele encoding PDL1 comprises an inversion of a sequence in the 3′-UTR. In some embodiments, a disruption in the 3′-UTR of an allele encoding PDL1 comprises an inversion of a fragment in the 3′-UTR. In some embodiments, a disruption in the 3′-UTR of an allele encoding PDL1 comprises an inversion of the entire 3′-UTR. In some embodiments, a disruption in the 3′-UTR of an allele encoding PDL1 comprises a translocation of a sequence in the entire 3′-UTR. In some embodiments, a disruption in the 3′-UTR of an allele encoding PDL1 comprises substitution of one or more nucleotides in the 3′-UTR. In some embodiments, a disruption in the 3′-UTR of an allele encoding PDL1 comprises a deletion resulting in the loss of a portion of the 3′ UTR spanning from any of the nucleotides corresponding to nucleotides 1000-1050 of SEQ ID NO: 1 to the nucleotide corresponding to 3634 of SEQ ID NO: 1. In some embodiments, a disruption in the 3′-UTR of an allele encoding PDL1 comprises a deletion resulting in the loss of a portion of the 3′ UTR spanning from any of the nucleotides corresponding to nucleotides 1010-1050 of SEQ ID NO: 1 to the nucleotide corresponding to 3634 of SEQ ID NO: 1. In some embodiments, a disruption in the 3′-UTR of an allele encoding PDL1 comprises a deletion resulting in the loss of a portion of the 3′ UTR spanning from any of the nucleotides corresponding to nucleotides 1010-1040 of SEQ ID NO: 1 to the nucleotide corresponding to 3634 of SEQ ID NO: 1. In some embodiments, at least 1, 2, 3, 4, 5, 6, 7, 8, 9, 10, 25, 50, 100, 200, 300, 400, 500, 600, 700, 800, 900, 1000, 1200, 1500, 1700, 2000, 2200, 2300, 2400, 2500, or 2600 nucleotides (e.g., consecutive nucleotides) of the 3′ UTR of an allele encoding PDL1 have been deleted. In some embodiments, at least 10, 25, 50, 100, 200, 300, 400, 500, 600, 700, 800, 900, 1000, 1200, 1500, 1700, 2000, 2200, 2300, 2400, 2500, or 2600 nucleotides (e.g., consecutive nucleotides) from the portion corresponding to nucleotides 943 to 3634 of a sequence that is at least 75%, 80%, 85%, 90%, 91%, 92%, 93%, 94%, 95%, 96%, 97%, 98%, 99% or 100% identical to SEQ ID NO: 1 have been deleted. In some embodiments, the disclosure provides for a cell (e.g., a stem cell) in which the 3′-UTR of the PDL1 gene has been disrupted by a complete or partial deletion (e.g., at least 10, 25, 50, 100, 200, 300, 400, 500, 600, 700, 800, 900, 1000, 1200, 1500, 1700, 2000, 2200, 2300, 2400, 2500, or 2600 nucleotides, such as consecutive nucleotides) in the cell's genome between sequences corresponding to SEQ ID NO: 13 and SEQ ID NO: 15. In some embodiments, the disclosure provides for a cell (e.g., a stem cell) in which the 3′-UTR of the PDL1 gene has been disrupted by a complete or partial deletion (e.g., at least 10, 25, 50, 100, 200, 300, 400, 500, 600, 700, 800, 900, 1000, 1200, 1500, 1700, 2000, 2200, 2300, 2400, 2500, or 2600 nucleotides, such as consecutive nucleotides) in the cell's genome between sequences corresponding to SEQ ID NO: 14 and SEQ ID NO: 15. In some embodiments, the disclosure contemplates a cell in which a portion of the cell's genome has been excised using a gene editing system comprising an RNA-guided endonuclease (e.g., a Cas9 or Cas12) and RNA guides targeting the sequences of SEQ ID NO:13 and SEQ ID NO: 15. In some embodiments, the disclosure contemplates a cell in which a portion of the cell's genome has been excised using a gene editing system comprising an RNA-guided endonuclease (e.g., a Cas9 or Cas12) and RNA guides targeting the sequences of SEQ ID NO:14 and SEQ ID NO: 15.

In some embodiments, the disclosure contemplates a cell in which SEQ ID NO: 31 (TCCAGCATTGGAACTTCTGATCT) or SEQ ID NO: 32 (TCTGATC) of the PDL1 3′-UTR comprises one or more nucleotide deletions, insertions, and/or substitutions. In some embodiments, the sequence of SEQ ID NO: 31 or 32 is mutated in the PDL1 3′-UTR such that miR-140 is unable to bind or has significantly reduced binding to SEQ ID NO: 32 or 32 or RNA equivalents or complementary sequences thereof. In some embodiments, the disclosure contemplates a cell in which SEQ ID NO: 33 (CCACCCTGTTGTGATAACCACTA) or SEQ ID NO: 34 (AACCACT) of the PDL1 3′-UTR comprises one or more nucleotide deletions, insertions, and/or substitutions. In some embodiments, the sequence of SEQ ID NO: 33 or 34 is mutated in the PDL1 3′-UTR such that miR-142 is unable to bind or has significantly reduced binding to SEQ ID NO: 33 or 34 or RNA equivalents and/or complementary sequences thereof.

In some embodiments, the disclosure contemplates a cell in which SEQ ID NO: 35 (GCCACCCACTGTCCTTTTATAAT) or 36 (TTTATAA) of the PDL1 3′-UTR comprises one or more nucleotide deletions, insertions, and/or substitutions. In some embodiments, the sequence of SEQ ID NO: 35 or 36 is mutated in the PDL1 3′-UTR such that miR-340 is unable to bind to or has significantly reduced binding to SEQ ID NO: 35 or 36 or RNA equivalents and/or complementary sequences thereof. In some embodiments, the disclosure contemplates a cell in which SEQ ID NO: 37 (TTGGATTTGTAAGGCACTTTAT) or 38 (ACTTTAT) of the PDL1 3′-UTR comprises one or more nucleotide deletions, insertions, and/or substitutions. In some embodiments, the sequence of SEQ ID NO: 37 or 38 is mutated in the PDL1 3′-UTR such that miR-383 is unable to bind to or has significantly reduced binding to SEQ ID NO: 37 or 38 or RNA equivalents and/or complementary sequences thereof. In some embodiments, the disclosure contemplates a cell in which SEQ ID NO: 39 (GGCTCATCGACGCCTGTGAC) or 40 (CCTGTGA) of the PDL1 3′-UTR comprises one or more nucleotide deletions, insertions, and/or substitutions. In some embodiments, the sequence of SEQ ID NO: 39 or 40 is mutated in the PDL1 3′-UTR such that miR-513 is unable to bind or has significantly reduced binding to SEQ ID NO: 39 or 40 or RNA equivalents and/or complementary sequences thereof. In some embodiments, the disclosure contemplates a cell in which SEQ ID NO: 41 (AATAGCAGACCTCAGACTGCCA) or 42 (ACTGCCA) of the PDL1 3′-UTR comprises one or more nucleotide deletions, insertions, and/or substitutions, e.g., to generate the sequence of SEQ ID NO: 43 (ACTCCCA). In some embodiments, the sequence of SEQ ID NO: 41 or 42 is mutated in the PDL1 3′-UTR such that miR-34a is unable to bind or has significantly reduced binding to SEQ ID NO: 41 or 42 or RNA equivalents and/or complementary sequences thereof. In some embodiments, the disclosure contemplates a cell in which SEQ ID NO: 44 (CAGTGTTGGAACGGGACAGTATTT), 45 (CAGTGTT), or 46 (CAGTATT) of the PDL1 3′-UTR comprises one or more nucleotide deletions, insertions, and/or substitutions. In some embodiments, the sequence of SEQ ID NO: 44, 45 or 46 is mutated in the PDL1 3′-UTR such that miR-200a and/or miR-200b/c are unable to bind or have significantly reduced binding to SEQ ID NO: 44, 45 or 46 or RNA equivalents and/or complementary sequences thereof. In some embodiments, the disclosure contemplates a cell in which SEQ ID NO: 47 (CCAAACTAAACTTGCTGCTT) or 48 (TTGCTGCT) of the PDL1 3′-UTR comprises one or more nucleotide deletions, insertions, and/or substitutions. In some embodiments, the sequence of SEQ ID NO: 47 or 48 is mutated in the PDL1 3′-UTR such that miR-424(322), miR-195 or miR497-5p is unable to bind or has significantly reduced binding to SEQ ID NO: 47 or 48 or RNA equivalents and/or complementary sequences thereof. In some embodiments, the disclosure contemplates a cell in which SEQ ID NO: 57 (TGTGAGCAAGACAAAGTAC) of the PDL1 3′-UTR comprises one or more nucleotide deletions, insertions, and/or substitutions. In some embodiments, the sequence of SEQ ID NO: 57 is mutated in the PDL1 3′-UTR such that miR-33a is unable to bind or has significantly reduced binding to SEQ ID NO: 57 or RNA equivalents and/or complementary sequences thereof. In some embodiments, the disclosure contemplates a cell in which SEQ ID NO: 58 (GCATTAA) or 59 (AGCATTA) of the PDL1 3′-UTR comprises one or more nucleotide deletions, insertions, and/or substitutions. In some embodiments, the sequence of SEQ ID NO: 58 and/or 59 is mutated in the PDL1 3′-UTR such that miR155 is unable to bind or has significantly reduced binding to SEQ ID NO: 58 and/or 59 or RNA equivalents and/or complementary sequences thereof. In some embodiments, the disclosure contemplates a cell in which SEQ ID NO: 60 (ACTTAAAAGGCCCAAGCACTGAA) or 61 (GCACTG) of the PDL1 3′-UTR comprises one or more nucleotide deletions, insertions, and/or substitutions. In some embodiments, the sequence of SEQ ID NO: 60 and/or 61 is mutated in the PDL1 3′-UTR such that miR-152 is unable to bind or has significantly reduced binding to SEQ ID NO: 60 and/or 61 or RNA equivalents and/or complementary sequences thereof. In some embodiments, the disclosure contemplates a cell in which SEQ ID 65, NO: 62 (TATTTTGTTACTTGGTACACCAGCA) or 63 (ACACCAGC) of the PDL1 3′-UTR comprises one or more nucleotide deletions, insertions, and/or substitutions. In some embodiments, the sequence of SEQ ID NO: 62 and/or 63 is mutated in the PDL1 3′-UTR such that miR-138-5p is unable to bind or has significantly reduced binding to SEQ ID NO: 62 and/or 63 or RNA equivalents and/or complementary sequences thereof. In some embodiments, the disclosure contemplates a cell in which SEQ ID NO: 64 (CGCCAAACTAAACTTGCTGCTT) or 65 (ACTTGCTGCT) of the PDL1 3′-UTR comprises one or more nucleotide deletions, insertions, and/or substitutions. In some embodiments, the sequence of SEQ ID NO: 64 and/or 65 is mutated in the PDL1 3′-UTR such that miR-16, miR-15a, and/or miR15b is unable to bind or has significantly reduced binding to SEQ ID NO: 64 and/or 65 or RNA equivalents and/or complementary sequences thereof. In some embodiments, the disclosure contemplates a cell in which SEQ ID NO: 66 (ATCCCTAATTTGAGGGTCAGTT) or 67 (TTTGAGGGTCAGT) of the PDL1 3′-UTR comprises one or more nucleotide deletions, insertions, and/or substitutions. In some embodiments, the sequence of SEQ ID NO: 66 and/or 67 is mutated in the PDL1 3′-UTR such that miR-193a is unable to bind or has significantly reduced binding to SEQ ID NO: 66 and/or 67 or RNA equivalents and/or complementary sequences thereof. In some embodiments, the disclosure contemplates a cell in which SEQ ID NO: 68 (GGTGTTGGATTTGTAAGGCACTTTA) or 69 (GCACTTT) of the PDL1 3′-UTR comprises one or more nucleotide deletions, insertions, and/or substitutions. In some embodiments, the sequence of SEQ ID NO: 68 and/or 69 is mutated in the PDL1 3′-UTR such that miR-17-5p is unable to bind or has significantly reduced binding to SEQ ID NO: 68 and/or 69 or RNA equivalents and/or complementary sequences thereof. In some embodiments, the disclosure contemplates a cell in which SEQ ID NO: 70 (AAGGAATGGGCCCGTGGGATGCA) or 71 (GGGATGC) of the PDL1 3′-UTR comprises one or more nucleotide deletions, insertions, and/or substitutions. In some embodiments, the sequence of SEQ ID NO: 70 and/or 71 is mutated in the PDL1 3′-UTR such that miR-324-5p is unable to bind or has significantly reduced binding to SEQ ID NO: 70 and/or 71 or RNA equivalents and/or complementary sequences thereof. In some embodiments, the disclosure contemplates a cell in which SEQ ID NO: 72 (ATTTCTTTTGAAGATATATTGTA) or 73 (ATATTGT) of the PDL1 3′-UTR comprises one or more nucleotide deletions, insertions, and/or substitutions. In some embodiments, the sequence of SEQ ID NO: 72 and/or 73 is mutated in the PDL1 3′-UTR such that miR-338-5p is unable to bind or has significantly reduced binding to SEQ ID NO: 72 and/or 73 or RNA equivalents and/or complementary sequences thereof. In some embodiments, the disclosure provides for a cell in which 1, 2, 3, 4, 5, 6, or 7 or all of the nucleotides have been deleted from any one or more of SEQ ID Nos: 32, 34, 36, 38, 40, 42, 45, 48, 36, 58, 59, 61, 63, 65, 67, 69, 71, and/or 73 of the PDL1 3′-UTR. In some embodiments, the disclosure provides for a cell in which 1, 2, 3, 4, 5, 6, 7, 8, 9, 10, 11, 12, 13, 14, 15, 16, 17, 18, 19, 20, 21, 22, 23 or 24 or all of the nucleotides have been deleted from any one of SEQ ID Nos: 31, 33, 35, 37, 39, 41, 44, 47, 57, 60, 62, 64, 66, 68, 70, and/or 72, of the PDL1 3′-UTR. See, e.g., Xie et al., 2017 PLOS One, DOI:10.1371/journal.pone.0168822; Zhao et al., 2016, Oncotarget, 7(29):45370-84; He et al., 2018 Biomedicine and Pharmacology, 98:95-101; Tao et al., 2018, Cell Physiol Biochem., 48:801-814; Kao et al., 2017, J. Thoracic Oncology, 12(9):1421-1433; Audrito et al., 2017, Oncotarget, 8(9):15894-15911; Holla et al., 2016, Scientific Reports, 6(24193); Danbaran et al., 2020, International Immunopharmacology, 84:106594; Gong et al., 2009, J Immunol., 182(3):1325-1333; Chen et al., 2014, Nat. Commun., 5:5241; Wang et al., 2015, Cellular Signaling, 27(3):443-452; Xu et al., 2016, Nat. Comm., 7:11406; and Dong et al., 2018, Oncogene, 37:5257-5268, each of which is incorporated by reference herein in its entirety. It should be noted that, because the sequences of SEQ ID Nos: 1, 2, 31, 32-42, 44-45 47-48, and/or 57-73 of the PDL1 3′-UTR are derived from naturally occurring nucleotide sequences in a cell, it is possible that the nucleic acids in the cell will have some differences (e.g., polymorphisms) as compared to these reference sequences. As such, the disclosure contemplates that the cell may comprise a nucleotide sequence having no more than one, two, three, four, five, or six nucleotide differences as compared to any of the reference sequences of SEQ ID Nos: 1, 2, 31, 32-42, 44-45 47-48, and/or 57-73 of the PDL1 3′-UTR prior to modification. In some embodiments, the cell is heterozygous for any one of or combination of the above genetic elements listed in this paragraph. In some embodiments, the cell is homozygous for any one of or combination of the above genetic elements listed in this paragraph.

In some embodiments, a disruption in the 3′-UTR of an allele encoding an PDL1 leads to increased expression (e.g., increased by at least 10%, at least 20%, at least 30%, at least 40%, at least 50%, at least 60%, at least 70%, at least 80%, at least 90%, at least 100%, at least 2-fold, at least 5-fold, at least 10-fold, at least 50-fold, at least 100-fold or higher) of PDL1 in the cell, the isolated stem cell, or cells differentiated from the isolated stem cell. In some embodiments, the increased expression of PDL1 is induced or increased by interferon gamma.

In some embodiments, a cell (e.g., an isolated stem cell) described herein comprises a disruption in the 3′-UTR of an allele encoding Cluster of Differentiation 47 (CD47). In some embodiments, the disruption is a homozygous modification. In some embodiments, the disruption is a heterozygous modification. “Cluster of Differentiation 47 (CD47)” belongs to the immunoglobulin superfamily and partners with membrane integrins and also binds the ligands thrombospondin-1 (TSP-1) and signal-regulatory protein alpha (SIRPα). CD47 acts as a “don't eat me” signal to macrophages of the immune system. Increased expression of CD47 in cells administered to a subject as cell-based therapy protects the cells from macrophage engulfment.

An example of a Homo sapiens CD47 gene sequence is provided in NCBI Gene ID: 961 (SEQ ID NO: 29; corresponding to a sequence complementary to positions 108043091 to 108094200 of Homo Sapiens chromosome 3 sequence as provided in NCBI Accession No.: NC_000003.12). In addition, examples of human CD47 transcript variants that encode different isoforms of CD47 proteins are provided in NCBI Accession Nos.: NM_001777.4 (SEQ ID NO: 4), NM_198793.3 (SEQ ID NO: 5), or NM_001382306.1 (SEQ ID NO: 6).

### Human CD47 Gene—NCBI Gene ID: 961 (SEQ ID NO: 29); 3′-UTR Underlined (SEQ ID NO: 88)

CTCATTCTATTTATTCTTTTAACAACGATTTGTTGAGTACATACTATGTGTCAGGCAAAGGGGCT
AGCAGTAAAGAAGACTCATATGGTCCCTCCCCTCATTGAGCTTACAGTATAGGAAACAGTTATTA
CTGAACACGAAGACAGCTATATAAACATAATTTCAATAAAAGATGGTAACTGATATGCACCAAAG
TAAGACTAAAACACAGAGAAGTGCACTTAACTTATACTGGCATTATCATGGGCCTTGATTTACTG
TGGGATCCTTTGCTGTTATGTTCCCTTACGTTGAAAGATGCATCTGGCCCTACACAAAGCACAGA
CTATGAGTAGGTAGGCACATACAGTTCTAAGCCAACCCCTTGGGGCACCAGGAGACAGCACTGTC
TCATGTCGTTGCATGACACACCACCATGGGGCAAAACATTTGCTGAATGAAGTTACAATAAGGAT
ACAATTGTATCTACATAAACATTTAAAGATACTATCATATCAATACCTACTATGTGGCAGGATCT
TGATGTTGTGTGGGGAGGAAGATAAAAGTAGATGTATCGTTGAGACAGAAATGAGGGCAAGGGAA
ATTTTTTATGCATTGAGAGAGGAAGTTTGTGAATTTTTTCGCTGGCCATGGAGTCAGGGAGACTT
CAGTCCAACCACCGTCCCTCCCTGAGCTTGTGACCAAGCCTCACTTTCCTTATCTATAAAGGTGA
CAAGAGTGATTTTTTTAAGTTTATTTATCTGTAAAGGGGAAACTGTAGCAACTTTGCTATTATAG
GGTAAAAAGAAAAAAGGATGAAAACCAGAACAAGGGAGAGAGACAAGAGAGAGAGCAGTGTCACT
TATAACTGTGGGCAAAGTGCTCACCATTCCTCTTTCCCTTTGTTTCCTCATCTCAAAAAGGGAGA
TCATAACATTTATCCTTTCTGAGGTTGTTATGAAGATTAACTTAGGTGATATAGCTAATGTGTAC
TTCCTGTGTTCAAAACATTCTAATATGTATTATCCCCTCCCTCTCTACCCAAGAAACTCCTTACA
TACAGATAAGATGAGCTCAACATCCAAATTAAAATGCTTTGAAAAGTTAAAAGGCAAACCTTTAA
AATGATTAAATACTGAAAAGAGCTTAAGGTTATCTCAGAGAGAGGATAATCAAACCTAGGCAGGC
TGCTTTGGACTGTCTCTCCTGGGATATGCCTGCTTTTGCCCCACCACCAAAACATACCCCAGCTA
GATTCCCAAGAGCAGCAGTGGTCACCCAGGTGGCCTCTCATTCCATTTTCTGCACAAAAAGACGT
TAAGCATATAGCTCAGTAGCTCCCAGTGTAAAACAGAACATCACCCGATCTCCACCTCTGAAGGT
TGGGGACAGTCCTTTCTAGTCACTCTTCAGCTTTAGGGGGGTTGGTTGGTATCAGGGACAGCTGA
CAGCCTTATTTAGCCAACCAGCTGCTCTGTGATACACAATGTCCCTAATGCTCAAAGTCTGGTGG
ATGATTCTTTTGTATCCACATAAGCAGCAGTTGGAGGAGAATCGATGAGTCCCTTTGGTTGCCTC
GGGGTGTCAAGGCTGCCATTCAAATCATTACCACCATAATTATTAACCATTGTTTTAGTGAGATG
TCTGCCAGGCAATTGTTTTTATTTTTCAATCATTAAAAAAGCAATCAAATTTCACTAGAGCAATG
CCTGCCTCACGCCGCATCAACACATTTATTAAACCCCTTCTGTTTGCCGAGCTCAATGGAAAGTC
TTGGAGGAGGGAATACTTAAAATGCTCTCGGATTTAAAAGAATTGATAATGCTGTGGGGAAGAGG
TTCACACAAAAAAGCAATTACAGGAAGAAGCTCGATAGTATACAATACACTGGTACAGAGTGTCA
TAGACAGTGCCACTTTCATACGCTGGATTTTATTTCTGTGGCAATGGGATGCTTGGGAGGAGCCG
CACTGTGTAGAGGATTTGGAGAAGTGGGGTATTGTGGTGGGAAATTGCTTTCTTTCCCAGGAGGT
AGGAGGAAAACAATCAAGGAGGTGGACAGGATGTGCACTCCATTAGAGCAGCCACCAGAGCCTGA
CTTTTTGATAAGAGAGTACATCAGTTAGGATAACGGTTAAAAGTATCTTTAAAAGACTTTTGCTT
CAGGATGAATGATGTGGCCTGTGTGATTCAGCGATAAATTCAAAAGCCTTGTCCCTATTGTGGCT
TGCGGCCACATTTCGAACCCATTTTTCAAGCATGTTAAACCCAAGCGCAGCGCAGAGGGCTGCAC
ATGGGGCAGTCACAAACCAAGCTCAATAACCTTGCTGGTGGGGATGTGTTGGATACGCTGCTAAT
GCCTGTTTGCGACAATGCTCGCTAGTCCCGGTGGTGGCGGTGTTCACAGGTAACAATGTTTACCA
CCGTGAATGGAACTTGTTTGATTAACCCTGATCAGAGGATGAAAACACTAAAGAACCAAGTGAGA
AAGAGGGAAGAGAACCGCATAGGGAAGAGCAGAGCGAGTAGACGAGCCGAACGCAGAGCCCGCGA
GGGGCGAGTGGAAGCTCCCTGCGGGCAGGTACCCGACCACCGCCCTGCCCTGGGCGTGGCGGCCT
CGGGCTCAGGGACCGCTTCGGCGCTAGACGGCCGCGTCCGGAGGAAACGGGCGCTGGTGAAAGCC
TAGGTGTCCTGGTCCACGCGCGCAGCCGGACGTCGGGTCCAGGGAGAGACGCGGGCTGGGGGGGG
ACGGGACCCGGCCCCTGAAGCGCGAGGGTGGGAGTGAAAGCAAAGAGGAGAAAAGTAGAGAGAGA
GGACAGTGGGGCCCAGCGCCGCGCGAAAGGCAGGAACCGACCCGCGGACAGGAACGGGTGCAATG
AGGTCCCCGGCGAGCGTGGGAACACAGGGTTCAGCCTCCTGCGGCGGGCGAGCACGCGGACCCCA
GGGGCGGGGCGGTGCGACAGGACGTGACCTGGAAGCGCGGCGCGTGCCACCGCCCTGGAGCAGGC
ATCCGGCCTCCGTGGAGCGGGCAGGCGGGCCCCGGGTCTGGAGCCTGCGACTGGGGAGGGCGCCG
CGTCAACAGCAGCGGTTGCGGGGGGGGGCCGAGTGCGCGTGCGCGGCTCTCGCGGGGGGGGAGCA
GGCGGGGGAGCGGGCGGGAAGCAGTGGGAGCGCGCGTGCGCGCGGCCGTGCAGCCTGGGCAGTGG
GTCCTGCCTGTGACGCGCGGCGGCGGTCGGTCCTGCCTGTAACGGCGGCGGCGGCTGCTGCTCCG
GACACCTGCGGCGGCGGCGGCGACCCCGCGGCGGGCGCGGAGATGTGGCCCCTGGTAGCGGCGCT
GTTGCTGGGCTCGGCGTGCTGCGGTGAGTGGCTCCTCGCTCCCAGCCCTGCGGCTGCTGTCGCTT
CGCCCCCGCGGGCGTGTGGGCTGCGCCCCAGCCAGCCCGGCGGCGCCCTGAAGAGGGTGGCCGGG
GCGCAGAACACTCGGGCCCTGAGCGCCCGAAGTGCAGACGTGGGAGGGCCCCACGGGGAATCGGG
CGCCCCCCTTCTTCCTCCCTTCCTTTCCCTGGTCGTCTTCTTCCCCCTGGGTGAGAGCGGGGCTC
ATCTCCTCCACCCGGTTCTCCATCTCCAAATGCACACACACAGGAAGACTCCAAACCGCGCACCT
CGCGCAAAAATTAACTAGCAAGAAAGGGCGCGATCAGAGGCAAGGGGCTGCAGCTGCTAGGGATC
CCCTTCTTTCGCACCCCTCTCCCTTCTCAGTGCTTAGACGCATTTGGGGTTGAGGGAGGGAGAGT
GGGAGGGCCCGGGCCTCTTGCGATGGAAGTGCGCATTTTGGCGAAGTCGGTGAGAAGGGGTTTCT
GCCGTTTGCCTCCCACATGAACATAGGAGCGAAGAGGACGTAAAGGACACAATTAAGTTTCATTT
TCAACTCAGCATTCAATCAGAAGAATCTTCCGGCCGTGTAATTTTTGCTGTCGTTTTTAATCCTA
AAAATAAGCTTGCTGGAAACTCTTCCTTTCTCGTGACCCTCACGCCCCATCAGCCACTTGCATAC
ATTCTAAATTGTACGTTGAAGTTTTTCCCACTTTATTTGGGGGAACCGTTTTAAAAGTAATCTGG
TTTTCGCCTGAAATAGGAAGACAGTAACCTCCAGTCAAAACATGTGCAGCAGAATGGGATTTGGT
GTTTTTCGACGCCAAAGAACCTCCACCCCCCCACCCCCACCCCGAGCTTTTGGAATCCATTTCGC
TTTTGTAAAACGTGTGCTTCGTCTGTAAAAACTGAGCAAGGAATAGAAACATTCGTAGCTCTACA
GTGAGTGCCTGTCACACTTCACATCCATAGAGCCTTTTGTGAACTTTTATAAGATTCAGAATTCA
GCTTGAGCACCAGTGACAGCAATTGTTACATTTATTCTAGAATGCTAAATTAATTCGATTTTAAA
CTGATTATTAGCCTCGGTGTGCCGTTCCTAAAGGCTTGTGACTTTAGGTATTTCCAAGATGCCCT
TAAGTCTCTGCTTACCACTTTCCTCCCTCCCCGAGCATCCTGGATGTTGGGACTGTGAATCCAGG
TCTCCGTTATCTAAATGGTTGATGTTAGTGTTTCCTGTCATCACGTTTAGTATGCTTGTTGCCTT
TACTATCATTATAGCTAAAATAATACTGCTTTCAGAGATGTGTTGTATAATCGCATAATAATTTC
AGAACGCCTTCATATTGAACCAGATATGAAGTGATACAGTATCATTTATTCAAACTGCCTAAAAA
GTAAAAAGTATAAACCATATACTATTTTTAAAACAGGTAGGTTAGATTTAAATCACGTAGTTTAG
AACTGTTGGAATGGTACTTTGAGTGATAGGATTTATGTTAGGCCTTCTTTAGTAATAGTTAATAT
CATGTAATTAGCACTCGTTTACACACAATTTTAATGTGTTCGAATCCCTAGAGTCATCGAATTCA
GAATTTATAGTATTTTATTTTACTTAGTTAATATTAACCTAAAAAAAAAGCAAAATACAGGCATT
ATGCAGTTGAGTTGTGTTAAGTGTGGACTGTAAAACAGGATAGTATTTGTTATAAAATATGTTTT
TGTATGTGTTTAATATATAGCTTCAAAAGGACATGTATGAAGAAAGATGTCTCAGCACATAGGTA
GTTATAAACCAAGGGTTTGAGAGACTGACTTTAGAATCTAGAATGAGTAAGAAAGTGATGGATCT
GTACATTCATTTGTATTGAAGTTTGATTTTGCTTTCAGTTTCGTTAATTAAGCCCCGGGAATCAG
GGACCTTTCCTGGCACCCTCTACAGTGTTAGTGGCCTTTGTGGTAAAAGAAGTTATCTCAGATAC
TCATTTCATGCAATTACAGGCAAACTGGAAGGCACCTTAATGGTATGCAAGATGTGAAATGAATT
ACTATGTTGCATTCCACTCTGCCCCCACTCATGTACAATTATACTTTGTTTAAAAGTGCATAGTT
TCAGTGGTATTTATTAGCTAGCAAATAAAACTTTAAATAAATAATGATGGTGTTGTGAACTGTCT
TTTCAGTGGTTGAATGATTCCTCTTTGTTCAAAATGAGTTGTTTTTTTTTTTAAATCAGGGTACA
TATGATTAAATAAAATTTTTTTCCATCGTCTAAGTCTAGTAGTAATTTGGCTTGTTTAAATAAAA
ATGTTTTCTCTTAAAGAATATATTATTTTTAATTACAAGTTGCCATTTTAAGGAAGTAAATGCCT
ATTTAAAAGTACGTATTTATGGGGCCGCCCCAGCAGTCTAGAGGCAGTGTTTTTTAAAGCATTAC
ACTAAATGCCTACCATTAGGACACTTGCTCATGCTGTCATCTGTGTTTTGGCAGATGTTTTATCT
TTGTCACTGAGTTGTTCATGGAGATTTGAAAGGCCAAGTTCTAAAGATGAGAGATAGATGACTTT
CCCCCCAAATTGATACATATTCTAAATCCAAAATAATCACGCATATTCGCAAAACTAGATTTGCA
TTTCATAGTATGAATTCAGCTATAGCTAGTATATAGGCATTGCTTTTAATATAGTAATGTTCTTT
TTGTGGCATAATTTTCAATCACTTTCCCTGTCTTAGGTTTCTGCCTACCTTTTTATACAAGATAA
ACATTTCTATTTTCATATCTTATCTCTCAGTCTGATTTTAATAATATCGTCTTGGGTTATCATAA
AGTTACTTTCACTTTGTATACTAGTGTAGATATTTCTTCTGTGATAAAAAGAACAAGAATAATAG
TGTAGGATCAGTTTTGTTAAACTCATTGTAGTACTAAAGTAGAAAATACAGAGCACCCAGGAAAT
TTGATTCTGATATACCTAGACTAAGACAATAGAATCTGGGTTTTTCCTCTTTACGATTATACAGT
GTTGAAACAGAAATCAAGCTACACCCACCTCTGAATATTTCATTCCAAATCCATGGAGCAGTTGC
ATAAGACTCAGAATCTGGTCAATCTCATGGTTTGAGATTCAGATTAGGAGAATTAATGTGCATAT
TTGAATATATATCACACTTGTAGGTTAAAAGGTAAAGCAAGGGGTTTGGCTAGTTCAAGGCCTGT
CAAAACCACTTTTTTCCATTTCAAGCAAGTATGAGCCTATACTACACACCAAGCAGTTTGCAAAG
CCCTCTTTGGCATATAGACAAATTTCAGACAGTATGTCAGTATGTCTCTTAATGACTTAAGATGT
AGTATATAGACAGTAGGGATATCTTGTTTTTATTCCCTCATGGTACTTAGGTGCTTGTTAAGGCC
ATAATTCTGGATGTTATTGACTTTAAACTGCCCTGCCCTTTTTAATTCTAAGTTGGCCTCTTCCA
TTCTTATAAGGCCACTTTTAAATTCATTAGGGGGATTGCGTTGGGTCAGAACTAAGTACAACTTG
TGGACACCCCACTCTACCCCAGACCTTTATTTTAGTGTCCATTGAGGAGAGATCTGACTTCATCC
TTTTTGTACGTAACCCAGTGTAGCTAGAGACTCCAGTTCTTCAAGCCAGACACTAGTTCTAAGGG
GCAAAAGTAGGTAGGATTGCTTTCTGGCATGTGTCTTTACTGATGATCTTTGCTTCTTTACCTAA
GAGGCTGCTGAGACTTCTTACTCCATTCCTGAACATCCATTCCCAGCAGATAATTAACTTCCTAC
TCTACTGCAAAAAATAGTGGTGTGTGGTTTGGACTCCCTCTGCTTCCCTCCCACCATTACTAAAC
TGATCTATAACATCTTAGATAACCTTCGTCTCATTCTCTTCCGTCATCCAGCTTCAGAAGAAAAC
CTACTCACTTTTCCAACGAAAGGACAACCTGTCTACCACCTTTTCACTTCTACCATCTTGGAGAC
CTGCTCAGTCAGCATAATTCTTCAGTCTTGCATCTTAGATCTTTCCCTCTCCATTGACTCCTTCC
CCGTAGCTTGCACACATGATCAAATTTTACCCCATATTAAAAAATAAAACAAAAAACCTCAAGAA
CCAAAATGTCCTTGCCCTTGTTCCTGCCTGCACATTGTCAGTTGTTGCCTTCTCTGCTTTCTTAC
ACTGCCAAATTTATTGTAAAAGAATCACTGCCACTTAATATATTTTAAGTGCTTGATAAAGCCAT
CTTGTTTCTCTCTCATTTCTCAAACATAGTGTGCATCAGATCTCACTAGTCCATTGACAGATGGA
AAGATATTCTCCCCAAATATTTAAGCCTCTTTTCTCTATAATTAGCCTACCATCTAACTAGCTAC
ACATACTATTTTGGTTCACAATTTTAATTTTCCCCTCTTTATCTCCCCTGCTTCCCACAACTAAC
TTCTGTTGATTACTGTAAGTTTTAGGTCTTCTCTCCAGTTGTTCTTTACCTGGACTATTGCAGTA
GTCTGTTAACGGATCTCCCTTTCTCTTTTCCCTTGCTGCCCTCAGTCCTTCCTTTACACTGCTGT
CAATGTTTTTTTTTTAATGAAATTAAATCTGAGTGGTTTACAGTTGTTTATGTTATCAAGTCTAA
ACTCAGCATGACATTCAAGGAAGCCCTTTAGAATTTTTCTTATTCATCTTTTCCAACCTCATTTC
CTAGAGCTCACCTAAATAATTCTCTGCTTTCAGTTCTTCACTTTCTTTATCTGGCTGTACACGTT
ACTCTGCCTAGAAAGCCCCTTCTTTCCCATAACATTCTTACTCATCCTTTAAGACCCAGTTTAAA
CATCACCCTCTAGGAAGATCATTCATAGACACATTCTTGTGATTTTTAAGGAACTTTTCTATCCA
ACCATACAAGTGACTTGAGATTTTCCATGAAAATATGCAGCTTCATGATTTCATAATCAAGTCTA
TACAAGTGAGAAGCAGTGGCAGGTTCTCTTGAAATACAGAAGAAAAATCTATCTTCTCCCACATG
ATTTTTAGATTTTTCTTCTATGGAATTTATACTTTAAACTTTTTACATTCACAAGGAAGGTATTG
AATCCTACTTTCTGGACCCTGTGTTAGATTCTTAGGATACCAAATGAAGACAGTGTCTTACTTTC
TGAGCCTGGAGAGATCAGTTAAGTAAAATAGTAATTACACAGTAGTAGGTACAGTGACAACATTT
GAACACAAGGCAATGGGAGCAATAAGGAGGGGAAGAATACAATTGAGTAATGGTAGGGAAGGGGA
ACAAGATTGAACAACTCAACTGTGCTTTAGTAGGAAGAGCAAAAGTTAGCAAACTGTAGCTGCCA
TTTATTGAGTACTTGCTGTATATGTGAAAGGTGATACAAAAACATGTTATCTTATTAAATCTTAA
CAATATCTCTATGAGGTATATACCATCACTATGCACATTTTATAGATGAGGAAACTGAGGAGCAG
AGGTAAGTAACTTGCCTAAGGTTATACAGCTAAGAAGTACATGAGGTGAATCTTAAACTAGGGTC
GACCCCTTAATGTGTGCACTTAACTATTATACACCCTATGTACTCAGTGGGGTAATAGTGTATAA
CAGTTAAGAGGCTATTTAGGTTTGAGGAACAGTATGTGCTAAGGCATGGGGATGAGAGTGCATTT
GCATAGTCATGGAATTGCAATAACGTCATTATGATTGGAGCTTAGTGTGGAAATGAGGGAGTACA
GGAGGTGGGGCTGGAAAGGATCTTGAAGGACCTCGTTTGTATTCCATGCTAATGTACTTAGAATT
TAGCCTGAAAAGGGTTGAGGGTGTTGTTAAAGTATCTTAGGCAAAGGAGTGCACATTTGCATTTA
GAACAGTTATTTTGGGAACTGTAGAAAATACATCAGGGCCAGGATGGAGAAACCTTGAGGATGGA
GGCAGGGAGCTGGTTAGAAGGGTATGGCAGTAGTTCGAGGGGGGAGGATGAGTGTCTAATTCATT
CATTCAACAGACATTGGATTTAGGAACTATTAATTAGGTTGAACCTACTAAGAATTCTCCATGGC
TAAGGAGAGGGAGTAATCTAATGTGATTCTTAGGTCCAGGTCTGGTTCACTGGTAGATGATAGAA
AATACTTTAGCAAAGTGATTTAGGCTGAGGGTAAGAAGTGGGGGTACGTAAGTTTTTGGACATAC
TGACTTTGAAGTAACTGTATTACATCCAAGTGGAAGTGCACAGCAGACAGTTGGCTGTCCAACAT
CTGCAGTTTAGAAGAAAGGCAGTGCATGATTCAGTTTTTTATTTAAAAGTTTGCAGGTGAAGAAA
ACCATGGGAGTCAGGGAAAATACTAGGGGGCAGAGGGTACTGAATCTGAAGCCCTTAGGGACACT
AACATTTAGTGTGAGTCAAGGAATAGATTTCAACAAAGAAAGCAAACTAGGATGGGGTGGACAAA
GCAGGAGGAAATTCGTAATCAGGAGATACCATGGAGGCCAAGAGAGAAGAGCAAGAAAGAGACTG
CCCACTGGCTCAAGAACTGTGGAAAAGTCAAGACAGAAAGTGAACCATGCATATTTGATTTTTCA
CAAGGGATTGGTGGTGACTTTGGCAAGAGCTGTAGTAATGAAGAAGTAGGGGTGAATGGGTGGAG
GCCACGTAACAGATTTAAGGAGTGAATTGGAAGTGAGGAAATGCTTATAGATTGTTAATTTGGGA
TGCTAGGGAGAGAGAAAGTATGAGGGATTTTTTTTAAGTTGGCAAATATTCAAGCAATTTTTAAA
GACACACACAAAAACAACACCTTGTAAATGGAATGGGGGATGTGGGAGGCTGATAATCAACTAAG
GAAAGTCGTTGAGGAGACTGATGGGTATGGAGGCAGAAATGGAAACTAGCTTTCAGGTGATGGGA
TTCATTTCAGAAAAGGAAGCAAAAAAAAAAAAAAGGTGTGGATAGTTGGGGTTACAGGTAGGTTT
ATAGGGAAGGAGTATTGGAAGCTTCAGGATTCTCCTCTGAAGGTTTCAGTTTGTATTAAGAAATA
GGGAGAGAGGACTGTTGGAGAGTATGGGATAAAGGGCAGAAGGGAGTAATTTAAGAGTTGTTAAA
AAGAAGCGAACATTTACATGAAATATGTAAAATGGTAAAGATTGAAGGCCCGGAGGAGCAGGGAC
TATGACATTCTTCTGTTTTTGCCTATTAGCAACATGAATTTTCTTAGAAATCTGTAAGACCAATT
ACTCTTCTGCCCATCCATAAGGACGTTGTCCATGCAGAAAAGATAACACTTGTAACCTTGTATTA
TATACTTATCATCGCCCATTTGAGAATTGTAAGCTCCTAAAAGATAATAACTATATTTTTTCATC
ACTATATCCCCACATCCTATCACAATATTTCATCACAGGTAATTCTTGACAAATGTTGATTGCAT
TTTTAAAATTTCTAACCTGAACTTGTGTGCTGTGACCACCATGGATTGAGTCTTCTCTGCCACTA
CAAAGCTCTTTTCTAGACTATGATATGAGATGGTTTGGGCTGATAGTCTATATTCACCAATACTT
GTACAGTTCCAATGAAGGTTTCAAGTCTAATACTTTTGGCATTTGATATAAAATCATTTTCCCAT
TTTATTTGCTAATTTATCTATAACTCTGGCATTACTCTTGGTTACATTTGTCTGCTGCTATCTGA
TTTAGACTGCTATAAGCATATCTTGTTTATTCAGAGTTTTCTTATTCATGTTAATTATCTGTGTT
TCTTTATTTGCTCTCTACATTTTAAATAGTTTCTTCACTTTGCTGTTTTATGAGAAGGGAGTAGG
CAAAAGGAAAAAACCCCAAATCAGACAGTTTTACTACTTAATAGTTTTTTAATGCATCTTTATAG
AGATTGAAGTTGTAGTTAACAGCTAGAGTGATATTTTTGGCCTGCCCTTATTTATTAATTACTAG
TGCAAAGGTTATTCAAATTGTGGTTTATCCAGGTCAATTTTACTGTTATTTTTACTAATAGCATT
TATTCTACTTAAATTGCTTCAGCTATAAAATGTTTTTATTGTAACAAATAATGCAGTATAATTAT
TGCTTTCTGTATTCCTTTGAAAATTCATTCTCTAAACATATGTTATGAAATGGTGGATTGTTCAC
CAACTACTTCTTACTTACTTAATTAAAGCCTTTGCAAAAAGTTTCATAGGATGACTGAGTTCTTC
ATTCGTACCTCTTTTTTTTAAGAATAATCTCTTGAAAGTCAAACCATGATTCATACAAATATAAA
ATGAACATGTGTCAAAGATTTTATTTCACTAATTAATTAACAAGCAAACTAGCAAGAAGGCAAAA
CCCTTTTAAAAGAAAATTGGAAAAACAGACACATTTATAGGCAATCAAGAATGCCTAAATGAATT
TGCTAATAAATGCAAAATTGGTCAATATCCTCAGGGCAGTAAAATGTAGTCTTTTGGAATCCTCT
CTTCCACCAGGTGGAAATCAATTGCAGAACAAGATTTATTTTTATCTGTATGGACAGCAATCCAT
TTGAATTACTCTCAGTTTTCTACAACTTATGAAACTGTAAAACAGCCCAGTCAAAGTCAGTGAAA
GGCACAGGCTTCTATAGAGTCAGATAATTCCAAAGCGTCTGTTAGACATTGCCCAGCACTTGACT
GAAAGGATACCCAGTAGTCTTTGTATCCCTTTCAAAATCTTCACCATATTTTCTGATACTTTCCT
TCCTTTATAATTTGAACATCTTCACCCCTTTATTCTTCTCTGGTATACTATGTCCTCTCTCCCTC
AGTGTTTCTCTCTCAGGGGAAATAATTCACAATTCCAAAGTTTTAAAGAATGCAACGGAAGTCCA
GGTTTTGCCTTGGGCTTCCTAGTATTTGGGTGGCTAGAAATGTAGAAAACTGGGAAGAGGTGAGC
TGTGGATGCCCACAATATAGGTTCAGACTGCAATTTCCCAGAAATATAACAATTTGGACTAGTCA
AAGAGGGCCCATATCATTACATTAAAATGCCAGATTATCTAGTTTTTATAGTATCACCCTACATT
TTTACAGCATAGTATTGTTTAGAGAGCACTTGCCGCTATGTTTTCCATGTTAATGCTCAACACAG
TTCTGTGAAGTAGCCCGGTGTTTGTTTTTGATCCTCCTACTTAAGAGATCCTCCTATTTTAGAGA
GTAAACCAAGGCATAGAAAAGTGAGTTGCTTAAGGCAGCATATTAAAAAGGGGCAGAAATAGTAT
TTTTACTCAGGTGTTTTGAACATTGTCCAGTACCTATATTCCAGTATGCATCCTTGCATTCAATT
CATGGGGTATTTATTGAGTAAACATAGTGTTCTTACAATAGAAACATTCTATGCCTCTGATTTTT
ATTCCAGTTTATGTAGAAGTAAATCTTAAGTGTGAACTATTAACAAAGTTGATATTTTATTTATA
TTTGTTAGTAATTTGTGTTTTGTTTTTGTTTATGTTTTGAGGGGAAGGCCAAGTAGCTACTTAGG
TAAAAGAGTTGCTGAGTGGCTGAAGAATATGGAAGACAACTACAATTCCTACACATTCTTGTACA
TTTTAGTTGAACAATGAGTGATTACATTTATTTACCCAGTGCCTCTTCTATAAGGCAACAACTGG
TAGTTTATCCTATTGAGAAGCGTAGATAGGATGCTTATTAGCAGTAACTGCTCTTGGTTTCAACT
TGCATCTTACTTAGCTTTTTCACCGTTTTGTGGTTTCTTGGAGGAAGAATACCCATAATATACAT
TTGGAGACTGTTGTTCTGTAGTGTCAATGAAATGTGGGGGTGGGAAAATGTCATTCAAGACTCCC
ATACAAAATGTCTATTGCTGCCTATATTTTGCTATGGGAAAGTAGCCACAGATAATGTTTTTTTT
TTCCTCATTAGTATTTTAAGATTTTCCATCCTAGTGGAAAGATATGATTTGATTCATCCTATTTA
CTTTGTATATTAAAGTACAGTAGAACCTGCCACTTTTTTTGGAAATGCAGCATAAGGATAAAGAT
AAATTTCATATCAGTTCAGCAAGTTCTATTTAGCAGTGTGTTGAAGTTGAGACTGAATAAAATAT
TTGGTTTGGTTTTCTGTTCAAAATTTTACCTTGATAAGGACAATATTTTTCTACATATATCAGTA
GGCAGTAATGATTACTTCAAAGCTTCCAAAGCCAGATACTACACCTGCATGTTCCAACATAGTTG
CTGAATTTATTCCCAAGATGCATGTAATGTATACTTTGTATTATTGAGAATGAATAAAGAAAAGT
CATAATGATGCCTTCCAGCTGTGCAAGTTAATATTAAAATATAATTTGTTTGCATATTTCACCTA
ATAGGTCTTCTTCATTGCTATACTGTTTACTTAAGTGAACAATGGAAATGTTGCTGTTTATCTTA
AGGATTTGTAACATGCCTAAGATCTTACAGTACAGACTTCTATAATTAATGAAACATTTTTCTTT
TTCCTTTCCAGGATCAGCTCAGCTACTATTTAATAAAACAAAATCTGTAGAATTCACGTTTTGTA
ATGACACTGTCGTCATTCCATGCTTTGTTACTAATATGGAGGCACAAAACACTACTGAAGTATAC
GTAAAGTGGAAATTTAAAGGAAGAGATATTTACACCTTTGATGGAGCTCTAAACAAGTCCACTGT
CCCCACTGACTTTAGTAGTGCAAAAATTGAAGTCTCACAATTACTAAAAGGAGATGCCTCTTTGA
AGATGGATAAGAGTGATGCTGTCTCACACACAGGAAACTACACTTGTGAAGTAACAGAATTAACC
AGAGAAGGTGAAACGATCATCGAGCTAAAATATCGTGTTGGTAAGACTTCTATGAAAGCTTCTTT
TTTTATTTGTCCTGGTGCAACCTGATCCTCTTTCGAGAGGAGGCCAAATGGGGATAGGTACTCCT
TTGAATCAAAAAGCAGGCTGTTATTAATAAATGTTGATGTAATGTTTAGCAAGTTTAAGATTGTG
ATTTCTATCCTATTTGTTAATCTACTCTTCATGGTAAAACACATTTACTATTTAATTTGTATTAC
TTGTTAATTTGTATATTGTGTGGTTCTCAAACTTTTGGTCTAAGGACCATTTTATTTGGCTGATT
GGTCCTGGGGTAATGTACTCTTCATTTCTGTTTGCTGCCATCCAAGTGCTTTCTTTCCTGTGACT
AATTTTCAAGTCTCTTTCCAATTATTCAGCCTTCTGTTTTTTGTGTTTTTTTTTTTTTTTTTTTT
TTTTTTTTAACCTTACACTTTGACCAAGGGATGACAAGTCCCAGCCATGCTCCCTCTGGAGAGAA
TAGAGGAGAGTCAGGAGATTAAATCACGTTATCATGGATGATTTCTTCAGATTTTCTGTGCTTAG
CCTTACTTTGGTCTTCTTTTTGTTGGCCAAAATATGAGAGAACTAGAGATTCAAATTCATTTATT
CAAATTCAGACTTGATGATAGCACCATGATTCTGCCCTTTTTATCACAAACTTGGGTTGCTGAGG
TGTAGAAGGTAGTAAAATTAGCTGAAGTAAATAGTCTCCTCTCCTTGCTGTTTCCTGCTGTGCTA
GCTGAGTTCTTCAACCCCATATGAGTATGTTCTTTCTGTTTTGAAATCTCTATTTAGAATTACCT
GTCATATTACCGATCATGGCTTAACCACATTTTGAGAACCTTAACTCTAGATAGGTTCAGTTCTG
TTCAGTTGGATCAACTATTTGCCTAGCTTTTATAAAATAATCTACTACCAGATTAGGAATGGGGT
GTTTGGTTTGATGAAATGTTTGCACATTACTTAGGATTGAGAATCAGGAGGTCTGAGTTACAGCC
CCTGTTCAGCAACTTACTATTCACCTGTGTGATCTTGAGAAGGTGCTTAACCGAGTCCAAGTTAC
CTCATGTCCGAAATGGAGATGACAATAGATACTTGCTTTTATGTATTCTACAGTCTAATGAGCAT
AAACCAAAGTGTGTTGTAAATGTTAGTTGTTTTCCTGGGATGTTTATTTTATGAAGTAAGGTATC
AAGTTGTTGAAGTTAGCCTAGAGCATTGTGAGAGGGATTTGTGTGCAGTGAGGAATGGGCAAGGG
ATGGCTCCGAGTAGGCAAGGAGAATTTGAAAGGTCTTTCTGCTCAGAAATCTCTCTTACATTGTA
GCTTTTACTATTGATATTAATTTGGGCAAAGGAGCTTTTTTTGTTGTTGTTAAGTATACCTTTAA
TGAAGGGTGTAATCAGTCAGTCATATCCACTGCAACTAACTGGTATTAGATCATCTAATACCTAG
TTGAAATTTTTCTGAGGTAATGTACAATTTTGTGGAAATGAAAGAATAAGATATGGGGGAACAGT
AACTTTTGGGGGGTAATGCAGGTCACTGAGAGGTAAACTGGAAAACTCAAAATATGAGATTTGAA
AGGCCAGAAGATGAAATGAAGATGGAAGAGAAAGGAATGCCAGGATCTGAGATAAAAACAGTTTA
TTATTTTGCGTGTGATTGAAAGACTGTTTTATCTTTGTTTGCAAAATACCCTATAAAAATAAAAA
CTAGACTTATTTTGGGAGCCATTAAAAGGAGAAAGTATTGTTTTATTATAGAGAATGTAGAAAAT
TCTACCAGAGGTTTGAGACATTTGGCTTTGCATCCATTTTAATTTTTTGAAATAATTATTGATTT
ACAGGACGATTCAAAGATAGAGAGCTTCCATGTGCCCTTCACCCAGTCCCCCCAATGGTTTCCTC
TTATGTGACACAATATGAAAATCAGGAAGTTCACAATGGTGCAGTGTGTGTTAATGGTTCGCTCT
CATTTTATCACGTGTTTATTTGCGTAACTACCTACACAATCAGGACATAGACCTATCCATCATAA
CAAACATCTCCCTCATGCTTATTCTTACTAGTCATACCGCCACCTCCCACTGTCCCTAACCTGGC
AAGCACCAATTTGTCCTCCATATCTATAATTTTGTCATTTCAACAATGCTATATAAATGGAATGA
TACAAGATGTGACCTTTTGAGAAGGGCTTTTTCACTAAGCACAATGCCCTTGAGATCTATCCAAG
TTTTTGCATCTATCAATAGTTCTTTTTTCCTTACTTTTTTTTTTGCTGAGTAGTAGTCCATAGTA
TGAATATATCACAGTTTGTTTAACCACTTACCTATAGTAGGACATTTTGGTTGTTTCCAAGTTTG
GGCTGTTACAAATGAAACTGCTCTGAACAATTGTGTACAGGTTCTTGTGTAGACACAGTTTTCAT
TTCTCTGGGATAAATGCTCAGAAGTATGGTTACTGGGCTGTATGGTAAGTATATGCTTAGTTTTT
AAAGAAACTGCCAGACTTTTCCAGAGTGGCTATTTCATTTTACATTTCCATCAACAATGTAAGAG
TGATCTGGTTTCTCCACATCCTCACCAGTATCTGGTGCCACTATGTTGTAACCATTTTTAACTGA
AAAAAACAACAAACAAACAAAAACCAGTTAAAAAGTTATCTGCACAAAAAGGTTAAATGGGGCAG
GACTCTTACTATGGAGTATAAGTTCTTTTCTAATAAGAATACTTACTTGTCACGGACTTTGAGGA
TTTAAGCATTAGAAACCATTTTTACTATGTCGTGATTTTTGCAAATACAGGCATAAATGAGAAAC
AGAATTTGCTCAATAGAGAAGTGAATTCTACTTAATAGAATGCAGATAATAGTGACCATTCTTTG
TGCCTTTTTAAATTTTTTGGAGGACTAGGACTGAATACATGGAAACACTATAAGATATAGTTTTA
TATACCTCTGTGCCTATGTATGATATAGTCCATTAGAAGGAGCACTGGACTTGAAATTAGAAAGC
TGTTTTCTAGACTTCACTGTTTAATAGCTTAAGAAAAGTTGGATAAAAAACTCAACCTATCTGTC
CCTCAAATTCTTCTTAGTACAGTAAAGATGGCATCACCATCATGGAAATGTTTGAGATATAAATA
ATACCTCTCTTACTTAACAAAATTTTACTAAGTGCCTACCATGTGCCAGTCACCGTACTAGGCAT
CACAGATTCTGTGATGAATAAGACATTATTGCTGATTTCAAGGAAGTGGTTGCAGTACAAATATG
AAGAGTGAAGTGACCTAACATTTATTTGCACATGTGCCACATACTTTATGTAAGTCGTGTCCTGT
TATCCTCTCAAGAACTCTAGGAGATAGATGGAGTTCTTACCATTTAACAAATAGGGAAGCAAAAG
AGATTCAAGGTATTGTTCAAGGTCAGAGTAATAGCCAAGAATTAAACCTAGATCTCTTTGGCATC
AAAACCCAGTATTTTTACCACCGTAATATGTGGTTCATGTGAAAACACCTTGTAAATAACTAAAT
ACTGAATGCAAATACTAGTAATCATAAAATTTATCATTAATATTTCTGTTCTATTAGCAATACAA
AAACTTGAAAACTTAAAGTTTATTTTTCTCTAAGCTATTAGAGTTTTGTTTAGAAAGGTCAGTTC
AAGTTCCGCAGTGTTGCCAATTCCATTATTATCAACAGGACATATGGTCTGTTTATAATGAAGAC
ATGAAGGCATTGCAAGATCTTTGTATTAGTTTTCTAGTGCTGCATAACAACTACAAATATAGCAG
CTTAAAACACTACCCATTTTTTAGCTCAAAGTTTTGTAGGTGGCAAGCCTGGTCATGGCATGACT
GGGTTCTCTGATCAAAGTCTTAAAAGACTAAAATCAAGGTGTTGCCCAGGGCATACTTTATTTGG
AGTTTGGGGTCTTGTTCTAGGCTCACATAATTGTGACAGAATTCAATTCCTTGTGATTGTAAGAC
TGGGGTCCCTGTTTCTTTGCTAAGTATCTGCCAAGGAATTGTAGCAGCTCCTAGAGATTGCCCTC
ATTTCTTGCCCCATGGCCCCTTGCATATTTAAAGCCAGCAAAGGAGAATCTTCCTCTTGTTGAAT
CCCTCTCACATATTGAGTTTATTTTGCCAGGAAGAACCCAGACCCTTTTAAGAGACCACTTGGTT
AGGTCAGCTCTCTCTCCCTCAAAAATAATCTCTCTTTCTTAAAGTCAGCTGATTTGGGATCTTAA
TTACATCAGCAAACTCCCTTTTGCTGGGTGATATAATCATGAAAATGAAATCCAATACATTCACA
GTCCTAGTCTCCCACACACAAGGGGAGGGAATTATAAAAGATGCATGGCTAGGGCAGGGGTTTTG
GGACCACCTTAGAATTCTGGGTTTGATTAAATAAATAATGGGACTGTAGCCTAGCAAGTCGACAC
ATCAGAAAAGCCATCACAATCCTGAAACATCAAGTAAAAATTTGGTTTGCATTTTAGGATTGTAA
ATGTTACTGTGGTGTATGCGTGTGTGTGTGTGTGTAACCTGGTGTTCCCTATGTTTGTGGGAGTT
TGAAGGAGACACTTTGTTGATAGGAATGGTGTCTTCACTTTTTTAGGTTGTCTCATTTTTGTACA
GTGATAAGACAAATGAGGTCCTGGGTTTTAATAACACTTCAGCTTGAAAGCAAAAATTAAACACT
TATTCATTGACTACACCCTTGTAATATCACTTCTTTGCCTTTCCACTAGCAAGAAGTTCATTTTC
GTGGAAGCCATTCTGCCTGTCCAGGAATTGGAGGAGAGTGATAGACACAGTTGTCAGCCATAGCT
TGGGTAGAATAAGGATGTGAATGTCCTTGGCTTATCTTTATTAATCTTGTGATGGAAAAATATCT
GACATTGTTCTTAGTCCATTTTAAGCTTAATTTATGTTCTTAGTGGCATAGAAATTCAGAGCTGA
AAGAAGTATCATGTCTCACTCTCCCCTTGAGGAACAAGAGTTAACGTCATCTGACAGTACTACCA
TAACAAATCAATAGCTTAATAGGCATATAAGTGGCTTTATATAAAATGTTGGTTTTTTTCCCCCA
GCATCTCAGTTGGTTCTTAAATATCTAATTCCATGATCCTCAAACTTTTCCCACTGTAACAAATT
AGAGAGAGGAGGAACATGTTCATCAGTGACTGTAGTTCAAATACAGCAGAAATGTGCAGCAGTGA
TTTCAAACTGAGAGAATCCTGGATGCCCTTCTCCATTGTGTGCCCCCCCCCCCCACCCGCCCCAC
ATATCAGGGAACAATTTAAAATCCTGGCACAATAATGAGAAGGGAGAGTGACAAACTGATAAGTT
CCAGTTAAGAATCACTTAGACAGGCCAGGTGTGGCAGCCCACGCCTGTAATACCAGCACTTTGGG
AGGCTGAGGCAGGTGGATCACCTGAGGTCAGGAATTTGAGACCAGCCTGGCCAACATGATAAAAC
CCTGTCTCTACCAAAATTACAAAATTTAGCTGGGCATGGTGGCGCCTGCCTATAATCCCAGCTAC
TTGGGAGGCTGAGACAGGAGAATTGCTTGAACCCGGGAGGTGGAGGTTGCAGTGAGCTGAGACCA
CACCACTGCACTCCAGCCTGGGCAACAGAGTGAGACTCCATCTCAAAAATAAAAAAAAAAGAATC
ACTTAGACAGTATTTTTTGTTCTGTAATCTTCCTCTCTGTCATTGAAATGTCTCATATTTCTTGT
TTCACATAGAATCTGGGAGTCATCAAGTAGTCATCTAGTCTATCTCATCATCTAATTTACGAATT
ATTTCTATCACACCCCAAGTAGCTGGCGATTCAGATTTTTTACTGAAAGCTTGACATGATGAGAC
ATTATTACTTCCCAGAGGATCTTGGTCTGTTATCAGGCAATTTCAGTTCTTCCTTAAATCAAGCT
AAAACTTGCTACTATTCTCACTCACTGGGAGGGCTGTAGTATTTCTCTCAAGTTTATACCTCTCT
GTGATGAAAATCTCTGTTCTTTTAGCCATTGCTCATGGTTTCGGGATCCCCATTCTAGTCATCTT
CCTTTGGAGCAATTCCCTGTTAGATCTTCAAATGTGATGAGTAAAACTGAGCTCAGGACTGCGTT
TGTTGGTCACACCCTTCTCCCTTCCAAATGACTGCTGCTGTAACTTTCTGACAGGTCCTGCTTCC
CTGTAATCCATTCTCCATGTGGCTGCCAAAGAAGATTTGAAATAAGTCAGGTTATTTTTCTTCCC
TATTTAAAGCCTTCCAGCCGCTTGTCATTGCTCTTAGAATAAAATCCAAACTCTCCCAACAAGCT
TTTAAGACTCTACATGGTCTTGCCCCAGCAAACTCTTCCCATTTGATCTCATCACTGAATTCCAG
TCACAATGGCCATCCTTTGGTTTCTCAAAGTTGCCGTTCCTTTGGCATCTCTTCCTCCCTGTCTT
CACAATGTAGGCTCCTTCCCATCCTAGGCTTAGTTTCGTTCCTCTCCCTAGAGGCCTTTCCTTAC
TGCCTCAGCTTCTCCCACTCTGCACACTCAGGGTCCTCTGCATTGTTCACTGCTGGCCTTCAAGA
GCCTAGAGGAGTTCCTCCCCATGGTGGGCTTTCAATAAGTGTTTGTGGAATAAGTGAAAAATGAG
TGGTCACACCAGGATCGAATGCACCTCTTACTTTTGTTAATATATTCAAATTAATATTAATATAT
TAATATTCTATTAATATATCCTAAAATTTTGTTGTGTATAGGGGCAGGGACCAATGAAAGAACAT
TTCATTGTCAGCTCATCTTGACTATGAGATCAGATATTGGCTATATTTTGAAGGTAGAGTCAGCA
GGATTTGCTGTTTAACTAGAGTGGGATGTGGGAGAAATCAGGGAGTCAGGTATAACTCCAAGGTT
TTCAGCCTCAAGCAACTGAAGAAATGGGGTCACCACGACTGAAATGAGGAAAATGAGAGGGGGAG
TTTAGTGGGGGATGGGGTGATGAAAATATCAGGATTCATTGTGGAACATGAACAGTTAAGATTAA
CTGAAGAGCCTGAATTCACTGTTCCTCAGTTTCTACAACTATAAGGAGGGGTTAATAATTTCTCA
CATCATAATTGTGAGGATTTGAGGAGTTGAGGTACACAATTAAAAACAAAAGCACAGGGGAAGTA
AGGAAAAATACATCAATTAAGCATGGCTATTAGACCATACAGCCTTACCAAATCCATTGGGGATT
AGATACAAAAATCCAAGCATGATCTCCAAAATTTTTTCCTGGTAGGGTTTTAGGCTATACTGAAG
TGACTTTTCTTTGGCATAAGAAGATATTCAGTTATACAGTTGGAAATAAAGGTATTGATTTGGAG
TATCCAAAAACATCTCTCAGTAGAGATCCACAACCAAAGAAGCATAAAAAAAAGTCTTCCATTCA
CTCGCAAAACTGTCTTATGACCATTGCAACCCTCAACAGCAAACATATGGAGTCTCTACTACATA
TAGAGGAAAATGCTAGATTCTGAAAAGCTAATATCTATAAAACAGAGTTTATGATGTTGTTATAT
TCTGGGGATTGATGTAAGCATTTTACCAGGACTTATTATAGCATTATAAGCTTAACACAAGAAAT
ATGTGGCTATCATTATGGAGTGAAAGGCTGGAAAATTCCTCACCTGTGACCTGAGAGATAGTGCG
TGGTAGAATTTGAAAGAAGTGTTGATTTCAGAGGAATGCTAGTGCTTGCTTAGGGCAGTATTCAG
AAGAACTCTTCCAATCACACAGCCCCTTGTACAGGCAAACTCCGGAATACTTTCAGTACATTTAC
TTGGCTTTATTGTTAGAGCAGAAACTTCACAGCTAAAATTATGGTCTTAGGGGTTTATAAGATGC
AATTAAACTTAATTTTTAAAAGTTCTCACAAATCTTATTTGAAGTCTTAATATCTTAATTTCCTT
TATATAGGATATGTGGATATATTTTTATGATAAAAGTAACATGTGACCAACTTAATAACAGCTTC
GTTAATATCCTAAAGCCACTCATCTTACTTTACATATAAACAGTCCTTCTGAAGCCTAATTATGC
ATGGATAATTGCAGAGTGAGTTTGGGAAAAGACCAACCGGGCACATCTCTCTCCATTTTAACCAC
AATGAGAAGAGAGTACCTAGTGGTGCATCTTCCTCGCTTAGGTTCCCTGAGTCTGTCTTTACAGG
AAGACTTCATTGTTACTTGAAGGTACATTCTTGGAACTTTACACACCCAGCCACTCAACACATGA
ATACATACTTATTTTAGTTAACTGAGTACTTAGTGAGTGCTAAGCTTTGTTTTAAGCCCTTTGCA
TGTAGTTTATTAACTTTGTTAATAGTTACAACAGTCCTTCAAGATACATGTACTGTGTTATACTA
GTGTAACAGTTGAGGACACCAAAGAACAAAGAGGTGTTGACACGTGGTCACGGTTCCATAGAGTG
TTAAGTTAGAGTTGGGTTCAAACCCTGGCAGTGTGGCCACAGAGCCTTGTTCTCAGCCACTGCAC
TGCACTACCTCCTCCCGTGAAACATAAGAAAAATGTGAGAAATGCTTAAGTAAGTGTAGTTTTTA
TTCATAAATAAAATTTACATAAGTACATTATGTGTAATTTGTTTTATGTATATGTGTGTATGTAT
AAATAAATACATGTAAAAATAAGGCCACAGTTTTAATTTTTTTCCATCTCTATAATAAAGCATGT
ATTATAGACCATTAGCAGAATTTAAAGTGTTATAGTAAATATTAATTGTGACTTTTGTTTTCTTC
TTCCCCAGTTTCATGGTTTTCTCCAAATGAAAATATTCTTATTGTTATTTTCCCAATTTTTGCTA
TACTCCTGTTCTGGGGACAGTTTGGTATTAAAAGTAAGTATTATTTCTACTTTTCATTTATGTTT
CAGTGATGATATAGTTATTTCTACGAGACATTGTCAGCGAAATATTTAAAGTTGTACTAGGAAAA
GTGCTATTATGATAAATATGAGTATGTAATTTGAATACTACTAGTCTCCTTGAAGTATATGTTGT
CGCCCACATTTTGCTGCAGTTCACTTTTAATTCCTAAGAAGGTTGTTTTCACTTGGTGTTTTTTT
AATCTCTTAAGAATGAATAGTAGGAATATTAGTACCAACACCTTAAACTCATGTCACATTTTAAT
ATTCACAGAACATCTACACACACATTATGTTATTAGGTAAACAGGTGGTGACAGCCTGCATTAGT
TTTAAGGTAGGACGTTATACTTTGGAGCATTTAGATTCCCCTCTTTTTATTTTCCCAGTTTGATT
TTCTCTGTGTACACGTGTTCACCCTTGGAAAAGTCCAGTCGGAACTATGTTTTGTCATCCTCTGC
GTGCAGTTCTGCAGCCTCTAAAGAAGCAGCCACCAGAGAGTTAGGTTCTTTGATCTTGCTTTCCT
ATAATAGTAACGTAACCAGACTTCTGAAGGCAGATCTTGATGCTGCATTAGATTTAGCTTCAACA
ACACAGAATTGTCATTACTAGGCAAATAGGTAATATGCATTACGGTTAATGTTTAATCAACCATA
TTTTCATATTTTGGTAAAGAAAATTTACAAAATTAATGAAGTTCTGAGGTGACAGTCTAAACTTT
TAAGCTTTTTAAATACAAGATTTATTCCTTCTTTCCTTGTAGCATCCTGAAGACCAGTAAACATT
TATATAAGCAAGGAATAAATACTGCTTATTTAATTTATTCTGCAACCTTTAAACACACAAAAGCT
AGTAAACTATTGCAGTGGATTGCCCTGTTGTATATTTTATGAATTTTACTTTTACTCAGCAGTTT
AAGCTGTCTATATCTATGGTGGTGTATAAACATGGAAGGGAGATGACTGATTGATTATATGTTTT
AAGCGCTTTTCTCAGTGTATGGCATTCTGGAAATGCTTAGTGATTTCAGAAATGTTCTCAACTTT
TGTCTGAAAGGAAAAAAGGGGGAAGAAAGGGGTGGCAGTGGCAACTGTCAAGACATTTTATAACT
TTTACTTTCAAGATAGTGTCTAGACTTCTTTTGGAAATTTTCTTATAATCTCTTAGTTTTTTATG
TCAAGAAAAGGACTGGTGTAGCATTAAGACCTATTCTGGCATCAATGATATTAGGGAAAGCTTTT
AACCATATTGGCAGAGCCAGACTTTAAGGGCTAAGTCATATGACTTGGGCAGGGAACAGCCTTTT
TCAAAGATCATGAAATTATTTCACATAGTGCGATTTTTTGAGTTTGGCTTGATGGATGTTTGCTG
ATCCAGACGTTATCATTGGTGACATTATTTTTAGTGAAATGAACCAGGAGTGAGTTTGTTCACTG
TTGGCTGTATTTTTACAAAATGAGCTTTACAATATTTTTTCTGACTTAAAAAAGTCATACATATC
ACTTTAGAACACCTCGTACAAAGTAGGGAGTTATTTAAAAAAAAAAAAAAAAAACTCATCTGTAG
TCCCCAAACCTAGAAATAAACTATTGGTATATTCTGGTGAATTTCCTGTTTCCCTTTCTGTCTTT
TTCTATTCATTTTTGTTATCTTTTTTTTAAAAAAAGGTAACCATAAATGGACTCGTACAGCTTAT
ATGCTTGTACGTTCTGATGTTCCCCCATGTCTTCAGAAATCTTGTTATAGCTGCCTGTGTTCTAC
TTTTGTGGATGCACTATAATTTACTTAACTCTCATCTTGATGGATTTTAAGGATGTTTCCAATTT
TTTTGATACTATGAAAATAACTCTGCTAAAGATATGAAGCCTGGAATTGGCAGAGATATTTTAAG
ACTCTAGATACATACTGGCAAAATGTTTTCCAGAAACGTTTGTATCAACTACTATTGCAGATGAG
AATACCTGTCTTTCACCTCAGTTATAATTCTGATATTATAACGCTAAAATCTCTGCAAATTTGAT
GGGTGAAAAAGCATCTTATTGTAATTGTAATTTTTGTGTTAGTAAGGTGGAATGTTCTTTTTTAT
ATTTTTTGTACTGGTCATATTTTAAGGAACGTGCTTATAAACCTAAAGAAATATTTTGGTGGGAA
TTTTTTGTTTTGGCTCATCTTGAAACAGGTAGATGTGTGTGTATGTGCATGGAAGAGGTATGTCT
ATACATGATTCACCCAGCCTGCGCTCACATTTAAAGGTGTTGATGATAATAGTAGCTAACCATTT
GTGGAGCTCTTGCTCTGCTTGACAGGTTCTGTGCAAAGTACTCTATATCTGAAATGACATTTATT
TCTCCCAGAAACTCTATGGGCATAGACACTGTTGTTATTCCCGTTTTGTAGATAAGAAACAGGCA
CAGATAGATTAGGCAATTTGCCCGCAATCACCCAGCCGTTTCCTGATAGTACTGGGATTTGAACT
AGTACTGTTTACCACTGCACTGTACTGCCTCCCCGTTTTGCATTTATTTTGAGGATTTTATTTCC
ATGAAGGGTGAACCTATATCTAAGCACATAATACTGAGTAGCTAAAACTTATTAGGAGAGCAGAA
TGTTGACCTGATTTGTTTACTTATTCTGCAAATACCTAGTGACAGGGTGCCTACTGATTGCTGGA
CACCAAGCTACATGCCTGAAATGTGGGTGTGATGTGCATATTGCCCTGGTTTTGTAGCACCCACA
TTTAAGCCAGGGAGACACGAATGTATGTGCATCCCATGCCTTGTCACATCTTTGAGATAGTCATG
GATCCAAATCTAACTTTTCTGTTAGTGCCTCTGTTGATGGCCTGAGCATTCCACCAAATAGAAAT
AGAAAGCACCTCTATTCTCCACCTGCTTAGATGTATATTTTTTAGAATCCAGTATGGTAAATCTT
CTAAAGCATTCATAATAACTCAGACCCATGACTTTTATTTTATAGATTTCTAGTTCTGCTTAACT
TTTCCTGATTACCATTTATGGTCTGTACTTGGCTACAGGGGATTTCTCTCAGTGGTTTTTCCAGC
TCTGCATGTGAGTCTTTGTGGTCCAAGACAAACCTGGACCTTTACCAGGCTAAACTTTCAGTAAA
GACAGCAGGTTATGCCCTCATTTGCTCACTCTGAGGAGAAAGAGTTTCTTTATACCAGAGCTGTA
TCTTGAAAGATGTCTCAGGATGCCATTGGTCCTACTGAGGAAGAAGCCTGAGAAGACTCTTAAAC
TCCCAGAGCCCAGCCAGCACTTGGTGAGCCCTGGACCACGTTTCAAAGATAAAGGCCTCTTACAG
GGAAATGTTCCTAAATACCTTCACCTTTAGCTTAGCTTTAACTTAGGAACTTTTAAGCAGAATCT
CTATGCTTAGCAAACAGCTCAGAGATTGCTAGAATCAAACACCAAGGCTTAACTGATAGTATTGA
ATTTCAGACGCTTTGTTTGTGTTCTGAAAACTACACCAACTCACAGTTTGCCACTCTACTGACAA
TTAAGTTCCTGGCTGATTTTCAGGATTCTTCTTTCTCACTCTGATATCATTTTAAGTGCTGTCCA
CCTAGTCCTCCAGTTCCTGCAGGATTAAGGTCCAACTGTATGTAAAGAACTGGCTAACATTTTGA
AATTCTTTGAGATAGGCCTATCTGTTCTTTCTTTGCCTTTGTAACTTTGTTTTATACAGGCAATA
CTGGCTTCGCACAAAACCGCAAGACCATAAACATGACCATGTAAGCTGAAATTCTGCAAAACAAT
CTTCATGATGAATGGGAAAAACTATTATTATATTGTTCCATGACCTTTAAAATTTTTTTTGTTGT
TAAAACCTTAAAAACTCTGTTATTATCAATAACAACGGCATTGGGAAATGAAAAATAGTAAAGCT
AGTATTTAGTATGTGGTAAATTAAATCATTAGAAACATCGAGAATGAAAGTGTGTTATCAAGAGT
AGTTTGAACAACACTTGTGTGTTCTTCTTGCATAACTTTGGATACAGAGCAAGCATCTTCTCTAT
GGCTTGGTGAATTGTCATACCCCTTTCTAAGTTTGGATCGGTTTCTACCGTTTTATCCTTTGCAC
TCTCAATGTCATGAAAGAATTCCTTTAATGTTTTTTTGAATGTTTAAAGTTTATTTTATTGCCAG
TCACTTCCTCTGGGACATCTTTATTCTTTTTGTTACATCCTCCTCACTTTCCTCATTTATATCAG
TAAGTCCACCTTCGCTGAGTTCTTCTGGCTTCATCTCTAGAGTTTCTTGCATTGCAGTAATGTCG
ACATTCGCACAATCAGCTATTTCTTCTCTTACTCCATTTATGGTCTATTCAAATTTCACTCCTAG
CATTTGTCAGTTTTTATTTCTTTACTCTTTCATCTTTCTTGCCTAGTTTCCTCTTTTGATTTATC
CATTATAAAATGTCACATGGGTTTATCACTGGGAAACAAGGAGGTAACAAAACTCCATACTTTGC
TGTCTGTGCATGACCTAAATATCAGATGTACAGTGACTAGTCACCAACAGTCTTCAAAAGAAGTG
ACATGGTTGATCACTGATCATGATGGGACATCTGCTATTTACATAGTTATTTTTGAACTGAAGAA
ATAGCAGTGAAGTTGTACTTTATGCAGTTACTCAGTTAATATATTGTGGTAATTGAAATTTGGAC
TGTTTATGAGGGATTTATTTGATTAAACCATGGTAACTGGAATTCATATCAGAATAGTGCAAAGT
GAGGACTGCTGTACTTGATTCCAAATTTAAAACTGTATTCTAGGCATCTTTAATTTTTTTTTTTC
AACTTTCCCCTCCCTGCTCAAAATTACTCCACAGAGATTTTGGTTACGGCTCAGATGTCTTTGAG
TAGATGTTCCTGTAGAAATCATTGTTAGAAAATTCAAGGAAAAAAGTTCTCATTACACTACCTTG
AGATCCTACCAAGTCCTTGAGTTTTGACTTGAGGACAGCTTAATAATGAAAGTAATTCAGCCATG
AAAGCATATTTAAAATAAGTAATGGAACAGGAAATTTCTTCATAGATTAGAAAAATTATTCTGAA
AAAGTGAAGACATAGCCTATCTTGGACTAGGAAATTTCCATCCAAGAAAGTAAAATATACAATAA
ATATTATCACAAAGAATCATTCTGTGACCTGGTTGATGGCCCCTGGTAATAAGACTTTGTTATTA
AGTTTACTGTGAATTCTTTTTTTTTTTTTTTTTTTTTTTTTTTTTTTTTGAGACGGAGTCTCGCT
CTGTTGCCCAGGCTGGAGTGCAGTGGCGTGATCTCGGCTCACTGCAACCTCCGCCTCCCAGGTTC
AAGCGATTCTTCTGTCTCAGCCTCCCGAGTAGCTACAGGCTACTGGCGTGCACCACCATGCCTGG
GTAATTTTTGTATTTTTAGTAGTGATGGGGTTTCACCATATTGGCCAGGCTGGTCTCGAACTCCT
GACCTCGTGATCCGTCTGTCTCGGCCTCCCAAAGTGCTGAGATTACAGGCATTAGCCACCATGCA
TGGCCCCTTACAGTGAATTCTGTTCAATAATCTTGAACTCCCAGTGCTTTCCCTTGGTCCTGTCC
ATATAATGATCACATTCTGTTATTAAATAATGTGCTTATGTGCTGATTTTATTGTAGGAGGAATT
GAACAAATCTATAGCTCAGCATTTTACTCTTTTGTAGAAATCCCTTGACCCTTTCCAAATCCAGA
CATCACAACCTTCTCCATATACTCAAAAATTGTGAAATCCACAAGGCAGGGAAAGTTGATAATGA
AAAAAAGGAAGTTTGGTCTGTTGGACATCCTCTTCTTTCATCTTTTATGTTCAAAGTACTTGATG
CACAAAGCCTGGACTCTTTTATCTCTGTCCTATAGACTGATGTCTAGTCAGTATTCATTGTGACA
AAATTGTTTCATTAGAGCAAGTTGGATAGTGTATGGAGATTATTATAAAATGATTTAACTTGTCT
TCTGTCAAGTAATTTTATAATTGGTATTAATGTTCATTTTTTTCCAGTACCAGTTTCAGAAGCTT
TCATTTGTGTATGCATGTGTGTGTTAAATAAGTGTATTAGATACTTGAAAAATAGTAATTTAAAT
TTAAACAATTTAAAAAATGAAATTGTAATTTTATGTGTAAAGGTGTCTGATGTGCTTTATTCTGC
ACTGAAAACAATATTCATTTACAGCTCAACAGACACCAAATATATTCTAAAATTACTTTCCTAGA
GTTATCAGAAAACAGCACTGTAATAATGAAATCAAACCCATCTTTCTTTATGATTTATTCTTAGT
CTGATACGCATCAGCCTGGTAAGCCTTCCTGTCTCTCTGCTTACTTACCAATCACTCCAAATGTC
ATGTCTTTGGGCAGGCATTAAATTCTTGGGTTTTGGGTTTTGTTGGATGGACTGCAGTCTGTGTG
AGCCTATATGGGTGTGTCAAATCCAGTCTTTGGGGTGTCATGGAAACTTAGCATGATAGACTTGA
TTTTATCCCCAAGTTGACTTGGTAATTTCATTAGATTTCATCAGTCACAACCTGCATTTTATCTT
GTATGTGCTGTCTATTGGTCACAAAATCAGAAAACCTTCTTGTCCATTCATAACATTAGCTGTTT
TTTCAGGTGGCTAGAGGGACATGTCATTGCTTCATCTGCATGAATTTGAAAGATTAAATGCATAA
AGGAATTTTCTTAGAGTAGAGTAGGCCTTCACCATCTCTTTAACTGGGAAAGAAGTTTTGGGAGT
AACATACTCATCACTCACACCCCCCTCCCCTCAAACACACACACTCACCCATTAGAATGTAAGGG
CCTTGAGGCAGGCCTTGTGTTTGCTTTTCGTGTTCACTGTCAGCATTTAGAATAGTGATAGTCAT
ATAGATGGATAGTGCTCAATCAATTATTGTCAAATAAAGTAATCTACTTGTTCCTTGATTTAGAC
TAGCAAAAGGGGCTGGTACATTGTAGGTACACAGTAAATATTTGTAGAACAGATGAATGAACCAA
CCCAACAGATTTCTTAGAAGTAGCCTGCTTTTGGTTACTAATTATTTTATAGAACATATAAAAGA
AAATTTTAGAATACCTAATTTGTTCACAAAAATGTTATATTGTCTCCCCTGATACTTGGTATTCC
TGTGTCAGGCTGAGCACTAAGGTTATACATTTTTTGTTAAGCATGTAATTACTGTTATTTCTCAT
GTACTTTATTTTGTGTATTGATTTCAGTAGTTGTAGCTGAGGGTAATCCTTAAGAGCATGCAGAT
TTTAAAAATTACATAGATTGCCTTGCCAAGCCGTCTGAAAGTCATACTAGAAAATTTGATAAGAG
CCTGCTGTTAACAACACATTGAGTTATTTTTATCTTAATGCTAAGTGGAGAATTACTTGAATTTA
TTTTTTTTGCTGCATTTAATGTTTTGATACATTCAATAAATAAGGATAAATCCACTTACTGAAGG
ACAAGAAAAGATCAATAAGAGATCCTTGAGGTTATTGGGTTCTATGGCATCTATTATATCAAATT
CCTTGCCCTTTCACTATACTGAGAAATTTGACGACTGGGCTAGCAACAAGATACCTACCCCACAT
TAGCTGTGGTACCTGCTGAAATGTGACCATGTGAGCAAAGAAAATGAGCCAAAAGAAACTCTTTC
TGAAGATTCACTGAAGAAAACCTGGAGATACTCTTTCATTAGTAGACCCCACAGAGACAGGTGGA
GTGTCCGTTATCCAGAATGCCTGGGACCAGAAGTGTTTTGGATTTCAGATTTTTTCACACTTGGG
ATCCTCAACATGTGTATTTGTTGGAAATGGCTACTTAATTTAAGGAAAAGTTTAAGGTGGCCTGG
AAAATAAGTAGTAGTTGAACTAATCAACAGGAACCAAAACTACTTTCAATATATAGTGTACTTAT
ACTCAAAGAGAGACGGACCATTTTATGGTGGAACCCTGTCTATGGTAGTATTTTGTGATGTTTTA
TTTTTGTTGCTATTGTTTGCACTGTTTTCTCCTATAACAGCTCTTCTAAGCCTTAAGAGGATAAA
TTTTATATGAGATTCAAACTCTTATTTTTGTTAAGAAGGATGTAAGTCACCAGGCATGGTGGCTC
ACACCTGTAATCCCAGCACTTTGGGAGGCCGTGGCAGGTGGATCATGAGGTAAGGAGCTCGAGAT
CAGCCTGGCCAACAATGTGAAACCCCGTCTCTACTAAAAAAAAAAAAAAAAGAATTCAAAAATTA
GCCAGGCCTGGTGGCGCATGCCTGTAGTCCCAGCTACTCGGGAGGCTGAGGCAGGAGAATCATTG
AACCTGGGAGGCAGAGTTTGCAGTGAGCCGAGATTGTGCCACTGCACTCCAGCCTGGGTAACGGA
GTGAGAGACTCTGTCTCCAAAAAAAAAAAAAAAGAAAAGAAAAAGAAAAATATAGAAGAAGAACA
TAAGTCAACCTTTTTCCATGAAATTATACTGTACTCTAAGGCAAATTCTTGTTGCTTGATCTTAG
TAGCTTTTTTATGCATTGACTTAAACCTGGAAGAGTTTCTTATGAATGTTTGATTGACTGTGAGG
TGCATTTGAAACAGCTTCTACTTTATAACCCTTTGCAGAACTTTCAAGCTCTATTTAGATAACAA
TATATATGGGATTAAAATGGAAAATGTGACATATGTCTAGAGAAAGTTCCTTTTTCTGTGTATGT
GCCTTTTGTGACTTATACACTCAGTTTCATTCTAGCCAGCATTTGAGCCTAGAAAATAGGGAAGT
TATGAAAACTCTGTGCCTTATAAGAAGGCAAGGCTATGACTGAGTATTAGACACATAAGTCCAGG
GCTGGGCGGAAGTAATGAATCAAATAAAAACTTGGGGAACTTGCCATAGGATGTTTTCTGATTAA
TATGGGTTTCTCTTCTCTGATTAATGTGGATTTCAGTTACTCAACTGTGTAACTGAAATCCACAT
TAATAAATTGGTTATTTATTGCTTAGGACTTATCTCCCCATAAAGAGACTAAAATTGAGGTTATA
AATTATGGGTAGATAGAAATTCCAGAAATATTTAGGTACCTGTCATATACCTTAAACATAGATTT
TTATCACAATCATTTAGGGGCTTATTTATTGCTTTTACTTTATTTAATGTTTGTCACTGTAGAAG
AAAAAAAACTAAATGCTAAATATAACATTTAAAATATTTTCCCCTTCATGACAGCCACCAATTGA
TTACTGTCATGGAGACTATCTCTATGTGGATGACACACAGCAGCATTTCAATCACACGCTGTTTC
TTTCCGCTACTCAGTTTGCTTTTAGGTTGCCTTAAACAACTTCCTTGGTGAAAAAATCAACTTCA
ATATTAACCAAAATTTAAAAGATTCATATATAGTAAAAGAACTAATATTCGCAGATTGACCCATT
CCATTCTTTCAGAGAAGGTATGAGATACTTGACAGTGGAGCTAGCAGCAGAAACAATAACATGTA
TGATGAATCCCATTGAGTCTTTGCAGTTTATTTTTATTAAAATATTTTAATTGAACAATTTAAGC
TTTTTTTCTTCATCAGGATTTCACAAGGGTGTAATCTGCCCAGTTTTATTCTGTCATTTTTTTCA
AAACAAAATAACTAGATTTCCAGATTGTTTACTAATTTTTTATAAGGTAGGACAAAATTCTCTTT
TTCTCAATATTGTTAATTAACCCATTATTTCCCATTAGTATTATGTACATCAGTGTTGTGTTGTC
ACTTGGAGTGGTTTTGCTCATCGGGGGCATTGACAATGTCTGAAAACAGTTTGATTAACATAACT
GGCATCTAGTTACTAGCATCTAGTATGTAGAGGCCAGGGATGCTCCTCAACATTCTATATTATGC
AGAGCAGTGTCCCCCCTCCCCCCAAAAACTTATCTAGCTCAATTTATTAGTAATACATCAACCGA
GAAAGACTGATGTATAGAACCTCATTTGTTAGTGTAGGAAAATAAGGTGTCACCTATAAATTCAC
CATCCATATTTATACTGCCGTGACGTTATCCATTTGCTTATGAAAGAGATGTGAGGTGACTTGAT
GATATTAAGGAGTTGTTCCTCATAAGTTATTACATTATAGTACTTCTGTCAGTTTGTCTCTGTAC
CTTACAAGTTATTAAAATGGCTTCACTTGTGATTGAGTTCATATAATTCTTTGTTTTTCTTTTTT
AAGCACTTAAATATAGATCCGGTGGTATGGATGAGAAAACAATTGCTTTACTTGTTGCTGGACTA
GTGATCACTGTCATTGTCATTGTTGGAGCCATTCTTTTCGTCCCAGGTAAGATGTGCAGTTCCTA
GGCAGGAACGCAGGAGGTAGATGAGTGCATTCCAAGGTGAGGAGGGCTGCATTAGTTTCCTAGGG
CTGTTGGAACAGATGACCACAAACTGGGTGGCTTAAACAATAGCAATTCCCTTTCCATGCTGGAG
GCCATAAGTCTGAAATCAGGACGTCATCAGGGCCACACTCCCTCTCAAGGCTGTATGGAAGAATC
CATTTCTTGGCTCTTCTAGCTTCTGGAATTTGCTGAAGAATGACAGTGTTTATCATTCCTTAGTT
TGCGGCAGCATAACTCCACCCTCTGCCTCTCTGGTCACATTGCCTCCTCCTTCCTCTATGTCTTT
GCCTCTGTGTTTTTGTTAAATCTCCCTCAGCCTCTCTCTTATGAGCACATTTGTCATTGGAATTA
GAGCCCACTCATATAATCCAGGGTAAGCTCCTCCTTTCAGATCCTTAACTTCATCATATCTTTTG
CCATATAATGCAGTATTCACTCTTTTGCTGTATTAGGTAATATTCACAGGTTTTAGGGATTAGGA
GGTAGAAATAATTTTAGGGCCATCATTCAACTCACTACAGAGGCAAACTTCATTGGCCAAGACAT
GAAAGTAGAATGAATGTGGGAACCAAGTCTGGAAATTGGCAGTTGCATTTGGAGGATGGGAATGT
GAGTGGGAAATGAGGATATGTAGCACATGTAAAGAAAAGCAAAGGAAGGCTGGGACTCAACCTAA
TAACTATAGCACACCATCGTTTGTGGAGAAGAATGCAGTGCAGGTGTAATATTTAGGAACATGGG
CTTGGCAGCTCTATTGGTATCTGAAGTTAGTGAAGACAGCAGGAAGAAGGAGGTGAATTCAGAAG
ACAACTTGAAGGAACAATTGATTTGACTTTGGGGCATACTGAATACAGCAGATGAAGGGAACAGT
AAAAGATGAATACACAGTTTCTCACTGGAATTCTGTAAACTAGTTAAGAAGGAGGCAGAGCCAAT
TCAAGGTTTCAGCATGTTGAAACTTAAATGTTGAGATTTAGATGTCTTAAGTTTGGTCTTAATCA
AAAAATAGTCACTAATTTTGTGTGAGGTTTTCAGGGAAACGTATATTTTTAAAATTTTCACAGTT
GTCAAGCACAGAATTTAGATTAGTACATTTAAAAAGTATGTAGGAAAATATTTTAAATGTTTTTA
TTTATTGACAGGTGAATATTCATTAAAGAATGCTACTGGCCTTGGTTTAATTGTGACTTCTACAG
GGATATTAATATTACTTCACTACTATGTGTTTAGTACAGGTGAGTTTTCATTGTTACAGTATGAT
TTTGTCTACCTTTTTCATTTACAAAACAGCAGTTTTGGTGAAAATGCTCATATAAATTTTTTACA
ACTCAATAAGAGTAGGTTTATTAAAAGATTTTTCATGCTATTCTTAGTGACATTTTCCCATTCAT
ATTAATTTTAAGGTTATTCTAGGTTAGCTGTTTTGTAAAAAGTGACTTTCATATGTATTTGATGC
CAATCAGCATAATTTTAAATTATGCCATATAACTTCTTAATGATTATTTTCATATTCTAATTTCA
GTTTTTTGAAGTTATAAGTGGATGTTAAACGCAGTCTTTCTCTTCTTTTGCTAAGCTTCTGCTTC
TGCATTAAGAAGACTGGTTCTATAAAATGGAATTATGAATCACAAATAGTAGAAACATATTTGTT
TTAATTATTAGCAAACCTTAATACTGTAGTTTTAAGAGATGGTATGGAAATCCAAACTATAATCA
GTATCATTTTCACTGCATTTTGAAAGTAGATCACTACCATATTTAGTTATTACTATTAAAGAGTC
ACATTTAACACTTGGTCTTAGAGCCTATCTCTGGCTCGTTGTACATTGGAAAGGTATTAGTTTGT
TATAAAATGACATCTAGAGATAGAGAGCATGATGTTTACAGTCAGGTATTTGCATAATGGTTCCC
TCAGCAAGAGCAATTCCATGTGCATGTGGAGAAGACATTCATAATAGCCATTCATATTGTAAAAT
GCACAAGTGTGGTAAAAGCAGCATTGTTCTAAGATTTAGGGTAAAAACTTCCAGTCCAGCTTAGC
TGACTGTGAAAATTAAAGGCTGCCGAATGTGTGAATTTGGAGCTGACTACTTGTGTGGAAGGGTT
AGATCACTGAGTTAACATCTACCGTCAAACAATGAACTCCAGAGTCAGTCTTTGGTCTTAGGAGA
TCCCTGATTATACCAAATGCAAGTGGAAGTTATTGCTTTTTAAATACTCTTGACATGCTTCTGTT
ACATCCTTTTCCTTCCTCCAGCGATTGGATTAACCTCCTTCGTCATTGCCATATTGGTTATTCAG
GTGATAGCCTATATCCTCGCTGTGGTTGGACTGAGTCTCTGTATTGCGGGTAAGAGTCATCTTTC
TGTAGACCTAATTTGGGTTACTTTTGGACAGAGCTCTTCTTCCTTTTTCTTTTTCTTTCTCTCTT
TTTAAAAATATATAGACTTGATTTTTTTTTTTAGTGCAGTTTTTGGTTCATGGCAAAATTGAGTG
GAAAGTACATTCACATGGATATTTTTATGTGGACATAAGTTTTCAATTCCATTTGGGCAAATACC
AAGGAGTGCAATTACGGGACTGTAAAGAGTATGTTGAAATAAACTGCCAAACTCTCTGTATCAAA
ATAGCTGTACCATTTTGTATTATCACCACCAATAAATAAGAGTTTTTGTTCATTCATATACTTGA
CAGCATTTAGCATTCTGAGCTTTTCTTTTTAAACTTCCAATTTACCCCAGTGGTAATAGTGCTTT
CCTCCTCGCTACAAAGATATTAGCTGTATATATGGCTTGGTGGCTGATTGTTCTAGCACCCAAAC
TGATATGCCTGTATTTGTGGAAGAGCTTTTAAAATAACTGGGCTCAAATTGGTTGGAGCCTTAGA
CTTGAAACACCAGTTCCCATTTCTTGATATGATAAGGTATGTGTTATGCAAAGGAGGGCTTTGTT
CTTCTAATAATTTTGAGTCATTTTACTGGTTAAGTTAATAAACATATATGGATAATTTTTGTTTT
TTGATCGTTAGAATAACTCTCTTAAAACTTGGGATTATTACTGTTTTTTAGTAAGTTATTTCATA
TGCTTTTCTAATACAGAATTTTATTTGTTTTTACAGCGTGTATACCAATGCATGGCCCTCTTCTG
ATTTCAGGTTTGAGTATCTTAGCTCTAGCACAATTACTTGGACTAGTTTATATGAAATTTGTGGG
TAAGTCAATCTTATTTTCATTAACCTATGCCAATAATTTCAGATATATCACTTAGAAAATGCTTT
TTAGTTTGTTCTTCCAGTTTAGGACCAAAAATGAGAAAATACAATTGGAGTGATTCGAGGATAAT
TAAAGAGGGTAGAAGACATATAGGATTTTTAGTTGGTTCTTCCAGTTTAAGACCAGAAGTCAGAA
AATACAATTGGAATGATTTGAGGGTAATTAAAGAGGGTAGAAGACATATAGGATTAATGAAAATT
TGGTTTCCAAAGTAGTTTAAAGGAAAATGGCTTTATCTATTAGAATGTGTACCTTTTTATACTAA
GTAAAAGGGGAGAGATCTTTGAGGATCCATTTTAAGTAATAGAATAGGATTTTTAATTGTTCCAG
TGTTTCTGTGATAGAGCTGTCCTGCACAGACCTGTTTGTTTGTCACTTGCTCTTTTTCTTGCAGA
CATAGACACCCCAGACAGGAATTAAAATTCACAATCTATCAATTTTGTTCATTTAAAGAGCAGTG
ACCTCTAATGCATGACTTTAAAACAGTTCTAGTTAAAACCAATATAATGAAAACATTGAGTTTCA
AAATTTAGGCTTTTACTCCTTTTAAAATCAATTATTAGTAAGTATGGAATTTACTTCATTGTTTC
TAACTTGTATATTTAATCTGCCAATTTTCAAGTAACATTTCTGCATAAATTCTTATTTTTTATTG
AGATATATGTACACAGAGAGATATTTTCAATTGTGCCTGAAACTAATGTTATCTTACCTAAGCTC
AAGATGTTCCCAATAATGTAATTTATATTAGTTTCCGTTTTTTAAAAAAATTATATTTTTATGAA
ATAAAACATACTCTTAACCACCTATCAAAATAATCAAAAGTTATAAATTAATGGAGTAAAAAAAT
AGTGTTTCTGCTTTGCTTTAGGTAAACTTTGCTGTATGTGTTTCTAAAACTTAATACGAAACTTG
AATTGTTATAGTCAAATAATTTCTCATATGACTCACATAATAGTTTCAAAAAACTTTTACCTTTA
TTTCTGAACTTTGGTTCTTGATGATTGTTAATTGAATTCAATTCTGTCATATATTCTGTGTCTTT
CTTTAATTAATGCTTATTAGATAAATAATTAAAATACTTAACTAAAATCTGCGTATCCTTAGCAT
ATGAGTTCATAAGTCTTAGTTGTTGCTCAATGAAATTTTCTAATTTTATACCACATAATGCCATA
AAATACAATGGAGACATCTAAAGCAGAATGGAATTCATGTGGTAGCTACAGTGAACATCTTGAAT
GTTGGTGCATATTCTATTTTTGTTACATCTTCCAATCACCATGTGTCTGGTTCTGGAAGATGACA
CTCCTGGTTTTGTTGCTCCCCACAAATGCCTGAGAATAGTGTGTGATTTGCAGTATCCATACAAC
TCTGGTGAAGTAGTATGAGATACCTTTGGCTGACGGGCAGCACGCTCTTATTTTTTCCTCACTAT
CTGGGTTGTCCTCCCTTTTACTCCCATGCCACCCCATGCCTTCCATATCTAGCATAGAATGATCT
TCAGTACAGTTGCCAGCAGGTCTGGTGACAATGTCTCAAGTGGAACTAAGCATTGTCTATCTGCC
ACCTCCTTAACTTCACTCTCCTGCCTTCTCCATCACTTACGTTCCTCCAAGCCTGTGAAACCACT
GTACTGTACCTGTCACTGTTCTGACATTAAAATTAAAATGACTTAAATCTTGACAAGTACCCCAA
ATTATTTTTTCTTTGTCATAGGTTAGCATATAAGTATACTATATGCTAAAATTTATGCTATATGT
TTAAAATTTAGTGCAATTTTATTGATAGTGTCCTAATTTTATTGATAGTATCCTAAATTAACTTT
TTAAATCAACTTGTCTGATGCCAGGGTTCAGAGGGACACCTACAGTCAGTTGAAAGGCAAGAAGA
GACAAGGTACAGGAAAGTTGCTCTTTAGATAACATGGTAGACTAGGAGGAACATTAATATGGTTT
GCTTATATAATCTGACTGTGTAAATCTGAATCTATGTAACATTAAGGTTGCAAATTTGAGCGTTT
ATATTAGGAAGTTAAAATTTTAAGTGTCTCCAAAATAATTTTTACTCATTGCACGTGTTCTGTTT
TAGAAAAGCCTAATGATTGTGTTTTGATTTAAATGCAATAAAATCCTCAAATAGTTAAAAATCCA
AGCTTTCTCTTCAAGAAGAAGTTAATGTCGCTATGAGATTTTTAACTTTTATAATTTTTATTATT
TCCAACTTTAAATTTGTAGCCTTAATTTGCTATTTTAAAGAGTAGGCCTTTCACTTTCTACAACT
TTCTGTGAAAGTGACTTTCACTTTCTATTTTTTAACTTTTTAAACTGTGTTGTATTTTTTTTCTT
TTATTGGAAGCATTTTAATTTTATAAGATGAGAAAAAGGACTGGGCACAATAACTTAATGTGAAA
GCATAGAAAAGATTACAAGAACCTAACCAAACTCACTAAAGTTGGGCTTGTTGTTTGTAGAGAAC
GTTTATATAATTATAAGGATCAATACTTTCTCATTTTTAAAGCCATTACCAGTTAGTTCAATATA
AGGGCATATAGTGTTTTGATACAAATCAATCTGGTAGCAGTAAGTACCATATTTACCACAACATC
CCAGATATTTTAGAATGATGCAGATGCAGAATATATACGTAGAATTTATATCTATGTATAGATAC
AAATTCAGATATTTTCTTGTTCAATTTAAGGAGAGGTAAATTTGGTATCAATAGAAAAAATGTTT
CTGAAAAATTTAAACCCTGGAAATGTATTTATGGCATGGAGTCAGATGTTTCAGGGAGAGAAGAA
CAAATCAAGAAGCATTGCAAGTATGCTCATATGGAATGCTTAAGGCTTGTGGTTAAAAAATATAT
ATATATGGCTGTCAATGTCTTAGGCTCATGGTAGCAGCAGAAATCGTAATAATTCTTTTGTCACA
TGGGTTATATCCATATTGGAGAGAATTAACTCAGGTGAAATTAACTTGTACACTGTTTGGTTTTA
TAATATTTAGAGGGATCACAACTGACTGATGTCCCTTTGAAGTACCATTCTTCATAAATCTTTTT
TTTTCAGAATGGGCCAGCCAACTGTGACATCCCTTGGATCGGAGATTTAGAACTAGAAAGTATTC
TTTCTACATTATTAGGGAAGAAAAGGAGTTACTTGGCGGTTAGCAATATTCTATTTTGTTTTGTT
TTGTTTTTAGAGACAGGGTCTCATTATGTTGACCAGGCTGGCCTCGAGCTCCTGGGCTCAAGCAA
TGCTCCCACCTCAGCCTCCCAAGTAGCTGGGACTACAGGCATGTGCCACTACACCTGGCAGTGTT
TATTCTGATAAATACATTTATGAGCTCAAAAATGTAACTCTAAAACCTTATCTCTGAACTTCCAT
ATTACCATCAGAAATTTAGATAGTTGTTTAGTTCTCTTTTTCTTTGTAGAACATAGATATAAGGC
ATGGTTTCATTGAAGTCAGTTGTATATACATGTAACTATCCTGATGTTCCCAAATAAAGCTCTGT
ATTTCTGCTTAGTTTATTGGGGAGGCTGCTAAATGTAGTGCATCCCAACCCATTTTACCCTGTTC
TACTTTAAAAAGAGGTTGGCTTCTTGTTTGGATACAAGGACCAAGTCACTCCCCCAGGTTCCTCC
ACAGTAAGGGAGGCCTATTTAAAGCCGCCCATGGCACTAACAGAAACTGGACTCCTATGAGCTCA
GATACATAACTGGGCCTCACAGGGGTGGGACAGTATGTAGTCTAGGAATTGGAAGGATCCATTCC
ATATCAAAGAACTGAAGCATCGTGTTGCCCTCTCAGCAGCAAGAGTAAGGTGATGCCCCTGTCAG
TTATAGTTCCTGAGTTCCTCTGTCTTTGATTCTTTGCCTATTAGCCAGCTAGCTCACCCTCTTGT
TTATGCCACTGTTTTTTATCCTATTCATGCCTTCTCACAGACAACTTTTCTTACCTACAGCTTTG
GACTCATCCTTGTCTCCTTTCTGTTTCTTTTTCACTTTCCCTTCCCATCACCAACTTTCTGGGTT
TTTTTCTGTTTCTTCTTAGAGTCCAGTGGCAGGGAGAAACTTGTCAGTCCAGTCTGTTGCCATTT
TTCCTGTTTGAGAAAGACTCACCAGCTTTTGGCTGGCTCACAGATTGGCTTTCCTTGGGTCAGGA
CCCACCCTTTTCCCTGCCAGCTTTGGAAGCTTGACAGAATTCGAGTGTGCAGTGGTGGTAAATAA
ATAGTAAGGAACACAGAGCAGTCCTGGAGGCGTGCCTCCATCTGCTGATGAGAAAATCCAGTGCT
GTCATCCAGCCCAGGTCCCAGCGGAATGGGCCTCTCTGTTCAGTAGGATCCCCCTCCTGCTGAGT
GGTTCATGGCATGTTTCTGTTCAACGCTTTTCCATCTGTAGGATTCTTATTCTGTATTTATTTGT
TTTTTTGGGTTTTTTTATTTTTTGAGATGGAGTCTCGCTCTGTCGCCCAGGCTGGAGTGCAGTGG
CACGACCCCAGCTCGCTGCAGCCTCTGCCTCCCAGGACGAGGGAGATCCTCCCACCTCAGCCTTC
CACGTAGCTGGGACTACAGGCATGCACCACAGGCATGCACCACCACGCCAGCTAATTTTTGTATT
TTTGGTAGAGACAGGGTTGCATCATGTTGCCCAGGCTGGTCTTGAATGCCTGAGCTCAAGCAATC
TATTTGCCTTGGCCTCCCAAAGTGCTGGGATTACAGGCATGAGCCACCACGGCCAGCCTTCTCAT
TTGTTTTTTTTATAAGGAAGCTATCTCTTCTTCCCTCCCCAACTAGGGTATTCTTTTTCCCTTTC
GTCACTTTGCTCATGTACTGTATTCCTTCAACTTCATTAATGAATCCATTTGGAAGCAGTGAAAA
AGGCAACTCAGAAAGCTAAGAAGAAATAGATAGAGGAATACTCAGAGCTATCTGAGTATTTTCTT
TAGTTTGTTAGCTCTTTGGAGCTTTGAAACTGGAAAGACCCAGGGAGTGATGTGGAGAAAGAGAC
TGAGCTTGTAAGACACAGGAGCAGTGAGCTAAGGGAGATGGAGTAGTGGGGACAAATTCTGGCAC
ATTCTGTCTACACTCTGGGTAGATAGAGGAGGGAGGATGGAGCACCCATGGTGGGGGTATGTTGG
TGACAGCATTTTCCCACCAGCCAGTGTAACAAGTGGCTGATTTGGGGGAAAGATGGCATAAACAA
ATGAGAGAATGTGTTTACTATTTGATGTAGATGGGTTATTTGCTTCATTTTTCAAATCAGTGTAT
ATAATCAAGAATATTCAGCATGTTTGAATAGACTGTCAGAGCTGGAACTCTTTCATTAACATCTC
TGGCACCTTTAGTTTTAGCCCTGAACATTTTATCTTAAAATTAAACATTACCAAATGCCTTAGTT
TATTTCATTTATTAAATTTATATTCTTATTTGTTATTTATATCAGCTTCCAATCAGAAGACTATA
CAACCTCCTAGGGTAAGTTAAAGTTTATTAAATGAATTGTGAATGATCATTTGAGGGATTAGACT
GAGGAACTTGGTAATTGAGATATTTTGCTATCTGTTTTGTCTCACGTCAAATTAAGAGAATGTTG
AAGTCATTGCATGACCTTTGCATGAATGGGTCCAGTTCTATTTTAAAACCTGTGTTTGGTCATTT
TAGTGTCAATGGGATGGAATAAATGATTTCTTAAGATTGTACTGACTTCTCACACCCAAAACTGG
AAAGTAGGAATAATGGCTATATTATCTCTGCAATCAGAAGGAAGCTGATTCCAATATATCACCTC
ACCTGTTGGATTCATTGGATGTGCATACACAGAATGACAATTTCAGGCTTAAAAATGAGGAGAAA
TCTATACTAAGTTGACATCACTGATAATTATAATCTATAAAATAAATGTAAATATTGCTGAAAAC
ATCTGTTCGGAGTTATGATTCGATCCTCTCCCATACAAATGTTTTATAAACATTTTTTCCCTTAA
AACTGTGCTTAAGGTTTCATTGTACCTTAGATACCTTATTAAGCCATCTGAGGAAATTGCAAGAA
AGGAGTAATTTTAGGAGGGCATAAATGAAGAGAAAAGCATATTTATAAACATAGACTTATCACAG
TGACAGGCCCAAGAGGTATGTTGTGGACATAAGCTCTGGAAAGGATTATATTGTACTTAGCTTCC
TATAACGGAGTGATGATAGATGTATCTGGAATGCCAAAGAGAGTCTTCTGTCTGTGGCCGGAGGT
AGAGGTCACATATGCTTCTAAGTCTGACAAGCCTCATTGTGCCTAAAGGGCAGGTGGGGGGGGTA
TGTGTGTCTACCACAGGGGTATATTCTAGAAAGATTGGTGCCATAGCTATGTTGGTCACAAGAGG
CCAGCAACTTAACTGGACCTGTGAATCCTAACACATTTTCTTTCCCAGTTACTGAGTTCAATTTG
CGATACTTAAAGATGATTCTGTTTTGCTTCCACCTCTTCACTGTGTTATTTATTCTGTTGTTGCT
ATTTATGCTTGCACTTTCATATTTTTAGAAGTTAGAATTTCTTGAGCTGAGAGTGGTGAAGTGGG
AAATTCTGTTTAGAAAAATAATATTAAGAGAATAATACAGTTATTATTAAACTATTAACCCGACT
TGGCAAGCTTTGCTTTAACATTTACAGGCTTATTTCGTTGTTTTGTTTTTTCTTTTTTTTTTGAC
TGTAGGATTTACTGCTTACTACGTAATTTAAATATTAGCATATATAAGTTTTACTATAAAATGAC
ATGACATAAATTATATTTTTATGTAAATATATTTTAAATATTTTTCAGAAAGCTGTAGAGGAACC
CCTTAATGGTATGTGGTTATTTCACTCTTAATCCTTTACCAGATCATAATTTGATCTGGCCCGCA
AAACAGTTAGAATGCCCTGTCTATGCCTTAGGAAGAATCTAGGTTTTTTTCCTCTTTTTTTCTTT
CCTTGGCATCTCTACTCTTGATTATTCATCAAGAATTATGGGCTGGGTGCGGTGGCTCACGCTTG
CGATCCCGGCACCTTGGGAGGCCAAGGCGGGCAGATCACGAGGTCAAGAGATTGAGACCATCCTG
GCCAACATGTTGAAACCCTGTCTCTACTGAAAATACAAAAATTACCTGGGCATGGTGGTGTGTAT
CTGCAGTCCCAGCTACTCGGGAGGCTGAGGCAGGAGAATTGCTTGAACCCGGAAGGCAGATGTTG
CAGTGAGTTGAGATCATGCCACCGCACTCCAGCCTGGTGACAGAGTGAGACTCTGTCTCAAAAAA
AATAAAAAAGAATTATGAATATTACTTTTATAATATTCTCACCACTGGGAAAAATGCACTATTCT
GTGTCTAAGTAGCTGCTTACCTTAACCAAGTGATATTTGGGCAAGGGGATCGTTGCCTTTTGCTA
CTGGTTGAGACGAAGCATGGCACCCCCTAGTAGAGAAGGATCCCAATTACTTCCAATTTGTGATG
TACACATTTTAGAAAGATACAGGCTATTGCCACAGAGATAGACCAAAACATCTCATTTTCTTTCT
TTGTAAACCTAGATCTGATTTCCCAACTAAGTCTGTTTCCTTTGTGAATGCTGTGGGTATGATCC
ACAGAAAGGCTACATAATGAAATGATAGCTTTACAATTAATTTGGCTGTAGAGTTGTAGACTAGT
TAGCATATCATTGCATATTTGTTTATTTAGAAATGATTTCCAATTGTGGAACCTCACTAACTGCC
TGCTTGGCTTGTTACTAATCCTAGCATTCAAAGAATCAAAAGGAATGATGAATGATGGTAAGTAT
AAAATGCACTTAATAATTATAGATCAGTTAAAACATGGACATTGGAAAACAAAAAAGCTTCTTGA
AAATGTGGCTCTTTTTTAGTAAAAGGGACACTGTCAGATGATAAAGGTTCACATTTCTTGATGTA
TACAACTTAAATCTACTTTGCTAAAAATTGCAAAACTACTACTGTAAAAACTGTAGGGTGTCACG
AATCAGACTCCAGTCATATGGCTCCCAGCAAAGAGAAATTACCACTTTTTGTAAAATGTTTTTCA
GATTCCGTGTCTGGTGACTGTAACTTTAAGATGCCTTTTATAAGGCACATAAATAATCTGGCACA
AATCTTTATCATTTTGACAGAGTTTCTTTTATGCTTGTGTTGGTGATTTTGTTGCATTTAACCCA
TGGGGACTTAACATCTCTGCTTTTCCAATCAGGAGCTTGGTTCTAACCTTTTGGGAGATGATTAA
AGAGAGAGGTTGAGAGAGAGAGAGAGAGAGAGAGAGAGAGAGAGAGAGAGAGAGAGAGAGATGTT
TCGTCCTCAGCTTTTGCTTCCATTTTTTTTTTAAGAACTCTGGGCTAATAACTTCTAATCTTTAT
AGAATATTTCAAAGAAATATATTTGTTCTTAAAGATACATAGGTTTGAGATATTGAGTGCTACAA
GCATTTATTTTGGTTTTACCTTAACATATTATGATTCCTCAGTTTTGTTGGCATTTAGTAATTAT
GTTTATGTTTTTATCTTATCAAAAAATGTCTTCTTACCTTTGATATTTATAATCACTCCTCGGTC
ATGTAAATAGTTTGCTTTATATTTTACTGTTTTAAAGTCTGTGACCTTACCTGCCCTCTTCTGTA
GCAAAGTGCAGCATTTAACTCAGGAAGCTATATTCCCCCCAAGTGTCATTAATATTTGCATAAGA
TTAAAAACACTCCAGTCGGCCGGGCGCGGTGGCTCACGCCTGTAATCCCAGCACTTTGGGAGGCC
GAGGCGGGCGGATCACAAGGTCAGGAGATCGAGACCATCCTGGCTAACACGGTGAAACCCCGTCT
CTACTAAAACTACAAAAAATTAGCCGGGCATGGTGGCGGGCACCTGTGGTCCCAGCTACTTGGGA
GGCTGAGGCAGGAGAATGGCGTGAACCCGGGAGGCAGAGCTTGCAGTGAGCCAAGATCGTGCCAC
TACACTCCAGCCTGGGTGACAGAGCAAGACTCCGTCTCAAGAAAAAAAAAAAAAAAAAAAAAAAA
AAAACACTCCAGTCATGCATTGGTGAACAAAGTTTAAAACAACGTGTATTCAGCATGGAGTCACA
GAATGATCCTACTTTTGTATGTTTGTGTCACAGCCTTTAAAAGCATGTCTTGTTATATAAGCCAT
TACCCTCCTAAAAAAGACTATAGTTCACAGGAATAAGTTAAAAGACATAACAAAACATAAAATGA
CTAGTACCAGGAATTGTGACCATGGTTTGTTCTGGTAACTGTGGCATGGCATGGTTTGTTCCAGG
AATTGTGGCATGGTTTGTTCATGTTTACATTCTGATGTCCTATTTTTTTTTTTTAATTTCTATGT
CCTTTCCTTTTCCTTGGTGGTGTCATTGTTCTGTAGCTGTATGAAGAAACTAAACTTTTCTCCAT
TTTCAGGAAAGCAATCTAAGAATCTTGAGTGCCTCTTCCTTTGTTAATTTCTCTTAAGATGTGAC
TTTTTTAAACTACTGCATCAGGAAATATTGTAAAACAGTTTTGCCTTGAATATTTGTGATGAAAT
CTACGATGATCTTCAAGATTCTCTTAATTTTGCTAATATTCAGCTGATCAGAATTTGTTTTTAAA
ATGTCTGGCTGGTGGGTACTTCCCACTGACAACTGCTTATTGCTTACAGTATGTCTGCCTTGTCA
ATGAATGAGGTTCAGGGTGCTTCCTAGGGATCAGAGTCAGTACCATTTTTCTCTTTCATCTACAG
CTGATCAGATGTTTATTTTACTTACATTAAATGAATGATGGAGATCCAAAGTGAATATTATAGAA
TATTATTCTAGGATCAACATCTTTTGCTTTGAAAAATCAACATCTCTTGGCTTTTCCTCAGCCAA
CCCAGCAAACAGAGATTATCAGACTCTGTTGATTTTTTACTTTCATTTGGCATTGGCCTTTTTCT
TACTGAAATTAAAAAGGCTAATGATTTGCCTGGTTTCTGTCTCTGACCTTTGCAGGTCTATTTTC
TTAATTTTTAGATACTATATATCTGAAACTTTTTTTAATGTGTCAACTTTTTAATGGATAGAAAA
TAGACACGAATAGTGATTATGTGTTCATTTTTCAATTTTCCAGAATAACTGAAGTGAAGTGATGG
ACTCCGATTTGGAGAGTAGTAAGACGTGAAAGGAATACACTTGTGTTTAAGCACCATGGCCTTGA
TGATTCACTGTTGGGGAGAAGAAACAAGAAAAGTAACTGGTTGTCACCTATGAGACCCTTACGTG
ATTGTTAGTTAAGTTTTTATTCAAAGCAGCTGTAATTTAGTTAATAAAATAATTATGATCTATGT
TGTTTGCCCAATTGAGATCCAGTTTTTTGTTGTTATTTTTAATCAATTAGGGGCAATAGTAGAAT
GGACAATTTCCAAGAATGATGCCTTTCAGGTCCTAGGGCCTCTGGCCTCTAGGTAACCAGTTTAA
ATTGGTTCAGGGTGATAACTACTTAGCACTGCCCTGGTGATTACCCAGAGATATCTATGAAAACC
AGTGGCTTCCATCAAACCTTTGCCAACTCAGGTTCACAGCAGCTTTGGGCAGTTATGGCAGTATG
GCATTAGCTGAGAGGTGTCTGCCACTTCTGGGTCAATGGAATAATAAATTAAGTACAGGCAGGAA
TTTGGTTGGGAGCATCTTGTATGATCTCCGTATGATGTGATATTGATGGAGATAGTGGTCCTCAT
TCTTGGGGGTTGCCATTCCCACATTCCCCCTTCAACAAACAGTGTAACAGGTCCTTCCCAGATTT
AGGGTACTTTTATTGATGGATATGTTTTCCTTTTATTCACATAACCCCTTGAAACCCTGTCTTGT
CCTCCTGTTACTTGCTTCTGCTGTACAAGATGTAGCACCTTTTCTCCTCTTTGAACATGGTCTAG
TGACACGGTAGCACCAGTTGCAGGAAGGAGCCAGACTTGTTCTCAGAGCACTGTGTTCACACTTT
TCAGCAAAAATAGCTATGGTTGTAACATATGTATTCCCTTCCTCTGATTTGAAGGCAAAAATCTA
CAGTGTTTCTTCACTTCTTTTCTGATCTGGGGCATGAAAAAAGCAAGATTGAAATTTGAACTATG
AGTCTCCTGCATGGCAACAAAATGTGTGTCACCATCAGGCCAACAGGCCAGCCCTTGAATGGGGA
TTTATTACTGTTGTATCTATGTTGCATGATAAACATTCATCACCTTCCTCCTGTAGTCCTGCCTC
GTACTCCCCTTCCCCTATGATTGAAAAGTAAACAAAACCCACATTTCCTATCCTGGTTAGAAGAA
AATTAATGTTCTGACAGTTGTGATCGCCTGGAGTACTTTTAGACTTTTAGCATTCGTTTTTTACC
TGTTTGTGGATGTGTGTTTGTATGTGCATACGTATGAGATAGGCACATGCATCTTCTGTATGGAC
AAAGGTGGGGTACCTACAGGAGAGCAAAGGTTAATTTTGTGCTTTTAGTAAAAACATTTAAATAC
AAAGTTCTTTATTGGGTGGAATTATATTTGATGCAAATATTTGATCACTTAAAACTTTTAAAACT
TCTAGGTAATTTGCCACGCTTTTTGACTGCTCACCAATACCCTGTAAAAATACGTAATTCTTCCT
GTTTGTGTAATAAGATATTCATATTTGTAGTTGCATTAATAATAGTTATTTCTTAGTCCATCAGA
TGTTCCCGTGTGCCTCTTTTATGCCAAATTGATTGTCATATTTCATGTTGGGACCAAGTAGTTTG
CCCATGGCAAACCTAAATTTATGACCTGCTGAGGCCTCTCAGAAAACTGAGCATACTAGCAAGAC
AGCTCTTCTTGAAAAAAAAAATATGTATACACAAATATATACGTATATCTATATATACGTATGTA
TATACACACATGTATATTCTTCCTTGATTGTGTAGCTGTCCAAAATAATAACATATATAGAGGGA
GCTGTATTCCTTTATACAAATCTGATGGCTCCTGCAGCACTTTTTCCTTCTGAAAATATTTACAT
TTTGCTAACCTAGTTTGTTACTTTAAAAATCAGTTTTGATGAAAGGAGGGAAAAGCAGATGGACT
TGAAAAAGATCCAAGCTCCTATTAGAAAAGGTATGAAAATCTTTATAGTAAAATTTTTTATAAAC
TAAAGTTGTACCTTTTAATATGTAGTAAACTCTCATTTATTTGGGGTTCGCTCTTGGATCTCATC
CATCCATTGTGTTCTCTTTAATGCTGCCTGCCTTTTGAGGCATTCACTGCCCTAGACAATGCCAC
CAGAGATAGTGGGGGAAATGCCAGATGAAACCAACTCTTGCTCTCACTAGTTGTCAGCTTCTCTG
GATAAGTGACCACAGAAGCAGGAGTCCTCCTGCTTGGGCATCATTGGGCCAGTTCCTTCTCTTTA
AATCAGATTTGTAATGGCTCCCAAATTCCATCACATCACATTTAAATTGCAGACAGTGTTTTGCA
CATCATGTATCTGTTTTGTCCCATAATATGCTTTTTACTCCCTGATCCCAGTTTCTGCTGTTGAC
TCTTCCATTCAGTTTTATTTATTGTGTGTTCTCACAGTGACACCATTTGTCCTTTTCTGCAACAA
CCTTTCCAGCTACTTTTGCCAAATTCTATTTGTCTTCTCCTTCAAAACATTCTCCTTTGCAGTTC
CTCTTCATCTGTGTAGCTGCTCTTTTGTCTCTTAACTTACCATTCCTATAGTACTTTATGCATCT
CTGCTTAGTTCTATTAGTTTTTTGGCCTTGCTCTTCTCCTTGATTTTAAAATTCCTTCTATAGCT
AGAGCTTTTCTTTCTTTCATTCTCTCTTCCTGCAGTGTTTTGCATACATCAGAAGCTAGGTACAT
AAGTTAAATGATTGAGAGTTGGCTGTATTTAGATTTATCACTTTTTAATAGGGTGAGCTTGAGAG
TTTTCTTTCTTTCTGTTTTTTTTTTTTGTTTTTTTTTTTTTTTTTTTTTTTTTTTTTTTTGACTA
ATTTCACATGCTCTAAAAACCTTCAAAGGTGATTATTTTTCTCCTGGAAACTCCAGGTCCATTCT
GTTTAAATCCCTAAGAATGTCAGAATTAAAATAACAGGGCTATCCCGTAATTGGAAATATTTCTT
TTTTCAGGATGCTATAGTCAATTTAGTAAGTGACCACCAAATTGTTATTTGCACTAACAAAGCTC
AAAACACGATAAGTTTACTCCTCCATCTCAGTAATAAAAATTAAGCTGTAATCAACCTTCTAGGT
TTCTCTTGTCTTAAAATGGGTATTCAAAAATGGGGATCTGTGGTGTATGTATGGAAACACATACT
CCTTAATTTACCTGTTGTTGGAAACTGGAGAAATGATTGTCGGGCAACCGTTTATTTTTTATTGT
ATTTTATTTGGTTGAGGGATTTTTTTATAAACAGTTTTACTTGTGTCATATTTTAAAATTACTAA
CTGCCATCACCTGCTGGGGTCCTTTGTTAGGTCATTTTCAGTGACTAATAGGGATAATCCAGGTA
ACTTTGAAGAGATGAGCAGTGAGTGACCAGGCAGTTTTTCTGCCTTTAGCTTTGACAGTTCTTAA
TTAAGATCATTGAAGACCAGCTTTCTCATAAATTTCTCTTTTTGAAAAAAAGAAAGCATTTGTAC
TAAGCTCCTCTGTAAGACAACATCTTAAATCTTAAAAGTGTTGTTATCATGACTGGTGAGAGAAG
AAAACATTTTGTTTTTATTAAATGGAGCATTATTTACAAAAAGCCATTGTTGAGAATTAGATCCC
ACATCGTATAAATATCTATTAACCATTCTAAATAAAGAGAACTCCAGTGTTGCTATGTGCAAGAT
CCTCTCTTGGAGCTTTTTTGCATAGCAATTAAAGGTGTGCTATTTGTCAGTAGCCATTTTTTTGC
AGTGATTTGAAGACCAAAGTTGTTTTACAGCTGTGTTACCGTTAAAGGTTTTTTTTTTTATATGT
ATTAAATCAATTTATCACTGTTTAAAGCTTTGAATATCTGCAATCTTTGCCAAGGTACTTTTTTA
TTTAAAAAAAAACATAACTTTGTAAATATTACCCTGTAATATTATATATACTTAATAAAACATTT
TAAGCTATTTTGTTGGGCTATTTCTATTGCTGCTACAGCAGACCACAAGCACATTTCTGAAAAAT
TTAATTTATTAATGTATTTTTAAGTTGCTTATATTCTAGGTAACAATGTAAAGAATGATTTAAAA
TATTAATTATGAATTTTTTGAGTATAATACCCAATAAGCTTTTAATTAGAGCAGAGTTTTAATTA
AAAGTTTTAAATCAGTCCAA

### Human CD47 Transcript Variant 1—NM_001777.4 (SEQ ID NO: 4); 3′-UTR Underlined (SEQ ID NO: 80)

GCAGCCTGGGCAGTGGGTCCTGCCTGTGACGCGCGGCGGCGGTCGGTCCTGCCTGTAACGGCGGC
GGCGGCTGCTGCTCCGGACACCTGCGGCGGCGGCGGCGACCCCGCGGCGGGCGCGGAGATGTGGC
CCCTGGTAGCGGCGCTGTTGCTGGGCTCGGCGTGCTGCGGATCAGCTCAGCTACTATTTAATAAA
ACAAAATCTGTAGAATTCACGTTTTGTAATGACACTGTCGTCATTCCATGCTTTGTTACTAATAT
GGAGGCACAAAACACTACTGAAGTATACGTAAAGTGGAAATTTAAAGGAAGAGATATTTACACCT
TTGATGGAGCTCTAAACAAGTCCACTGTCCCCACTGACTTTAGTAGTGCAAAAATTGAAGTCTCA
CAATTACTAAAAGGAGATGCCTCTTTGAAGATGGATAAGAGTGATGCTGTCTCACACACAGGAAA
CTACACTTGTGAAGTAACAGAATTAACCAGAGAAGGTGAAACGATCATCGAGCTAAAATATCGTG
TTGTTTCATGGTTTTCTCCAAATGAAAATATTCTTATTGTTATTTTCCCAATTTTTGCTATACTC
CTGTTCTGGGGACAGTTTGGTATTAAAACACTTAAATATAGATCCGGTGGTATGGATGAGAAAAC
AATTGCTTTACTTGTTGCTGGACTAGTGATCACTGTCATTGTCATTGTTGGAGCCATTCTTTTCG
TCCCAGGTGAATATTCATTAAAGAATGCTACTGGCCTTGGTTTAATTGTGACTTCTACAGGGATA
TTAATATTACTTCACTACTATGTGTTTAGTACAGCGATTGGATTAACCTCCTTCGTCATTGCCAT
ATTGGTTATTCAGGTGATAGCCTATATCCTCGCTGTGGTTGGACTGAGTCTCTGTATTGCGGCGT
GTATACCAATGCATGGCCCTCTTCTGATTTCAGGTTTGAGTATCTTAGCTCTAGCACAATTACTT
GGACTAGTTTATATGAAATTTGTGGCTTCCAATCAGAAGACTATACAACCTCCTAGGAAAGCTGT
AGAGGAACCCCTTAATGCATTCAAAGAATCAAAAGGAATGATGAATGATGAATAACTGAAGTGAA
GTGATGGACTCCGATTTGGAGAGTAGTAAGACGTGAAAGGAATACACTTGTGTTTAAGCACCATG
GCCTTGATGATTCACTGTTGGGGAGAAGAAACAAGAAAAGTAACTGGTTGTCACCTATGAGACCC
TTACGTGATTGTTAGTTAAGTTTTTATTCAAAGCAGCTGTAATTTAGTTAATAAAATAATTATGA
TCTATGTTGTTTGCCCAATTGAGATCCAGTTTTTTGTTGTTATTTTTAATCAATTAGGGGCAATA
GTAGAATGGACAATTTCCAAGAATGATGCCTTTCAGGTCCTAGGGCCTCTGGCCTCTAGGTAACC
AGTTTAAATTGGTTCAGGGTGATAACTACTTAGCACTGCCCTGGTGATTACCCAGAGATATCTAT
GAAAACCAGTGGCTTCCATCAAACCTTTGCCAACTCAGGTTCACAGCAGCTTTGGGCAGTTATGG
CAGTATGGCATTAGCTGAGAGGTGTCTGCCACTTCTGGGTCAATGGAATAATAAATTAAGTACAG
GCAGGAATTTGGTTGGGAGCATCTTGTATGATCTCCGTATGATGTGATATTGATGGAGATAGTGG
TCCTCATTCTTGGGGGTTGCCATTCCCACATTCCCCCTTCAACAAACAGTGTAACAGGTCCTTCC
CAGATTTAGGGTACTTTTATTGATGGATATGTTTTCCTTTTATTCACATAACCCCTTGAAACCCT
GTCTTGTCCTCCTGTTACTTGCTTCTGCTGTACAAGATGTAGCACCTTTTCTCCTCTTTGAACAT
GGTCTAGTGACACGGTAGCACCAGTTGCAGGAAGGAGCCAGACTTGTTCTCAGAGCACTGTGTTC
ACACTTTTCAGCAAAAATAGCTATGGTTGTAACATATGTATTCCCTTCCTCTGATTTGAAGGCAA
AAATCTACAGTGTTTCTTCACTTCTTTTCTGATCTGGGGCATGAAAAAAGCAAGATTGAAATTTG
AACTATGAGTCTCCTGCATGGCAACAAAATGTGTGTCACCATCAGGCCAACAGGCCAGCCCTTGA
ATGGGGATTTATTACTGTTGTATCTATGTTGCATGATAAACATTCATCACCTTCCTCCTGTAGTC
CTGCCTCGTACTCCCCTTCCCCTATGATTGAAAAGTAAACAAAACCCACATTTCCTATCCTGGTT
AGAAGAAAATTAATGTTCTGACAGTTGTGATCGCCTGGAGTACTTTTAGACTTTTAGCATTCGTT
TTTTACCTGTTTGTGGATGTGTGTTTGTATGTGCATACGTATGAGATAGGCACATGCATCTTCTG
TATGGACAAAGGTGGGGTACCTACAGGAGAGCAAAGGTTAATTTTGTGCTTTTAGTAAAAACATT
TAAATACAAAGTTCTTTATTGGGTGGAATTATATTTGATGCAAATATTTGATCACTTAAAACTTT
TAAAACTTCTAGGTAATTTGCCACGCTTTTTGACTGCTCACCAATACCCTGTAAAAATACGTAAT
TCTTCCTGTTTGTGTAATAAGATATTCATATTTGTAGTTGCATTAATAATAGTTATTTCTTAGTC
CATCAGATGTTCCCGTGTGCCTCTTTTATGCCAAATTGATTGTCATATTTCATGTTGGGACCAAG
TAGTTTGCCCATGGCAAACCTAAATTTATGACCTGCTGAGGCCTCTCAGAAAACTGAGCATACTA
GCAAGACAGCTCTTCTTGAAAAAAAAAATATGTATACACAAATATATACGTATATCTATATATAC
GTATGTATATACACACATGTATATTCTTCCTTGATTGTGTAGCTGTCCAAAATAATAACATATAT
AGAGGGAGCTGTATTCCTTTATACAAATCTGATGGCTCCTGCAGCACTTTTTCCTTCTGAAAATA
TTTACATTTTGCTAACCTAGTTTGTTACTTTAAAAATCAGTTTTGATGAAAGGAGGGAAAAGCAG
ATGGACTTGAAAAAGATCCAAGCTCCTATTAGAAAAGGTATGAAAATCTTTATAGTAAAATTTTT
TATAAACTAAAGTTGTACCTTTTAATATGTAGTAAACTCTCATTTATTTGGGGTTCGCTCTTGGA
TCTCATCCATCCATTGTGTTCTCTTTAATGCTGCCTGCCTTTTGAGGCATTCACTGCCCTAGACA
ATGCCACCAGAGATAGTGGGGGAAATGCCAGATGAAACCAACTCTTGCTCTCACTAGTTGTCAGC
TTCTCTGGATAAGTGACCACAGAAGCAGGAGTCCTCCTGCTTGGGCATCATTGGGCCAGTTCCTT
CTCTTTAAATCAGATTTGTAATGGCTCCCAAATTCCATCACATCACATTTAAATTGCAGACAGTG
TTTTGCACATCATGTATCTGTTTTGTCCCATAATATGCTTTTTACTCCCTGATCCCAGTTTCTGC
TGTTGACTCTTCCATTCAGTTTTATTTATTGTGTGTTCTCACAGTGACACCATTTGTCCTTTTCT
GCAACAACCTTTCCAGCTACTTTTGCCAAATTCTATTTGTCTTCTCCTTCAAAACATTCTCCTTT
GCAGTTCCTCTTCATCTGTGTAGCTGCTCTTTTGTCTCTTAACTTACCATTCCTATAGTACTTTA
TGCATCTCTGCTTAGTTCTATTAGTTTTTTGGCCTTGCTCTTCTCCTTGATTTTAAAATTCCTTC
TATAGCTAGAGCTTTTCTTTCTTTCATTCTCTCTTCCTGCAGTGTTTTGCATACATCAGAAGCTA
GGTACATAAGTTAAATGATTGAGAGTTGGCTGTATTTAGATTTATCACTTTTTAATAGGGTGAGC
TTGAGAGTTTTCTTTCTTTCTGTTTTTTTTTTTTGTTTTTTTTTTTTTTTTTTTTTTTTTTTTTT
TTGACTAATTTCACATGCTCTAAAAACCTTCAAAGGTGATTATTTTTCTCCTGGAAACTCCAGGT
CCATTCTGTTTAAATCCCTAAGAATGTCAGAATTAAAATAACAGGGCTATCCCGTAATTGGAAAT
ATTTCTTTTTTCAGGATGCTATAGTCAATTTAGTAAGTGACCACCAAATTGTTATTTGCACTAAC
AAAGCTCAAAACACGATAAGTTTACTCCTCCATCTCAGTAATAAAAATTAAGCTGTAATCAACCT
TCTAGGTTTCTCTTGTCTTAAAATGGGTATTCAAAAATGGGGATCTGTGGTGTATGTATGGAAAC
ACATACTCCTTAATTTACCTGTTGTTGGAAACTGGAGAAATGATTGTCGGGCAACCGTTTATTTT
TTATTGTATTTTATTTGGTTGAGGGATTTTTTTATAAACAGTTTTACTTGTGTCATATTTTAAAA
TTACTAACTGCCATCACCTGCTGGGGTCCTTTGTTAGGTCATTTTCAGTGACTAATAGGGATAAT
CCAGGTAACTTTGAAGAGATGAGCAGTGAGTGACCAGGCAGTTTTTCTGCCTTTAGCTTTGACAG
TTCTTAATTAAGATCATTGAAGACCAGCTTTCTCATAAATTTCTCTTTTTGAAAAAAAGAAAGCA
TTTGTACTAAGCTCCTCTGTAAGACAACATCTTAAATCTTAAAAGTGTTGTTATCATGACTGGTG
AGAGAAGAAAACATTTTGTTTTTATTAAATGGAGCATTATTTACAAAAAGCCATTGTTGAGAATT
AGATCCCACATCGTATAAATATCTATTAACCATTCTAAATAAAGAGAACTCCAGTGTTGCTATGT
GCAAGATCCTCTCTTGGAGCTTTTTTGCATAGCAATTAAAGGTGTGCTATTTGTCAGTAGCCATT
TTTTTGCAGTGATTTGAAGACCAAAGTTGTTTTACAGCTGTGTTACCGTTAAAGGTTTTTTTTTT
TATATGTATTAAATCAATTTATCACTGTTTAAAGCTTTGAATATCTGCAATCTTTGCCAAGGTAC
TTTTTTATTTAAAAAAAAACATAACTTTGTAAATATTACCCTGTAATATTATATATACTTAATAA
AACATTTTAAGCTATTTTGTTGGGCTATTTCTATTGCTGCTACAGCAGACCACAAGCACATTTCT
GAAAAATTTAATTTATTAATGTATTTTTAAGTTGCTTATATTCTAGGTAACAATGTAAAGAATGA
TTTAAAATATTAATTATGAATTTTTTGAGTATAATACCCAATAAGCTTTTAATTAGAGCAGAGTT
TTAATTAAAAGTTTTAAATCAGTCCAA

### Human CD47 Transcript Variant 2—NM_198793.3 (SEQ ID NO: 5); 3′-UTR Underlined (SEQ ID NO: 81)

GCAGCCTGGGCAGTGGGTCCTGCCTGTGACGCGCGGCGGCGGTCGGTCCTGCCTGTAACGGCGGC
GGCGGCTGCTGCTCCGGACACCTGCGGCGGCGGCGGCGACCCCGCGGCGGGCGCGGAGATGTGGC
CCCTGGTAGCGGCGCTGTTGCTGGGCTCGGCGTGCTGCGGATCAGCTCAGCTACTATTTAATAAA
ACAAAATCTGTAGAATTCACGTTTTGTAATGACACTGTCGTCATTCCATGCTTTGTTACTAATAT
GGAGGCACAAAACACTACTGAAGTATACGTAAAGTGGAAATTTAAAGGAAGAGATATTTACACCT
TTGATGGAGCTCTAAACAAGTCCACTGTCCCCACTGACTTTAGTAGTGCAAAAATTGAAGTCTCA
CAATTACTAAAAGGAGATGCCTCTTTGAAGATGGATAAGAGTGATGCTGTCTCACACACAGGAAA
CTACACTTGTGAAGTAACAGAATTAACCAGAGAAGGTGAAACGATCATCGAGCTAAAATATCGTG
TTGTTTCATGGTTTTCTCCAAATGAAAATATTCTTATTGTTATTTTCCCAATTTTTGCTATACTC
CTGTTCTGGGGACAGTTTGGTATTAAAACACTTAAATATAGATCCGGTGGTATGGATGAGAAAAC
AATTGCTTTACTTGTTGCTGGACTAGTGATCACTGTCATTGTCATTGTTGGAGCCATTCTTTTCG
TCCCAGGTGAATATTCATTAAAGAATGCTACTGGCCTTGGTTTAATTGTGACTTCTACAGGGATA
TTAATATTACTTCACTACTATGTGTTTAGTACAGCGATTGGATTAACCTCCTTCGTCATTGCCAT
ATTGGTTATTCAGGTGATAGCCTATATCCTCGCTGTGGTTGGACTGAGTCTCTGTATTGCGGCGT
GTATACCAATGCATGGCCCTCTTCTGATTTCAGGTTTGAGTATCTTAGCTCTAGCACAATTACTT
GGACTAGTTTATATGAAATTTGTGGCTTCCAATCAGAAGACTATACAACCTCCTAGGAATAACTG
AAGTGAAGTGATGGACTCCGATTTGGAGAGTAGTAAGACGTGAAAGGAATACACTTGTGTTTAAG
CACCATGGCCTTGATGATTCACTGTTGGGGAGAAGAAACAAGAAAAGTAACTGGTTGTCACCTAT
GAGACCCTTACGTGATTGTTAGTTAAGTTTTTATTCAAAGCAGCTGTAATTTAGTTAATAAAATA
ATTATGATCTATGTTGTTTGCCCAATTGAGATCCAGTTTTTTGTTGTTATTTTTAATCAATTAGG
GGCAATAGTAGAATGGACAATTTCCAAGAATGATGCCTTTCAGGTCCTAGGGCCTCTGGCCTCTA
GGTAACCAGTTTAAATTGGTTCAGGGTGATAACTACTTAGCACTGCCCTGGTGATTACCCAGAGA
TATCTATGAAAACCAGTGGCTTCCATCAAACCTTTGCCAACTCAGGTTCACAGCAGCTTTGGGCA
GTTATGGCAGTATGGCATTAGCTGAGAGGTGTCTGCCACTTCTGGGTCAATGGAATAATAAATTA
AGTACAGGCAGGAATTTGGTTGGGAGCATCTTGTATGATCTCCGTATGATGTGATATTGATGGAG
ATAGTGGTCCTCATTCTTGGGGGTTGCCATTCCCACATTCCCCCTTCAACAAACAGTGTAACAGG
TCCTTCCCAGATTTAGGGTACTTTTATTGATGGATATGTTTTCCTTTTATTCACATAACCCCTTG
AAACCCTGTCTTGTCCTCCTGTTACTTGCTTCTGCTGTACAAGATGTAGCACCTTTTCTCCTCTT
TGAACATGGTCTAGTGACACGGTAGCACCAGTTGCAGGAAGGAGCCAGACTTGTTCTCAGAGCAC
TGTGTTCACACTTTTCAGCAAAAATAGCTATGGTTGTAACATATGTATTCCCTTCCTCTGATTTG
AAGGCAAAAATCTACAGTGTTTCTTCACTTCTTTTCTGATCTGGGGCATGAAAAAAGCAAGATTG
AAATTTGAACTATGAGTCTCCTGCATGGCAACAAAATGTGTGTCACCATCAGGCCAACAGGCCAG
CCCTTGAATGGGGATTTATTACTGTTGTATCTATGTTGCATGATAAACATTCATCACCTTCCTCC
TGTAGTCCTGCCTCGTACTCCCCTTCCCCTATGATTGAAAAGTAAACAAAACCCACATTTCCTAT
CCTGGTTAGAAGAAAATTAATGTTCTGACAGTTGTGATCGCCTGGAGTACTTTTAGACTTTTAGC
ATTCGTTTTTTACCTGTTTGTGGATGTGTGTTTGTATGTGCATACGTATGAGATAGGCACATGCA
TCTTCTGTATGGACAAAGGTGGGGTACCTACAGGAGAGCAAAGGTTAATTTTGTGCTTTTAGTAA
AAACATTTAAATACAAAGTTCTTTATTGGGTGGAATTATATTTGATGCAAATATTTGATCACTTA
AAACTTTTAAAACTTCTAGGTAATTTGCCACGCTTTTTGACTGCTCACCAATACCCTGTAAAAAT
ACGTAATTCTTCCTGTTTGTGTAATAAGATATTCATATTTGTAGTTGCATTAATAATAGTTATTT
CTTAGTCCATCAGATGTTCCCGTGTGCCTCTTTTATGCCAAATTGATTGTCATATTTCATGTTGG
GACCAAGTAGTTTGCCCATGGCAAACCTAAATTTATGACCTGCTGAGGCCTCTCAGAAAACTGAG
CATACTAGCAAGACAGCTCTTCTTGAAAAAAAAAATATGTATACACAAATATATACGTATATCTA
TATATACGTATGTATATACACACATGTATATTCTTCCTTGATTGTGTAGCTGTCCAAAATAATAA
CATATATAGAGGGAGCTGTATTCCTTTATACAAATCTGATGGCTCCTGCAGCACTTTTTCCTTCT
GAAAATATTTACATTTTGCTAACCTAGTTTGTTACTTTAAAAATCAGTTTTGATGAAAGGAGGGA
AAAGCAGATGGACTTGAAAAAGATCCAAGCTCCTATTAGAAAAGGTATGAAAATCTTTATAGTAA
AATTTTTTATAAACTAAAGTTGTACCTTTTAATATGTAGTAAACTCTCATTTATTTGGGGTTCGC
TCTTGGATCTCATCCATCCATTGTGTTCTCTTTAATGCTGCCTGCCTTTTGAGGCATTCACTGCC
CTAGACAATGCCACCAGAGATAGTGGGGGAAATGCCAGATGAAACCAACTCTTGCTCTCACTAGT
TGTCAGCTTCTCTGGATAAGTGACCACAGAAGCAGGAGTCCTCCTGCTTGGGCATCATTGGGCCA
GTTCCTTCTCTTTAAATCAGATTTGTAATGGCTCCCAAATTCCATCACATCACATTTAAATTGCA
GACAGTGTTTTGCACATCATGTATCTGTTTTGTCCCATAATATGCTTTTTACTCCCTGATCCCAG
TTTCTGCTGTTGACTCTTCCATTCAGTTTTATTTATTGTGTGTTCTCACAGTGACACCATTTGTC
CTTTTCTGCAACAACCTTTCCAGCTACTTTTGCCAAATTCTATTTGTCTTCTCCTTCAAAACATT
CTCCTTTGCAGTTCCTCTTCATCTGTGTAGCTGCTCTTTTGTCTCTTAACTTACCATTCCTATAG
TACTTTATGCATCTCTGCTTAGTTCTATTAGTTTTTTGGCCTTGCTCTTCTCCTTGATTTTAAAA
TTCCTTCTATAGCTAGAGCTTTTCTTTCTTTCATTCTCTCTTCCTGCAGTGTTTTGCATACATCA
GAAGCTAGGTACATAAGTTAAATGATTGAGAGTTGGCTGTATTTAGATTTATCACTTTTTAATAG
GGTGAGCTTGAGAGTTTTCTTTCTTTCTGTTTTTTTTTTTTGTTTTTTTTTTTTTTTTTTTTTTT
TTTTTTTTTGACTAATTTCACATGCTCTAAAAACCTTCAAAGGTGATTATTTTTCTCCTGGAAAC
TCCAGGTCCATTCTGTTTAAATCCCTAAGAATGTCAGAATTAAAATAACAGGGCTATCCCGTAAT
TGGAAATATTTCTTTTTTCAGGATGCTATAGTCAATTTAGTAAGTGACCACCAAATTGTTATTTG
CACTAACAAAGCTCAAAACACGATAAGTTTACTCCTCCATCTCAGTAATAAAAATTAAGCTGTAA
TCAACCTTCTAGGTTTCTCTTGTCTTAAAATGGGTATTCAAAAATGGGGATCTGTGGTGTATGTA
TGGAAACACATACTCCTTAATTTACCTGTTGTTGGAAACTGGAGAAATGATTGTCGGGCAACCGT
TTATTTTTTATTGTATTTTATTTGGTTGAGGGATTTTTTTATAAACAGTTTTACTTGTGTCATAT
TTTAAAATTACTAACTGCCATCACCTGCTGGGGTCCTTTGTTAGGTCATTTTCAGTGACTAATAG
GGATAATCCAGGTAACTTTGAAGAGATGAGCAGTGAGTGACCAGGCAGTTTTTCTGCCTTTAGCT
TTGACAGTTCTTAATTAAGATCATTGAAGACCAGCTTTCTCATAAATTTCTCTTTTTGAAAAAAA
GAAAGCATTTGTACTAAGCTCCTCTGTAAGACAACATCTTAAATCTTAAAAGTGTTGTTATCATG
ACTGGTGAGAGAAGAAAACATTTTGTTTTTATTAAATGGAGCATTATTTACAAAAAGCCATTGTT
GAGAATTAGATCCCACATCGTATAAATATCTATTAACCATTCTAAATAAAGAGAACTCCAGTGTT
GCTATGTGCAAGATCCTCTCTTGGAGCTTTTTTGCATAGCAATTAAAGGTGTGCTATTTGTCAGT
AGCCATTTTTTTGCAGTGATTTGAAGACCAAAGTTGTTTTACAGCTGTGTTACCGTTAAAGGTTT
TTTTTTTTATATGTATTAAATCAATTTATCACTGTTTAAAGCTTTGAATATCTGCAATCTTTGCC
AAGGTACTTTTTTATTTAAAAAAAAACATAACTTTGTAAATATTACCCTGTAATATTATATATAC
TTAATAAAACATTTTAAGCTATTTTGTTGGGCTATTTCTATTGCTGCTACAGCAGACCACAAGCA
CATTTCTGAAAAATTTAATTTATTAATGTATTTTTAAGTTGCTTATATTCTAGGTAACAATGTAA
AGAATGATTTAAAATATTAATTATGAATTTTTTGAGTATAATACCCAATAAGCTTTTAATTAGAG
CAGAGTTTTAATTAAAAGTTTTAAATCAGTCCAA

### Human CD47 Transcript Variant 3—NM_001382306.1 (SEQ ID NO: 6); 3′-UTR Underlined (SEQ ID NO: 82)

GCAGCCTGGGCAGTGGGTCCTGCCTGTGACGCGCGGCGGCGGTCGGTCCTGCCTGTAACGGCGGC
GGCGGCTGCTGCTCCGGACACCTGCGGCGGCGGCGGCGACCCCGCGGCGGGCGCGGAGATGTGGC
CCCTGGTAGCGGCGCTGTTGCTGGGCTCGGCGTGCTGCGGATCAGCTCAGCTACTATTTAATAAA
ACAAAATCTGTAGAATTCACGTTTTGTAATGACACTGTCGTCATTCCATGCTTTGTTACTAATAT
GGAGGCACAAAACACTACTGAAGTATACGTAAAGTGGAAATTTAAAGGAAGAGATATTTACACCT
TTGATGGAGCTCTAAACAAGTCCACTGTCCCCACTGACTTTAGTAGTGCAAAAATTGAAGTCTCA
CAATTACTAAAAGGAGATGCCTCTTTGAAGATGGATAAGAGTGATGCTGTCTCACACACAGGAAA
CTACACTTGTGAAGTAACAGAATTAACCAGAGAAGGTGAAACGATCATCGAGCTAAAATATCGTG
TTGTTTCATGGTTTTCTCCAAATGAAAATATTCTTATTGTTATTTTCCCAATTTTTGCTATACTC
CTGTTCTGGGGACAGTTTGGTATTAAAACACTTAAATATAGATCCGGTGGTATGGATGAGAAAAC
AATTGCTTTACTTGTTGCTGGACTAGTGATCACTGTCATTGTCATTGTTGGAGCCATTCTTTTCG
TCCCAGGTGAATATTCATTAAAGAATGCTACTGGCCTTGGTTTAATTGTGACTTCTACAGGGATA
TTAATATTACTTCACTACTATGTGTTTAGTACAGCGATTGGATTAACCTCCTTCGTCATTGCCAT
ATTGGTTATTCAGGTGATAGCCTATATCCTCGCTGTGGTTGGACTGAGTCTCTGTATTGCGGCGT
GTATACCAATGCATGGCCCTCTTCTGATTTCAGGTTTGAGTATCTTAGCTCTAGCACAATTACTT
GGACTAGTTTATATGAAATTTGTGGCTTCCAATCAGAAGACTATACAACCTCCTAGGAAAGCTGT
AGAGGAACCCCTTAATGAATAACTGAAGTGAAGTGATGGACTCCGATTTGGAGAGTAGTAAGACG
TGAAAGGAATACACTTGTGTTTAAGCACCATGGCCTTGATGATTCACTGTTGGGGAGAAGAAACA
AGAAAAGTAACTGGTTGTCACCTATGAGACCCTTACGTGATTGTTAGTTAAGTTTTTATTCAAAG
CAGCTGTAATTTAGTTAATAAAATAATTATGATCTATGTTGTTTGCCCAATTGAGATCCAGTTTT
TTGTTGTTATTTTTAATCAATTAGGGGCAATAGTAGAATGGACAATTTCCAAGAATGATGCCTTT
CAGGTCCTAGGGCCTCTGGCCTCTAGGTAACCAGTTTAAATTGGTTCAGGGTGATAACTACTTAG
CACTGCCCTGGTGATTACCCAGAGATATCTATGAAAACCAGTGGCTTCCATCAAACCTTTGCCAA
CTCAGGTTCACAGCAGCTTTGGGCAGTTATGGCAGTATGGCATTAGCTGAGAGGTGTCTGCCACT
TCTGGGTCAATGGAATAATAAATTAAGTACAGGCAGGAATTTGGTTGGGAGCATCTTGTATGATC
TCCGTATGATGTGATATTGATGGAGATAGTGGTCCTCATTCTTGGGGGTTGCCATTCCCACATTC
CCCCTTCAACAAACAGTGTAACAGGTCCTTCCCAGATTTAGGGTACTTTTATTGATGGATATGTT
TTCCTTTTATTCACATAACCCCTTGAAACCCTGTCTTGTCCTCCTGTTACTTGCTTCTGCTGTAC
AAGATGTAGCACCTTTTCTCCTCTTTGAACATGGTCTAGTGACACGGTAGCACCAGTTGCAGGAA
GGAGCCAGACTTGTTCTCAGAGCACTGTGTTCACACTTTTCAGCAAAAATAGCTATGGTTGTAAC
ATATGTATTCCCTTCCTCTGATTTGAAGGCAAAAATCTACAGTGTTTCTTCACTTCTTTTCTGAT
CTGGGGCATGAAAAAAGCAAGATTGAAATTTGAACTATGAGTCTCCTGCATGGCAACAAAATGTG
TGTCACCATCAGGCCAACAGGCCAGCCCTTGAATGGGGATTTATTACTGTTGTATCTATGTTGCA
TGATAAACATTCATCACCTTCCTCCTGTAGTCCTGCCTCGTACTCCCCTTCCCCTATGATTGAAA
AGTAAACAAAACCCACATTTCCTATCCTGGTTAGAAGAAAATTAATGTTCTGACAGTTGTGATCG
CCTGGAGTACTTTTAGACTTTTAGCATTCGTTTTTTACCTGTTTGTGGATGTGTGTTTGTATGTG
CATACGTATGAGATAGGCACATGCATCTTCTGTATGGACAAAGGTGGGGTACCTACAGGAGAGCA
AAGGTTAATTTTGTGCTTTTAGTAAAAACATTTAAATACAAAGTTCTTTATTGGGTGGAATTATA
TTTGATGCAAATATTTGATCACTTAAAACTTTTAAAACTTCTAGGTAATTTGCCACGCTTTTTGA
CTGCTCACCAATACCCTGTAAAAATACGTAATTCTTCCTGTTTGTGTAATAAGATATTCATATTT
GTAGTTGCATTAATAATAGTTATTTCTTAGTCCATCAGATGTTCCCGTGTGCCTCTTTTATGCCA
AATTGATTGTCATATTTCATGTTGGGACCAAGTAGTTTGCCCATGGCAAACCTAAATTTATGACC
TGCTGAGGCCTCTCAGAAAACTGAGCATACTAGCAAGACAGCTCTTCTTGAAAAAAAAAATATGT
ATACACAAATATATACGTATATCTATATATACGTATGTATATACACACATGTATATTCTTCCTTG
ATTGTGTAGCTGTCCAAAATAATAACATATATAGAGGGAGCTGTATTCCTTTATACAAATCTGAT
GGCTCCTGCAGCACTTTTTCCTTCTGAAAATATTTACATTTTGCTAACCTAGTTTGTTACTTTAA
AAATCAGTTTTGATGAAAGGAGGGAAAAGCAGATGGACTTGAAAAAGATCCAAGCTCCTATTAGA
AAAGGTATGAAAATCTTTATAGTAAAATTTTTTATAAACTAAAGTTGTACCTTTTAATATGTAGT
AAACTCTCATTTATTTGGGGTTCGCTCTTGGATCTCATCCATCCATTGTGTTCTCTTTAATGCTG
CCTGCCTTTTGAGGCATTCACTGCCCTAGACAATGCCACCAGAGATAGTGGGGGAAATGCCAGAT
GAAACCAACTCTTGCTCTCACTAGTTGTCAGCTTCTCTGGATAAGTGACCACAGAAGCAGGAGTC
CTCCTGCTTGGGCATCATTGGGCCAGTTCCTTCTCTTTAAATCAGATTTGTAATGGCTCCCAAAT
TCCATCACATCACATTTAAATTGCAGACAGTGTTTTGCACATCATGTATCTGTTTTGTCCCATAA
TATGCTTTTTACTCCCTGATCCCAGTTTCTGCTGTTGACTCTTCCATTCAGTTTTATTTATTGTG
TGTTCTCACAGTGACACCATTTGTCCTTTTCTGCAACAACCTTTCCAGCTACTTTTGCCAAATTC
TATTTGTCTTCTCCTTCAAAACATTCTCCTTTGCAGTTCCTCTTCATCTGTGTAGCTGCTCTTTT
GTCTCTTAACTTACCATTCCTATAGTACTTTATGCATCTCTGCTTAGTTCTATTAGTTTTTTGGC
CTTGCTCTTCTCCTTGATTTTAAAATTCCTTCTATAGCTAGAGCTTTTCTTTCTTTCATTCTCTC
TTCCTGCAGTGTTTTGCATACATCAGAAGCTAGGTACATAAGTTAAATGATTGAGAGTTGGCTGT
ATTTAGATTTATCACTTTTTAATAGGGTGAGCTTGAGAGTTTTCTTTCTTTCTGTTTTTTTTTTT
TGTTTTTTTTTTTTTTTTTTTTTTTTTTTTTTTTGACTAATTTCACATGCTCTAAAAACCTTCAA
AGGTGATTATTTTTCTCCTGGAAACTCCAGGTCCATTCTGTTTAAATCCCTAAGAATGTCAGAAT
TAAAATAACAGGGCTATCCCGTAATTGGAAATATTTCTTTTTTCAGGATGCTATAGTCAATTTAG
TAAGTGACCACCAAATTGTTATTTGCACTAACAAAGCTCAAAACACGATAAGTTTACTCCTCCAT
CTCAGTAATAAAAATTAAGCTGTAATCAACCTTCTAGGTTTCTCTTGTCTTAAAATGGGTATTCA
AAAATGGGGATCTGTGGTGTATGTATGGAAACACATACTCCTTAATTTACCTGTTGTTGGAAACT
GGAGAAATGATTGTCGGGCAACCGTTTATTTTTTATTGTATTTTATTTGGTTGAGGGATTTTTTT
ATAAACAGTTTTACTTGTGTCATATTTTAAAATTACTAACTGCCATCACCTGCTGGGGTCCTTTG
TTAGGTCATTTTCAGTGACTAATAGGGATAATCCAGGTAACTTTGAAGAGATGAGCAGTGAGTGA
CCAGGCAGTTTTTCTGCCTTTAGCTTTGACAGTTCTTAATTAAGATCATTGAAGACCAGCTTTCT
CATAAATTTCTCTTTTTGAAAAAAAGAAAGCATTTGTACTAAGCTCCTCTGTAAGACAACATCTT
AAATCTTAAAAGTGTTGTTATCATGACTGGTGAGAGAAGAAAACATTTTGTTTTTATTAAATGGA
GCATTATTTACAAAAAGCCATTGTTGAGAATTAGATCCCACATCGTATAAATATCTATTAACCAT
TCTAAATAAAGAGAACTCCAGTGTTGCTATGTGCAAGATCCTCTCTTGGAGCTTTTTTGCATAGC
AATTAAAGGTGTGCTATTTGTCAGTAGCCATTTTTTTGCAGTGATTTGAAGACCAAAGTTGTTTT
ACAGCTGTGTTACCGTTAAAGGTTTTTTTTTTTATATGTATTAAATCAATTTATCACTGTTTAAA
GCTTTGAATATCTGCAATCTTTGCCAAGGTACTTTTTTATTTAAAAAAAAACATAACTTTGTAAA
TATTACCCTGTAATATTATATATACTTAATAAAACATTTTAAGCTATTTTGTTGGGCTATTTCTA
TTGCTGCTACAGCAGACCACAAGCACATTTCTGAAAAATTTAATTTATTAATGTATTTTTAAGTT
GCTTATATTCTAGGTAACAATGTAAAGAATGATTTAAAATATTAATTATGAATTTTTTGAGTATA
ATACCCAATAAGCTTTTAATTAGAGCAGAGTTTTAATTAAAAGTTTTAAATCAGTCCAA

In some embodiments, a disruption in the 3′-UTR of an allele encoding CD47 comprises a deletion of the 3′-UTR. In some embodiments, a deletion comprises deletion of one or more fragments of the 3′-UTR. In some embodiments, a deletion is a complete deletion of the 3′-UTR. In some embodiments, a disruption in the 3′-UTR of an allele encoding CD47 comprises an inversion of a sequence in the 3′-UTR. In some embodiments, a disruption in the 3′-UTR of an allele encoding CD47 comprises an inversion of a fragment in the 3′-UTR. In some embodiments, a disruption in the 3′-UTR of an allele encoding CD47 comprises an inversion of the entire 3′-UTR. In some embodiments, a disruption in the 3′-UTR of an allele encoding CD47 comprises a translocation of a sequence in the entire 3′-UTR. In some embodiments, a disruption in the 3′-UTR of an allele encoding CD47 comprises substitution of one or more nucleotides in the 3′-UTR. In some embodiments, a disruption in the 3′-UTR of an allele encoding CD47 comprises an insertion of one or more nucleotides in the 3′-UTR.

In some embodiments, a disruption in the 3′-UTR of an allele encoding CD47 leads to increased expression (e.g., increased by at least 10%, at least 20%, at least 30%, at least 40%, at least 50%, at least 60%, at least 70%, at least 80%, at least 90%, at least 100%, at least 2-fold, at least 5-fold, at least 10-fold, at least 50-fold, at least 100-fold or higher) of CD47 in the cell, the isolated stem cell and cells differentiated from the isolated stem cell. In some embodiments, the increased expression of CD47 is induced or increased by interferon gamma.

In some embodiments, a cell (e.g., an isolated stem cell) described herein comprises a disruption in the 3′-UTR of an allele encoding Histocompatibility Antigen, Class I, G (HLA-G). In some embodiments, the disruption is a homozygous modification. In some embodiments, the disruption is a heterozygous modification. “Histocompatibility Antigen, Class I, G (HLA-G)” belongs to the HLA nonclassical class I heavy chain paralogues. It is a heterodimer consisting of a heavy chain and a light chain. The heavy chain is anchored in the membrane. HLA-G plays a role in immunosuppression. HLA-G is a ligand for the natural killer (NK) cell inhibitory receptor KIR2DL4. Expression of HLA-G defends the expressing cell against NK cell-mediated death.

An example of a Homo sapiens HLA-G gene sequence is provided in NCBI Gene ID: 3135 (SEQ ID NO: 30; corresponding to positions 29826474 to 29831130 of Homo Sapiens chromosome 6 sequence as provided in NCBI Accession No.: NC_000006.12). In addition, examples of human HLA-G transcript variants that encode different isoforms of HLA-G proteins are provided in NCBI Accession Nos.: NM_001363567.2 (SEQ ID NO: 7), NM_002127.6 (SEQ ID NO: 8), NM_001384280.1 (SEQ ID NO: 9), or NM_001384290.1 (SEQ ID NO: 10).

### Human HLA-G Gene—NCBI Gene ID: 3151 (SEQ ID NO: 30); 3′-UTR Underlined (SEQ ID NO: 89)

ATAGTAGCAGGACCACTATAGAGAGAACACTCATGTAGCAGGTCATGGAACAGTGCTAGAGCCAC
AGTTCAGGAGTGAGAGGGTGGTGGGGATTAAGGGGAGAAGAGGGCCTGAGGGATGAGAGGGACGG
AGGGAAGGGCTGGAGGAGCAGGAGGTGAGGAAAAGGAGCAGAGGAAAGAATTCCAAAGCAGCAGA
ACTCTTAGGTTTAAACACATTGTTTTATAGATTTTAATACATCCATCTACAGAGCTTCGCTGGGT
GTTCTTTGCAGTTGGCCTTTAATATCTTATGTGGGTCTGCCTAGAAACTAATTGTTTTTTATGTT
AATCAGGTTTAAAAAATACTAAGTATTCCTAAAAAATATACACTCCACTCACATGTGGATACTTC
CTAAAAACAGGCAGTGCGTGAGCACTAGTGAGGGGCATTGTGACTGCACTGAACACTTACAACTG
TGAGGTGAATAAAGTTTGTGCTGGCTCCTGGTTGCAACATATAGTAACATAGTGTGGTACTTTGT
CTTGAGGAGATGTCCTGGACTCACACGGAAACTTAGGGCTACGGAATGAAGGTAAATTTAAAATA
AAACAAGCGGGAGTCACAGATACACTGTCTGGGAAAGTGAAACTTAAGAGCTTTGTGAGTCGTGT
TGTAATGCTTTTAGATGCATTTATATACCAACAGGCCAAAGTCACATTTTTTACCGATTAGATTC
CTGATCATTCAGGGGTTACCAAGGTTATGCTACCCACTATAGTTAATAAACAAAAAGCAAACTGG
TCTCTATTCTATCTCATGCACTCAGGCACAACTTTTCCAGATTTAAGGGGGAAAAAAAACCCTGT
CTTTACACCTACAATCCCAGGGCGAGCTCACTCTCTGGCAACAAGCTCCCTGGGGTGATTTTTCT
TCTAGAAGAGTACAGGAGGACAGGCAAGGAGTGGGAGGCAGGGAGTCCAGTTCAGGGACAGGGAT
TCCGGGATGAAAAGTGAAGGGAGAGGGCCAGGGACCTTGCCGAGGGTTTCTCCCTGGTTTCTCAG
ACAGCTCCTGGGCCAAGACTCAGGGAGACACTGAGACAGAACGCTTGGCACAAGAGTAGCGGGGT
CAGGGCGAAGTCCCAGGGCCTCAAGCGTGGCTCTCAGGGTCTCAGGCCCCACAGGCGGTGTATGG
GTTGGGGAGGCCCCGCGTTGGGGATTCTCTCCTCCTTCTCCTAACCTGTGTCGGGTCCTTCTTCC
TGGATACTCACCGGGCGGCCCCAGTTCTCACTCCCATTAGGTGACAGGTTTTTAGAGAAGCCAAT
CAGCGTCGCCGCGGTCCTGGTTCTAAAGTCCTCGCTCACCCACCCGGACTCATTCTCCCCAGACG
CCAAGGATGGTGGTCATGGCGCCCCGAACCCTCTTCCTGCTGCTCTCGGGGGCCCTGACCCTGAC
CGAGACCTGGGCGGGTGAGTGCGGGGTCAGGAGGGAAACAGCCCCTGCGCGGAGGAGGGAGGGGC
CGGCCCGGCGGGGGCGCAGGACTCGGCAGCCGCGCCGGGAGGAGGGTCGGGCGGGTCTCAACCCC
TCCTCGCCCCCAGGCTCCCACTCCATGAGGTATTTCAGCGCCGCCGTGTCCCGGCCCGGCCGCGG
GGAGCCCCGCTTCATCGCCATGGGCTACGTGGACGACACGCAGTTCGTGCGGTTCGACAGCGACT
CGGCGTGTCCGAGGATGGAGCCGCGGGCGCCGTGGGTGGAGCAGGAGGGGCCGGAGTATTGGGAA
GAGGAGACACGGAACACCAAGGCCCACGCACAGACTGACAGAATGAACCTGCAGACCCTGCGCGG
CTACTACAACCAGAGCGAGGCCAGTGAGTAACTCCGGCCCAGGGAGCAGATCACGACCCCCACCT
CCATGCCCCACGGACGGCCCGGGTACTCCCGAGTCTCCGGGTCTGGGATCCACCCCGAGGCCGCG
GGACCCGCCCAGACCCTCTACCTGGGAGAACCCCAAGGCGCCTTTACCAAAATCCCCGCGGGTGG
GTCCGGGCGAGGGCGAGGCTCGGTGGGCGGGGCTGACCGAGGGGGTGGGGCCAGGTTCTCACACC
CTCCAGTGGATGATTGGCTGCGACCTGGGGTCCGACGGACGCCTCCTCCGCGGGTATGAACAGTA
TGCCTACGATGGCAAGGATTACCTCGCCCTGAACGAGGACCTGCGCTCCTGGACCGCAGCGGACA
CTGCGGCTCAGATCTCCAAGCGCAAGTGTGAGGCGGCCAATGTGGCTGAACAAAGGAGAGCCTAC
CTGGAGGGCACGTGCGTGGAGTGGCTCCACAGATACCTGGAGAACGGGAAGGAGATGCTGCAGCG
CGCGGGTACCAGGGGCAGTGGGGCGCCTCCCTGATCTCCTGTAGACCTCTCAGCCTGGCCTAGCA
CAAGGAGAGGAGGAAAATGGGACCAACACTAGAATATCGCCCTCCCTCTGGTCCTGAGGGAGAGG
AATCCTCCTGGGTTTCCAGATCCTGTACCAGAGAGTGATTCTGAGGGTCCGTCCTGCTCTCTGGG
ACAATTAAGGGATGAAGTCTCTGAGGGAGTGGAGGGGAAGACAATCCCTGGAAGACTGATCAGGG
GTTCCCTTTGACCCCACAGCAGCCTTGGCACCAGGACTTTTCCCCTCAGGCCTTGTTCTCTGCCT
CACACTCAATGTGTGTGGGGGTCTGACTCCAGCTCCTCTGAGTCCCTTGGCCTCCACTCAGGTCA
GAACCGGAGGTCCCTGCTCCCCCGCTCAGAGACTAGAACTTTCCAAGGAATAGGAGATTATCCCA
GGTGCCCGTGTCCAGGCTGGTGTCTGGGTTCTGTGCTCCCTTCCCCACCCCAGGTATCTGGTTCA
TTCTTAGGATGGTCACATCCAGGTGCTGCTGGAGTGTCCCATGAGAGATGCAAAGTGCTTGAATT
TTCTGACTCTTCCTTTCAGACCCCCCCAAGACACACGTGACCCACCACCCTGTCTTTGACTATGA
GGCCACCCTGAGGTGCTGGGCCCTGGGCTTCTACCCTGCGGAGATCATACTGACCTGGCAGCGGG
ATGGGGAGGACCAGACCCAGGACGTGGAGCTCGTGGAGACCAGGCCTGCAGGGGATGGAACCTTC
CAGAAGTGGGCAGCTGTGGTGGTGCCTTCTGGAGAGGAGCAGAGATACACGTGCCATGTGCAGCA
TGAGGGGCTGCCGGAGCCCCTCATGCTGAGATGGAGTAAGGAGGGAGATGGAGGCATCATGTCTG
TTAGGGAAAGCAGGAGCCTCTCTGAAGACCTTTAACAGGGTCGGTGGTGAGGGCTGGGGGTCAGA
GACCCTCACCTTCACCTCCTTTCCCAGAGCAGTCTTCCCTGCCCACCATCCCCATCATGGGTATC
GTTGCTGGCCTGGTTGTCCTTGCAGCTGTAGTCACTGGAGCTGCGGTCGCTGCTGTGCTGTGGAG
AAAGAAGAGCTCAGGTAAGGAAGGGGTGACAAGTGGGGTCTGAGTTTTCTTGTCCCACTGGGGGT
TTCAAGCCCCAGGTAGAAGTGTGCCCTGCCTGGTTACTGGGAAGCACCATCCACACTCATGGGCC
TACCCAGCCTGGGCCCTGTGTGCCAGCACCTTCTCTTTTGTAAAGCACCTGTGACAATGAAGGAC
AGATTTATTACCTTGATGATTGTAGTGATGGGGACCTGATCCCAGTAATCACAGGTCAGGAGAAG
GTCCCTGGCTAAGGACAGACCTTAGGAGGGCAGTTGGTCGAGGACCCACATCTGCTTTCCTTGTT
TTTCCTGATCCCGCCCTGGGTCTGCAGTCACACATTTCTGGAAACTTCTCGAGGGTCCAAGACTA
GGAGGTTCCTCTAGGACCTCATGGCCCTGCCACCTTTCTGGCCTCTCACAGGACATTTTCTTCCC
ACAGATTGAAAAGGAGGGAGCTACTCTCAGGCTGCAAGTAAGTATGAAGGAGGCTGATCCCTGAG
ATCCTTGGGATCTTGTGTTTGGGAGCCCATGGGGGAGCTCACCCACCCCACAATTCCTCCTCTGG
CCACATCTCCTGTGGTCTCTGACCAGGTGCTGTTTTTGTTCTACTCTAGGCAGTGACAGTGCCCA
GGGCTCTAATGTGTCTCTCACGGCTTGTAAATGTGACACCCCGGGGGGCCTGATGTGTGTGGGTT
GTTGAGGGGAACAGGGGACATAGCTGTGCTATGAGGTTTCTTTGACTTCAATGTATTGAGCATGT
GATGGGCTGTTTAAAGTGTCACCCCTCACTGTGACTGATATGAATTTGTTCATGAATATTTTTCT
GTAGTGTGAAACAGCTGCCCTGTGTGGGACTGAGTGGCAAGTCCCTTTGTGACTTCAAGAACCCT
GACTCCTCTTTGTGCAGAGACCAGCCCACCCCTGTGCCCACCATGACCCTCTTCCTCATGCTGAA
CTGCATTCCTTCCCCAATCACCTTTCCTGTTCCAGAAAAGGGGCTGGGATGTCTCCGTCTCTGTC
TCAAATTTGTGGTCCACTGAGCTATAACTTACTTCTGTATTAAAATTAGAATCTGAGTATAAATT
TACTTTTTCAAATTATTTCCAAGAGAGATTGATGGGTTAATTAAAGGAGAAGATTCCTGAAATTT
GAGAGACAAAATAAATGGAAGACATGAGAACTTTCCACAGTA

### Human HLA-G Transcript Variant 1—NM_001363567.2 (SEQ ID NO: 7); 3′-UTR Underlined (SEQ ID NO: 83)

ATATAGTAACATAGTGTGGTACTTTGTCTTGAGGAGATGTCCTGGACTCACACGGAAACTTAGGG
CTACGGAATGAAGACGCCAAGGATGGTGGTCATGGCGCCCCGAACCCTCTTCCTGCTGCTCTCGG
GGGCCCTGACCCTGACCGAGACCTGGGCGGGCTCCCACTCCATGAGGTATTTCAGCGCCGCCGTG
TCCCGGCCCGGCCGCGGGGAGCCCCGCTTCATCGCCATGGGCTACGTGGACGACACGCAGTTCGT
GCGGTTCGACAGCGACTCGGCGTGTCCGAGGATGGAGCCGCGGGCGCCGTGGGTGGAGCAGGAGG
GGCCGGAGTATTGGGAAGAGGAGACACGGAACACCAAGGCCCACGCACAGACTGACAGAATGAAC
CTGCAGACCCTGCGCGGCTACTACAACCAGAGCGAGGCCAGTTCTCACACCCTCCAGTGGATGAT
TGGCTGCGACCTGGGGTCCGACGGACGCCTCCTCCGCGGGTATGAACAGTATGCCTACGATGGCA
AGGATTACCTCGCCCTGAACGAGGACCTGCGCTCCTGGACCGCAGCGGACACTGCGGCTCAGATC
TCCAAGCGCAAGTGTGAGGCGGCCAATGTGGCTGAACAAAGGAGAGCCTACCTGGAGGGCACGTG
CGTGGAGTGGCTCCACAGATACCTGGAGAACGGGAAGGAGATGCTGCAGCGCGCGGACCCCCCCA
AGACACACGTGACCCACCACCCTGTCTTTGACTATGAGGCCACCCTGAGGTGCTGGGCCCTGGGC
TTCTACCCTGCGGAGATCATACTGACCTGGCAGCGGGATGGGGAGGACCAGACCCAGGACGTGGA
GCTCGTGGAGACCAGGCCTGCAGGGGATGGAACCTTCCAGAAGTGGGCAGCTGTGGTGGTGCCTT
CTGGAGAGGAGCAGAGATACACGTGCCATGTGCAGCATGAGGGGCTGCCGGAGCCCCTCATGCTG
AGATGGAAGCAGTCTTCCCTGCCCACCATCCCCATCATGGGTATCGTTGCTGGCCTGGTTGTCCT
TGCAGCTGTAGTCACTGGAGCTGCGGTCGCTGCTGTGCTGTGGAGAAAGAAGAGCTCAGATTGAA
AAGGAGGGAGCTACTCTCAGGCTGCAATGTGAAACAGCTGCCCTGTGTGGGACTGAGTGGCAAGT
CCCTTTGTGACTTCAAGAACCCTGACTCCTCTTTGTGCAGAGACCAGCCCACCCCTGTGCCCACC
ATGACCCTCTTCCTCATGCTGAACTGCATTCCTTCCCCAATCACCTTTCCTGTTCCAGAAAAGGG
GCTGGGATGTCTCCGTCTCTGTCTCAAATTTGTGGTCCACTGAGCTATAACTTACTTCTGTATTA
AAATTAGAATCTGAGTATAAA

### Human HLA-G Transcript Variant 2—NM_002127.6 (SEQ ID NO: 8); 3′-UTR Underlined (SEQ ID NO: 84)

ATATAGTAACATAGTGTGGTACTTTGTCTTGAGGAGATGTCCTGGACTCACACGGAAACTTAGGG
CTACGGAATGAAGTTCTCACTCCCATTAGGTGACAGGTTTTTAGAGAAGCCAATCAGCGTCGCCG
CGGTCCTGGTTCTAAAGTCCTCGCTCACCCACCCGGACTCATTCTCCCCAGACGCCAAGGATGGT
GGTCATGGCGCCCCGAACCCTCTTCCTGCTGCTCTCGGGGGCCCTGACCCTGACCGAGACCTGGG
CGGGCTCCCACTCCATGAGGTATTTCAGCGCCGCCGTGTCCCGGCCCGGCCGCGGGGAGCCCCGC
TTCATCGCCATGGGCTACGTGGACGACACGCAGTTCGTGCGGTTCGACAGCGACTCGGCGTGTCC
GAGGATGGAGCCGCGGGCGCCGTGGGTGGAGCAGGAGGGGCCGGAGTATTGGGAAGAGGAGACAC
GGAACACCAAGGCCCACGCACAGACTGACAGAATGAACCTGCAGACCCTGCGCGGCTACTACAAC
CAGAGCGAGGCCAGTTCTCACACCCTCCAGTGGATGATTGGCTGCGACCTGGGGTCCGACGGACG
CCTCCTCCGCGGGTATGAACAGTATGCCTACGATGGCAAGGATTACCTCGCCCTGAACGAGGACC
TGCGCTCCTGGACCGCAGCGGACACTGCGGCTCAGATCTCCAAGCGCAAGTGTGAGGCGGCCAAT
GTGGCTGAACAAAGGAGAGCCTACCTGGAGGGCACGTGCGTGGAGTGGCTCCACAGATACCTGGA
GAACGGGAAGGAGATGCTGCAGCGCGCGGACCCCCCCAAGACACACGTGACCCACCACCCTGTCT
TTGACTATGAGGCCACCCTGAGGTGCTGGGCCCTGGGCTTCTACCCTGCGGAGATCATACTGACC
TGGCAGCGGGATGGGGAGGACCAGACCCAGGACGTGGAGCTCGTGGAGACCAGGCCTGCAGGGGA
TGGAACCTTCCAGAAGTGGGCAGCTGTGGTGGTGCCTTCTGGAGAGGAGCAGAGATACACGTGCC
ATGTGCAGCATGAGGGGCTGCCGGAGCCCCTCATGCTGAGATGGAAGCAGTCTTCCCTGCCCACC
ATCCCCATCATGGGTATCGTTGCTGGCCTGGTTGTCCTTGCAGCTGTAGTCACTGGAGCTGCGGT
CGCTGCTGTGCTGTGGAGAAAGAAGAGCTCAGATTGAAAAGGAGGGAGCTACTCTCAGGCTGCAA
TGTGAAACAGCTGCCCTGTGTGGGACTGAGTGGCAAGTCCCTTTGTGACTTCAAGAACCCTGACT
CCTCTTTGTGCAGAGACCAGCCCACCCCTGTGCCCACCATGACCCTCTTCCTCATGCTGAACTGC
ATTCCTTCCCCAATCACCTTTCCTGTTCCAGAAAAGGGGCTGGGATGTCTCCGTCTCTGTCTCAA
ATTTGTGGTCCACTGAGCTATAACTTACTTCTGTATTAAAATTAGAATCTGAGTATAAA

### Human HLA-G Transcript Variant 3—NM_001384280.1 (SEQ ID NO: 9); 3′-UTR Underlined (SEQ ID NO: 85)

ATAGTAGCAGGACCACTATAGAGAGAACACTCATGTAGCAGGTCATGGAACAGTGCTAGAGCCAC
AGTTCAGGAATGTCCTGGACTCACACGGAAACTTAGGGCTACGGAATGAAGACGCCAAGGATGGT
GGTCATGGCGCCCCGAACCCTCTTCCTGCTGCTCTCGGGGGCCCTGACCCTGACCGAGACCTGGG
CGGGCTCCCACTCCATGAGGTATTTCAGCGCCGCCGTGTCCCGGCCCGGCCGCGGGGAGCCCCGC
TTCATCGCCATGGGCTACGTGGACGACACGCAGTTCGTGCGGTTCGACAGCGACTCGGCGTGTCC
GAGGATGGAGCCGCGGGCGCCGTGGGTGGAGCAGGAGGGGCCGGAGTATTGGGAAGAGGAGACAC
GGAACACCAAGGCCCACGCACAGACTGACAGAATGAACCTGCAGACCCTGCGCGGCTACTACAAC
CAGAGCGAGGCCAGTTCTCACACCCTCCAGTGGATGATTGGCTGCGACCTGGGGTCCGACGGACG
CCTCCTCCGCGGGTATGAACAGTATGCCTACGATGGCAAGGATTACCTCGCCCTGAACGAGGACC
TGCGCTCCTGGACCGCAGCGGACACTGCGGCTCAGATCTCCAAGCGCAAGTGTGAGGCGGCCAAT
GTGGCTGAACAAAGGAGAGCCTACCTGGAGGGCACGTGCGTGGAGTGGCTCCACAGATACCTGGA
GAACGGGAAGGAGATGCTGCAGCGCGCGGACCCCCCCAAGACACACGTGACCCACCACCCTGTCT
TTGACTATGAGGCCACCCTGAGGTGCTGGGCCCTGGGCTTCTACCCTGCGGAGATCATACTGACC
TGGCAGCGGGATGGGGAGGACCAGACCCAGGACGTGGAGCTCGTGGAGACCAGGCCTGCAGGGGA
TGGAACCTTCCAGAAGTGGGCAGCTGTGGTGGTGCCTTCTGGAGAGGAGCAGAGATACACGTGCC
ATGTGCAGCATGAGGGGCTGCCGGAGCCCCTCATGCTGAGATGGAAGCAGTCTTCCCTGCCCACC
ATCCCCATCATGGGTATCGTTGCTGGCCTGGTTGTCCTTGCAGCTGTAGTCACTGGAGCTGCGGT
CGCTGCTGTGCTGTGGAGAAAGAAGAGCTCAGATTGAAAAGGAGGGAGCTACTCTCAGGCTGCAA
TGTGAAACAGCTGCCCTGTGTGGGACTGAGTGGCAAGTCCCTTTGTGACTTCAAGAACCCTGACT
CCTCTTTGTGCAGAGACCAGCCCACCCCTGTGCCCACCATGACCCTCTTCCTCATGCTGAACTGC
ATTCCTTCCCCAATCACCTTTCCTGTTCCAGAAAAGGGGCTGGGATGTCTCCGTCTCTGTCTCAA
ATTTGTGGTCCACTGAGCTATAACTTACTTCTGTATTAAAATTAGAATCTGAGTATAAA

### Human HLA-G Transcript Variant 4—NM_001384290.1 (SEQ ID NO: 10); 3′-UTR Underlined (SEQ ID NO: 86)

ATTCTCCCCAGACGCCAAGGATGGTGGTCATGGCGCCCCGAACCCTCTTCCTGCTGCTCTCGGGG
GCCCTGACCCTGACCGAGACCTGGGCGGGCTCCCACTCCATGAGGTATTTCAGCGCCGCCGTGTC
CCGGCCCGGCCGCGGGGAGCCCCGCTTCATCGCCATGGGCTACGTGGACGACACGCAGTTCGTGC
GGTTCGACAGCGACTCGGCGTGTCCGAGGATGGAGCCGCGGGCGCCGTGGGTGGAGCAGGAGGGG
CCGGAGTATTGGGAAGAGGAGACACGGAACACCAAGGCCCACGCACAGACTGACAGAATGAACCT
GCAGACCCTGCGCGGCTACTACAACCAGAGCGAGGCCAGTTCTCACACCCTCCAGTGGATGATTG
GCTGCGACCTGGGGTCCGACGGACGCCTCCTCCGCGGGTATGAACAGTATGCCTACGATGGCAAG
GATTACCTCGCCCTGAACGAGGACCTGCGCTCCTGGACCGCAGCGGACACTGCGGCTCAGATCTC
CAAGCGCAAGTGTGAGGCGGCCAATGTGGCTGAACAAAGGAGAGCCTACCTGGAGGGCACGTGCG
TGGAGTGGCTCCACAGATACCTGGAGAACGGGAAGGAGATGCTGCAGCGCGCGGACCCCCCCAAG
ACACACGTGACCCACCACCCTGTCTTTGACTATGAGGCCACCCTGAGGTGCTGGGCCCTGGGCTT
CTACCCTGCGGAGATCATACTGACCTGGCAGCGGGATGGGGAGGACCAGACCCAGGACGTGGAGC
TCGTGGAGACCAGGCCTGCAGGGGATGGAACCTTCCAGAAGTGGGCAGCTGTGGTGGTGCCTTCT
GGAGAGGAGCAGAGATACACGTGCCATGTGCAGCATGAGGGGCTGCCGGAGCCCCTCATGCTGAG
ATGGAAGCAGTCTTCCCTGCCCACCATCCCCATCATGGGTATCGTTGCTGGCCTGGTTGTCCTTG
CAGCTGTAGTCACTGGAGCTGCGGTCGCTGCTGTGCTGTGGAGAAAGAAGAGCTCAGATTGAAAA
GGAGGGAGCTACTCTCAGGCTGCAATGTGAAACAGCTGCCCTGTGTGGGACTGAGTGGCAAGTCC
CTTTGTGACTTCAAGAACCCTGACTCCTCTTTGTGCAGAGACCAGCCCACCCCTGTGCCCACCAT
GACCCTCTTCCTCATGCTGAACTGCATTCCTTCCCCAATCACCTTTCCTGTTCCAGAAAAGGGGC
TGGGATGTCTCCGTCTCTGTCTCAAATTTGTGGTCCACTGAGCTATAACTTACTTCTGTATTAAA
ATTAGAATCTGAGTATAAA

In some embodiments, a disruption in the 3′-UTR of an allele encoding HLA-G comprises a deletion of the 3′-UTR. In some embodiments, a deletion comprises deletion of one or more fragments of the 3′-UTR. In some embodiments, a deletion is a complete deletion of the 3′-UTR. In some embodiments, a disruption in the 3′-UTR of an allele encoding HLA-G comprises an inversion of a sequence in the 3′-UTR. In some embodiments, a disruption in the 3′-UTR of an allele encoding HLA-G comprises an inversion of a fragment in the 3′-UTR. In some embodiments, a disruption in the 3′-UTR of an allele encoding HLA-G comprises an inversion of the entire 3′-UTR. In some embodiments, a disruption in the 3′-UTR of an allele encoding HLA-G comprises a translocation of a sequence in the entire 3′-UTR. In some embodiments, a disruption in the 3′-UTR of an allele encoding HLA-G comprises substitution of one or more nucleotides in the 3′-UTR. In some embodiments, a disruption in the 3′-UTR of an allele encoding HLA-G comprises an insertion of one or more nucleotides in the 3′-UTR.

In some embodiments, the disclosure contemplates a cell in which any of nucleotide positions +3001, +3003, +3010, +3027, +3032, +3035, +3052, +3092, +3111, +3121, +3142, +3177, +3183, +3187, +3196, and +3227 of the HLA-G 3′-UTR has been deleted or substituted with an alternative nucleotide. In some embodiments, the disclosure contemplates a cell in which one or both copies of the cell's HLA-G 3′-UTR comprise any one of or combination of: +3003T, +3010G, +3010C, +3035C, +3142C, +3142G, +3187G, +3187A, +3196C, +3196G, +3227G, +3227A. In some embodiments, the disclosure contemplates a cell in which any of nucleotide positions +3001, +3003, +3010, +3027, +3032, +3035, +3052, +3092, +3111, +3121, +3142, +3177, +3183, +3187, +3196, and +3227 of the HLA-G 3′-UTR has been deleted or substituted with an alternative nucleotide such that a microRNA (e.g., any one or more of miR-133A, miR-148A, miR-148B, miR-152, miR-548q and/or miR-628-5p) is unable to bind to or have significantly reduced binding to the 3′-UTR of an HLA-G RNA transcript. In some embodiments, the disclosure contemplates a cell in which at least 5, 8, 10, 12, 14, 20 consecutive nucleotides have been deleted beginning at and inclusive of position +2961 of the HLA-G 3′-UTR, and/or wherein at least 5, 8, 10, 12, 14, 20 nucleotides have been inserted at position +2961. In some embodiments, the disclosure contemplates a cell in which at least one insertion, deletion, substitution, translocation has been introduced in a nucleic acid sequence corresponding to a sequence that is at least 75%, 80%, 85%, 90%, 91%, 92%, 93%, 94%, 95%, 96%, 97%, 98%, 99% or 100% identical to SEQ ID NO: 74. In some embodiments, the disclosure contemplates a cell in which at least 1, 3, 5, 8, 10, 12 or 14 nucleotides of SEQ ID NO: 75 (ATTTGTTCATGCCT) are not present in (e.g., have been deleted from) a nucleic acid comprising a sequence that is at least 75%, 80%, 85%, 90%, 91%, 92%, 93%, 94%, 95%, 96%, 97%, 98%, 99% or 100% identical to SEQ ID NO: 74 (e.g., all of SEQ ID NO: 75 has been deleted from a nucleic acid comprising a sequence that is at least 75%, 80%, 85%, 90%, 91%, 92%, 93%, 94%, 95%, 96%, 97%, 98%, 99% or 100% identical to SEQ ID NO: 74). In some embodiments, the cell comprises a nucleic acid in which a G is present at the position corresponding to position 120 of a sequence that is at least 75%, 80%, 85%, 90%, 91%, 92%, 93%, 94%, 95%, 96%, 97%, 98%, 99% or 100% identical to SEQ ID NO: 74. In some embodiments, the cell comprises a nucleic acid in which a C is present at the position corresponding to position 252 of a sequence that is at least 75%, 80%, 85%, 90%, 91%, 92%, 93%, 94%, 95%, 96%, 97%, 98%, 99% or 100% identical to SEQ ID NO: 74. In some embodiments, the cell comprises a nucleic acid in which a G is present at the position corresponding to position 297 of a sequence that is at least 75%, 80%, 85%, 90%, 91%, 92%, 93%, 94%, 95%, 96%, 97%, 98%, 99% or 100% identical to SEQ ID NO: 74. In some embodiments, the cell comprises a nucleic acid in which a G is present at the position corresponding to position 306 of a sequence that is at least 75%, 80%, 85%, 90%, 91%, 92%, 93%, 94%, 95%, 96%, 97%, 98%, 99% or 100% identical to SEQ ID NO: 74. See, e.g., Poras et al., 2017, PLOS One, DOI:10.1371/journal.pone.0169032; Schwich et al., 2019, Scientific Reports, 9:5407. In some embodiments, the cell is heterozygous for any one of or combination of the above genetic elements listed in this paragraph. In some embodiments, the cell is homozygous for any one of or combination of the above genetic elements listed in this paragraph.

SEQ ID NO: 74
ATTGAAAAGGAGGGAGCTACTCTCAGGCTGCAATGTGAAACAGCTGCCCTGTGTGG
GACTGAGTGGCAAGATTTGTTCATGCCTTCCCTTTGTGACTTCAAGAACCCTGACTTC
TCTTTCTGCAGAGACCAGCCCACCCCTGTGCCCACCATGACCCTCTTCCTCATGCTGA
ACTGCATTCCTTCCCCAATCACCTTTCCTGTTCCAGAAAAGGGGCTGGGATGTCTCC
GTCTCTGTCTCAAATTTGTGGTGCACTGAGCTATAACTTACTTCTGTATTAAAATTAG
AATCTGAGTATAAATTTACTTTTTCAAATTATTTCCAAGAGAGATTGATGGGTTAATT
AAAGGAGAAGATTCCTGAAATTTGAGAGACAAAATAAATGGAAGAC

In some embodiments, a disruption in the 3′-UTR of an allele HLA-G leads to increased expression (e.g., increased by at least 10%, at least 20%, at least 30%, at least 40%, at least 50%, at least 60%, at least 70%, at least 80%, at least 90%, at least 100%, at least 2-fold, at least 5-fold, at least 10-fold, at least 50-fold, at least 100-fold or higher) of HLA-G in the cell, the isolated stem cell and cells differentiated from the isolated stem cell. In some embodiments, the increased expression of HLA-G is induced or increased by interferon gamma.

In some embodiments, a cell (e.g., an isolated stem cell) described herein does not comprise additional exogenous expression of any factors (e.g., protein or RNA). In some embodiments, an isolated cell (e.g., stem cell) described herein does not comprise an insertion of an exogenous nucleotide sequence anywhere in its genome. In some embodiments, the disclosure provides for isolated cells (e.g., stem cells) that comprise disruptions in the 3′-UTRs of more than one alleles in the cells' genomes, e.g., in the 3′-UTRs of more than one of the alleles encoding for any of PD-L1, CD47, or HLA-G.

In some embodiments, a cell (e.g., an isolated stem cell) described herein comprises a disruption in the 3′-UTR of an allele encoding PDL2. In some embodiments, the disruption is a homozygous modification. In some embodiments, the disruption is a heterozygous modification. In some embodiments, a disruption in the 3′-UTR of an allele encoding PDL2 comprises a deletion of the 3′-UTR. In some embodiments, a deletion comprises deletion of one or more fragments of the 3′-UTR. In some embodiments, a deletion is a complete deletion of the 3′-UTR. In some embodiments, a disruption in the 3′-UTR of an allele encoding PDL2 comprises an inversion of a sequence in the 3′-UTR. In some embodiments, a disruption in the 3′-UTR of an allele encoding PDL2 comprises an inversion of a fragment in the 3′-UTR. In some embodiments, a disruption in the 3′-UTR of an allele encoding PDL2 comprises an inversion of the entire 3′-UTR. In some embodiments, a disruption in the 3′-UTR of an allele encoding PDL2 comprises a translocation of a sequence in the entire 3′-UTR. In some embodiments, a disruption in the 3′-UTR of an allele encoding PDL2 comprises substitution of one or more nucleotides in the 3′-UTR. In some embodiments, a disruption in the 3′-UTR of an allele encoding PDL2 comprises an insertion of one or more nucleotides in the 3′-UTR. In some embodiments, the 3′-UTR of PDL2, as referenced herein, comprises a sequence that is at least 75%, 80%, 85%, 90%, 91%, 92%, 93%, 94%, 95%, 96%, 97%, 98%, 99%, or 100% identical to the nucleotide sequence of SEQ ID NO: 76, and 90-96 or a portion thereof.

(Accession No. NM_025239)
SEQ ID NO: 76
GGCAAGTTGGACGCCCGCAAGATCCGCGAGATTCTCATTAAGGCCAAGAAGGGCGG
AAAGATCGCCGTGTAACAATTGGCAGAGCTCAGAATTCAAGCGATCGCCACAAAGA
GGGAAGTGAACAGTGCTATCTGAACCTGTGGTCTTGGGAGCCAGGGTGACCTGATA
TGACATCTAAAGAAGCTTCTGGACTCTGAACAAGAATTCGGTGGCCTGCAGAGCTTG
CCATTTGCACTTTTCAAATGCCTTTGGATGACCCAGCACTTTAATCTGAAACCTGCAA
CAAGACTAGCCAACACCTGGCCATGAAACTTGCCCCTTCACTGATCTGGACTCACCT
CTGGAGCCTATGGCTTTAAGCAAGCACTACTGCACTTTACAGAATTACCCCACTGGA
TCCTGGACCCACAGAATTCCTTCAGGATCCTTCTTGCTGCCAGACTGAAAGCAAAAG
GAATTATTTCCCCTCAAGTTTTCTAAGTGATTTCCAAAAGCAGAGGTGTGTGGAAAT
TTCCAGTAACAGAAACAGATGGGTTGCCAATAGAGTTATTTTTTATCTATAGCTTCCT
CTGGGTACTAGAAGAGGCTATTGAGACTATGAGCTCACAGACAGGGCTTCGCACAA
ACTCAAATCATAATTGACATGTTTTATGGATTACTGGAATCTTGATAGCATAATGAA
GTTGTTCTAATTAACAGAGAGCATTTAAATATACACTAAGTGCACAAATTGTGGAGT
AAAGTCATCAAGCTCTGTTTTTGAGGTCTAAGTCACAAAGCATTTGTTTTAACCTGTA
ATGGCACCATGTTTAATGGTGGTTTTTTTTTTGAACTACATCTTTCCTTTAAAAATTAT
TGGTTTCTTTTTATTTGTTTTTACCTTAGAAATCAATTATATACAGTCAAAAATATTTG
ATATGCTCATACGTTGTATCTGCAGCAATTTCAGATAAGTAGCTAAAATGGCCAAAG
CCCCAAACTAAGCCTCCTTTTCTGGCCCTCAATATGACTTTAAATTTGACTTTTCAGT
GCCTCAGTTTGCACATCTGTAATACAGCAATGCTAAGTAGTCAAGGCCTTTGATAAT
TGGCACTATGGAAATCCTGCAAGATCCCACTACATATGTGTGGAGCAGAAGGGTAA
CTCGGCTACAGTAACAGCTTAATTTTGTTAAATTTGTTCTTTATACTGGAGCCATGAA
GCTCAGAGCATTAGCTGACCCTTGAACTATTCAAATGGGCACATTAGCTAGTATAAC
AGACTTACATAGGTGGGCCTAAAGCAAGCTCCTTAACTGAGCAAAATTTGGGGCTTA
TGAGAATGAAAGGGTGTGAAATTGACTAACAGACAAATCATACATCTCAGTTTCTCA
ATTCTCATGTAAATCAGAGAATGCCTTTAAAGAATAAAACTCAATTGTTATTCTTCA
ACGTTCTTTATATATTCTACTTTTGGGTAACGCGTAAGCGGCCGCGGCATCTAGATTC
GAAGAAAATGACCGACCAAGCGACGCCCAACCTGCCATCACGAGATTTCGATTCCA
CCGCCGCCTTCTATGAAAGG

In some embodiments, the cell comprises a disruption of the nucleotides corresponding to nucleotides 581-603 of SEQ ID NO: 76. In some embodiments, the cell comprises a disruption of the nucleotides corresponding to nucleotides 362-387 of SEQ ID NO: 76. In some embodiments, the cell comprises a disruption of the nucleotides corresponding to nucleotides 394-416 of SEQ ID NO: 76. In some embodiments, the cell comprises a disruption of the nucleotides corresponding to nucleotides 699-723 of SEQ ID NO: 76. In some embodiments, the cell comprises a disruption the nucleotides corresponding to of nucleotides 1333-1353 of SEQ ID NO: 76. In some embodiments, the cell comprises a disruption of the nucleotides corresponding to nucleotides 686-709 of SEQ ID NO: 76. In some embodiments, the cell comprises a disruption of the nucleotides corresponding to nucleotides 764-785 of SEQ ID NO: 76. In some embodiments, the disclosure contemplates a cell in which SEQ ID NO: 90 (AAGAGGCTATTGAGACTATGAGC) of the PDL2 3′-UTR comprises one or more nucleotide deletions, insertions, and/or substitutions. In some embodiments, the disclosure contemplates a cell in which SEQ ID NO: 91 (AAGCACTACTGCACTTTACAGAATTA) of the PDL2 3′-UTR comprises one or more nucleotide deletions, insertions, and/or substitutions. In some embodiments, the disclosure contemplates a cell in which SEQ ID NO: 92 (TGGATCCTGGACCCACAGAATTC) of the PDL2 3′-UTR comprises one or more nucleotide deletions, insertions, and/or substitutions. In some embodiments, the disclosure contemplates a cell in which SEQ ID NO:93 (GAGAGCATTTAAATATACACTAAGT) of the PDL2 3′-UTR comprises one or more nucleotide deletions, insertions, and/or substitutions. In some embodiments, the disclosure contemplates a cell in which SEQ ID NO: 94 (GAAATTGACTAACAGACAAAT) of the PDL2 3′-UTR comprises one or more nucleotide deletions, insertions, and/or substitutions. In some embodiments, the disclosure contemplates a cell in which SEQ ID NO: 95 (GTTCTAATTAACAGAGAGCATTTA) of the PDL2 3′-UTR comprises one or more nucleotide deletions, insertions, and/or substitutions. In some embodiments, the disclosure contemplates a cell in which SEQ ID NO: 96 (GGTCTAAGTCACAAAGCATTTG) of the PDL2 3′-UTR comprises one or more nucleotide deletions, insertions, and/or substitutions. In some embodiments, the cell comprises one or more nucleotide deletions, insertions, and/or substitutions in any one of 76 or 90-96 such that an endogenous microRNA has reduced or ablated binding in the cell. In some embodiments, the endogenous microRNA is any one or more of: miR-BHRF1-2-5p, miR-BART1-5p, miR-BART7-3p, and/or miR-BART14-3p.

In some embodiments, the disclosure provides for a cell in which 1, 2, 3, 4, 5, 6, 7, 8, 9, 10, 11, 12, 13, 14, 15, 16, 17, 18, 19, 20, 21, 22, 23 or 24 or all of the nucleotides have been deleted from any one of SEQ ID Nos: 76 and 90-96 of the PDL2 3′-UTR. In some embodiments, the disclosure provides for a cell in which 1, 2, 3, 4, 5, 6, 7, 8, 9, 10, 11, 12, 13, 14, 15, 16, 17, 18, 19, 20, 21, 22, 23 or 24 or all of the nucleotides have been substituted in any one of SEQ ID Nos: 76 and 90-96 of the PDL2 3′-UTR. In some embodiments, the disclosure provides for a cell in which 1, 2, 3, 4, 5, 6, 7, 8, 9, 10, 11, 12, 13, 14, 15, 16, 17, 18, 19, 20, 21, 22, 23 or 24 or all of the nucleotides have been in inserted in any one of SEQ ID Nos: 76 and 90-96 of the PDL2 3′-UTR. See, e.g., Cristino, 2019, Blood, 134(25):2261-2270, which is incorporated by reference herein in its entirety. It should be noted that, because the sequences of SEQ ID Nos: 76 and 90-96 of the PDL2 3′-UTR are derived from naturally occurring nucleotide sequences in a cell, it is possible that the nucleic acids in the cell will have some differences (e.g., polymorphisms) as compared to these reference sequences. As such, the disclosure contemplates that the cell may comprise a nucleotide sequence having no more than one, two, three, four, five, or six nucleotide differences as compared to any of the reference sequences of SEQ ID Nos: 76 and 90-96 of the PDL2 3′-UTR prior to modification. In some embodiments, the cell is heterozygous for any one of or combination of the above genetic elements listed in this paragraph. In some embodiments, the cell is homozygous for any one of or combination of the above genetic elements listed in this paragraph.

In some embodiments, a disruption in the 3′-UTR of an allele encoding an PDL2 leads to increased expression (e.g., increased by at least 10%, at least 20%, at least 30%, at least 40%, at least 50%, at least 60%, at least 70%, at least 80%, at least 90%, at least 100%, at least 2-fold, at least 5-fold, at least 10-fold, at least 50-fold, at least 100-fold or higher) of PDL2 in the cell, the isolated stem cell, or cells differentiated from the isolated stem cell. In some embodiments, the increased expression of PDL2 is induced or increased by interferon gamma.

In some embodiments, a cell (e.g., an isolated stem cell) described herein comprises a disruption in the 3′-UTR of an allele encoding IL-10. In some embodiments, the disruption is a homozygous modification. In some embodiments, the disruption is a heterozygous modification. In some embodiments, a disruption in the 3′-UTR of an allele encoding IL-10 comprises a deletion of the 3′-UTR. In some embodiments, a deletion comprises deletion of one or more fragments of the 3′-UTR. In some embodiments, a deletion is a complete deletion of the 3′-UTR. In some embodiments, a disruption in the 3′-UTR of an allele encoding IL-10 comprises an inversion of a sequence in the 3′-UTR. In some embodiments, a disruption in the 3′-UTR of an allele encoding IL-10 comprises an inversion of a fragment in the 3′-UTR. In some embodiments, a disruption in the 3′-UTR of an allele encoding IL-10 comprises an inversion of the entire 3′-UTR. In some embodiments, a disruption in the 3′-UTR of an allele encoding IL-10 comprises a translocation of a sequence in the entire 3′-UTR. In some embodiments, a disruption in the 3′-UTR of an allele encoding IL-10 comprises substitution of one or more nucleotides in the 3′-UTR. In some embodiments, a disruption in the 3′-UTR of an allele encoding IL-10 comprises an insertion of one or more nucleotides in the 3′-UTR. In some embodiments, the 3′-UTR of IL-10, as referenced herein, comprises a sequence that is at least 75%, 80%, 85%, 90%, 91%, 92%, 93%, 94%, 95%, 96%, 97%, 98%, 99%, or 100% identical to the nucleotide sequence of SEQ ID NO: 77, 97, or TACCTCA or a portion thereof.

SEQ ID NO: 77
GACATCAGGGTGGCGACTCTATAGACTCTAGGACATAAATTAGAGGTCTCCAAAAT
CGGATCTGGGGCTCTGGGATAGCTGACCCAGCCCCTTGAGAAACCTTATTGTACCTC
TCTTATAGAATATTTATTACCTCTGATACCTCAACCCCCATTTCTATTTATTTACTGA
GCTTCTCTGTGAACGATTTAGAAAGAAGCCCAATATTATAATTTTTTTCAATATTTAT
TATTTTCACCTGTTTTTAAGCTGTTTCCATAGGGTGACACACTATGGTATTTGAGTGT
TTTAAGATAAATTATAAGTTACATAAGGGAGGAAAAAAAATGTTCTTTGGGGAGCC
AACAGAAGCTTCCATTCCAAGCCTGACCACGCTTTCTAGCTGTTGAGCTGTTTTCCCT
GACCTCCCTCTAATTTATCTTGTCTCTGGGCTTGGGGCTTCCTAACTGCTACAAATAC
TCTTAGGAAGAGAAACCAGGGAGCCCCTTTGATGATTAATTCACCTTCCAGTGTCTC
GGAGGGATTCCCCTAACCTCATTCCCCAACCACTTCATTCTTGAAAGCTGTGGCCAG
CTTGTTATTTATAACAACCTAAATTTGGTTCTAGGCCGGGCGCGGTGGCTCACGCCT
GTAATCCCAGCACTTTGGGAGGCTGAGGCGGGTGGATCACTTGAGGTCAGGAGTTC
CTAACCAGCCTGGTCAACATGGTGAAACCCCGTCTCTACTAAAAATACAAAAATTAG
CCGGGCATGGTGGCGCGCACCTGTAATCCCAGCTACTTGGGAGGCTGAGGCAAGAG
AATTGCTTGAACCCAGGAGATGGAAGTTGCAGTGAGCTGATATCATGCCCCTGTACT
CCAGCCTGGGTGACAGAGCAAGACTCTGTCTCAAAAAATAAAAATAAAAATAAATT
TGGTTCTAATAGAACTCAGTTTTAACTAGAATTTATTCAATTCCTCTGGGAATGTTAC
ATTGTTTGTCTGTCTTCATAGCAGATTTTAATTTTGAATAAATAAATGTATCTTATTC
ACATC

In some embodiments, the cell comprises a disruption of the nucleotides corresponding to nucleotides 125-147 of SEQ ID NO: 77. In some embodiments, the disclosure contemplates a cell in which SEQ ID NO: 97 (ATTTATTACCTCTGATACCTCAA) of the IL-10 3′-UTR comprises one or more nucleotide deletions, insertions, and/or substitutions. In some embodiments, the disclosure contemplates a cell in which TACCTCA of the IL-10 3′-UTR comprises one or more nucleotide deletions, insertions, and/or substitutions. In some embodiments, the cell comprises one or more nucleotide deletions, insertions, and/or substitutions in any one of 77, 97, or TACCTCA such that an endogenous microRNA has reduced or ablated binding in the cell. In some embodiments, the endogenous microRNA is any one or more of: let-7b, let-7c, or let-7f.

In some embodiments, the disclosure provides for a cell in which 1, 2, 3, 4, 5, 6, 7, 8, 9, 10, 11, 12, 13, 14, 15, 16, 17, 18, 19, 20, 21, 22, 23 or 24 or all of the nucleotides have been deleted from any one of SEQ ID Nos: 77, 97, or TACCTCA of the IL-10 3′-UTR. In some embodiments, the disclosure provides for a cell in which 1, 2, 3, 4, 5, 6, 7, 8, 9, 10, 11, 12, 13, 14, 15, 16, 17, 18, 19, 20, 21, 22, 23 or 24 or all of the nucleotides have been substituted in any one of SEQ ID Nos: 77, 97, or TACCTCA of the IL-10 3′-UTR. In some embodiments, the disclosure provides for a cell in which 1, 2, 3, 4, 5, 6, 7, 8, 9, 10, 11, 12, 13, 14, 15, 16, 17, 18, 19, 20, 21, 22, 23 or 24 or all of the nucleotides have been in inserted in any one of SEQ ID Nos: 77, 97, or TACCTCA of the IL-10 3′-UTR. See, e.g., Swaminathan et al., 2012, J. Immunol., 188(12):6238-6246, which is incorporated by reference herein in its entirety. It should be noted that, because the sequences of SEQ ID Nos: 77, 97, or TACCTCA of the IL-10 3′-UTR are derived from naturally occurring nucleotide sequences in a cell, it is possible that the nucleic acids in the cell will have some differences (e.g., polymorphisms) as compared to these reference sequences. As such, the disclosure contemplates that the cell may comprise a nucleotide sequence having no more than one, two, three, four, five, or six nucleotide differences as compared to any of the reference sequences of SEQ ID Nos: 77, 97, or TACCTCA of the IL-10 3′-UTR prior to modification. In some embodiments, the cell is heterozygous for any one of or combination of the above genetic elements listed in this paragraph. In some embodiments, the cell is homozygous for any one of or combination of the above genetic elements listed in this paragraph.

In some embodiments, a disruption in the 3′-UTR of an allele encoding an IL-10 leads to increased expression (e.g., increased by at least 10%, at least 20%, at least 30%, at least 40%, at least 50%, at least 60%, at least 70%, at least 80%, at least 90%, at least 100%, at least 2-fold, at least 5-fold, at least 10-fold, at least 50-fold, at least 100-fold or higher) of IL-10 in the cell, the isolated stem cell, or cells differentiated from the isolated stem cell. In some embodiments, the increased expression of IL-10 is induced or increased by interferon gamma.

In some embodiments, a cell (e.g., an isolated stem cell) described herein further comprises exogenous expression of one or more immunosuppressors. In some embodiments, a cell (e.g., an isolated stem cell) described herein further comprises an insertion of a polynucleotide comprising a nucleotide sequence encoding one or more immunosuppressors in its genome. Non-limiting examples of the one or more immunosuppressor for exogenous expression include: CD47, PDL1, PDL2, CTLA-4, HLA-C, HLA-E, HLA-G, C1-inhibitor, IL-35, DUX4, IDO1, IL10, CCL21, CCL22, CD16, CD52, H2-M3, CD200, FASLG, MFGE8, and SERPINB9. Nucleotide sequences encoding an immunosuppressor (e.g., CD47, PDL1, CTLA-4, HLA-C, HLA-E, HLA-G, C1-inhibitor, or IL-35) are known in the art. In some embodiments, a cell comprises an insertion of a polynucleotide comprising a nucleotide sequence encoding one or more of the following: TGFβ, CD73, CD39, LAG3, IL1R2, ACKR2, TNFRSF22, TNFRSF23, TNFRS10, DAD1, and/or IFNγRI d39. In some embodiments, a nucleotide sequence encoding the one or more immunosuppressors that is inserted in the genome of a cell (e.g., an isolated stem cell) described herein is modified (e.g., codon optimized).

In some embodiments, the one or more immunosuppressor for exogenous expression is CD47, PDL1, and/or CTLA-4. Non-limiting examples of nucleotide sequences encoding different isoforms of CD47 proteins are provided in NCBI Accession Nos.: NM_001777.4 (SEQ ID NO: 4), NM_198793.3 (SEQ ID NO: 5), or NM_001382306.1 (SEQ ID NO: 6). Non-limiting examples of nucleotide sequences encoding different isoforms of PDL1 proteins are provided in NCBI Accession Nos.: NM_014143.4 (SEQ ID NO: 1), NM_001267706.2 (SEQ ID NO: 2), or NM_001314029.2 (SEQ ID NO: 3). Non-limiting examples of human nucleotide sequences encoding different isoforms of HLA-G proteins are provided in NCBI Accession Nos.: NM_001363567.2 (SEQ ID NO: 7), NM_002127.6 (SEQ ID NO: 8), NM_001384280.1 (SEQ ID NO: 9), or NM_001384290.1 (SEQ ID NO: 10). Non-limiting examples of human nucleotide sequences encoding different isoforms of CTLA-4 proteins are provided in NCBI Accession Nos.: NM_001037631.3 (SEQ ID NO: 11) or NM_005214.5 (SEQ ID NO: 12).

Non-limiting examples of amino acid sequences of different isoforms of CD47 proteins are provided in NCBI Accession Nos.: NP_001369235.1 (SEQ ID NO: 49), NP_001768.1 (SEQ ID NO: 50), or NP_942088.1 (SEQ ID NO: 51). Non-limiting examples of amino acid sequences of different isoforms of PDL1 proteins are provided in NCBI Accession Nos.: NP_001254635.1 (SEQ ID NO: 52), NP_001300958.1 (SEQ ID NO: 53), or NP_054862.1 (SEQ ID NO: 54). Non-limiting examples of amino acid sequences of different isoforms of CTLA-4 proteins are provided in NCBI Accession Nos.: NP_001032720.1 (SEQ ID NO: 55) or NP_005205.2 (SEQ ID NO: 56).

In some embodiments, an isolated cell (e.g., an isolated stem cell) described herein comprises an insertion of an exogenous polynucleotide comprising a nucleotide sequence encoding a polypeptide that is at least 75% (e.g., at least 75%, at least 80%, at least 85%, at least 90%, at least 95%, at least 99%, or 100%) identical to the amino acid sequence of any one of SEQ ID NOs: 49-56, or fragments thereof. In some embodiments, an isolated cell (e.g., an isolated stem cell) described herein comprises an insertion of an exogenous polynucleotide comprising a nucleotide sequence encoding a polypeptide comprising the amino acid sequence of any one of SEQ ID NOs: 49-56, or fragments or variants thereof.

In some embodiments, an isolated cell (e.g., an isolated stem cell) described herein further comprises an insertion of an exogenous polynucleotide comprising a nucleotide sequence encoding one or more immunosuppressors (e.g., CD47, CTLA-4, PDL1, PDL2, HLA-C, HLA-E, HLA-G, C1-inhibitor, IL-35, DUX4, IDO1, IL10, CCL21, CCL22, CD16, CD52, H2-M3, CD200, FASLG, MFGE8, and/or SERPINB9) in the disrupted 3′-UTR locus of an endogenous immunosuppressor gene (e.g., PDL1, CD47 or HLA-G) in a cell (e.g., a stem cell). In some embodiments, insertion of an exogenous polynucleotide sequence encoding one or more immunosuppressor (e.g., CD47, CTLA-4, PDL1, PDL2, HLA-C, HLA-E, HLA-G, C1-inhibitor, IL-35, DUX4, IDO1, IL10, CCL21, CCL22, CD16, CD52, H2-M3, CD200, FASLG, MFGE8, and/or SERPINB9) in the disrupted 3′-UTR locus of an endogenous immunosuppressor gene (e.g., PDL1, CD47 or HLA-G) in a cell results in an RNA (e.g., mRNA) comprising the coding sequence for both immunosuppressors. In some embodiments, insertion of an exogenous polynucleotide encoding one or more immunosuppressors (e.g., CD47, CTLA-4, PDL1, PDL2, HLA-C, HLA-E, HLA-G, C1-inhibitor, IL-35, DUX4, IDO1, IL10, CCL21, CCL22, CD16, CD52, H2-M3, CD200, FASLG, MFGE8, and/or SERPINB9) in the disrupted 3′-UTR locus of an endogenous immunosuppressor gene (e.g., PDL1, CD47 or HLA-G) in a cell results in the cell expressing increased levels (e.g., increased by at least 10%, at least 20%, at least 30%, at least 40%, at least 50%, at least 60%, at least 70%, at least 80%, at least 90%, at least 100%, at least 2-fold, at least 5-fold, at least 10-fold, at least 50-fold, at least 100-fold or higher) of the exogenous polynucleotide and the endogenous immunosuppressor gene as compared to a cell of the same cell type lacking the exogenous polynucleotide and the disrupted 3′-UTR locus of the endogenous immunosuppressor gene.

In some embodiments, an isolated cell (e.g., stem cell) described herein further comprises an insertion of a nucleotide encoding one or more immunosuppressors (e.g., CD47, CTLA-4, PDL1, PDL2, HLA-C, HLA-E, HLA-G, C1-inhibitor, IL-35, DUX4, IDO1, IL10, CCL21, CCL22, CD16, CD52, H2-M3, CD200, FASLG, MFGE8, and/or SERPINB9) into a safe harbor locus (e.g., the AAVS1 locus). A “safe harbor locus,” as used herein, refers to a genomic locus in which genes or other genetic elements can be safely inserted and expressed. Genes or genetic elements randomly inserted into a host genome may interact with host genes and host genetic elements in unpredictable ways. A safe harbor locus is a known site for safe insertion of foreign genes or genetic elements that ensures proper expression and function of the foreign genes or genetic element without significantly compromising the health of the cell.

In some embodiments, any of the isolated cells disclosed herein (e.g., any of the stem cells disclosed herein) comprises a “safety switch.” In some embodiments, the safety switches are nucleic acid constructs encoding a switch protein that inducibly causes cell death or stops cell proliferation. In some embodiments, the safety switch is inserted at a defined, specific target locus (e.g., a safe harbor locus) in the genome of an engineered cell, usually at both alleles of the target locus. In some embodiments, the target locus is a safe harbor locus, such as ActB or CLYBL. In some embodiments, the switch protein is activated by contacting with an effective dose of a clinically acceptable orthologous small molecule. In some embodiments, when activated, the safety switch causes the cell to stop proliferation, in some embodiments by activating apoptosis of the cell. In some embodiments, the switch protein comprises herpes-simplex-thymidine-kinase. In some embodiments the switch protein comprises a human caspase protein, e.g., caspase 1, caspase 2, caspase 3, caspase 4, caspase 5, caspase 6, caspase 7, caspase 8, caspase 9, caspase 10, caspase 14, etc. In certain embodiments the protein is human caspase 9.

In some embodiments, the caspase protein is fused to a sequence that provides for chemically induced dimerization (CID), in which dimerization occurs only in the presence of the orthologous activating agent. One or more CID domains may be fused to the caspase protein, e.g. two different CID domains may be fused to the caspase protein. In some embodiments the CID domain is a dimerization domain of FKBP or FRB (FKBP-rapamycin-binding) domain of mTOR, which are activated with rapamycin analogs. In some embodiments, the safety switch is any of the safety switches described in WO2021173449 and Jones et al., 2014, Frontiers in Pharmacology, 5(254):1-8, each of which is incorporated herein in its entirety.

In some embodiments, any of the isolated cells (e.g., stem cells) described herein comprises a disruption (e.g., deletion, insertion, translocation, inversion, or substitution) in the 3′-UTR of an allele encoding PDL1 and further comprises an insertion of a polynucleotide encoding CD47 in the disrupted 3′-UTR locus of PDL1. In some embodiments, any of the isolated cells (e.g., stem cells) described herein comprises a disruption (e.g., deletion, insertion, translocation, inversion, or substitution) in the 3′-UTR of an allele encoding PDL1 and further comprises an insertion of a polynucleotide encoding CD47 in a safe harbor locus.

In some embodiments, any of the isolated cells (e.g., stem cells) described herein comprises a disruption (e.g., deletion, insertion, translocation, inversion, or substitution) in the 3′-UTR of an allele encoding PDL1 and further comprises an insertion of a polynucleotide encoding CTLA-4 in the disrupted 3′-UTR locus of PDL1. In some embodiments, any of the isolated cells (e.g., stem cells) described herein comprises a disruption (e.g., deletion, insertion, translocation, inversion, or substitution) in the 3′-UTR of an allele encoding PDL1 and further comprises an insertion of a polynucleotide encoding CTLA-4 in a safe harbor locus.

In some embodiments, any of the isolated cells (e.g., stem cells) described herein comprises a disruption (e.g., deletion, insertion, translocation, inversion, or substitution) in the 3′-UTR of an allele encoding PDL1 and further comprises an insertion of a polynucleotide encoding PDL1 in the disrupted 3′-UTR locus of PDL1. In some embodiments, any of the isolated cells (e.g., stem cells) described herein comprises a disruption (e.g., deletion, insertion, translocation, inversion, or substitution) in the 3′-UTR of an allele encoding PDL1 and further comprises an insertion of a polynucleotide encoding PDL1 in a safe harbor locus.

In some embodiments, any of the isolated cells (e.g., stem cells) described herein comprises a disruption (e.g., deletion, insertion, translocation, inversion, or substitution) in the 3′-UTR of an allele encoding CD47 and further comprises an insertion of a polynucleotide encoding CD47 in the disrupted 3′-UTR locus of CD47. In some embodiments, any of the isolated cells (e.g., stem cells) described herein comprises a disruption (e.g., deletion, insertion, translocation, inversion, or substitution) in the 3′-UTR of an allele encoding CD47 and further comprises an insertion of a polynucleotide encoding CD47 in a safe harbor locus.

In some embodiments, any of the isolated cells (e.g., stem cells) described herein comprises a disruption (e.g., deletion, insertion, translocation, inversion, or substitution) in the 3′-UTR of an allele encoding CD47 and further comprises an insertion of a polynucleotide encoding CTLA-4 in the disrupted 3′-UTR locus of CD47. In some embodiments, any of the isolated cells (e.g., stem cells) described herein comprises a disruption (e.g., deletion, insertion, translocation, inversion, or substitution) in the 3′-UTR of an allele encoding CD47 and further comprises an insertion of a polynucleotide encoding CTLA-4 in a safe harbor locus.

In some embodiments, any of the isolated cells (e.g., stem cells) described herein comprises a disruption (e.g., deletion, insertion, translocation, inversion, or substitution) in the 3′-UTR of an allele encoding CD47 and further comprises an insertion of a polynucleotide encoding PDL1 in the disrupted 3′-UTR locus of CD47. In some embodiments, any of the isolated cells (e.g., stem cells) described herein comprises a disruption (e.g., deletion, insertion, translocation, inversion, or substitution) in the 3′-UTR of an allele encoding CD47 and further comprises an insertion of a polynucleotide encoding PDL1 in a safe harbor locus.

In some embodiments, any of the isolated cells (e.g., stem cells) described herein comprises a disruption (e.g., deletion, insertion, translocation, inversion, or substitution) in the 3′-UTR of an allele encoding HLA-G and further comprises an insertion of a polynucleotide encoding CD47 in the disrupted 3′-UTR locus of HLA-G. In some embodiments, any of the isolated cells (e.g., stem cells) described herein comprises a disruption (e.g., deletion, insertion, translocation, inversion, or substitution) in the 3′-UTR of an allele encoding HLA-G and further comprises an insertion of a polynucleotide encoding CD47 in a safe harbor locus.

In some embodiments, any of the isolated cells (e.g., stem cells) described herein comprises a disruption (e.g., deletion, insertion, translocation, inversion, or substitution) in the 3′-UTR of an allele encoding HLA-G and further comprises an insertion of a polynucleotide encoding CTLA-4 in the disrupted 3′-UTR locus of HLA-G. In some embodiments, any of the isolated cells (e.g., stem cells) described herein comprises a disruption (e.g., deletion, insertion, translocation, inversion, or substitution) in the 3′-UTR of an allele encoding HLA-G and further comprises an insertion of a polynucleotide encoding CTLA-4 in a safe harbor locus.

In some embodiments, any of the isolated cells (e.g., stem cells) described herein comprises a disruption (e.g., deletion, insertion, translocation, inversion, or substitution) in the 3′-UTR of an allele encoding HLA-G and further comprises an insertion of a polynucleotide encoding PDL1 in the disrupted 3′-UTR locus of HLA-G. In some embodiments, any of the isolated cells (e.g., stem cells) described herein comprises a disruption (e.g., deletion, insertion, translocation, inversion, or substitution) in the 3′-UTR of an allele encoding HLA-G and further comprises an insertion of a polynucleotide encoding PDL1 in a safe harbor locus.

In some embodiments, any of the isolated cells (e.g., any of the stem cells) described herein further comprises reduced expression (e.g., reduced by at least 10%, at least 20%, at least 30%, at least 40%, at least 50%, at least 60%, at least 70%, at least 80%, at least 90%, or 100%) of MHC-I and MHC-II human leukocyte antigens (HLA) relative to a wild type cell of the same cell type. Major histocompatibility complex (MHC) is a locus on human Chr. 6p21, which encodes a highly polymorphic gene family of surface molecules that define donor compatibility during organ transplantation. MHC class I (MHC-I) and MHC class II (MHCII) play essential roles in the activation of adaptive immune responses by presenting antigens to T lymphocytes.

Humans have three classical MHC-Ia molecules (HLA-A, HLA-B, and HLA-C), which are vital to the detection and elimination of viruses, cancerous cells, and transplanted cells. In addition, there are three non-classical MHC-Ib molecules (HLA-E, HLA-F, and HLA-G), which have immune regulatory functions. While MHC's serve a vital function, in certain contexts, such as cell-based transplantation therapies, they may also contribute to immune rejection.

MHC-I molecules are composed of MHC-encoded heavy chains and the invariant subunit 02-microglobulin (B2M). Antigen-derived peptides are presented by MHC-I-B2M complexes at the cell surface to CD8 T cells carrying an antigen-specific T cell receptor. Peptides are mostly produced from the degradation of cytoplasmic proteins by a specialized proteasome or immunoproteasome, which is optimized to generate MHC class I peptides and contains several IFN-γ-inducible subunits. Unlike MHC-II, which is found mainly in antigen-presenting cells, MHC-Ia is ubiquitously expressed in almost all nucleated cells (see, e.g., Pamer, et al., Annu Rev Immunol (1998) 16:323-358, incorporated herein by reference). Both MHC-I and MHC-II genes are highly inducible by IFN-γ stimulation.

In certain embodiments, reduced expression of MHC-I and MHC-II HLAs results from targeting individual HLAs (e.g., disrupting genes encoding HLA-A, HLA-B and/or HLA-C), targeting transcriptional regulators of HLA expression (e.g., disruption genes encoding NLRC5, CIITA, RFX5, RFXAP, RFXANK, NFY-A, NFY-B, NFY-C and/or IRF-1), or blocking surface trafficking of MHC class I molecules (e.g., disruption genes encoding of B2M and/or TAP1), and/or targeting HLA-Razor. In particular embodiments, the genes encoding HLA-A and HLA-B are individually disrupted, and the gene encoding HLA-C is not disrupted. In particular embodiments, the genes encoding HLA-A and HLA-C are individually disrupted, and the gene encoding HLA-B is not disrupted. In particular embodiments, the genes encoding HLA-B and HLA-C are individually disrupted, and the gene encoding HLA-A is not disrupted.

In some embodiments, the reduced expression of the MHC-I human leukocyte antigens results from a disruption in an allele encoding 0-2 microglobulin (B2M). Accordingly, in some embodiments, any of the isolated cells (e.g., stem cells) described herein further comprises a disruption (e.g., deletion, translocation, inversion, or substitution) in an allele encoding B2M. In some embodiments, the disruption (e.g., deletion, insertion, translocation, inversion, or substitution) in an allele encoding B2M results in a reduction in B2M expression by at least 10%, at least 20%, at least 30%, at least 40%, at least 50%, at least 60%, at least 70%, at least 80%, at least 90%, or 100%. In some embodiments, any of the cells disclosed herein does not comprise a disruption in an allele encoding B2M.

In some embodiments, the reduced expression of the MHC-II human leukocyte antigens results from a disruption in an allele encoding class II major histocompatibility complex transactivator (CIITA). Accordingly, in some embodiments, any of the isolated cells (e.g., stem cells) described herein further comprises a disruption (e.g., deletion, insertion, translocation, inversion, or substitution) in an allele encoding CIITA. In some embodiments, the disruption (e.g., deletion, insertion, translocation, inversion, or substitution) in an allele encoding CIITA results in a reduction in CIITA expression by at least 10%, at least 20%, at least 30%, at least 40%, at least 50%, at least 60%, at least 70%, at least 80%, at least 90%, or 100%. In some embodiments, any of the cells disclosed herein does not comprise a disruption in an allele encoding CIITA.

In some embodiments, any of the isolated cells (e.g., stem cells) described herein further comprises a disruption (e.g., deletion, insertion, translocation, inversion, or substitution) in an allele encoding B2M and a disruption (e.g., deletion, insertion, translocation, inversion, or substitution) in an allele encoding CIITA.

In some embodiments, any of the isolated cells (e.g., stem cells) described herein comprises a disruption (e.g., deletion, insertion, translocation, inversion, or substitution) in any one or more of the genes encoding: B2M, CIITA, HLA-A, HLA-B, HLA-C, RFX-ANK, NFY-A, NLRC5, RFX5, RFX-AP, HLA-G, HLA-E, NFY-B, PD-L1, NFY-C, IRF1, TAPI, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, IDO1, TRAC, TRB, NFY-A, CCR5, F3, CD142, MICA, MICB, LRP1, HMGB1, ABO, RHD, FUT1, KDM5D, PDGFRa, OLIG2, and/or GFAP.

In some embodiments, any of the isolated cells (e.g., stem cells) described herein does not comprise reduced expression of MHC-I and MHC-II human leukocyte antigens (HLA) relative to a wild type stem cell of the same cell type. In some embodiments, any of the cell (e.g., an isolated stem cell) described herein does not comprise a disruption (e.g., deletion, translocation, inversion, or substitution) in an allele encoding B2M or an allele encoding CIITA.

In some embodiments, a cell (e.g., an isolated stem cell) described herein is negative for A antigen and negative for B antigen. In some embodiments, the cell described herein is negative for A antigen. In some embodiments, the cell described herein is negative for B antigen. In some embodiments, a cell (e.g., an isolated stem cell) described herein is negative for Rh antigen. In some embodiments, a cell (e.g., an isolated stem cell) described herein is negative for A antigen, negative for B antigen, and negative for Rh antigen. An “A antigen,” as used herein, refers to a histo-blood group antigen produced by 3α-N-acetylgalactosaminyltransferase and expressed as a cell-surface antigen. A “B antigen,” as used herein, refers to a histo-blood group antigen produced by 3α-galactosaminyltransferase and expressed as a cell-surface antigen.

In some embodiments, the cell comprises a disruption in the ABO gene. In some embodiments, the cell comprises a disruption in the ABO gene such that the cell has reduced or absent levels of A and B antigens. In some embodiments, the cell comprises a disruption in the FUT1 gene. In some embodiments, the cell comprises a disruption in the FUT1 gene such that Galactoside 2-alpha-L-fucosyltransferase 1 expression is reduced or absent. An “Rh antigen,” as used herein, refers to a highly immunogenic antigen encoded by two highly polymorphic genes, RHD and RHCE. Rh antigen proteins are transmembrane proteins. In some embodiments, the cell comprises a disruption in the RHAG gene. In some embodiments, the cell comprises a disruption in the RHAG gene such that the cell has reduced or absent levels of Rh-associated glycoprotein. In some embodiments, the cell has a reduced or eliminated Rh protein antigen expression selected from the group consisting of Rh C antigen, Rh E antigen, Kell K antigen (KEL), Duffy (FY) Fya antigen, Duffy Fy3 antigen, Kidd (JK) Jkb antigen, MNS antigen U, and MNS antigen S.

In some embodiments, a cell (e.g., an isolated stem cell) described herein is an embryonic stem cell. In some embodiments, a cell (e.g., an isolated stem cell) described herein is embryonic germ stem cell (EGSC). In some embodiments, a cell (e.g., an isolated stem cell) described herein is a pluripotent stem cell. In some embodiments, a cell (e.g., an isolated stem cell) described herein is an induced pluripotent stem cell. In some embodiments, a cell (e.g., an isolated stem cell) described herein is a reprogrammed stem cell derived from a somatic cell. In some embodiments, a cell (e.g., an isolated stem cell) described herein is a human stem cell (e.g., a human embryonic stem cell, or a human pluripotent stem cell such as a human induced pluripotent stem cell).

The term “stem cell” as used herein can refer to a cell (e.g., vertebrate stem cell, mammalian stem cell) that has the ability both to self-renew and to generate a differentiated cell type (Morrison et al., (1997) Cell 88:287-298). In the context of cell ontogeny, the adjective “differentiated”, or “differentiating” is a relative term. A “differentiated cell” can be a cell that has progressed further down the developmental pathway than the cell it is being compared with. Thus, pluripotent stem cells can differentiate into lineage-restricted progenitor cells (e.g., mesodermal stem cells), which in turn can differentiate into cells that are further restricted (e.g., neuron progenitors), which can differentiate into end-stage cells (e.g., terminally differentiated cells, e.g., neurons, cardiomyocytes, etc.), which play a characteristic role in a certain tissue type, and can or cannot retain the capacity to proliferate further. Stem cells can be characterized by both the presence of specific markers (e.g., proteins, RNAs, etc.) and the absence of specific markers. Stem cells can also be identified by functional assays both in vitro and in vivo, particularly assays relating to the ability of stem cells to give rise to multiple differentiated progeny. In an embodiment, the host cell is an adult stem cell, a somatic stem cell, a non-embryonic stem cell, an embryonic stem cell, hematopoietic stem cell, an include pluripotent stem cells, and a trophoblast stem cell.

Stem cells of interest, e.g., that can be used in in accordance with the present disclosure, can include pluripotent stem cells (PSCs). The term “pluripotent stem cell” or “PSC” as used herein can refer to a stem cell capable of producing all cell types of the organism. Therefore, a PSC can give rise to cells of all germ layers of the organism (e.g., the endoderm, mesoderm, and ectoderm of a vertebrate). Pluripotent cells can be capable of forming teratomas and of contributing to ectoderm, mesoderm, or endoderm tissues in a living organism. Pluripotent stem cells of plants can be capable of giving rise to all cell types of the plant (e.g., cells of the root, stem, leaves, etc.).

The term “embryonic stem cell” (ESC) refers to pluripotent stem cells that are isolated from an embryo, typically from the inner cell mass of the blastocyst. ESC lines are listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). Stem cells of interest also include embryonic stem cells from other primates, such as Rhesus stem cells and marmoset stem cells. The stem cells can be obtained from any mammalian species, e.g., human, equine, bovine, porcine, canine, feline, rodent, e.g. mice, rats, hamster, primate, etc. (Thomson et al. (1998) Science 282:1145; Thomson et al. (1995) Proc. Natl. Acad. Sci USA 92:7844; Thomson et al. (1996) Biol. Reprod. 55:254; Shamblott et al., Proc. Natl. Acad. Sci. USA 95:13726, 1998). In culture, ESCs can grow as flat colonies with large nucleo-cytoplasmic ratios, defined borders and prominent nucleoli. In addition, ESCs can express SSEA-3, SSEA-4, TRA-1-60, TRA-1-81, and Alkaline Phosphatase, but not SSEA-1. Examples of methods of generating and characterizing ESCs can be found in, for example, U.S. Pat. Nos. 7,029,913, 5,843,780, and 6,200,806, each of which is incorporated herein by its entirety. Methods for proliferating hESCs in the undifferentiated form are described in WO 99/20741, WO 01/51616, and WO 03/020920, each of which is incorporated herein by its entirety.

The term “embryonic germ stem cell” (EGSC) or “embryonic germ cell” or “EG cell” refers to a pluripotent stem cell that is derived from germ cells and/or germ cell progenitors, e.g. primordial germ cells, e.g. those that can become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, each of which are incorporated herein by its entirety.

The term “induced pluripotent stem cell” or “iPSC” refers to a pluripotent cell that is derived from a cell that is not a PSC (e.g., from a cell this is differentiated relative to a PSC). iPSCs can be derived from multiple different cell types, including terminally differentiated cells. iPSCs can have an ES cell-like morphology, growing as flat colonies with large nucleo-cytoplasmic ratios, defined borders and prominent nuclei. In addition, iPSCs can express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPSCs can be found in, for example, U.S. Patent Publication Nos. US20090047263, US20090068742, US20090191159, US20090227032, US20090246875, and US20090304646, each of which are incorporated herein by its entirety. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells. In some embodiments, the de-differentiated stem cells can be for example, but not limited to, neoplastic cells, tumor cells and cancer cells or alternatively induced reprogrammed cells such as induced pluripotent stem cells or iPS cells.

In some embodiments, stem cells used in accordance with the present disclosure can be obtained from mammalian species, e.g., human, equine, bovine, porcine, canine, feline, rodent, e.g. mice, rats, hamster, primate, etc. In some embodiments, a mixture of cells from a suitable source of endothelial, muscle, and/or neural stem cells are harvested from a mammalian donor for the purpose of the present disclosure. A suitable source is the hematopoietic microenvironment. For example, circulating peripheral blood, preferably mobilized (e.g., recruited), may be removed from a subject.

Other aspects of the present disclosure provide isolated cells other than stem cells and cells differentiated from any of the isolated stem cells described herein. The cells other than stem cells and isolated stem cells may be differentiated into any cell type. In some embodiments, a cell differentiated from an isolated stem cell described herein or a cell other than a stem cell is a fibroblast cell, an endothelial cell, a definitive endoderm cell, a primitive gut tube cell, a pancreatic endoderm cell, a pancreatic progenitor cell, a pancreatic endocrine cell, a pancreatic islet cell (e.g., a β cell, an α cell, a δ cell, or an enterochromaffin (EC) cell), a stem cell-derived β cell, a stem cell-derived α cell, a stem cell-derived δ cell, a stem cell-derived enterochromaffin (EC) cell, an insulin producing cell, an insulin-positive β-like cell, a hematopoietic stem cell, a hematopoietic progenitor cell, a muscle cell (e.g., a cardiac muscle cell, a skeletal muscle cell, or a smooth muscle cell), a satellite stem cell, a liver cell (e.g., a hepatocyte or a hepatic stellate cell), a neuron cell (e.g., dopaminergic neurons), or an immune cell (e.g., T cell, B cell, a macrophage, a natural killer cell). The cells differentiated from an isolated stem cell or the cells other than stem cells described herein have reduced immunogenicity relative to a wild-type cell if the same cell type.

In some embodiments, a cell (e.g., a cell differentiated from an isolated stem cell) described herein is of the pancreatic lineage. In some embodiments, cells of the pancreatic lineage include: definitive endoderm cells, primitive gut tube cells, pancreatic endoderm cells, pancreatic progenitor cell, pancreatic endocrine cells, and pancreatic islet cells (e.g., β cells, an cells, a δ cells, enterochromaffin (EC) cells), and combinations thereof. Methods of differentiating stem cells into the pancreatic lineage are known in the art, e.g., as described at least in U.S. Patent Application Publication No. US2015/0240212, US2015/0218522, US2022/0090020, U.S. Pat. No. 11,466,256, WO2022/147056, and WO2022/192300 each of which is incorporated herein by reference.

In some embodiments, a cell (e.g., a cell differentiated from an isolated stem cell) described herein is an immune cell (e.g., T cell, or a natural killer cell). In some embodiments, the immune cell is further modified to express a chimeric antigen receptor (CAR) or an engineered T-cell receptor (TCR). “Chimeric antigen receptor T cells (CART cells),” as used herein, refer to T cells that have been genetically engineered to produce an artificial T cell receptor for use in immunotherapy. “Chimeric antigen receptors (CARs),” as used herein, refer to immunoreceptor proteins that have been engineered to give T cells the new ability to target a specific protein. The receptors are chimeric because they combine both antigen-binding and T cell activity into a single receptor. “T-cell receptor (TCR),” as used herein, refers to a protein complex found on the surface of T cells, or T lymphocytes. TCRs are responsible for recognizing fragments of antigen as peptides bound to MHC molecules. When the TCR engages with an antigenic peptide bound to an MHC molecules, the T-cell is activated through signal transduction, resulting in an adaptive immune response.

In some embodiments, a cell differentiated from isolated stem cells described herein comprises the same genetic modifications as the isolated stem cells from which it is differentiated, e.g., a disruption in the 3′-UTR of an allele encoding an immunosuppressor (e.g., PDL1, CD47, or HLA-G), and optionally exogenous expression of one or more immunosuppressors (e.g., CD47, CTLA-4, PDL1, PDL2, HLA-C, HLA-E, HLA-G, C1-inhibitor, IL-35, DUX4, IDO1, IL10, CCL21, CCL22, CD16, CD52, H2-M3, CD200, FASLG, MFGE8, and/or SERPINB9) and/or reduced expression of MHC-I and MHC-II. In some embodiments, cells (e.g., cells differentiated from the isolated stem cells) described herein comprise increased expression (e.g., increased by at least 10%, at least 20%, at least 30%, at least 40%, at least 50%, at least 60%, at least 70%, at least 80%, at least 90%, at least 100%, at least 2-fold, at least 5-fold, at least 10-fold, at least 50-fold, at least 100-fold or higher) of the immunosuppressor (e.g., e.g., PDL1, CD47, or HLA-G), relative to a wild-type cell of the cell type. In some embodiments, the increased expression of the immunosuppressor (e.g., e.g., PDL1, CD47, or HLA-G) is induced or increased by interferon gamma. In some embodiments, a cell differentiated from an isolated cell (e.g., stem cell) described herein (e.g., a pancreatic islet cell or an immune cell) is less immunogenic (e.g., at least 10%, at least 20%, at least 30%, at least 40%, at least 50%, at least 60%, at least 70%, at least 80%, at least 90%, at least 95%, or at least 99% less), relative to a wild-type cell of the same cell type.

In some aspects, the present disclosure also contemplates isolated immune cells with any of the genetic modifications disclosed herein, and has reduced immunogenicity relative to unmodified isolated immune cells of the same type. Such isolated immune cells can be further modified to express a CAR or a TCR.

### Compositions and Method of Treatment

Further provided herein, in some aspects, are compositions comprising any of the cells disclosed herein (e.g., the cells differentiated from any of the isolated stem cells disclosed herein). In some embodiments, a composition comprises a population of pancreatic islet cells (e.g., human pancreatic islet cells). In some embodiments, the pancreatic islet cells are differentiated from any of the isolated stem cells disclosed herein.

In some embodiments, a population of pancreatic islet cells (e.g., human pancreatic islet cells) described herein comprises NKX6.1-positive, ISL-positive cells and NKX6.1-negative, ISL-positive cells. In some embodiments, a population of pancreatic islet cells (e.g., human pancreatic islet cells) differentiated from the isolated stem cells described herein comprises more NKX6.1-positive, ISL-positive cells than NKX6.1-negative, ISL-positive cells. In some embodiments, the cells in the population are differentiated from any of the isolated stem cells described herein.

In some embodiments, a population of pancreatic islet cells (e.g., human pancreatic islet cells) differentiated from the isolated stem cells described herein comprises NKX6.1-positive, ISL1-positive cells and NKX6.1-negative, ISL1-positive cells, wherein less than 12% of the cells (e.g., about 11%, about 10%, about 9%, about 8%, about 7%, about 6%, about 5%, about 4%, about 3%, about 2%, about 1%, or less) in the population are NKX6.1-negative, ISL1-negative cells. In some embodiments, less than 10%, less than 8%, less than 6%, less than 4%, 1-11%, 2-10%, 2-12%, 4-12%, 6-12%, 8-12%, 2-8%, 4-8%, 3-6% or 3-5% of the cells in the population are NKX6.1-negative, ISL1-negative cells. In some embodiments, 2-12%, 4-12%, 6-12%, 8-12%, 2-8%, 4-8%, 3-6% or 3-5% of the cells in the population are NKX6.1-negative, ISL1-negative cells. In some embodiments, the cells in the population are differentiated from any of the isolated stem cells described herein.

In some embodiments, at least 60%, at least 65%, at least 70%, at least 73%, at least 74%, at least 75%, at least 80%, at least 85%, at least 90%, about 85-95%, or about 90-95% of the cells in the population are ISL1-positive cells. In some embodiments, 50-90%, 50-85%, 50-80%, 50-75%, 50-70%, 50-60%, 60-90%, 60-85%, 60-80%, 60-75%, 60-70%, 65-90%, 65-85%, 65-80%, 65-75%, 65-70%, 70-90%, 70-85%, 70-80%, 70-75%, 75-90%, 75-85%, 75-80%, 80-90%, 80-85%, or 85-90% of the cells in the population are ISL1-positive cells. In some embodiments, at least 74%, at least 75%, at least 80%, at least 85%, at least 90%, about 85-95%, or about 90-95% of the cells in the population are ISL1-positive cells. In some embodiments, about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or about 99% of the cells in the population are ISL1-positive cells. In some embodiments, the cells in the population are differentiated from any of the isolated stem cells described herein.

In some embodiments, a population of pancreatic islet cells (e.g., human pancreatic islet cells) described herein comprises more NKX6.1-negative, ISL1-positive cells than NKX6.1-positive, ISL1-positive cells. In some embodiments, at least 40% of the cells in the population are NKX6.1-negative, ISL1-positive cells. In some embodiments, at least 45%, at least 50%, about 40-50%, about 45-55%, or about 50-55% of the cells in the population are NKX6.1-negative, ISL1-positive cells. In some embodiments, about 40%, 41%, 42%, 43%, 44%, 45%, 46%, 47%, 48%, 49%, 50%, 51%, 52%, 53%, 54%, or about 55% of the cells in the population are NKX6.1-negative, ISL1-positive cells. In some embodiments, the cells in the population are differentiated from any of the isolated stem cells described herein.

In some embodiments, a population of pancreatic islet cells (e.g., human pancreatic islet cells) described herein comprises cells that express insulin (e.g., cells that express insulin but not glucagon or somatostatin), cells that express glucagon (e.g., cells that express glucagon but not insulin or somatostatin), and cells that express somatostatin (e.g., cells that express somatostatin but not insulin or glucagon). In some embodiments, the expression of insulin in a cell of the compositions suggests that the cell is a SC-β cell. In some cases, the expression of glucagon and not expressing somatostatin in a cell of the composition suggests that the cell is a SC-α cell. In some embodiments, the expression of somatostatin and not expressing glucagon in a cell of the composition suggests that the cell is a SC-6 cell. In some embodiments, cells that express insulin are also glucose responsive insulin producing cells. In some embodiments, the cells in the population are differentiated from any of the isolated stem cells described herein.

In some embodiments, a population of pancreatic islet cells (e.g., human pancreatic islet cells) described herein comprises: (a) 30-90%, 30-80%, 30-70%, 30-60%, 30-50%, 30-40%, 40-90%, 40-80%, 40-70%, 40-60%, 40-50%, 50-90%, 50-80%, 50-70%, 50-60%, 60-90%, 60-80%, 60-70%, 70-90%, 70-80%, 70-90%, 70-80%, or 80-90% of the cells in the population of cells express insulin; (b) 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, 30-35% or 35-40% of the cells in the population of cells express glucagon but not somatostatin; and/or (c) 3-20%, 3-15%, 3-12%, 3-10%, 3-8%, 3-5%, 4-20%, 4-15%, 4-12%, 4-10%, 4-8%, 4-5%, 5-20%, 5-15%, 5-12%, 5-10%, 5-8%, 7-20%, 7-15%, 7-12%, 7-10%, 9-20%, 9-15%, 9-12%, 8-10%, 8-12%, 8-15%, 8-20%, 10-20%, 10-12%, 10-15%, 12-20%, 12-15% or 15-20% of the cells in the population of cells express somatostatin but not glucagon. In some embodiments, the cells in the population are differentiated from any of the isolated stem cells described herein.

In some embodiments, a population of pancreatic islet cells (e.g., human pancreatic islet cells) differentiated from the isolated stem cells described herein comprises: (a) 30-90%, 30-80%, 30-70%, 30-60%, 30-50%, 30-40%, 40-90%, 40-80%, 40-70%, 40-60%, 40-50%, 50-90%, 50-80%, 50-70%, 50-60%, 60-90%, 60-80%, 60-70%, 70-90%, 70-80%, 70-90%, 70-80%, or 80-90% of the cells in the population of cells express insulin; (b) 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, 30-35% or 35-40% of the cells in the population of cells express glucagon but not somatostatin; and (c) 3-20%, 3-15%, 3-12%, 3-10%, 3-8%, 3-5%, 4-20%, 4-15%, 4-12%, 4-10%, 4-8%, 4-5%, 5-20%, 5-15%, 5-12%, 5-10%, 5-8%, 7-20%, 7-15%, 7-12%, 7-10%, 9-20%, 9-15%, 9-12%, 8-10%, 8-12%, 8-15%, 8-20%, 10-20%, 10-12%, 10-15%, 12-20%, 12-15% or 15-20% of the cells in the population of cells express somatostatin but not glucagon. In some embodiments, the cells in the population are differentiated from any of the isolated stem cells described herein.

In some embodiments, the percentage of cells expressing a marker provided herein is measured by flow cytometry. In some embodiments, the percentage of cells expressing a marker provided herein is measured by immunohistochemical analysis.

In some embodiments, cells that express insulin (i.e., SC-β cells) in a population of pancreatic islet cells (e.g., human pancreatic islet cells) described herein exhibit glucose stimulated insulin secretion (GSIS). In some embodiments, cells that express insulin (i.e., SC-β cells) in a population of pancreatic islet cells (e.g., human pancreatic islet cells) described herein further mature (e.g., further maturing in a subject after transplantation) into cells that exhibit glucose stimulated insulin secretion (GSIS). In some embodiments, the cells in the population are differentiated from any of the isolated stem cells described herein.

In some embodiments, a composition comprising the cells (e.g., cells differentiated from the isolated stem cells described herein) described herein (e.g., pancreatic islet cells or immune cells) are therapeutic compositions. The therapeutic compositions can further comprise a physiologically compatible solution including, for example, artificial cerebrospinal fluid or phosphate-buffered saline. The therapeutic composition can be used to treat, prevent, or stabilize a disease (e.g., diabetes or cancer).

In some embodiments, a therapeutic composition further comprises other active agents, such as anti-inflammatory agents, exogenous small molecule agonists, exogenous small molecule antagonists, anti-apoptotic agents, antioxidants, and/or growth factors known to a person having skill in the art.

In some embodiments, a therapeutic composition further comprises a pharmaceutically acceptable carrier (e.g., a medium or an excipient). The term pharmaceutically acceptable carrier (or medium), which may be used interchangeably with the term biologically compatible carrier or medium, can refer to reagents, cells, compounds, materials, compositions, and/or dosage forms that are not only compatible with the cells and other agents to be administered therapeutically, but also are suitable for use in contact with the tissues of human beings and animals without excessive toxicity, irritation, allergic response, or other complication. Suitable pharmaceutically acceptable carriers can include water, salt solution (such as Ringer's solution), alcohols, oils, gelatins, and carbohydrates, such as lactose, amylose, or starch, fatty acid esters, hydroxymethylcellulose, and polyvinyl pyrolidine. Such preparations can be sterilized, and if desired, mixed with auxiliary agents such as lubricants, preservatives, stabilizers, wetting agents, emulsifiers, salts for influencing osmotic pressure, buffers, and coloring. Pharmaceutical compositions comprising cellular components or products, but not live cells, can be formulated as liquids. Pharmaceutical compositions comprising living non-native pancreatic β cells can be formulated as liquids, semisolids (e.g., gels, gel capsules, or liposomes) or solids (e.g., matrices, scaffolds and the like).

In some embodiments, a therapeutic composition is formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some embodiments, a therapeutic composition is optionally manufactured in a conventional manner, such as, by way of example only, by means of conventional mixing, dissolving, granulating, dragee-making, levigating, emulsifying, encapsulating, entrapping or compression processes.

In some embodiments, a therapeutic composition comprises one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, a therapeutic composition further comprises one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, a therapeutic composition is suitable for administration by any administration route, including but not limited to, oral, parenteral (e.g., intravenous, subcutaneous, intramuscular, intracerebral, intracerebroventricular, intra-articular, intraperitoneal, or intracranial), intranasal, buccal, sublingual, or rectal administration routes. In some embodiments, a therapeutic composition is formulated for parenteral (e.g., intravenous, subcutaneous, intramuscular, intracerebral, intracerebroventricular, intra-articular, intraperitoneal, or intracranial) administration.

In some embodiments, a therapeutic composition further comprises one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, a therapeutic composition comprises a population of pancreatic islet cells (e.g., the population of pancreatic islet cells differentiated from any of the isolated stem cells described herein) described herein in an amount that is effective to treat or prevent e.g., diabetes. In some embodiments, a therapeutic composition further comprises one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In some embodiments, a therapeutic composition comprising cells, cell components or cell products may be delivered to the kidney of a patient in one or more of several methods of delivery known in the art. In some embodiments, the compositions are delivered to the kidney (e.g., on the renal capsule and/or underneath the renal capsule). In another embodiment, the compositions may be delivered to various locations within the kidney via periodic intraperitoneal or intrarenal injection. Alternatively, the compositions may be applied in other dosage forms known to those skilled in the art, such as pre-formed or in situ-formed gels or liposomes.

In some embodiments, therapeutic compositions comprising live cells in a semi-solid or solid carrier may be formulated for surgical implantation on or beneath the renal capsule. It should be appreciated that liquid compositions also may be administered by surgical procedures. In particular cases, semi-solid or solid pharmaceutical compositions may comprise semipermeable gels, lattices, cellular scaffolds and the like, which may be non-biodegradable or biodegradable. For example, in certain cases, it may be desirable or appropriate to sequester the exogenous cells from their surroundings, yet enable the cells to secrete and deliver biological molecules (e.g., insulin) to surrounding cells or the blood stream. In these cases, cells may be formulated as autonomous implants comprising living cells by a non-degradable, selectively permeable barrier that physically separates the transplanted cells from host tissue. Such implants are sometimes referred to as “immunoprotective,” as they have the capacity to prevent immune cells and macromolecules from killing the transplanted cells in the absence of pharmacologically induced immunosuppression. Various encapsulation devices, degradable gels and networks can be used for the pharmaceutical compositions of the present disclosure. For example, degradable materials particularly suitable for sustained release formulations include biocompatible polymers, such as poly(lactic acid), poly (lactic-co-glycolic acid), methylcellulose, hyaluronic acid, collagen, and the like.

In some embodiments, it may be desirable or appropriate to deliver the cells on or in a biodegradable, preferably bioresorbable or bioabsorbable, scaffold or matrix. These typically three-dimensional biomaterials contain the living cells attached to the scaffold, dispersed within the scaffold, or incorporated in an extracellular matrix entrapped in the scaffold. Once implanted into the target region of the body, these implants become integrated with the host tissue, wherein the transplanted cells gradually become established. Examples of scaffold or matrix (sometimes referred to collectively as “framework”) material that may be used in the present disclosure include nonwoven mats, porous foams, or self-assembling peptides. Nonwoven mats, for example, may be formed using fibers comprising a synthetic absorbable copolymer of glycolic and lactic acids (PGA/PLA), foams, and/or poly(epsilon-caprolactone)/poly(glycolic acid) (PCL/PGA) copolymer.

In some embodiments, the framework is a felt, which can be composed of a multifilament yarn made from a bioabsorbable material, e.g., PGA, PLA, PCL copolymers or blends, or hyaluronic acid. The yarn is made into a felt using standard textile processing techniques consisting of crimping, cutting, carding and needling. In another embodiment, cells are seeded onto foam scaffolds that may be composite structures. In many of the abovementioned cases, the framework may be molded into a useful shape. Furthermore, it will be appreciated that non-native pancreatic β cells may be cultured on pre-formed, non-degradable surgical or implantable devices.

In some embodiments, the matrix, scaffold or device may be treated prior to inoculation of cells in order to enhance cell attachment. For example, prior to inoculation, nylon matrices can be treated with 0.1 molar acetic acid and incubated in polylysine, PBS, and/or collagen to coat the nylon. Polystyrene can be similarly treated using sulfuric acid. The external surfaces of a framework may also be modified to improve the attachment or growth of cells and differentiation of tissue, such as by plasma coating the framework or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate), a cellular matrix, and/or other materials such as, but not limited to, gelatin, alginates, agar, agarose, and plant gums, among others.

In some aspects, the present disclosure provides devices comprising a population of pancreatic islet cells described herein (e.g., a population of pancreatic islet cells differentiated from any of the isolated stem cells described herein). In some embodiments, the pancreatic islet cells form cell clusters. A device can be configured to house the cells described herein which, in particular embodiments, produce and release insulin when implanted into a subject. In some embodiment, a device can further comprise a semipermeable membrane. The semipermeable membrane can be configured to retain the cell cluster in the device and permit passage of insulin secreted by the cells. In some cases of the device, the cells can be encapsulated by the semipermeable membrane. The encapsulation can be performed by any technique available to one skilled in the art. The semipermeable membrane can also be made of any suitable material as one skilled in the art would appreciate and verify. For example, the semipermeable membrane can be made of polysaccharide or polycation. In some cases, the semipermeable membrane can be made of poly(lactide) (PLA), poly(glycolic acid) (PGA), poly(lactide-co-glycolide) (PLGA), and other polyhydroxyacids, poly(caprolactone), polycarbonates, polyamides, polyanhydrides, polyphosphazene, polyamino acids, polyortho esters, polyacetals, polycyanoacrylates, biodegradable polyurethanes, albumin, collagen, fibrin, polyamino acids, prolamines, alginate, agarose, agarose with gelatin, dextran, polyacrylates, ethylene-vinyl acetate polymers and other acyl-substituted cellulose acetates and derivatives thereof, polyurethanes, polystyrenes, polyvinyl chloride, polyvinyl fluoride, poly(vinyl imidazole), chlorosulphonated polyolefins, polyethylene oxide, or any combinations thereof. In some cases, the semipermeable membrane comprises alginate. In some embodiments, the cells are encapsulated in a microcapsule that comprises an alginate core surrounded by the semipermeable membrane. In some embodiments, the alginate core is modified, for example, to produce a scaffold comprising an alginate core having covalently conjugated oligopeptides with an RGD sequence (arginine, glycine, aspartic acid). In some cases, the alginate core is modified, for example, to produce a covalently reinforced microcapsule having a chemoenzymatically engineered alginate of enhanced stability. In some embodiments, the alginate core is modified, for example, to produce membrane-mimetic films assembled by in-situ polymerization of acrylate functionalized phospholipids. In some cases, microcapsules are composed of enzymatically modified alginates using epimerases. In some cases, microcapsules comprise covalent links between adjacent layers of the microcapsule membrane. In some embodiment, the microcapsule comprises a subsieve-size capsule comprising alginate coupled with phenol moieties. In some cases, the microcapsule comprises a scaffold comprising alginate-agarose. In some embodiments, the cells are modified with PEG before being encapsulated within alginate. In some embodiments, the cells are encapsulated in photoreactive liposomes and alginate. It should be appreciated that the alginate employed in the microcapsules can be replaced with other suitable biomaterials, including, without limitation, polyethylene glycol (PEG), chitosan, polyester hollow fibers, collagen, hyaluronic acid, dextran with ROD, BHD and polyethylene glycol-diacrylate (PEGDA), poly(MPC-co-n-butyl methacrylate-co-4-vinylphenyl boronic acid) (PMBV) and poly(vinyl alcohol) (PVA), agarose, agarose with gelatin, and multilayer cases of these. In some embodiments, the device provided herein comprise extracorporeal segment, e.g., part of the device can be outside a subject's body when the device is implanted in the subject. The extracorporeal segment can comprise any functional component of the device, with or without the cells or cell cluster provided herein.

Further provided herein are methods for treating or preventing a disease in a subject. A composition comprising the pancreatic islet cells differentiated from the isolated stem cells described herein can be administered into a subject to restore a degree of pancreatic function in the subject. In some embodiments, such composition is transplanted in a subject. The term “transplant” can refer to the placement of cells or cell clusters, any portion of the cells or cell clusters thereof, any compositions comprising cells, cell clusters or any portion thereof, into a subject, by a method or route which results in at least partial localization of the introduced cells or cell clusters at a desired site. In some embodiments, the desired site is the pancreas. In some embodiments, the desired site is a non-pancreatic location, such as in the liver or subcutaneously, for example, in a capsule (e.g., microcapsule) to maintain the implanted cells at the implant location and avoid migration. In some embodiments, the transplanted cells release insulin in an amount sufficient for a reduction of blood glucose levels in the subject.

In some embodiments, a composition comprising pancreatic islet cells disclosed herein (e.g., pancreatic islet cells differentiated from any of the isolated stem cells described herein) are housed in a device that is implanted in a subject. In some embodiments, the device upon implantation in a subject releases insulin while retaining the cells in the device, and facilitates tissue vascularization in and around the device. Exemplary devices are described, for example in WO2018/232180, WO2019/068059, WO2019/178134, WO2020/206150, and WO2020/206157, each of which is incorporated-by-reference in its entirety. In some embodiments, a subject is not administered an immune suppression agent during the implantation or vascularization of the device. In some embodiments, the device has a thickness of at least about 300 pm. In some embodiments, the device comprises a membrane comprising a plurality of nodes interconnected by a plurality of fibrils.

In some embodiments, the device comprises a first membrane having a first surface comprising a plurality of channels, and a plurality of second surfaces opposing the first surface; and a second membrane opposite and attached to the plurality of the second surfaces of the first membrane; wherein the first membrane and the second membrane form an enclosed compartment having a surface area to volume ratio of at least about 40 cm-1, and wherein the enclosed compartment provides a volume for housing a cell within the device.

In some embodiments, the enclosed compartment comprises a single continuous open chamber. In some embodiments, the volume is about 8 μL to about 1,000 μL. In some embodiments, the device has at least one of a length and a width of about 0.25 cm to about 3 cm. In some embodiments, the device has a thickness of at least about 300 pm.

In some embodiments, the plurality of channels is generally perpendicular with respect to the first membrane. In some embodiments, the plurality of channels is arranged in a rectilinear array. In some embodiments, the plurality of channels is arranged in a polar array. In some embodiments, the channel has an average diameter of about 400 pm to about 3,000 pm. In some embodiments, the diameter is measured at a narrowest point in the channel. In some embodiments, a center of each channel is separated from the center of another channel by a distance of about 75 pm to about 500 pm. In some embodiments, the channel has a height to diameter ratio of at least about 0.2. In some embodiments, the device has a number of channels per area along a transverse plane, and in some cases the number is greater than about 50/cm2.

In some embodiments, at least one of the first membrane and the second membrane comprise a plurality of nodes interconnected by a plurality of fibrils. In some embodiments, at least one of the first membrane and the second membrane comprise PVDF, PTFE, ePTFE, PCL, PE/PES, PP, PS, PMMA, PLGA, PLLA, or any combination thereof. In some embodiments, the device further comprises an opening through the first membrane and/or the second membrane within the channel. In some embodiments, the opening has a concentricity with respect to the channel of at most about 25% the diameter of the channel. In some embodiments is a frame configured to receive the device described herein. In some embodiments, the frame is configured to receive a plurality of cell housing devices. In some embodiments, the frame comprises a flexing mechanism configured to prevent buckling of the cell housing device.

In some embodiments, a method described herein comprises transplanting pancreatic islet cells described herein (e.g., pancreatic islet cells differentiated from any of the isolated stem cells described herein) to a subject using any means in the art. For example, the methods can comprise transplanting the cell cluster via the intraperitoneal space, portal vein, renal subcapsule, renal capsule, omentum, subcutaneous space, or via pancreatic bed infusion. For example, transplanting can be subcapsular transplanting, intramuscular transplanting, or intraportal transplanting, e.g., intraportal infusion. Immunoprotective encapsulation can be implemented to provide immunoprotection to the cell clusters. In some cases, the methods of treatment provided herein can comprise administering one or more immune response modulators for modulating or reducing transplant rejection response or other immune response against the implant (e.g., the cells or the device). Examples of immune response modulator that can be used in the methods can include purine synthesis inhibitors like Azathioprine and Mycophenolic acid, pyrimidine synthesis inhibitors like Leflunomide and Teriflunomide, antifolate like Methotrexate, Tacrolimus, Ciclosporin, Pimecrolimus, Abetimus, Gusperimus, Lenalidomide, Pomalidomide, Thalidomide, PDE4 inhibitor, Apremilast, Anakinra, Sirolimus, Everolimus, Ridaforolimus, Temsirolimus, Umirolimus, Zotarolimus, Anti-thymocyte globulin antibodies, Anti-lymphocyte globulin antibodies, CTLA-4, fragment thereof, and fusion proteins thereof like Abatacept and Belatacept, TNF inhibitor like Etanercept and Pegsunercept, Aflibercept, Alefacept, Rilonacept, antibodies against complement component 5 like Eculizumab, anti-TNF antibodies like Adalimumab, Afelimomab, Certolizumab pegol, Golimumab, Infliximab, and Nerelimomab, antibodies against Interleukin 5 like Mepolizumab, anti-Ig E antibodies like Omalizumab, anti-Interferon antibodies like Faralimomab, anti-IL-6 antibodies like Elsilimomab, antibodies against IL-12 and IL-23 like Lebrikizumab and Ustekinumab, anti-IL-17A antibodies like Secukinumab, anti-CD3 antibodies like Muromonab-CD3, Otelixizumab, Teplizumab, and Visilizumab, anti-CD4 antibodies like Clenoliximab, Keliximab, and Zanolimumab, anti-CD11a antibodies like Efalizumab, anti-CD18 antibodies like Erlizumab, anti-CD20 antibodies like Obinutuzumab, Rituximab, Ocrelizumab and Pascolizumab, anti-CD23 antibodies like Gomiliximab and Lumiliximab, anti-CD40 antibodies like Teneliximab and Toralizumab, antibodies against CD62L/L-selectin like Aselizumab, anti-CD80 antibodies like Galiximab, anti-CD147/Basigin antibodies like Gavilimomab, anti-CD154 antibodies like Ruplizumab, anti-BLyS antibodies like Belimumab and Blisibimod, anti-CTLA-4 antibodies like Ipilimumab and Tremelimumab, anti-CAT antibodies like Bertilimumab, Lerdelimumab, and Metelimumab, anti-Integrin antibodies like Natalizumab, antibodies against Interleukin-6 receptor like Tocilizumab, anti-LFA-1 antibodies like Odulimomab, antibodies against IL-2 receptor/CD25 like Basiliximab, Daclizumab, and Inolimomab, antibodies against T-lymphocyte (Zolimomab aritox) like Atorolimumab, Cedelizumab, Fontolizumab, Maslimomab, Morolimumab, Pexelizumab, Reslizumab, Rovelizumab, Siplizumab, Talizumab, Telimomab aritox, Vapaliximab, and Vepalimomab.

As used herein, the term “treating” and “treatment” can refer to administering to a subject an effective amount of a composition (e.g., cell clusters or a portion thereof) so that the subject has a reduction in at least one symptom of the disease or an improvement in the disease, for example, beneficial or desired clinical results. For purposes of this disclosure, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (e.g., partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection and infusion. In preferred embodiments, the compositions are administered by intravenous infusion or injection.

By “treatment,” “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, one or more symptoms of a disease or disorder are alleviated by at least 5%, at least 10%, at least 20%, at least 30%, at least 40%, or at least 50% in comparison to a non-treated subject.

Treatment of Diabetes is determined by standard medical methods. A goal of Diabetes treatment is to bring sugar levels down to as close to normal as is safely possible. Commonly set goals are 80-120 milligrams per deciliter (mg/dl) before meals and 100-140 mg/dl at bedtime. A particular physician may set different targets for the patent, depending on other factors, such as how often the patient has low blood sugar reactions. Useful medical tests include tests on the patient's blood and urine to determine blood sugar level, tests for glycosylated hemoglobin level (HbA1c; a measure of average blood glucose levels over the past 2-3 months, normal range being 4-6%), tests for cholesterol and fat levels, and tests for urine protein level. Such tests are standard tests known to those of skill in the art (see, for example, American Diabetes Association, 1998). A successful treatment program can also be determined by having fewer patients in the program with complications relating to Diabetes, such as diseases of the eye, kidney disease, or nerve disease.

Delaying the onset of diabetes in a subject refers to delay of onset of at least one symptom of diabetes, e.g., hyperglycemia, hypoinsulinemia, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, or combinations thereof, for at least 1 week, at least 2 weeks, at least 1 month, at least 2 months, at least 6 months, at least 1 year, at least 2 years, at least 5 years, at least 10 years, at least 20 years, at least 30 years, at least 40 years or more, and can include the entire lifespan of the subject.

In some embodiments, the reduction of blood glucose levels in the subject, as induced by the transplantation of the cell, or the composition or device provided herein, results in an amount of glucose which is lower than the diabetes threshold. In some embodiments, the subject is a mammalian subject. In some embodiments, the mammalian subject is human. In some embodiments, the amount of glucose is reduced to lower than the diabetes threshold in 1, 2, 3, 4, 5, 6, 7, 8, 9, or 10 days after the implanting.

A subject that can be treated by the methods herein can be a human or a non-human animal. In some cases, a subject can be a mammal. Examples of a subject include but are not limited to primates, e.g., a monkey, a chimpanzee, a bamboo, or a human. In some cases, a subject is a human. A subject can be non-primate animals, including, but not limited to, a dog, a cat, a horse, a cow, a pig, a sheep, a goat, a rabbit, and the like. In some cases, a subject receiving the treatment is a subject in need thereof, e.g., a human in need thereof.

The terms, “patient” and “subject” are used interchangeably herein. Preferably, the subject is a mammal. The mammal can be a human, non-human primate, mouse, rat, dog, cat, horse, or cow, but are not limited to these examples. Mammals other than humans can be advantageously used as subjects that represent animal models of Type 1 diabetes, Type 2 Diabetes Mellitus, or pre-diabetic conditions. In addition, the methods described herein can be used to treat domesticated animals and/or pets. A subject can be male or female. A subject can be one who has been previously diagnosed with or identified as suffering from or having Diabetes (e.g., Type 1 or Type 2), one or more complications related to Diabetes, or a pre-diabetic condition, and optionally, but need not have already undergone treatment for the Diabetes, the one or more complications related to Diabetes, or the pre-diabetic condition. A subject can also be one who is not suffering from Diabetes or a pre-diabetic condition. A subject can also be one who has been diagnosed with or identified as suffering from Diabetes, one or more complications related to Diabetes, or a pre-diabetic condition, but who show improvements in known Diabetes risk factors as a result of receiving one or more treatments for Diabetes, one or more complications related to Diabetes, or the pre-diabetic condition. Alternatively, a subject can also be one who has not been previously diagnosed as having Diabetes, one or more complications related to Diabetes, or a pre-diabetic condition. For example, a subject can be one who exhibits one or more risk factors for Diabetes, complications related to Diabetes, or a pre-diabetic condition, or a subject who does not exhibit Diabetes risk factors, or a subject who is asymptomatic for Diabetes, one or more Diabetes-related complications, or a pre-diabetic condition. A subject can also be one who is suffering from or at risk of developing Diabetes or a pre-diabetic condition. A subject can also be one who has been diagnosed with or identified as having one or more complications related to Diabetes or a pre-diabetic condition as defined herein, or alternatively, a subject can be one who has not been previously diagnosed with or identified as having one or more complications related to Diabetes or a pre-diabetic condition.

In some aspects, the present disclosure provides methods of treating cancer by administering to a subject immune cells described herein (e.g., immune cells differentiated from the isolated stem cells described herein), or immune cells that comprise the genetic modifications described herein or are genetically modified using the methods described herein, or compositions comprising such immune cells. In some embodiments, the immune cells further express a chimeric antigen receptor or an engineered T-cell receptor. In certain embodiments, the subject is a mammal, e.g., a primate, e.g., a human. Non-limiting examples of cancers that may be treated in accordance with the present disclosure include: adult and pediatric acute lymphoblastic leukemia, acute myeloid leukemia, adrenocortical carcinoma, AIDS-related cancers, anal cancer, cancer of the appendix, astrocytoma, basal cell carcinoma, bile duct cancer, bladder cancer, bone cancer, biliary tract cancer, osteosarcoma, fibrous histiocytoma, brain cancer, brain stem glioma, cerebellar astrocytoma, malignant glioma, glioblastoma, ependymoma, medulloblastoma, supratentorial primitive neuroectodermal tumors, hypothalamic glioma, breast cancer, male breast cancer, bronchial adenomas, Burkitt lymphoma, carcinoid tumor, carcinoma of unknown origin, central nervous system lymphoma, cerebellar astrocytoma, malignant glioma, cervical cancer, childhood cancers, chronic lymphocytic leukemia, chronic myelogenous leukemia, acute lymphocytic and myelogenous leukemia, chronic myeloproliferative disorders, colorectal cancer, cutaneous T-cell lymphoma, endometrial cancer, ependymoma, esophageal cancer, Ewing family tumors, extracranial germ cell tumor, extragonadal germ cell tumor, extrahepatic bile duct cancer, intraocular melanoma, retinoblastoma, gallbladder cancer, gastric cancer, gastrointestinal stromal tumor, extracranial germ cell tumor, extragonadal germ cell tumor, ovarian germ cell tumor, gestational trophoblastic tumor, glioma, hairy cell leukemia, head and neck cancer, hepatocellular cancer, Hodgkin lymphoma, non-Hodgkin lymphoma, hypopharyngeal cancer, hypothalamic and visual pathway glioma, intraocular melanoma, islet cell tumors, Kaposi sarcoma, kidney cancer, renal cell cancer, laryngeal cancer, lip and oral cavity cancer, small cell lung cancer, non-small cell lung cancer, primary central nervous system lymphoma, Waldenstrom macroglobulinemia, malignant fibrous histiocytoma, medulloblastoma, melanoma, Merkel cell carcinoma, malignant mesothelioma, squamous neck cancer, multiple endocrine neoplasia syndrome, multiple myeloma, mycosis fungoides, myelodysplastic syndromes, myeloproliferative disorders, chronic myeloproliferative disorders, nasal cavity and paranasal sinus cancer, nasopharyngeal cancer, neuroblastoma, oropharyngeal cancer, ovarian cancer, pancreatic cancer, parathyroid cancer, penile cancer, pharyngeal cancer, pheochromocytoma, pineoblastoma and supratentorial primitive neuroectodermal tumors, pituitary cancer, plasma cell neoplasms, pleuropulmonary blastoma, prostate cancer, rectal cancer, rhabdomyosarcoma, salivary gland cancer, soft tissue sarcoma, uterine sarcoma, Sezary syndrome, non-melanoma skin cancer, small intestine cancer, squamous cell carcinoma, squamous neck cancer, supratentorial primitive neuroectodermal tumors, testicular cancer, throat cancer, thymoma and thymic carcinoma, thyroid cancer, transitional cell cancer, trophoblastic tumors, urethral cancer, uterine cancer, uterine sarcoma, vaginal cancer, vulvar cancer, choriocarcinoma, hematological neoplasm, adult T-cell leukemia, lymphoma, lymphocytic lymphoma, stromal tumors and germ cell tumors, or Wilms tumor. In some embodiments, the cancer is metastatic cancer.

In some aspects, the present disclosure provides methods of treating a muscle disorder (e.g., Duchenne's Muscular Dystrophy or Myotonic Dystrophy) by administering to a subject any of the muscle cells described herein such as cardiac muscle cells, skeletal muscle cells, smooth muscle cells, and/or satellite stem cells (e.g., muscle cells or satellite stem cells differentiated from the isolated stem cells described herein), or compositions comprising such muscle cells or satellite stem cells.

In some aspects, the present disclosure provides methods of treating a blood disorder (e.g., beta-thalassemia or sickle cell disease) by administering to a subject a hematopoietic progenitor cell (e.g., a hematopoietic progenitor cell differentiated from any of the stem cells disclosed herein), or compositions comprising such hematopoietic progenitor cells.

In some aspects, the present disclosure provides methods of treating a liver disorder (e.g., Alpha-1 Antitrypsin Deficiency Disease) by administering to a subject a liver cell such as a hepatocyte or hepatic stellate cell (e.g., a liver cell differentiated from any of the stem cells disclosed herein), or compositions comprising such liver cells.

In some aspects, the present disclosure provides methods of treating a neurological disorder (e.g., Parkinson's Disease) by administering to a subject a neuronal cell such as a dopaminergic neuron (e.g., a dopaminergic neuron differentiated from any of the stem cells disclosed herein), or compositions comprising such neuronal cells.

### Method of Producing Hypoimmune Cells

In some aspects, the present disclosure provides methods of producing isolated cells (e.g., an isolated stem cell) described herein. In some embodiments, a method of producing an isolated cell comprises altering the genome of a cell to introduce the genetic modifications described herein (e.g., to disrupt the 3′-UTR of an allele encoding an immunosuppressor, to insert an exogenous polynucleotide sequence encoding one or more immunosuppressors, to insert an exogenous polynucleotide sequence encoding an anti-CRISPR protein, and/or to disrupt genes reduce MCH-I or MHC-II expression). The genetic modifications described herein may be made in any manner which is available to the skilled artisan,

In some embodiments, a method of producing a cell (e.g., an isolated stem cell) described herein comprises delivering to a cell (e.g., a human embryonic stem cell, a human pluripotent stem cell, or a human induced pluripotent stem cell) a gene editing system that is capable of making the generic modifications described herein. For example, in some embodiments, the gene editing system is a CRISPR-Cas gene editing system. Such systems comprise, for example, one or more endonucleases and one or more guide RNAs that target the genetic sequence of interest. In some embodiments, the hypoimmune cells described herein are produced by introducing a CRISPR-Cas gene editing system into a stem cell to create one or more disruptions in the 3′-UTR of one or more immunosuppressor genes.

In some embodiments, the disclosure provides for a method in which hypoimmune cells are produced by delivering to a cell (e.g., a stem cell) a composition comprising one or more guide RNAs (gRNA) comprising a nucleotide sequence that targets the 3′-UTR of an allele encoding the immunosuppressor, or one or more nucleic acids encoding the gRNAs. In some embodiments, the gene editing system comprises nucleases (e.g., endonucleases) or recombinases (e.g., site specific recombinases) that are capable of disrupting the targeted genomic sites. Non-limiting examples of nucleases (e.g., endonucleases) that may be used in accordance with the present disclosure include zinc-finger nucleases (ZFN), transcription activator-like effector nucleases (TALEN), meganucleases, and RNA-guided endonucleases (e.g., CRISPR-Cas9 or CRISPR/Cas9; Clustered Regular Interspaced Short Palindromic Repeats Associated 9 nucleases). Non-limiting examples of recombinases (e.g., site specific recombinases) that may be used in accordance with the present disclosure include: Cre, Bxb1, FLPe, phiC31 Integrase, phiC31 excisionase, R4, PhiBTI, WP integrase, SPBc, and TP901-1. In some embodiments, the gene editing system comprises nucleic acids encoding the nucleases (e.g., endonucleases) or recombinases (e.g., site specific recombinases) that are capable of disrupting the targeted genomic sites. In some embodiments, the gene-editing system comprises a transposon system, such as the piggyBac transposon system.

In some embodiments, the gene editing system comprises a zinc finger nuclease (ZFN) or a nucleic acid encoding a ZFN. Zinc finger nucleases (ZFNs) are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the FokI nuclease with a zinc finger DNA binding domain.

In some embodiments, the gene editing system comprises a transcription activator-like effector nucleases (TALEN) or a nucleic acid encoding a TALEN. a transcription activator-like effector nucleases (TALEN) is a targeted nuclease comprising a nuclease fused to a transcription activator-like effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the FokI nuclease to a TAL effector DNA binding domain.

In some embodiments, the gene editing system comprises an RNA-guided nuclease or a nucleic acid encoding an RNA-guided nuclease. RNA-guided endonucleases are enzymes that utilize RNA:DNA base-pairing to target and cleave a polynucleotide. RNA-guided endonuclease may cleave single-stranded polynucleic acids or at least one strand of a double-stranded polynucleotide. A gene editing-system may comprise one RNA-guided endonuclease. Alternatively, a gene-editing system may comprise at least two (e.g., two, three, four, five, six, seven, eight, nine, ten, or more than ten) RNA-guided endonucleases. In some embodiments, the gene editing system further comprises one or more (e.g., 1, 2, 3, 4, 5, or more) guide-RNAs (gRNAs) or one or more nucleic acids encoding the one or more gRNAs. In some embodiments, the gene editing system comprises a nucleotide acid encoding both the RNA-guided nuclease and the one or more gRNAs. In some embodiments, the gene editing system comprises one or more (e.g., 1, 2, 3, 4, 5, or more) RNA-guided nuclease and gRNA complexes.

In some embodiments, the gene editing system comprises a CRISPR/Cas system and the RNA-guided nuclease is a Cas protein. In some embodiments, a Cas protein comprises a core Cas protein. Exemplary Cas core proteins include, but are not limited to Cas 1, Cas2, Cas3, Cas4, Cas5, Cas6, Cas7, Cas8, Cas9, Cas10, Cas11, Cas12, Cas12i, Cas14, Casϕ, and modified versions thereof. Cas proteins and variants that may be used in gene editing are known in the art, e.g., as described in Xu et al., Computational and Structural Biotechnology Journal, Volume 18, 2020, Pages 2401-2415 and in International Application Publication No. WO2019178427, the entire contents of each of which is incorporated herein by reference.

In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6.

In some embodiments, the Cas protein is a Streptococcus pyogenes Cas9 protein or a functional portion thereof. In some embodiments, the Cas protein is a Staphylococcus aureus Cas9 protein or a functional portion thereof. In some embodiments, the Cas protein is a Streptococcus thermophilus Cas9 protein or a functional portion thereof. In some embodiments, the Cas protein is a Neisseria meningitides Cas9 protein or a functional portion thereof. In some embodiments, the Cas protein is a Treponema denticola Cas9 protein or a functional portion thereof. In some embodiments, the Cas protein is Cas9 protein from any bacterial species or functional portion thereof. Cas9 protein is a member of the type II CRISPR systems which typically include a trans-coded small RNA (tracrRNA), endogenous ribonuclease 3 (rnc) and a Cas protein. One example of a Cas9 protein from Streptococcus pyogenes is a polypeptide comprising 1368 amino acids (as provided in Uniprot Accession No. Q99ZW2). Cas9 contains 2 endonuclease domains, including an RuvC-like domain (residues 7-22, 759-766 and 982-989) which cleaves target DNA that is noncomplementary to crRNA, and an HNH nuclease domain (residues 810-872) which cleave target DNA complementary to crRNA. In some embodiments, one or both of the HNH or RuvC-like domain are not functional.

In some embodiments, the Cas protein comprises a catalytically inactive Cas (e.g., Cas9) domain fused to an enzyme capable of introducing mutations without generating double strand breaks (e.g., adenosine deaminase), e.g., as described in Komor et al., Nature, 2016 May 19; 533(7603):420-4.

In some embodiments, the Cas protein comprises a catalytically inactive Cas (e.g., Cas9) domain fused to a reverse transcriptase. In some embodiments, such Cas proteins may be used with a prime editing guide RNA (pegRNA) that both specifies the target site and encodes the desired edit, e.g., as described in Anzalone et al., Nature volume 576, pages 149-157 (2019).

In some embodiments, the Cas protein is Cpf1 protein or a functional portion thereof. In some embodiments, the Cas protein is Cpf1 from any bacterial species or functional portion thereof. In some embodiments, Cpf1 is a Francisella novicida U112 protein or a functional portion thereof. In some embodiments, Cpf1 is a Acidaminococcus sp. BV3L6 protein or a functional portion thereof. In some embodiments, Cpf1 is a Lachnospiraceae bacterium ND2006 protein or a function portion thereof. Cpf1 protein is a member of the type V CRISPR systems. Cpf1 protein is a polypeptide comprising about 1300 amino acids. Cpf1 contains a RuvC-like endonuclease domain. Cpf1 cleaves target DNA in a staggered pattern using a single ribonuclease domain. The staggered DNA double-stranded break results in a 4 or 5-nt 5′ overhang.

In some embodiments, the Cas protein is Cas12i protein as described in International Application Publication No. WO2019178427, or a functional portion thereof. In some embodiments, the Cas12i protein is a Type V-I (CLUST.029130) Cas protein. In some embodiments, the Cas12i protein is about 1100 amino acids or less in length (and includes at least one RuvC domain.

In some embodiments, the Cas protein is a CasPhi or Cas14 protein.

As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cpf1 protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cpf1 protein comprises a functional portion of a RuvC-like domain.

The RNA-guided nucleases (e.g., a Cas protein) described herein are directed to a target genomic site by one or more gRNAs. Naturally, two noncoding RNAs—crisprRNA (crRNA) and trans-activating RNA (tracrRNA) target a mRNA-guided nuclease (e.g., a Cas protein) to a target genomic site. crRNA drives sequence recognition and specificity of the CRISPR-Cas9 complex through Watson-Crick base pairing typically with a 20 nucleotide (nt) sequence in the target DNA. Changing the sequence of the 5′ 20nt in the crRNA allows targeting of the CRISPR-Cas9 complex to specific loci. The CRISPR-Cas9 complex only binds DNA sequences that contain a sequence match to the first 20 nt of the crRNA if the target sequence is followed by a specific short DNA motif referred to as a protospacer adjacent motif (PAM). TracrRNA hybridizes with the 3′ end of crRNA to form an RNA-duplex structure that is bound by the Cas9 endonuclease to form the catalytically active CRISPR-Cas9 complex, which can then cleave the target DNA. Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

In some embodiments, the gRNA is a dual-guide RNA or a single guide RNA (sgRNA). In some embodiments, the guide RNA is a single-guide RNA (sgRNA) comprising aspects of both a tracrRNA and a crRNA.

In some embodiments, the gene editing system used in the methods of producing the isolated cells (e.g., isolated stem cells) described herein comprises one or more gRNAs or one or more nucleic acids encoding the one or more gRNAs. The gRNAs can be selected to hybridize to a variety of different target motifs, depending on the particular CRISPR/Cas system employed, and the sequence of the target polynucleotide, as will be appreciated by those skilled in the art.

As is understood by the person of ordinary skill in the art, each gRNA is designed to include a spacer sequence complementary to its genomic target sequence. See Jinek et al., Science, 337, 816-821 (2012) and Deltcheva et al., Nature, 471, 602-607 (2011). A spacer sequence is a sequence (e.g., a 20 nucleotide sequence) that defines the target sequence (e.g., a DNA target sequences, such as a genomic target sequence) of a target nucleic acid of interest. The gRNA can comprise a variable length spacer sequence with 17-30 nucleotides at the 5′ end of the gRNA sequence. In some embodiments, the spacer sequence is 15 to 30 nucleotides. In some embodiments, the spacer sequence is 15, 16, 17, 18, 19, 29, 21, 22, 23, 24, 25, 26, 27, 28, 29, or 30 nucleotides. In some embodiments, a spacer sequence is 20 nucleotides.

The “target sequence” is adjacent to a PAM sequence and is the sequence modified by an RNA-guided nuclease (e.g., Cas9). The “target nucleic acid” is a double-stranded molecule: one strand comprises the target sequence and is referred to as the “PAM strand,” and the other complementary strand is referred to as the “non-PAM strand.” One of skill in the art recognizes that the gRNA spacer sequence hybridizes to the reverse complement of the target sequence, which is located in the non-PAM strand of the target nucleic acid of interest. Thus, the gRNA spacer sequence is the RNA equivalent of the target sequence. The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

The spacer sequence is designed to hybridize to a region of the target nucleic acid that is located 5′ of a PAM of the Cas9 enzyme used in the system. The spacer may perfectly match the target sequence or may have mismatches. Each Cas protein has a particular PAM sequence that it recognizes in a target DNA, e.g., as described in Xu et al., Computational and Structural Biotechnology Journal, Volume 18, 2020, Pages 2401-2415, incorporated herein by reference. For example, S. pyogenes Cas9 recognizes in a target nucleic acid a PAM that comprises the sequence 5′-NRG-3′, where R comprises either A or G, where N is any nucleotide and N is immediately 3′ of the target nucleic acid sequence targeted by the spacer sequence. In another example, a Cas12i protein recognizes in a target nucleic acid a PAM that comprises the sequence of 5′-TTN-3′ or 5′-TTH-3′ or 5′-TTY-3′ or 5′-TTC-3′, wherein N is any nucleotide, H is adenine, cytosine, or thymine, Y is cytosine, thymine, or pyrimidine.

In some embodiments, the target nucleic acid sequence comprises 20 nucleotides. In some embodiments, the target nucleic acid comprises less than 20 nucleotides. In some embodiments, the target nucleic acid comprises more than 20 nucleotides. In some embodiments, the target nucleic acid comprises at least: 5, 10, 15, 16, 17, 18, 19, 20, 21, 22, 23, 24, 25, 30 or more nucleotides. In some embodiments, the target nucleic acid comprises at most: 5, 10, 15, 16, 17, 18, 19, 20, 21, 22, 23, 24, 25, 30 or more nucleotides. In some embodiments, the target nucleic acid sequence comprises 20 bases immediately 5′ of the first nucleotide of the PAM. For example, in a sequence comprising 5′-NNNNNNNNNNNNNNNNNNNNNRG-3′, the target nucleic acid comprises the sequence that corresponds to the Ns, wherein N is any nucleotide, and the underlined NRG sequence is the S. aureus PAM.

In some embodiments, a gRNA that targets the 3′-UTR of PDL1 for disrupting the 3′-UTR of PDL1 as described herein targets a sequence between positions 990-1050 of a PDL1 sequence as set forth in SEQ ID NO: 1, positions 17368-17429 of a PDL1 sequence as set forth in SEQ ID NO: 28, or positions 648-708 of a PDL1 sequence as set forth in SEQ ID NO: 2. In some embodiments, a gRNA that targets the 3′-UTR of PDL1 as described herein targets a sequence between positions 1003-1022 of a PDL1 sequence as set forth in SEQ ID NO: 1, positions 17382-17401 of a PDL1 sequence as set forth in SEQ ID NO: 28, or positions 662-680 of a PDL1 sequence as set forth in SEQ ID NO: 2. In some embodiments, a gRNA that targets the 3′-UTR of PDL1 as described herein targets a sequence between positions 1021-1040 of a PDL1 sequence as set forth in SEQ ID NO: 1, positions 17400-17419 of a PDL1 sequence as set forth in SEQ ID NO: 28, or positions 679-698 of a PDL1 sequence as set forth in SEQ ID NO: 2. In some embodiments, a gRNA that targets the 3′-UTR of PDL1 as described herein targets a sequence downstream (e.g., at least 5 nucleotides, at least 10 nucleotides, at least 50 nucleotides, at least 100 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1000 nucleotides, or more downstream) of the 3′-UTR of PDL1 on the opposite strand. In some embodiments, a gRNA that targets the 3′-UTR of PDL1 for disrupting the 3′-UTR of PDL1 as described herein targets a target sequence comprising the nucleotide sequence of AGAGGAAGGAATGGGCCCGT (SEQ ID NO: 13), TCGGGGCTGAGCGTGACAAG (SEQ ID NO: 14), or TCTTCTTGGTATGGTCCTAA (SEQ ID NO: 15). In some embodiments, delivering to a stem cell a Cas protein (e.g., Cas9), a gRNA that targets a target sequence as set forth in SEQ ID NO: 13 or SEQ ID NO: 15, and a gRNA that targets a target sequence as set forth in SEQ ID NO: 15, or one or more nucleic acids encoding these components results in a deletion of the 3′-UTR of PDL1.

In some embodiments, a gRNA for use in the gene-editing system disclosed herein further comprises a scaffold sequence. A scaffold sequence may comprise the sequence of a minimum CRISPR repeat sequence, a single-molecule guide linker, a minimum tracrRNA sequence, a 3′ tracrRNA sequence, and/or an optional tracrRNA extension sequence. The scaffold sequence may be connected to the 5′ and/or 3′ end of a spacer sequence. In some embodiments the scaffold sequence is connected to the 3′ end of the spacer sequence. In other embodiments, the scaffold sequence is connected to the 5′ end of the spacer sequence.

In some embodiments, a gRNA for use in the gene-editing system disclosed herein may comprise, consist essentially of (e.g., contain up to 20 extra nucleotides at the 5′ end and/or the 3′ end of the following sequences), or consist of one of the following scaffold nucleotide sequences:

(i)
(SEQ ID NO: 16)
acccagcctgacaccaaatttaGUUUUAGUACUCUGGAAACAGAAUCUA
CUAAAACAAGGCAAAAUGCCGUGUUUAUCUCGUCAACUUGUUGGCGAGA
UUUU;
(ii)
(SEQ ID NO: 17)
tactaaaaggcagcctcctagaGUUUUAGUACUCUGGAAACAGAAUCUA
CUAAAACAAGGCAAAAUGCCGUGUUUAUCUCGUCAACUUGUUGGCGAGA
UUUU;
(iii)
(SEQ ID NO: 18)
attggctaccttggttggatgaGUUUUAGUACUCUGGAAACAGAAUCUA
CUAAAACAAGGCAAAAUGCCGUGUUUAUCUCGUCAACUUGUUGGCGAGA
UUUU;
(iv)
(SEQ ID NO: 19)
gacagctggctatccaggattcGUUUUAGUACUCUGGAAACAGAAUCUA
CUAAAACAAGGCAAAAUGCCGUGUUUAUCUCGUCAACUUGUUGGCGAGA
UUUU;
(v)
(SEQ ID NO: 20)
acttgcaggaggtgagggattaGUUUUAGUACUCUGGAAACAGAAUCU
ACUAAAACAAGGCAAAAUGCCGUGUUUAUCUCGUCAACUUGUUGGCGAG
AUUUU;
(vi)
(SEQ ID NO: 21)
attagggaatgcagactctgggGUUUUAGUACUCUGGAAACAGAAUCUA
CUAAAACAAGGCAAAAUGCCGUGUUUAUCUCGUCAACUUGUUGGCGAGA
UUUU;
(vii)
(SEQ ID NO: 22)
tgggtgagattagaggccactgGUUUUAGUACUCUGGAAACAGAAUCUA
CUAAAACAAGGCAAAAUGCCGUGUUUAUCUCGUCAACUUGUUGGCGAGA
UUUU;
(viii)
(SEQ ID NO: 23)
tgcttcctcccttgtctccctaGUUUUAGUACUCUGGAAACAGAAUCUA
CUAAAACAAGGCAAAAUGCCGUGUUUAUCUCGUCAACUUGUUGGCGAGA
UUUU;
(iv)
(SEQ ID NO: 24)
tggcatatgagaaaagtcacagGUUUUAGUACUCUGGAAACAGAAUCUA
CUAAAACAAGGCAAAAUGCCGUGUUUAUCUCGUCAACUUGUUGGCGAGA
UUUU;
and
(x)
(SEQ ID NO: 25)
ccttattcttttgatatactccGUUUUAGUACUCUGGAAACAGAAUCUA
CUAAAACAAGGCAAAAUGCCGUGUUUAUCUCGUCAACUUGUUGGCGAGA
UUUU.

In some embodiments, a gRNA for a Cas12i protein comprises a direct repeat sequence that comprises a stem-loop structure proximal to the 3′ end (immediately adjacent to the spacer sequence). In some embodiments, a gRNA for a Cas12i protein comprises a stem loop proximal to the 3′ end where the stem is 5-8 nucleotides in length. In some embodiments, the Type V-I RNA guide comprising a direct repeat sequence that comprises the sequence 5′-CCGUCNNNNNNUGACGG-3′ (SEQ ID NO: 26) or 5′-GUGCCNNNNNNUGGCAC-3′ (SEQ ID NO: 27) proximal to the 3′ end, wherein N refers to any nucleobase. In some embodiments, the Type V-I RNA guide direct repeat includes the sequence proximal to the 3′ end, wherein N refers to any nucleobase.

It is understood that because the gRNA sequences described above are RNA sequences. Any T (thymine) in the sequences referring to gRNAs would refer to U (or uracil) in the context of RNA molecules. Sequences containing T (thymine) herein would encompass both DNA molecules and RNA molecules (wherein T refers to U).

Moreover, the single-molecule gRNA can comprise no uracil at the 3′ end of the gRNA sequence. The gRNA can comprise one or more uracil at the 3′ end of the gRNA sequence. For example, the gRNA can comprise 1 uracil (U) at the 3′ end of the gRNA sequence. The gRNA can comprise 2 uracil (UU) at the 3′ end of the gRNA sequence. The gRNA can comprise 3 uracil (UUU) at the 3′ end of the gRNA sequence. The gRNA can comprise 4 uracil (UUUU) at the 3′ end of the gRNA sequence. The gRNA can comprise 5 uracil (UUUUU) at the 3′ end of the gRNA sequence. The gRNA can comprise 6 uracil (UUUUUU) at the 3′ end of the gRNA sequence. The gRNA can comprise 7 uracil (UUUUUUU) at the 3′ end of the gRNA sequence. The gRNA can comprise 8 uracil (UUUUUUUU) at the 3′ end of the gRNA sequence.

In some embodiments, the gene-editing system disclosed herein may comprise nucleic acids (e.g., vectors) encoding the gene editing system components or viral particles comprising such. In some embodiments, the gene-editing system comprises one nucleic acid capable of producing all components of the gene-editing system, including a nuclease and one or more gRNAs. In other examples, the gene-editing system comprises two or more nucleic acids.

The nucleic acid (or at least one nucleic acid in the set of nucleic acids) may be a vector such as a viral vector, such as a retroviral vector, an adenovirus vector, an adeno-associated viral (AAV) vector, and a herpes simplex virus (HSV) vector.

In some examples, the gene-editing system may comprise one or more viral particles that carry genetic materials for producing the components of the gene-editing system as disclosed herein. A viral particle (e.g., AAV particle) may comprise one or more components (or agents for producing one or more components) of a gene-editing system (e.g., as described herein). A viral particle (or virion) comprises a nucleic acid, which encodes the viral genome, and an outer shell of protein (i.e., a capsid). In some instances, a viral particle further comprises an envelope of lipids that surround the protein shell.

In some examples, a viral particle comprises a nucleic acid capable of producing all components of the gene-editing system, including a nuclease and one or more gRNAs. In other examples, a viral particle comprises a nucleic acid capable of producing one or more components of the gene-editing system. For example, a viral particle may comprise a nucleic acid capable of producing the nuclease and the gRNA. Alternatively, a viral particle may comprise a nucleic acid capable of producing the one or more gRNAs. In another example, a viral particle may comprise a nucleic acid capable of producing only one of the nucleases or any one of the gRNAs.

The viral particles described herein may be derived from any viral particle known in the art including, but not limited to, a retroviral particle, an adenovirus particle, an adeno-associated viral (AAV) particle, or a herpes simplex virus (HSV) particle. In some embodiments, the viral particle is an AAV particle. In some embodiments, the AAV vector is an AAV1, AAV2, AAV3, AAV4, AAV5, AAV6, AAV7, AAV8, AAVrhlO (see, e.g., U.S. Pat. No. 9,790,472, which is incorporated by reference herein in its entirety), AAVrh74 (see, e.g., US 2015/0111955, which is incorporated by reference herein in its entirety), or AAV9 vector, wherein the number following AAV indicates the AAV serotype. Any variant of an AAV vector or serotype thereof, such as a self-complementary AAV (sc AAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors.

In some embodiments, a set of viral particles comprises more than one gene-editing system. In some embodiments, each viral particle in the set of viral particles is an AAV particle. In other embodiments, a set of viral particles comprises more than one type of viral particle (e.g., a retroviral particle, an adenovirus particle, an adeno-associated viral (AAV) particle, or a herpes simplex virus (HSV) particle).

In some embodiments, a gRNA used in accordance with the present disclosure is synthetic and/or chemically modified, and may be delivered to a stem cell via methods known in the art (e.g., via transfection or a lipid nanoparticle). Lipid nanoparticles (LNPs) are a known means for delivery of nucleotide and protein cargo, and may be used for delivery of the guide RNAs, compositions, or pharmaceutical formulations disclosed herein. In some embodiments, the LNPs deliver nucleic acid, protein, or nucleic acid together with protein. In some embodiments, the gene editing system comprises one or more ribonucleoproteins comprising a Cas protein (e.g., a Cas9 or Cas12i2 protein) and a guide RNA. In some embodiments, the ribonucleoproteins are administered to any of the cells disclosed herein (e.g., any of the stem cells disclosed herein) by a lipid nanoparticle. In some embodiments, the disclosure provides for a nucleic acid encoding an endonuclease (e.g., a Cas9 or Cas12i2 protein) and a nucleic acid encoding one or more gRNAs, which are optionally administered to any of the cells disclosed herein (e.g., any of the stem cells disclosed herein) by a lipid nanoparticle.

In some embodiments, the gRNA is chemically modified. A gRNA comprising one or more modified nucleosides or nucleotides is called a “modified” gRNA or “chemically modified” gRNA, to describe the presence of one or more non-naturally and/or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified gRNAs comprising nucleosides and nucleotides (collectively “residues”) that can have two, three, four, or more modifications. For example, a modified residue can have a modified sugar and a modified nucleobase, or a modified sugar and a modified phosphodiester. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxy lamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e., at sugar modification. For example, the 2′ hydroxyl group (OH) can be modified, e.g., replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂₀)nCH₂CH₂₀R wherein R can be, e.g., H or optionally substituted alkyl, and n can be an integer from 0 to 20 (e.g, from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a Ci-e alkylene or Ci-e heteroalky lene bridge, to the 4′ carbon of the same ribose sugar, where exemplary bridges can include methylene, propylene, ether, or amino bridges; O-amino (wherein amino can be, e.g., N %; alkylamino, dialky lamino, heterocyclyl, arylamino, diarylamino, heteroarylamino, or diheteroarylamino, ethylenediamine, or polyamino) and aminoalkoxy, 0(CH2)n-amino, (wherein amino can be, e.g., N3/4; alkylamino, dialky lamino, heterocyclyl, arylamino, diarylamino, heteroarylamino, or diheteroarylamino, ethylenediamine, or poly amino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH2CH2OCH3, e.g., a PEG derivative).

“Deoxy” 2′ modifications can include hydrogen (i.e., deoxyribose sugars, e.g., at the overhang portions of partially dsRNA); halo (e.g., bromo, chloro, fluoro, or iodo); amino (wherein amino can be, e.g., NH2; alkylamino, dialkylamino, heterocyclyl, arylamino, diary lamino, heteroarylamino, diheteroarylamino, or amino acid); NH(CH2CH2NH)nCH2CH2-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein. The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g., L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising an sgRNA, one or more residues at one or both ends of the sgRNA may be chemically modified, and/or internal nucleosides may be modified, and/or the entire sgRNA may be chemically modified. Certain embodiments comprise a 5′ end modification. Certain embodiments comprise a 3′ end modification. In some embodiments, the modifications comprise a 2′-O-methyl modification.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Modifications of 2′-fluoro (2′-F) are encompassed. Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

Abasic nucleotides refer to those which lack nitrogenous bases.

Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide.

In some embodiments, a method of producing a hypoimmune cell described herein results in a knock out of the target polynucleotide sequence or a portion thereof (e.g., knock out of the 3′-UTR of an immunosuppressor, B2M, and/or CIITA gene). In some embodiments, a method of producing a hypoimmune cell described herein results in a knock in of the target polynucleotide sequence or a portion thereof (e.g., knock in of an immunosuppressor or an anti-CRISPR protein). In some embodiments, the method can be performed in vitro, in vivo or ex vivo for both therapeutic and research purposes. In some embodiments, any genetic modification described herein is a homozygous modification. In some embodiments, genetic modification described herein is a heterozygous modification.

In some embodiments, a method of producing a hypoimmune cell described herein is carried out using a CRISPR/Cas system. In some embodiments, CRISPR/Cas systems can alter target polynucleotides with high efficiency. In certain embodiments, the efficiency of alteration is at least about 5%. In certain embodiments, the efficiency of alteration is at least about 10%. In certain embodiments, the efficiency of alteration is from about 10% to about 80%. In certain embodiments, the efficiency of alteration is from about 30% to about 80%. In certain embodiments, the efficiency of alteration is from about 50% to about 80%. In some embodiments, the efficiency of alteration is greater than or equal to about 80%. In some embodiments, the efficiency of alteration is greater than or equal to about 85%. In some embodiments, the efficiency of alteration is greater than or equal to about 90%, about 91%, about 92%, about 93%, about 94%, about 95%, about 96%, about 97%, about 98%, or about 99%. In some embodiments, the efficiency of alteration is equal to about 100%.

### EXAMPLES

These examples are provided for illustrative purposes only and not to limit the scope of the claims provided herein.

### Example 1. Design and Validation of PDL1 3′ UTR-Targeting CRISPR/Cas9 Knock-Out Constructs

PDL1 has been shown to play a major role in suppression of the adaptive immune system. Normal activity of PDL1 is responsible for inhibiting overstimulation of immune cells (e.g., CD8+/CD4+ T cells). As such, PDL1 is a therapeutic target in stem cell research: Overexpression of PDL1 or reduced restrictions on PDL1 expression may facilitate effective stem cell transplantation by inhibiting a detrimental immune response. The present disclosure relates, in part, to methods of manipulating the endogenous PDL1 3′-UTR to drive overexpression of the PDL1 gene, either constitutively or in response to a specific cue (e.g., a cytokine such as interferon-gamma).

Manipulation of the endogenous PDL1 gene locus offers several potential solutions to some of the problems encountered within the traditional transgene knock-in paradigm. In particular, the traditional transgene knock-in approach may be susceptible to epigenetic silencing of the knock-in transgene. The present disclosure addresses this particular problem by using a representative gene editing system, i.e., the CRISPR/Cas9 system, to excise the majority of the endogenous 3′ UTR of the endogenous PDL1 gene. The PDL1 3′ UTR has a known post-transcriptional regulatory function that modulates subsequent translation of PDL1 mRNA (e.g., as described in Kataoka et al., Nature, vol. 534, 401-418, 2016). Specific disruption of the 3′ UTR of the endogenous PDL1 gene by the CRISPR/Cas9 system will enable increased expression of the PDL1 gene in cell types of interest (e.g., stem cells and stem cell derivatives).

To identify CRISPR/Cas9 knock-out constructs effective in excising the endogenous 3′ UTR of the endogenous PDL1 gene, human embryonic cells (hESC) were nucleofected with CRISPR guide RNAs targeting the PDL1 3′ UTR or NLRC5 and the Cas9 endonuclease (3 μL of guide RNA (300 pmols) was mixed with 2 μL of Cas9 (40 pmols) to produce a 7.5:1 ratio). The cells were incubated for 2 days, then stimulated with IFNγ for 3 days. Analysis of PDL1 expression levels following IFNγ stimulation indicated subtle upregulation of PDL1 gene expression. The guide RNA target sequences in PDL1 3′-UTR are provided in Table 1.

TABLE 1
Target sequences by the CRISPR-Cas9 System (Kataoka et al.)

| Target positions in PDL1 | | |
|---|---|---|
| mRNA as set forth in | | |
| SEQ ID NO: 1 | Target Sequence | PAM |
| 1021-1040 | Fw: AGAGGAAGGAATGGGCCCGT | GGG |
| | (SEQ ID NO: 13) | |
| 1003-1022 | Fw: TCGGGGCTGAGCGTGACAAG | AGG |
| | (SEQ ID NO: 14) | |
| targets downstream of | Rev: TCTTCTTGGTATGGTCCTAA | AGG |
| PDL1 3′-UTR on the | (SEQ ID NO: 15) | |
| opposition strand | | |

Clones from the previous experiment were next selected for further analysis. Twelve clones were picked from a 96-well plate and their DNA was extracted (Lucigen DNA Quick Extract). The DNA for each of the 12 clones was analyzed for the presence of the endogenous 3′ UTR of the endogenous PDL1 gene to determine whether the 3′ UTR had been excised in any of the clones. Primers that span the 3′ UTR of the PDL1 gene were used in a PCR assay with the extracted DNA. Of the clones generated, three clones (#12, #19, and #25) were found to have disruption of the endogenous 3′ UTR of the endogenous PDL1 gene.

The three clones were tested again at the stem cell state for gross dysregulation of the PDL1 gene without INFγ stimulation. Surprisingly, PDL1 gene expression was upregulated in all three clones.

### Example 2. Confirmation of PDL1 Overexpression Effect

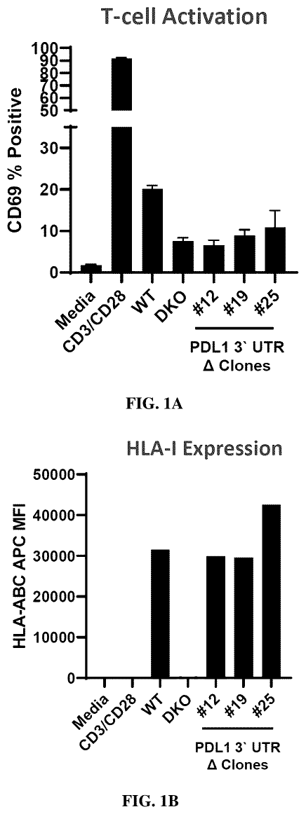

To confirm the efficacy of the three clones containing the CRISPR/Cas9 constructs in excising the 3′ UTR and increasing expression of the endogenous PDL1 gene, wild-type cells, B2M/CIITA double knock-out (DKO) cells, and the three PDL1 3′ UTR deletion clones (#12, #19, and #25) were differentiated into endothelial cells and mixed with human CD8+ T cells to be assayed for CD69 surface expression (FIG. 1A). Media alone (negative control) did not activate CD69 surface expression, while media containing CD3/CD28 activating beads (positive control) did activate CD69 surface expression. The wild-type cells appeared to activate CD69 surface expression, while the DKO cells did not. Surprisingly, disruption of the PDL1 3′-UTR led to reduced T-cell activation in all three clones, as measured by reduced activation of CD69 surface expression. To further verify the composition of the cells used in FIG. 1A, unstimulated wild-type cells, B2M/CIITA DKO cells, and the three PDL1 3′ UTR deletion clones were probed for HLA-I expression (FIG. 1B). The three PDL1 3′ UTR deletion clones expressed HLA-I, while the B2M/CIITA DKO cells did not.

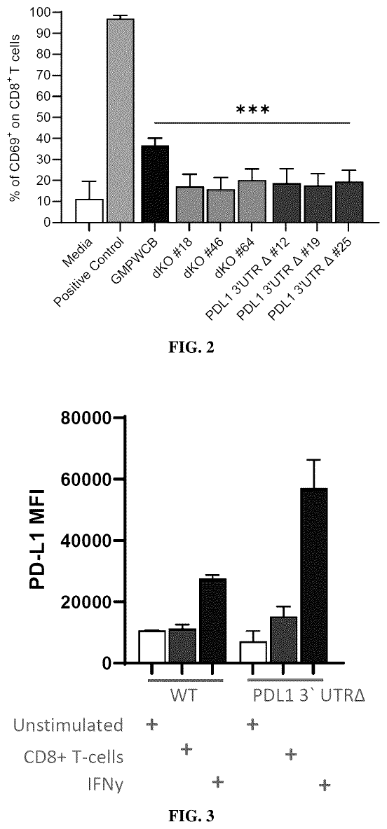

To further confirm the PDL1 overexpression effect, purified human CD8+ T cells were co-cultured with either B2M/CIITA DKO clones (DKO #18, DKO #46, and DKO #64) or the three PDL1 3′ UTR deletion clones (#12, #19, and #25) and assessed for T-cell activation by assaying CD69 surface expression (FIG. 2). The three PDL1 3′ UTR deletion clones appeared to reduce T-cell activation as effectively as the DKO clones in this experiment.

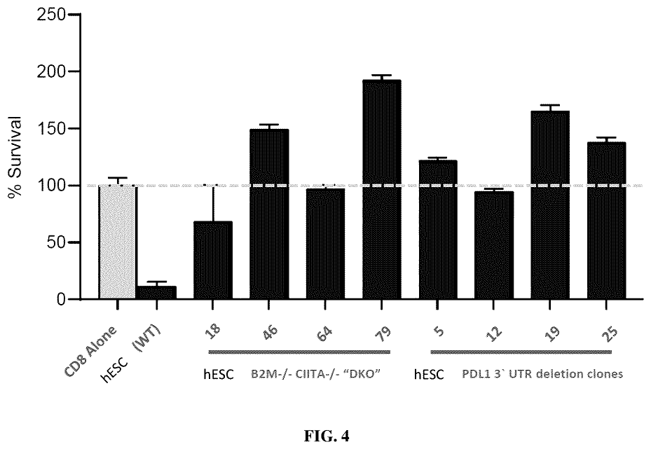

To determine whether increased expression of PDL1 following 3′ UTR excision requires stimulation, wild-type cells and PDL1 3′ UTR deletion clones were differentiated into endothelial cells and probed (flow cytometry measured by mean fluorescence intensity (MFI)) for PDL1 surface expression with or without stimulation with a cytokine such as IFN-gamma (FIG. 3). In the absence of stimulation, PDL1 surface expression was similar between the wild-type cells and the PDL1 3′ UTR deletion clones. When co-cultured with CD8+ T-cells, the PDL1 3′ UTR deletion clones exhibited a modest upregulation of PDL1 surface expression. When exposed to INFγ stimulation, the PDL1 3′ UTR deletion clones exhibited a greater induction of PDL1 surface expression compared to the wild-type cells.

To determine whether PDL1 3′ UTR deletion protects cells from T-cell mediated cell death, purified human CD8+ T-cells were co-cultured with wild-type cells, B2M/CIITA DKO clones, and PDL1 3′ UTR deletion clones (FIG. 4). The wild-type cells were susceptible to T-cell mediated cell death, with nearly all cells undergoing cell death. Three of the four DKO clones were resistant to T-cell mediated cell death. Surprisingly, each of the PDL1 3′ UTR deletion clones were resistant to T-cell mediated cell death.

What is claimed is:

1. An isolated stem cell comprising a disruption in the 3′-untranslated region (3′-UTR) of an allele encoding an immunosuppressor.

2. The isolated stem cell of claim 1, wherein the disruption comprises a deletion, an insertion, a translocation, an inversion, or a substitution in the 3′-UTR.

3. The isolated stem cell of claim 1 or claim 2, wherein the disruption reduces binding of the 3′-UTR to endogenous RNA-binding proteins and/or microRNAs.

4. The isolated stem cell of any one of claims 1-3, wherein the immunosuppressor is selected from the group consisting of: PDL1, CD47, HLA-G, and combinations thereof.

5. The isolated stem cell of any one of claims 1-4, wherein the deletion in the 3′-UTR results in increased expression of the immunosuppressor.

6. The isolated stem cell of claim 5, wherein the increased expression of the immunosuppressor is induced or increased by a cytokine, optionally wherein the cytokine is interferon gamma.

7. The isolated stem cell of any one of claims 1-6, wherein the immunosuppressor is PDL1.

8. The isolated stem cell of claim 7, wherein the disruption results in a deletion of the PDL1 3′-UTR.

9. The isolated stem cell of claim 7, wherein the disruption results in an inversion of the PDL1 3′-UTR.

10. The isolated stem cell of claim 7, wherein the disruption results in one or more substitutions of nucleotide in the PD-L1 3′-UTR.

11. The isolated stem cell of any one of claims 7-10, wherein the disruption reduces binding of one or more of endogenous microRNAs to PDL1 3′-UTR, optionally wherein the one or more of endogenous microRNA are selected from the group consisting of: miR-34a, miR-140, miR-200a, miR-200b/c, miR-142, miR-340, miR-383, miR-424(322), miR-338-5p, miR-324-5p, miR-152, miR-200b, miR-138-5p, miR-195, miR-16, miR-15a, miR15b miR-193a-3p, miR-497-5p, miR-33a, miR17-5p, miR-155, and miR-513.

12. The isolated stem cell of any one of claims 1-11, wherein the disruption results in deletion of 1-7 nucleotides in one or more of PDL1 3′-UTR sequences as set forth in any one of SEQ ID NOs: 32, 34, 36, 38, 40, 42, 45, 48, 36, 58, 59, 61, 63, 65, 67, 69, 71, and 73.

13. The isolated stem cell of any one of claims 1-12, wherein the disruption results in deletion of 1-24 nucleotides in one or more of PDL1 3′-UTR sequences as set forth in any one of SEQ ID NOs: 31, 33, 35, 37, 39, 41, 44, 47, 57, 60, 62, 64, 66, 68, 70, and 72.

14. The isolated stem cell of any one of claims 1-6, wherein the immunosuppressor is HLA-G.

15. The isolated stem cell of claim 14, wherein the disruption reduces binding of one or more of endogenous microRNAs to HLA-G 3′-UTR, optionally wherein the one or more of endogenous microRNA are selected from the group consisting of: miR-133A, miR-148A, miR-148B, miR-152, miR-548q and/or miR-628-5p.

16. The isolated stem cell of claim 15, wherein the disruption results in deletion of at least 5 consecutive nucleotides beginning at and inclusive of position +2961 of the HLA-G 3′-UTR, and/or insertion of at least 5 nucleotides at position +2961.

17. The isolated stem cell of claim 15, wherein the disruption is in an HLA-G 3′-UTR sequence as set forth in SEQ ID NO: 74.

18. The isolated stem cell of claim 17, wherein the disruption results in a deletion of at least 1 nucleotide of an HLA-G 3′-UTR sequence as set forth in SEQ ID NO: 75.

19. The isolated stem cell of claim 17, wherein the disruption results in one or more mutations selected from C120G, G252C, A297G, and/or C306G in an HLA-G 3′-UTR sequence as set forth in SEQ ID NO: 74.

20. The isolated stem cell of any one of claims 1-19, further comprising an insertion of a sequence encoding CD47, CTLA-4, PDL1, PDL2, HLA-C, HLA-E, HLA-G, C1-inhibitor, IL-35, DUX4, IDO1, IL10, CCL21, CCL22, CD16, CD52, H2-M3, CD200, FASLG, MFGE8, and/or SERPINB9 into the disrupted 3′-UTR locus.

21. The isolated stem cell of claim 20, wherein insertion of the sequence encoding CD47 into the PDL1 3′-UTR locus results in an RNA comprising coding sequences for the immunosuppressor and CD47.

22. The isolated stem cell of claim 20, further comprising an insertion of a sequence encoding CD47, CTLA-4, PDL1, PDL2, HLA-C, HLA-E, HLA-G, C1-inhibitor, IL-35, DUX4, IDO1, IL10, CCL21, CCL22, CD16, CD52, H2-M3, CD200, FASLG, MFGE8, and/or SERPINB9 into a safe harbor locus.

23. The isolated stem cell of any one of claims 1-19, wherein the isolated stem cell does not contain an insertion of an exogenous coding sequence in its genome.

24. The isolated stem cell of any one of claims 1-23, wherein the isolated stem cell has reduced expression of MHC-I and MHC-II human leukocyte antigens (HLA) relative to a wild type stem cell of the same cell type.

25. The isolated stem cell of claim 24, wherein the reduced expression of MHC-I HLA results from a disruption in an allele encoding 3-2 microglobulin (B2M).

26. The isolated stem cell of claim 24 or claim 25, wherein the reduced expression of MHC-II HLA results from a disruption in an allele encoding class II major histocompatibility complex transactivator (CIITA).

27. The isolated stem cell of any one of claims 1-26, wherein the stem cell is an embryonic stem cell.

28. The isolated stem cell of any one of claims 1-26, wherein the stem cell is a pluripotent stem cell.

29. The isolated stem cell of any one of claims 1-28, wherein the stem cell is a human stem cell.

30. The isolated stem cell of any one of claims 1-29, wherein the stem cell is negative for A antigen and negative for B antigen.

31. The isolated stem cell of any one of claims 1-30, wherein the stem cell is negative for Rh antigen.

32. A cell differentiated from the isolated stem cell of any one of claims 1-31.

33. The cell of claim 32, wherein the cell is selected from the group consisting of: a fibroblast cell, an endothelial cell, a definitive endoderm cell, a primitive gut tube cell, a pancreatic progenitor cell, a pancreatic endocrine cell, a pancreatic islet cell, a stem cell-derived β cell, a stem cell-derived α cell, a stem cell-derived δ cell, a stem cell-derived enterochromaffin (EC) cell, an insulin producing cell, an insulin-positive β-like cell, a hematopoietic stem cell, a hematopoietic progenitor cell, a muscle cell, a satellite stem cell, a liver cell, a neuron, or an immune cell.

34. The cell of claim 32 or claim 33, wherein the cell is an immune cell, optionally wherein the immune cell expresses a chimeric antigen receptor (CAR) or an engineered T-cell receptor (TCR).

35. The cell of any one of claims 32-34, wherein the cell is less immunogenic relative to a cell of the same cell type.

36. A composition comprising the isolated stem cell of any one of claims 1-31, or the cell of any one of claims 32-35.

37. The composition of claim 36, comprising NKX6.1-positive, ISL-positive cells and NKX6.1-negative, ISL-positive cells; wherein the population comprises more NKX6.1-positive, ISL-positive cells than NKX6.1-negative, ISL-positive cells; wherein at least 15% of the cells in the population are NKX6.1-negative, ISL-positive cells; and wherein less than 12% of the cells in the population are NKX6.1-negative, ISL-negative cells.

38. A method comprising administering to a subject in need thereof the isolated stem cell of any one of claims 1-31, or the cell of any one of claims 32-37.

39. A method of treating diabetes, comprising administering to a subject in need thereof pancreatic islet cells differentiated from the isolated stem cell of any one of claims 1-31, or the composition of claim 37.

40. A method of treating cancer, comprising administering to a subject in need thereof immune cells differentiated from the isolated stem cell of any one of claims 1-31. or the cell of claim 34.

41. The method of claim 40, wherein the cancer is a hematologic cancer.

42. A method of producing the isolated stem cell of any one of claims 1-31, comprising delivering to a stem cell a CRISPR system comprising an RNA-targeted endonuclease and one or more guide RNAs (gRNA) comprising a nucleotide sequence that targets the 3′-UTR of an allele encoding the immunosuppressor.

43. The method of claim 42, wherein the RNA-targeted endonuclease is a Cas protein.

44. The method of claim 43, wherein the Cas protein is a Cas9 protein or a Cas12i protein.

45. The method of any one of claims 42-44, wherein the immunosuppressor is PDL1, CD47, or HLA-G.

46. The method of any one of claims 42-45, wherein the immunosuppressor is PDL1.

47. The method of claim 46, wherein the gRNA targets a target sequence corresponding to positions 1003-1022 or positions 1021-1040 of a PDL1 sequence as set forth in SEQ ID NO: 1, or targets a target sequence downstream of the 3′-UTR of PDL1 on opposite strand.

48. The method of claim 46 or claim 47, wherein the composition comprises a first gRNA that targets a target sequence corresponding to positions 1003-1022 or positions 1021-1040 of a PDL1 sequence as set forth in SEQ ID NO: 1 and a second gRNA that targets a target sequence downstream of the 3′-UTR of PDL1 on opposite strand.

49. The method of any one of claims 42-48, wherein the gRNA is modified.

50. The method of claim 49, wherein the gRNA is delivered in a lipid nanoparticle (LNP).

51. The method of any one of claims 42-50, wherein the gRNA is delivered via a nucleic acid comprising a nucleotide sequence encoding the gRNAs, optionally wherein the nucleic acid is a viral vector.

52. The method of any one of claims 42-51, wherein RNA-targeted endonuclease is delivered via a nucleic acid comprising a nucleotide sequence encoding the RNA-targeted endonuclease, optionally wherein the nucleic acid is a viral vector.


SEQUENCE LISTING (97 sequences)

SEQ ID NO: 1 - DNA, 3634 bp, Homo sapiens
  source 1..3634: mol_type = genomic DNA
agttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 2 - DNA, 3292 bp, Homo sapiens
  source 1..3292: mol_type = genomic DNA
agttctgcgcagcttcccgaggctccgcaccagccgcgcttctgtccgcctgcagggcattccagaaagatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 3 - DNA, 868 bp, Homo sapiens
  source 1..868: mol_type = genomic DNA
agttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccaggtaatattctgaatgtgtccattaaaatatgtctaacactgtcccctagcacctagcatgatgtctgcctatcatagtcattcagtgattgttgaataaatgaatgaatgaataaca

SEQ ID NO: 4 - DNA, 5292 bp, Homo sapiens
  source 1..5292: mol_type = genomic DNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 5 - DNA, 5234 bp, Homo sapiens
  source 1..5234: mol_type = genomic DNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 6 - DNA, 5259 bp, Homo sapiens
  source 1..5259: mol_type = genomic DNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 7 - DNA, 1386 bp, Homo sapiens
  source 1..1386: mol_type = genomic DNA
atatagtaacatagtgtggtactttgtcttgaggagatgtcctggactcacacggaaacttagggctacggaatgaagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaa

SEQ ID NO: 8 - DNA, 1489 bp, Homo sapiens
  source 1..1489: mol_type = genomic DNA
atatagtaacatagtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaa

SEQ ID NO: 9 - DNA, 1424 bp, Homo sapiens
  source 1..1424: mol_type = genomic DNA
atagtagcaggaccactatagagagaacactcatgtagcaggtcatggaacagtgctagagccacagttcaggaatgtcctggactcacacggaaacttagggctacggaatgaagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaa

SEQ ID NO: 10 - DNA, 1319 bp, Homo sapiens
  source 1..1319: mol_type = genomic DNA
attctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaa

SEQ ID NO: 11 - DNA, 1887 bp, Homo sapiens
  source 1..1887: mol_type = genomic DNA
gctttctattcaagtgccttctgtgtgtgcacatgtgtaatacatatctgggatcaaagctatctatataaagtccttgattctgtgtgggttcaaacacatttcaaagcttcaggatcctgaaaggttttgctctacttcctgaagacctgaacaccgctcccataaagccatggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgctaaagaaaagaagccctcttacaacaggggtctatgtgaaaatgcccccaacagagccagaatgtgaaaagcaatttcagccttattttattcccatcaattgagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttccgga

SEQ ID NO: 12 - DNA, 1997 bp, Homo sapiens
  source 1..1997: mol_type = genomic DNA
gctttctattcaagtgccttctgtgtgtgcacatgtgtaatacatatctgggatcaaagctatctatataaagtccttgattctgtgtgggttcaaacacatttcaaagcttcaggatcctgaaaggttttgctctacttcctgaagacctgaacaccgctcccataaagccatggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattgagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttccgga

SEQ ID NO: 13 - DNA, 20 bp, synthetic construct
  misc_feature 1..20: note = Synthetic
  source 1..20: mol_type = other DNA
agaggaaggaatgggcccgt

SEQ ID NO: 14 - DNA, 20 bp, synthetic construct
  misc_feature 1..20: note = Synthetic
  source 1..20: mol_type = other DNA
tcggggctgagcgtgacaag

SEQ ID NO: 15 - DNA, 20 bp, synthetic construct
  misc_feature 1..20: note = Synthetic
  source 1..20: mol_type = other DNA
tcttcttggtatggtcctaa

SEQ ID NO: 16 - RNA, 102 nt, synthetic construct
  source 1..102: mol_type = other RNA
acccagcctgacaccaaatttagttttagtactctggaaacagaatctactaaaacaaggcaaaatgccgtgtttatctcgtcaacttgttggcgagatttt

SEQ ID NO: 17 - RNA, 102 nt, synthetic construct
  source 1..102: mol_type = other RNA
tactaaaaggcagcctcctagagttttagtactctggaaacagaatctactaaaacaaggcaaaatgccgtgtttatctcgtcaacttgttggcgagatttt

SEQ ID NO: 18 - RNA, 102 nt, synthetic construct
  source 1..102: mol_type = other RNA
attggctaccttggttggatgagttttagtactctggaaacagaatctactaaaacaaggcaaaatgccgtgtttatctcgtcaacttgttggcgagatttt

SEQ ID NO: 19 - RNA, 102 nt, synthetic construct
  source 1..102: mol_type = other RNA
gacagctggctatccaggattcgttttagtactctggaaacagaatctactaaaacaaggcaaaatgccgtgtttatctcgtcaacttgttggcgagatttt

SEQ ID NO: 20 - RNA, 102 nt, synthetic construct
  source 1..102: mol_type = other RNA
acttgcaggaggtgagggattagttttagtactctggaaacagaatctactaaaacaaggcaaaatgccgtgtttatctcgtcaacttgttggcgagatttt

SEQ ID NO: 21 - RNA, 102 nt, synthetic construct
  source 1..102: mol_type = other RNA
attagggaatgcagactctggggttttagtactctggaaacagaatctactaaaacaaggcaaaatgccgtgtttatctcgtcaacttgttggcgagatttt

SEQ ID NO: 22 - RNA, 102 nt, synthetic construct
  source 1..102: mol_type = other RNA
tgggtgagattagaggccactggttttagtactctggaaacagaatctactaaaacaaggcaaaatgccgtgtttatctcgtcaacttgttggcgagatttt

SEQ ID NO: 23 - RNA, 102 nt, synthetic construct
  source 1..102: mol_type = other RNA
tgcttcctcccttgtctccctagttttagtactctggaaacagaatctactaaaacaaggcaaaatgccgtgtttatctcgtcaacttgttggcgagatttt

SEQ ID NO: 24 - RNA, 102 nt, synthetic construct
  source 1..102: mol_type = other RNA
tggcatatgagaaaagtcacaggttttagtactctggaaacagaatctactaaaacaaggcaaaatgccgtgtttatctcgtcaacttgttggcgagatttt

SEQ ID NO: 25 - RNA, 102 nt, synthetic construct
  source 1..102: mol_type = other RNA
ccttattcttttgatatactccgttttagtactctggaaacagaatctactaaaacaaggcaaaatgccgtgtttatctcgtcaacttgttggcgagatttt

SEQ ID NO: 26 - RNA, 17 nt, synthetic construct
  misc_feature 1..17: note = Synthetic
  source 1..17: mol_type = other RNA
ccgtcnnnnnntgacgg

SEQ ID NO: 27 - RNA, 17 nt, synthetic construct
  misc_feature 1..17: note = Synthetic
  source 1..17: mol_type = other RNA
gtgccnnnnnntggcac

SEQ ID NO: 28 - DNA, 20013 bp, Homo sapiens
  source 1..20013: mol_type = genomic DNA
agttctgcgcagcttcccgaggctccgcaccagccgcgcttctgtccgcctgcaggtagggagcgttgttcctccgcgggtgcccacggcccagtatctctggctagctcgctgggcactttaggacggagggtctctacaccctttctttgggatggagagaggagaagggaaagggaacgcgatggtctagggggcagtagagccaattacctgttggggttaataagaacaggcaatgcatctggccttcctccaggcgcgattcagttttgctctaaaaataatttatacctctaaaaataaataagataggtagtataggataggtagtcattcttatgcgactgtgtgttcagaatatagctctgatgctaggctggaggtctggacacgggtccaagtccaccgccagctgcttgctagtaacatgacttgtgtaagttatcccagctgcagcatctaagtaagtctcttcctgcgctaagcaggtccaggatccctgaacggaatttatttgctctgtccattctgagaacccaaaggagtcctaaaagaggaatggaggagcctaagaataaaaatagtataataaaacatttcttagacacattgaccttggcctatgtcaaagttcagtctgggtttgtcttataacacaaggagtaaaagtaccattgttctacctctttttttaatacttgaaaaaaatttactgtggatgcttttctatgaattaaataaccttctaaaaaatgttttcattgctgcattcgattagattgggtaactaaatgaaattaattcctcactgttgggtataaaggttatttacagtggttctgtcttagccattcactgaactcattgcatatatatctctggaatattgctgattgtttccttcaagtaaacttagaagtgtaactacttagtcaaagagcctgaatattttaaaggccttttgaagaaaactgaaaatgctttccagaaaggatgtatcagttgacaatgacagtcgtcaacagtatttaaggagaactatgatactctgaagaaaaacttagcctttctcagtaaaagtaggtaggcagaggccacatgacagcagttagagtgtggtcttcaaggaagtcacagaaatactgtggggaattgaaaccccatgtggaaaatgtacaagagtgtctcagtgtgactgagaaggaggttgggcatggggtttcatggagtttaataaagtttggtcacttagtagaggtttaataaatcaactgtcttaatctttgatcctacttaagaattttttttttgtttttgtagagatggggctcttgttatgttgcccaggctgttctcgaactcctagcctcaggcgatcctccctcctcaggctccagaagtcctgggattactggcgggagccaccatgcaggcctcttgctcctacttttgagaaaggaagtttaaccggttttttttgtctttttttttttttttttgagacagagtctcactctgttgcccatgctggagtgcagtggtgcaatctcagctcactgcctcccgggttcaagtgattctcctgcctcagcctcccgagtagctgggactacaggcacctgccaccacgcccagctaatttttgtatttttagtagaaatggggtttcaccatattggccaggctgatctcgaactcctgacctcaggtgatccgcctgcctcggcctcccaaagtgctgggattacaggcatgagccactgctcctggctgcttaactttttctctatctcatcctcctacccatcctacccttggaagatagagaagtagtattagttccatagtgttatactgggcttcccccagggacaaacccacttccccaacctgaatgagccatcacttcttccccagtttacatttcattgctctttaaatgtctccattcggatatgggaattcacatatggtcataattcttacctgaagaagatgtcagtcttcttctcttagaccaactgccctgatatgaggtttagaggttaaagaacatgtgtgtatttacatgatctttgtattctgccttttcgtccctcactaatgacagctgcaccccaaggaaatggagctgtggaagagagggtttgataagaaattaagtaaatattggatctaatccatcaccctccaggaagcctttattactcctaaaaatttcaaccaaattcattaaaggacaagaactccaccagagtaggccataaacattggcaaaattagttgtaatccatgactagatttaatgtccctttgttttattcccatatggttataatgctttgcttggcattaggggtattttaagttttcttctgcctagtaagtgaatttgtgtttataatacaataatcataaaatatcacattaatattttataactgtacagttataaaatattttataagtaatatttatattttataagtaatattttataactgtacagttaactctggcccaaggaaaagatagtctgatagatgctgcagccccattttagcaaatgtgacctcacaggcctgaatgccatcgctattccacatctacaggatagacggaaaggaaagaaataaaaaaataggtacctaacactggcaagaggatgatgactcatgttatttcacttaacctttttatcttttaacatgaaggactcatacaggttgataagaaaccagtgacataaacagaccaaaaaatgatcagatctttcaaattagcaaaaaaataatattttttaaacaatgggtgaaaatacagtgtaacagtaccaattatcaacatgtgttgagaaccagaaaaatgttctttttctttgatcagcaacactatttgggaaaatctatcctcagggcctagcctggggccctggcacacagtaggcactcaacgaatatttgctgaacacacaaatacttatgatattttaaaaaattggcaacaatctgatacctaacaatagagggattaaatattatggaactgttaaataagatgcttatgaataccatgcagtaagatgggcaatatttatgccataagctttaatgaaacaaatgggtattaaatgtatgataaggttataaattactttttaaaagattacagggaaaaaaattgaaagatatacactgaaatgttttttgctcacagtggtgacaaggtttctcagcactggcactgttgacgttttaggctgtatgtctttgctgtgggaggctggcctgtgcactgcagggtgtttggcagcactcttggcctctgcccctagatagcaatagcagtcctccctcaaccagcccaattttgacaaccaaaaatgtttccaggcatcaccagatgctccctgggtgagagtgatgaaatagtaggggattttccccttcttttcttattttctgtaattccattatattactttaataataaagaaaaaaacataaaaaataaacgaatgttattattctacgtcagtttggatgtttggactccattttggggttctttccattatatcacttggtctgctaaacattctacggtttggtaaggtgaagtgattcatgaaattttggttttatttttttcctgatactaaaaataaaacattctttcacttggaaatttggacacagaacaccaaaaaaaatccataatctcatctctctttttctgtcttttccttccttttttccctttaaaaacaataaagagtgaaacctacctgttctccctctaatttaattcctaaatataatcactgtcaatatcttggacatttcctgtgtctaaacacacacacacacttttttttttcagcaaaagtggatttctgctacatgtagtgttctgcaacttactttctatgtgtttacaaaatcagtacatgtacatatgctgaattcagtccttaatggtattatattttgtgaatataccaaaatttgtttaaccacttagacaatctaggatattctcagtttgctgttatgagcaatgctcttcctttacatatacagacatatatatatatatgtgtgtgtgtgtgtttttgttttagtaggatagatttctaggagagggtgaaaggtcttatgacatccgcatttacgattgtaataggaagtatcaaagtgccccctaaagaaaaaaatcctcccattagtgggtaagaaagcctatttgttcatatcttcacaaacactaaatattagaaatatttacaattgtggtcaagctcataagtgaaaatggtatttcatatcttatattttttattgtgagattgaacatctttcatatgtttacatgtcacctgtatttcttattctctgaactatatgttatgacctttcactttttttcctcatgggttatgtgtagtttgtatagttgtcttattgattgttaggagctatttatatattaggaacattaatctcctgtcttatatatacgtggcatcgattagttgatcatttgtgagttcatgtctgtatacaaagattggagaggcactaagagggaaaacttacctctttcttatcaaagtttgtaaatatatgtataacagaagagggagaaaatattaataaatgcacagattggctgaaatagagtataaatcttttactcccctacttcaacataaactgcaaaaggagagtgacttttctttcactctgacttccgtattcctcatgcttaaaatagtgcctagcacagaagaggtgctcaatcagtgtttgctaaacgaaataattagtcacatttcaagcaggatgactaaatgaagaatagaatctaggcagatactctggaagagtggctgtgagtcattcatatcttagtatgaattagtcaaatccaactctctccccttcccactccccactgttagtagaagaatctgtttattgagagaatagatttataatttagaataagtgagaggggcagaagaggagattttgaaggatggcacctgaaggaggactagcatggctgagacagtgaagtggaagccttgaatagctaaagggtaagatgaaagtatttagctgtagggggaaaaagcattgacaggttggaaaagtaaaagtcagattctccttgctctgaaattttgtacagggcaggttctactaggtatgttacaatgcagaaaaaacatgaaataattgagaggaatttggtgcaatattatcttcttggcttcttttgagtgggcagatttttttcacggcctgtaactataataaatttgaaacttctcatcttttagtaacttttttcacttaagtttatgtggctgtgggcaatggaatgaagatattgaacttccaattccctgttgggtttccacaattacaagtcaatcatgactggttattagaagactatttcagttagaaccaccaagtcccatattgtcatattgtatgtttaattattaagtgaagcagtcttcttttcgtgttttccataattagggcattccagaaagatgaggatatttgctgtctttatattcatgacctactggcatttgctgaacggtaagacaccaaatccttccattaggttctatattttaaatattttaaccatgagtttaaaactaaaatgatcatttaaaatgcatgcaattttcttatagagagaacattctattctttcttctactttacacaatggcaaagtcttctttctactttacgcaatgataaagttacctgtgtcattttgtaaaaatatagagaatatagacaaattgaaagacacaaaataatctattacccatttcccagggttaactactgaaaatatctggggaaatggcctgtatgtatacatttatttgtttgctttcaacaaggccaagatcctttgatctttcagtcttggttgctctgtgacatgcctttcctgatgaggatactttaaggaagaattgtaagatacatggaaaatgtcaggctaacacagtactggcatcaccctgtgctctttcctgaactccataccaatgtacttcttgccagaaaactgatcaaaagtttagggaagtaaaaagagatgactgttagaatctaccattccctctatgtaggaagcaaataggtgtcctgtcaaaggacattctggggatgtctacatgaaaccaagtctccctggttgtaaggactccatctccatataatatttatacagtaatatatgtttataaattgtgggggcaacttgtttagctaattttattattctgctattgggacactgtgtctcagcatgagatatagtgtcccaaaacatatttcaagcccattggataaaatatgtgtttagcaagttcttaaatataatgataacataaccgaccagataaagtgatttataaacgctgtgccaattttgtaaatgtttcgaggaattttcccttttctgaagattgtccttctttctttttagcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatggtaagaattattatagatgagaggcctgatctttattgaaaacatattccaagtgttgaagacttttcattcttgtaagtccatacttattttcaaacagaacagcatagtctgttcattcattcattcaattcatgaattcattcacataattatccaatttcttgagcacctatttgatagtcactggaaatccagagacaaacaacacagagccatgttctacagtatgtacagttttccaaaaagaatttctagtctttacttttttattacaaatggaatacgtatacttgcaaataattcagatactgtggaagagatcaaatgaattgcaaaagtgtccctcctcccttcaccactatctcccatggcatgcagagagagtaaccattatttgtgtgtccctccagaaatttttttattcaactactatttttttattttattaggtccgtcagttttccttttttgagcctctctatatcaaatgcaaataaatatattcagaacaaaccccactgtaaggttcacattaaaaaagacttgaagtcaccctatgaagacaaaaaataatcacattaagtgtgaaagaacctattcttccagtacaggataagccatacttactgggcatatattcatcttgaaaatctatactgatgttgtcttggggaattgaaaaggaactaggagtgttagttcctcggtattgacccacagttatgttatcaggtcacttgagttcaaagttttgtgttggcactagctaagtaaaggaaaacacctctgctttcattgttgagtttcacagaattgagagctgaaaggatcccaggcaggagcagctaatccaaactcccacaaagaacaaaaatcccccagaggatcttctgttcttatatttcctgcaatggcgtccctgtcatatcccacaatggcctccctgccatttggatatcccttccatatcctgttgaaattactccctaatagtaagctgaaatctgcccctctagttgtagtcttgggattatttcatttacatgatgaccttttaatatttgactagaattaaatcatctccccttggtctttccattcctgggctaactaccatcaatctgagggctaacaatacaagtagaaaaagtatacatttgtcactgatcactgatcaattattaatcaatgatcactgataactataaactcaaaaacaaaatcatgtggggattaagagaaatgtatcagttttatgttgtatttctggtccctgatactggctcaggtaatgccactattgtcaagaagataccacttgtaaagtagatttaattttcattatattttaccatatgcttctccattcatgacatctcttgagatgttgtggtttatactttcagtttttctccagtccatccgcaaatatcaggcatctactgtgttccaagatattaaagaaatcatcatgacttagcctcatcaacagcattgctagatctgggatggaaaggaagagtataatcctggcagtcaggaagaaggcagcataaagtataagtttctgcttccaaaaaaggtctctcatcagcctgtagggagtgtgtagggaagggacagctgtccttgtagtagggaagggttttattcaggtcgtctgggctccataatatcccttgtgtatctgcagtctcctttgccatggatcaacacaataggaaatcttccggcactgatggtttttccaagggggagttcttcctggagcaaagcaaatgaccaaccaggtttgaggacctgatttgtttgacaattccattttgtattgtaaattacttaattggcattctactcccaatccatcttgtcatttgcatacagtggttttgggattgagttcagctataccaaaagtctgaaccttctgcacttagaacaaggcaaccaccaagcttcacttgcactgaggccgtgtctccaatggaaatgaggcagctggcttgcaggagcttcccaactcagggaagtagaactcctgagtcacctccatatgcaaatgatttcacagtaatgctgttgaacttcacttcccatcacagcaaatgtgtggtaacatagcttccccacaggagtttactcaccatggtattttaaaggtgaaacatttcaaaactgaaatttgaaagaatttagttttggattcactcaattatcactatcacttcgggtgttattgcacctttcttgtttgtgagtttaaatgccagactctcaggccactaactttcaattaaaagtgtttttctttaatcgctgaacctaacagcagggaaaacgaaatgttcattcagactttcagaaccttcaatgagattaggcagctgaaagatcaaagtgttgcatagttgtcccgataaagctatttggatcatatggaccaaatcgactgctgtcattccccaccaaccccatctctccccaaaattcccagccctgtttaagtgttctctgtagcatttatctctatctagtatattgtgtagcatatcatatcatacttttctgttttgtttattgtctctctcctcctagaatataaactccacaagcacaaagatttgggcctgttttataatattgttgcatccccagggcctgatatacagcagagtggtggtacgaaaagagcacacaaaaaaatatttgttgagtcaatgaatgaatgatttcctcaaataggattagcctaaaattttggaaacatgaacagatttggatatgtgaaaatttatttccagactgttcatcaggaactgttagcagcttctaaagggtacactggagcagcagtagtaaaaggaggaagaggagcagctctgctactgctactatcgagtactactacaattagcacttgcttattctgtgtgttaggccctgtactgaacactctgtctaaattagttcatttcctcctggaaatgactctagggggtaagtgcttcatcatgtaagatgagtatttttcacattttgttgtgtctgaaatctgagtgtgtctttcaatgatggaatctttgattccatgataagtggtattattcccattttaaggatgaggaaactgaggtccaaagaaattaagtaatttgcccaaattcacccagcctagaaaatgataaagctagttctaaacccaagcagattagctctgaagtctgggcccttaataaccactttttattgcctatatttgtacctctggtgtacgtatcaagttatatgttgacttcaaaactatcatgaccttttcttggttttgattgtccaacattagtatagtgttctgggtctgcaaaaattttgattactcatctcatctgtaaaacattttgaactcgtgtgtttgtgcatgcacatttgtgtgtaattataaaaattttactttctgttaatatataagttgtatcataagaaactgccgtttttgaagagcaaaaaaaggttgaatgttaccagttacatctggttcaacctaatagacatttgtacaaaaacagacattttaagaggttgaaataaaaatttaataaacaatattttcagtttttactaattgtgatgcttcactatcattagctaatatgtcaaggcataatataccttagggtgaactttatcattaacaaaggtggatggtgtcaataatcttgaggtttgtgtttttttatataacactgcgaggtctaattaagtacttactgtttaccacctcatacagtggccgataaaaagtgtcacttctgctgtttcctctgggttgtgcttgaattattagtattatcttcagtcctcagtttctttgtgggaaactttttaattagttgtttaattttgtaagatggttagtttagtcaaaattagataagagaatttgaaaatccgtagctaccccaaagcaacctacacataagaactattatttttgtgttttgaaatcataattttattgatttccagtgtttccactggtagtggtttcattgatataggagtatcaaaacatcactcattatttatttcagtttcatttgatcctagccgttttgtattaactctctgtgaagaaattacctcacaaatctattgctgtccttggtaaaggaatggagaattaaggctctagatcattagtggttacactatagtattagaagtaaaaaaaagattataccaacaaaataagaacatgttaatgtacttgtaatgaataaacatgaataaagctcttatgctatataggtgcactaaacaatctactagaattgtcagcaaactacgtatcttaatcctgaaagggtcccaaaccaatgatctaaaattgaatcaaactttcttccttgagcataattacttaaatgatttattaaaatagccagcatttaaaagcttaaaatgtaaatatcataatgtggtatcctagatagcatcccagaacagaaaaaggatattagggaaaaactggaggaatggaataaattatgcagtttagttattaataatgtactaacgtccttagttatgacgattgtaccatggtaatgtaagatactaacaatagaggaaaccgggtaaggagtatacagtaactctatactatctttgcaacttttttgtaaatttaaaacttctaaaataaagaacaaatttaaacattaaaaagtatcaccaggaacatatatcactgtttacagatgaaatactatgtattttcatatctaatttctgatcattgacttcaaatcagaaaagtgaatgacacctcaaaatcaggttttctgtttactgaagtctaagaaaagaaagcataccagctggagagattcatgtttataaagacagatttataacaacaaaaataaaatatccaagaataaatttaagaagaagcactttactgagaaacatatgaaaacctgaacaaatggagagggatattttgtatttgaatagaaagacttctggtttaaagataattctctttaaattattttttgtagaaatttaaggggtacaagagcagtgttgtcacatggatatattacatagtggtgaagtctggggttttagtgtaaattaatctttacattttgtttgagcccaataaatgtaccaacatgatttttatagaaagatagtcattcctattaatccaaacttgtcccaactttgaattgaattgaggcagagctagcaggtgttccccacggctgaggcatctgaacattaagcatatccctctgagaaccagcctgcattgatactctttctaatgtggacagcatcaagctatgtacgtagttctgtgctcagcaaaagccctgacttctttttgtttatgtcctagccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccaggtaatattctgaatgtgtccattaaaatatgtctaacactgtcccctagcacctagcatgatgtctgcctatcatagtcattcagtgattgttgaataaatgaatgaatgaataacactatgtttacaaaatatatcctaattcctcacctccattcatccaaaccatattgttacttaataaacattcagcagatatttatggaatataccttttgttccatgcattgtagtactcattggatacacatagaataataagactcagttcacactcttcaggaaacagataaaaaactaagaaacaaacaaaaaacaggcaatccaacaccatgtgggaaatgctttcatagccgggaaacctggggaatacctgagaggaatactcaattcaggccttgtttcaggaatccaaatcctggcacatcagagctgcttccctctttccagggtggcaggaaataaatggaacatatttttctatcttatgccaaacatgagggaccctttctccccggtgcctctcccaaggtagtctacaatatttcaactctagcagtctgcttagtgcatagaacatgaggctgtgtgtccctgggcaaattactagacttctgtgtgcttcactttccctgtaggattataatctactgagcaagcttattgtaagggtcagattagcaacagtgtatgaaaatgatttgagaccattgcctgcacaaattcaactatttttttttatctcactactctacagaagtaggtagggtgggagacagagtctgatgagaggctcagaatgtgaaagaaagtgaggcgagtgagcatgatatttaatataaacacaaagatattctgagaagagctgctcactgccccctcccccaatacatgttgataggaaaatgccacgtacttcagcaaaaacaactgaaaaattagatagaaaagtcaatcaataggaaaagataatccaggacggtgttgtgaacagaaagagggggaaaaaactttagaaaatgatggggatgctcttactggggtacgagtcctcaggtattgaactggctttcagtaaaagctagattagtgggttcctgccatttacaagctgttttatgacaacttacttgttgggtggcctacagtaactcacctaactgcactgagtctgtttcctcatctgtaaattggggatttttttttaaatacctggcatgcctaactcataaagttgttctgaaactgaaataaaacatacgtgaacaggcattgtaaactgtaagttacggaaaaagctggctgttgttgtgtctttaaagtttcacctgggtagtcaaagatggatcatgggtctcagtggagagctgagccaggcaggagctgactaagggtgagaggtgggagttagcagcctctgaacatctgtgtaccatgggaccccctttcctcctgcatggtaccccagacaaggagcctagtaagagatactaatggcttgttgtccagagatgttcaaactgcagagaaagataagacaacaagcattggcctccaatcatgatgacagataggaggaggtgggagctccttagcagtgctggttggccttccatgttctactgtgggccatctctgccatgtactgtaggctactagcttctatattaaagaatgcaagaggggccaggagcggaggctcatgcctgtaatctcagcactttgggaggccaaggtgggcagatcacttgaggtcaggagtttgtgaccagcctggccaacatggtgaaactctgcctttactaaaaatataaaaattagctgggtgtggtggtgtgcacctgtaatcccagctactcgggagactgaggcacaagaattgcttgaacctgggaggcggaagttgcagtgagcccagattgcgccactgcactccaccctgggcaacagagaaagactctgcctcaaaaaaaaaaaaaaaaagcaagaggaagtgaaataatcaaggccgccatttaatagtgagcagccactccatgtggtactgtgcaagcacattataaatattagcctcacaagaaatgtattagcatttgtattttgtacactggttaagtatcttgcccaagacctcaaaactggttaagggcagcagaatttagccccagcaccaccttttcaaagcctgggcttctcacacttctccatgctgttcccattttaacacaggtatctcgccattccagccactcaaactttggcatttaagaaaattatcctaaagctaaactaaacttcaaggatgaccattctcctgaccccttcccatcaaaattttatctttagtcagtttgttttcgttttgttttgtttttcagaactacctctggcacatcctccaaatgaaaggactcacttggtaattctgggagccatcttattatgccttggtgtagcactgacattcatcttccgtttaagaaaaggtagtatttccttaattgcagtggtctccactgggggtgaggaaggggtgagaattggatcatggctgcaaggaaacccgacttaacctctgcaaggtggtgcaaaggcattccactgttcaacagcaattatattgaagctgagtgggatcactgggtgaagatgaagcgtaaggggtgaggggcaggagaatgggtatggatggaggtagaagatgcagtgtcatacagtttttttctatcatgaaaataaccacagacttacagaagagaaagagctaaaatgcccgtcattttcagttgcattttagtcttgcattagttgcaaccagctggtttctgggtaccctaagtaataaaaatagttcctctgtagaactgtagtatgtttaccatagagtattttgcaaaatttttggtagaggatgttacataatttgcatgtgttcatttctccatttacctgtgggaacaattaaaatccaggaaaatgagtatattcaaataatttcctcccatttaagatgagtcagagtaaataattcctccaatacttagagaagtataccaagagatccagtgatggtatagagttgtctgatgttaaatagggaagtagaatatggaaggggattccaatagtcgttgaaaaattccccataaccccttacatgggggaaagtagtgttaactgagagagtagagataagctgtttccaaaaattatattcttaacaggactgagatagccagaatataaggatcaagtttcaatgacagtaagatcctgagatggagttgatttgcacaaagaaataattgttgccagcatgcattttgaatatttctctggaaaaaaagattagttggcagtagaaatggatagaaatcaatagatattaaaatacctcagaatttggttcatctctgggaaaagatgaaaaataaaagtgtatactcctcaagaacatctaggatcaaaagcatgtgccctacactattgaattaattaacctcataagttgggacctgtggaataaggatgtccaccagacttcctagggattacaaatgtttcacagaacttgaaatttaaacttgggtcactgtatgggatgtagagctgtgctatatggaaataaaaatgatttctttttctcaagggagaatgatggatgtgaaaaaatgtggcatccaagatacaaactcaaagaagcaaagtggtaagaatatcagaaggaattgggaagtaaaagtcaaaggaaacaaaaagctaaagcaataacaaagagaaatccatcagtcataatctcctctccttttaaagaatgctggttcccctttgcctcacagctaacacaagaactcctccaccgtctgaggaggtttaggagcagggaaggggaaggagtcagcttcatttgctaatcttctgttgccctgcaccctagcagctccttgcagcaggggacaaggatgacttaggtggatggataattaattgattctaaaatattgtgtgtcagtattgtaatactatgttaattgcaccatgcacggtatctcatttaatcccccaccccttgccattaccaaagagagagagagagagagagagagaaatactagaatttatcctcattttacagtagagaaaacagagggtcaagaagataatgtaaagtgcccaagaacacacagctgatcacaaaaatcaagcttgggggccattagcctaaccacagacccttactcttaacccatctgcttcaatccattttgctacaaatgtttacatttataagcagggcagaaaaacctcatccaggttattgaactaagaagaaagttatattaaggtttctaatttttttaatgtagttagaaaccaaacttaacaatgagcccaagtttaaagcagtctaattaacctggacaagctcaggcaagtttcattctgtggcccatagcatcatctgtgttgtaaagctaagtagcaaatgttgtttgggtcatgctgggggacaagccatcccaatttgctcaggactgaggggttttccaggatatcatgtaaggataattgggtacaaatataacctgctgctttctctcatttcaaatttatcatttatcatatcagcaactatgagttatgttttttattagatttcttgttactttttccccagaccacttcccatgaaattaatatactattatcactctccagatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 29 - DNA, 51110 bp, Homo sapiens
  source 1..51110: mol_type = genomic DNA
ctcattctatttattcttttaacaacgatttgttgagtacatactatgtgtcaggcaaaggggctagcagtaaagaagactcatatggtccctcccctcattgagcttacagtataggaaacagttattactgaacacgaagacagctatataaacataatttcaataaaagatggtaactgatatgcaccaaagtaagactaaaacacagagaagtgcacttaacttatactggcattatcatgggccttgatttactgtgggatcctttgctgttatgttcccttacgttgaaagatgcatctggccctacacaaagcacagactatgagtaggtaggcacatacagttctaagccaaccccttggggcaccaggagacagcactgtctcatgtcgttgcatgacacaccaccatggggcaaaacatttgctgaatgaagttacaataaggatacaattgtatctacataaacatttaaagatactatcatatcaatacctactatgtggcaggatcttgatgttgtgtggggaggaagataaaagtagatgtatcgttgagacagaaatgagggcaagggaaattttttatgcattgagagaggaagtttgtgaattttttcgctggccatggagtcagggagacttcagtccaaccaccgtccctccctgagcttgtgaccaagcctcactttccttatctataaaggtgacaagagtgatttttttaagtttatttatctgtaaaggggaaactgtagcaactttgctattatagggtaaaaagaaaaaaggatgaaaaccagaacaagggagagagacaagagagagagcagtgtcacttataactgtgggcaaagtgctcaccattcctctttccctttgtttcctcatctcaaaaagggagatcataacatttatcctttctgaggttgttatgaagattaacttaggtgatatagctaatgtgtacttcctgtgttcaaaacattctaatatgtattatcccctccctctctacccaagaaactccttacatacagataagatgagctcaacatccaaattaaaatgctttgaaaagttaaaaggcaaacctttaaaatgattaaatactgaaaagagcttaaggttatctcagagagaggataatcaaacctaggcaggctgctttggactgtctctcctgggatatgcctgcttttgccccaccaccaaaacataccccagctagattcccaagagcagcagtggtcacccaggtggcctctcattccattttctgcacaaaaagacgttaagcatatagctcagtagctcccagtgtaaaacagaacatcacccgatctccacctctgaaggttggggacagtcctttctagtcactcttcagctttaggggggttggttggtatcagggacagctgacagccttatttagccaaccagctgctctgtgatacacaatgtccctaatgctcaaagtctggtggatgattcttttgtatccacataagcagcagttggaggagaatcgatgagtccctttggttgcctcggggtgtcaaggctgccattcaaatcattaccaccataattattaaccattgttttagtgagatgtctgccaggcaattgtttttatttttcaatcattaaaaaagcaatcaaatttcactagagcaatgcctgcctcacgccgcatcaacacatttattaaaccccttctgtttgccgagctcaatggaaagtcttggaggagggaatacttaaaatgctctcggatttaaaagaattgataatgctgtggggaagaggttcacacaaaaaagcaattacaggaagaagctcgatagtatacaatacactggtacagagtgtcatagacagtgccactttcatacgctggattttatttctgtggcaatgggatgcttgggaggagccgcactgtgtagaggatttggagaagtggggtattgtggtgggaaattgctttctttcccaggaggtaggaggaaaacaatcaaggaggtggacaggatgtgcactccattagagcagccaccagagcctgactttttgataagagagtacatcagttaggataacggttaaaagtatctttaaaagacttttgcttcaggatgaatgatgtggcctgtgtgattcagcgataaattcaaaagccttgtccctattgtggcttgcggccacatttcgaacccatttttcaagcatgttaaacccaagcgcagcgcagagggctgcacatggggcagtcacaaaccaagctcaataaccttgctggtggggatgtgttggatacgctgctaatgcctgtttgcgacaatgctcgctagtcccggtggtggcggtgttcacaggtaacaatgtttaccaccgtgaatggaacttgtttgattaaccctgatcagaggatgaaaacactaaagaaccaagtgagaaagagggaagagaaccgcatagggaagagcagagcgagtagacgagccgaacgcagagcccgcgaggggcgagtggaagctccctgcgggcaggtacccgaccaccgccctgccctgggcgtggcggcctcgggctcagggaccgcttcggcgctagacggccgcgtccggaggaaacgggcgctggtgaaagcctaggtgtcctggtccacgcgcgcagccggacgtcgggtccagggagagacgcgggctggggcgggacgggacccggcccctgaagcgcgagggtgggagtgaaagcaaagaggagaaaagtagagagagaggacagtggggcccagcgccgcgcgaaaggcaggaaccgacccgcggacaggaacgggtgcaatgaggtccccggcgagcgtgggaacacagggttcagcctcctgcggcgggcgagcacgcggaccccaggggcgggcgggtgcgacaggacgtgacctggaagcgcggcgcgtgccaccgccctggagcaggcatccggcctccgtggagcgggcaggcgggccccgggtctggagcctgcgactggggagggcgccgcgtcaacagcagcggttgcggggcggggccgagtgcgcgtgcgcggctctcgcgggcggggagcaggcgggggagcgggcgggaagcagtgggagcgcgcgtgcgcgcggccgtgcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggtgagtggctcctcgctcccagccctgcggctgctgtcgcttcgcccccgcgggcgtgtgggctgcgccccagccagcccggcggcgccctgaagagggtggccggggcgcagaacactcgggccctgagcgcccgaagtgcagacgtgggagggccccacggggaatcgggcgccccccttcttcctcccttcctttccctggtcgtcttcttccccctgggtgagagcggggctcatctcctccacccggttctccatctccaaatgcacacacacaggaagactccaaaccgcgcacctcgcgcaaaaattaactagcaagaaagggcgcgatcagaggcaaggggctgcagctgctagggatccccttctttcgcacccctctcccttctcagtgcttagacgcatttggggttgagggagggagagtgggagggcccgggcctcttgcgatggaagtgcgcattttggcgaagtcggtgagaaggggtttctgccgtttgcctcccacatgaacataggagcgaagaggacgtaaaggacacaattaagtttcattttcaactcagcattcaatcagaagaatcttccggccgtgtaatttttgctgtcgtttttaatcctaaaaataagcttgctggaaactcttcctttctcgtgaccctcacgccccatcagccacttgcatacattctaaattgtacgttgaagtttttcccactttatttgggggaaccgttttaaaagtaatctggttttcgcctgaaataggaagacagtaacctccagtcaaaacatgtgcagcagaatgggatttggtgtttttcgacgccaaagaacctccacccccccacccccaccccgagcttttggaatccatttcgcttttgtaaaacgtgtgcttcgtctgtaaaaactgagcaaggaatagaaacattcgtagctctacagtgagtgcctgtcacacttcacatccatagagccttttgtgaacttttataagattcagaattcagcttgagcaccagtgacagcaattgttacatttattctagaatgctaaattaattcgattttaaactgattattagcctcggtgtgccgttcctaaaggcttgtgactttaggtatttccaagatgcccttaagtctctgcttaccactttcctccctccccgagcatcctggatgttgggactgtgaatccaggtctccgttatctaaatggttgatgttagtgtttcctgtcatcacgtttagtatgcttgttgcctttactatcattatagctaaaataatactgctttcagagatgtgttgtataatcgcataataatttcagaacgccttcatattgaaccagatatgaagtgatacagtatcatttattcaaactgcctaaaaagtaaaaagtataaaccatatactatttttaaaacaggtaggttagatttaaatcacgtagtttagaactgttggaatggtactttgagtgataggatttatgttaggccttctttagtaatagttaatatcatgtaattagcactcgtttacacacaattttaatgtgttcgaatccctagagtcatcgaattcagaatttatagtattttattttacttagttaatattaacctaaaaaaaaagcaaaatacaggcattatgcagttgagttgtgttaagtgtggactgtaaaacaggatagtatttgttataaaatatgtttttgtatgtgtttaatatatagcttcaaaaggacatgtatgaagaaagatgtctcagcacataggtagttataaaccaagggtttgagagactgactttagaatctagaatgagtaagaaagtgatggatctgtacattcatttgtattgaagtttgattttgctttcagtttcgttaattaagccccgggaatcagggacctttcctggcaccctctacagtgttagtggcctttgtggtaaaagaagttatctcagatactcatttcatgcaattacaggcaaactggaaggcaccttaatggtatgcaagatgtgaaatgaattactatgttgcattccactctgcccccactcatgtacaattatactttgtttaaaagtgcatagtttcagtggtatttattagctagcaaataaaactttaaataaataatgatggtgttgtgaactgtcttttcagtggttgaatgattcctctttgttcaaaatgagttgtttttttttttaaatcagggtacatatgattaaataaaatttttttccatcgtctaagtctagtagtaatttggcttgtttaaataaaaatgttttctcttaaagaatatattatttttaattacaagttgccattttaaggaagtaaatgcctatttaaaagtacgtatttatggggccgccccagcagtctagaggcagtgttttttaaagcattacactaaatgcctaccattaggacacttgctcatgctgtcatctgtgttttggcagatgttttatctttgtcactgagttgttcatggagatttgaaaggccaagttctaaagatgagagatagatgactttccccccaaattgatacatattctaaatccaaaataatcacgcatattcgcaaaactagatttgcatttcatagtatgaattcagctatagctagtatataggcattgcttttaatatagtaatgttctttttgtggcataattttcaatcactttccctgtcttaggtttctgcctacctttttatacaagataaacatttctattttcatatcttatctctcagtctgattttaataatatcgtcttgggttatcataaagttactttcactttgtatactagtgtagatatttcttctgtgataaaaagaacaagaataatagtgtaggatcagttttgttaaactcattgtagtactaaagtagaaaatacagagcacccaggaaatttgattctgatatacctagactaagacaatagaatctgggtttttcctctttacgattatacagtgttgaaacagaaatcaagctacacccacctctgaatatttcattccaaatccatggagcagttgcataagactcagaatctggtcaatctcatggtttgagattcagattaggagaattaatgtgcatatttgaatatatatcacacttgtaggttaaaaggtaaagcaaggggtttggctagttcaaggcctgtcaaaaccacttttttccatttcaagcaagtatgagcctatactacacaccaagcagtttgcaaagccctctttggcatatagacaaatttcagacagtatgtcagtatgtctcttaatgacttaagatgtagtatatagacagtagggatatcttgtttttattccctcatggtacttaggtgcttgttaaggccataattctggatgttattgactttaaactgccctgccctttttaattctaagttggcctcttccattcttataaggccacttttaaattcattagggggattgcgttgggtcagaactaagtacaacttgtggacaccccactctaccccagacctttattttagtgtccattgaggagagatctgacttcatcctttttgtacgtaacccagtgtagctagagactccagttcttcaagccagacactagttctaaggggcaaaagtaggtaggattgctttctggcatgtgtctttactgatgatctttgcttctttacctaagaggctgctgagacttcttactccattcctgaacatccattcccagcagataattaacttcctactctactgcaaaaaatagtggtgtgtggtttggactccctctgcttccctcccaccattactaaactgatctataacatcttagataaccttcgtctcattctcttccgtcatccagcttcagaagaaaacctactcacttttccaacgaaaggacaacctgtctaccaccttttcacttctaccatcttggagacctgctcagtcagcataattcttcagtcttgcatcttagatctttccctctccattgactccttccccgtagcttgcacacatgatcaaattttaccccatattaaaaaataaaacaaaaaacctcaagaaccaaaatgtccttgcccttgttcctgcctgcacattgtcagttgttgccttctctgctttcttacactgccaaatttattgtaaaagaatcactgccacttaatatattttaagtgcttgataaagccatcttgtttctctctcatttctcaaacatagtgtgcatcagatctcactagtccattgacagatggaaagatattctccccaaatatttaagcctcttttctctataattagcctaccatctaactagctacacatactattttggttcacaattttaattttcccctctttatctcccctgcttcccacaactaacttctgttgattactgtaagttttaggtcttctctccagttgttctttacctggactattgcagtagtctgttaacggatctccctttctcttttcccttgctgccctcagtccttcctttacactgctgtcaatgttttttttttaatgaaattaaatctgagtggtttacagttgtttatgttatcaagtctaaactcagcatgacattcaaggaagccctttagaatttttcttattcatcttttccaacctcatttcctagagctcacctaaataattctctgctttcagttcttcactttctttatctggctgtacacgttactctgcctagaaagccccttctttcccataacattcttactcatcctttaagacccagtttaaacatcaccctctaggaagatcattcatagacacattcttgtgatttttaaggaacttttctatccaaccatacaagtgacttgagattttccatgaaaatatgcagcttcatgatttcataatcaagtctatacaagtgagaagcagtggcaggttctcttgaaatacagaagaaaaatctatcttctcccacatgatttttagatttttcttctatggaatttatactttaaactttttacattcacaaggaaggtattgaatcctactttctggaccctgtgttagattcttaggataccaaatgaagacagtgtcttactttctgagcctggagagatcagttaagtaaaatagtaattacacagtagtaggtacagtgacaacatttgaacacaaggcaatgggagcaataaggaggggaagaatacaattgagtaatggtagggaaggggaacaagattgaacaactcaactgtgctttagtaggaagagcaaaagttagcaaactgtagctgccatttattgagtacttgctgtatatgtgaaaggtgatacaaaaacatgttatcttattaaatcttaacaatatctctatgaggtatataccatcactatgcacattttatagatgaggaaactgaggagcagaggtaagtaacttgcctaaggttatacagctaagaagtacatgaggtgaatcttaaactagggtcgaccccttaatgtgtgcacttaactattatacaccctatgtactcagtggggtaatagtgtataacagttaagaggctatttaggtttgaggaacagtatgtgctaaggcatggggatgagagtgcatttgcatagtcatggaattgcaataacgtcattatgattggagcttagtgtggaaatgagggagtacaggaggtggggctggaaaggatcttgaaggacctcgtttgtattccatgctaatgtacttagaatttagcctgaaaagggttgagggtgttgttaaagtatcttaggcaaaggagtgcacatttgcatttagaacagttattttgggaactgtagaaaatacatcagggccaggatggagaaaccttgaggatggaggcagggagctggttagaagggtatggcagtagttcgaggggggaggatgagtgtctaattcattcattcaacagacattggatttaggaactattaattaggttgaacctactaagaattctccatggctaaggagagggagtaatctaatgtgattcttaggtccaggtctggttcactggtagatgatagaaaatactttagcaaagtgatttaggctgagggtaagaagtgggggtacgtaagtttttggacatactgactttgaagtaactgtattacatccaagtggaagtgcacagcagacagttggctgtccaacatctgcagtttagaagaaaggcagtgcatgattcagttttttatttaaaagtttgcaggtgaagaaaaccatgggagtcagggaaaatactagggggcagagggtactgaatctgaagcccttagggacactaacatttagtgtgagtcaaggaatagatttcaacaaagaaagcaaactaggatggggtggacaaagcaggaggaaattcgtaatcaggagataccatggaggccaagagagaagagcaagaaagagactgcccactggctcaagaactgtggaaaagtcaagacagaaagtgaaccatgcatatttgatttttcacaagggattggtggtgactttggcaagagctgtagtaatgaagaagtaggggtgaatgggtggaggccacgtaacagatttaaggagtgaattggaagtgaggaaatgcttatagattgttaatttgggatgctagggagagagaaagtatgagggattttttttaagttggcaaatattcaagcaatttttaaagacacacacaaaaacaacaccttgtaaatggaatgggggatgtgggaggctgataatcaactaaggaaagtcgttgaggagactgatgggtatggaggcagaaatggaaactagctttcaggtgatgggattcatttcagaaaaggaagcaaaaaaaaaaaaaaggtgtggatagttggggttacaggtaggtttatagggaaggagtattggaagcttcaggattctcctctgaaggtttcagtttgtattaagaaatagggagagaggactgttggagagtatgggataaagggcagaagggagtaatttaagagttgttaaaaagaagcgaacatttacatgaaatatgtaaaatggtaaagattgaaggcccggaggagcagggactatgacattcttctgtttttgcctattagcaacatgaattttcttagaaatctgtaagaccaattactcttctgcccatccataaggacgttgtccatgcagaaaagataacacttgtaaccttgtattatatacttatcatcgcccatttgagaattgtaagctcctaaaagataataactatattttttcatcactatatccccacatcctatcacaatatttcatcacaggtaattcttgacaaatgttgattgcatttttaaaatttctaacctgaacttgtgtgctgtgaccaccatggattgagtcttctctgccactacaaagctcttttctagactatgatatgagatggtttgggctgatagtctatattcaccaatacttgtacagttccaatgaaggtttcaagtctaatacttttggcatttgatataaaatcattttcccattttatttgctaatttatctataactctggcattactcttggttacatttgtctgctgctatctgatttagactgctataagcatatcttgtttattcagagttttcttattcatgttaattatctgtgtttctttatttgctctctacattttaaatagtttcttcactttgctgttttatgagaagggagtaggcaaaaggaaaaaaccccaaatcagacagttttactacttaatagttttttaatgcatctttatagagattgaagttgtagttaacagctagagtgatatttttggcctgcccttatttattaattactagtgcaaaggttattcaaattgtggtttatccaggtcaattttactgttatttttactaatagcatttattctacttaaattgcttcagctataaaatgtttttattgtaacaaataatgcagtataattattgctttctgtattcctttgaaaattcattctctaaacatatgttatgaaatggtggattgttcaccaactacttcttacttacttaattaaagcctttgcaaaaagtttcataggatgactgagttcttcattcgtacctcttttttttaagaataatctcttgaaagtcaaaccatgattcatacaaatataaaatgaacatgtgtcaaagattttatttcactaattaattaacaagcaaactagcaagaaggcaaaacccttttaaaagaaaattggaaaaacagacacatttataggcaatcaagaatgcctaaatgaatttgctaataaatgcaaaattggtcaatatcctcagggcagtaaaatgtagtcttttggaatcctctcttccaccaggtggaaatcaattgcagaacaagatttatttttatctgtatggacagcaatccatttgaattactctcagttttctacaacttatgaaactgtaaaacagcccagtcaaagtcagtgaaaggcacaggcttctatagagtcagataattccaaagcgtctgttagacattgcccagcacttgactgaaaggatacccagtagtctttgtatccctttcaaaatcttcaccatattttctgatactttccttcctttataatttgaacatcttcacccctttattcttctctggtatactatgtcctctctccctcagtgtttctctctcaggggaaataattcacaattccaaagttttaaagaatgcaacggaagtccaggttttgccttgggcttcctagtatttgggtggctagaaatgtagaaaactgggaagaggtgagctgtggatgcccacaatataggttcagactgcaatttcccagaaatataacaatttggactagtcaaagagggcccatatcattacattaaaatgccagattatctagtttttatagtatcaccctacatttttacagcatagtattgtttagagagcacttgccgctatgttttccatgttaatgctcaacacagttctgtgaagtagcccggtgtttgtttttgatcctcctacttaagagatcctcctattttagagagtaaaccaaggcatagaaaagtgagttgcttaaggcagcatattaaaaaggggcagaaatagtatttttactcaggtgttttgaacattgtccagtacctatattccagtatgcatccttgcattcaattcatggggtatttattgagtaaacatagtgttcttacaatagaaacattctatgcctctgatttttattccagtttatgtagaagtaaatcttaagtgtgaactattaacaaagttgatattttatttatatttgttagtaatttgtgttttgtttttgtttatgttttgaggggaaggccaagtagctacttaggtaaaagagttgctgagtggctgaagaatatggaagacaactacaattcctacacattcttgtacattttagttgaacaatgagtgattacatttatttacccagtgcctcttctataaggcaacaactggtagtttatcctattgagaagcgtagataggatgcttattagcagtaactgctcttggtttcaacttgcatcttacttagctttttcaccgttttgtggtttcttggaggaagaatacccataatatacatttggagactgttgttctgtagtgtcaatgaaatgtgggggtgggaaaatgtcattcaagactcccatacaaaatgtctattgctgcctatattttgctatgggaaagtagccacagataatgttttttttttcctcattagtattttaagattttccatcctagtggaaagatatgatttgattcatcctatttactttgtatattaaagtacagtagaacctgccactttttttggaaatgcagcataaggataaagataaatttcatatcagttcagcaagttctatttagcagtgtgttgaagttgagactgaataaaatatttggtttggttttctgttcaaaattttaccttgataaggacaatatttttctacatatatcagtaggcagtaatgattacttcaaagcttccaaagccagatactacacctgcatgttccaacatagttgctgaatttattcccaagatgcatgtaatgtatactttgtattattgagaatgaataaagaaaagtcataatgatgccttccagctgtgcaagttaatattaaaatataatttgtttgcatatttcacctaataggtcttcttcattgctatactgtttacttaagtgaacaatggaaatgttgctgtttatcttaaggatttgtaacatgcctaagatcttacagtacagacttctataattaatgaaacatttttctttttcctttccaggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttggtaagacttctatgaaagcttctttttttatttgtcctggtgcaacctgatcctctttcgagaggaggccaaatggggataggtactcctttgaatcaaaaagcaggctgttattaataaatgttgatgtaatgtttagcaagtttaagattgtgatttctatcctatttgttaatctactcttcatggtaaaacacatttactatttaatttgtattacttgttaatttgtatattgtgtggttctcaaacttttggtctaaggaccattttatttggctgattggtcctggggtaatgtactcttcatttctgtttgctgccatccaagtgctttctttcctgtgactaattttcaagtctctttccaattattcagccttctgttttttgtgttttttttttttttttttttttttttttaaccttacactttgaccaagggatgacaagtcccagccatgctccctctggagagaatagaggagagtcaggagattaaatcacgttatcatggatgatttcttcagattttctgtgcttagccttactttggtcttctttttgttggccaaaatatgagagaactagagattcaaattcatttattcaaattcagacttgatgatagcaccatgattctgccctttttatcacaaacttgggttgctgaggtgtagaaggtagtaaaattagctgaagtaaatagtctcctctccttgctgtttcctgctgtgctagctgagttcttcaaccccatatgagtatgttctttctgttttgaaatctctatttagaattacctgtcatattaccgatcatggcttaaccacattttgagaaccttaactctagataggttcagttctgttcagttggatcaactatttgcctagcttttataaaataatctactaccagattaggaatggggtgtttggtttgatgaaatgtttgcacattacttaggattgagaatcaggaggtctgagttacagcccctgttcagcaacttactattcacctgtgtgatcttgagaaggtgcttaaccgagtccaagttacctcatgtccgaaatggagatgacaatagatacttgcttttatgtattctacagtctaatgagcataaaccaaagtgtgttgtaaatgttagttgttttcctgggatgtttattttatgaagtaaggtatcaagttgttgaagttagcctagagcattgtgagagggatttgtgtgcagtgaggaatgggcaagggatggctccgagtaggcaaggagaatttgaaaggtctttctgctcagaaatctctcttacattgtagcttttactattgatattaatttgggcaaaggagctttttttgttgttgttaagtatacctttaatgaagggtgtaatcagtcagtcatatccactgcaactaactggtattagatcatctaatacctagttgaaatttttctgaggtaatgtacaattttgtggaaatgaaagaataagatatgggggaacagtaacttttggggggtaatgcaggtcactgagaggtaaactggaaaactcaaaatatgagatttgaaaggccagaagatgaaatgaagatggaagagaaaggaatgccaggatctgagataaaaacagtttattattttgcgtgtgattgaaagactgttttatctttgtttgcaaaataccctataaaaataaaaactagacttattttgggagccattaaaaggagaaagtattgttttattatagagaatgtagaaaattctaccagaggtttgagacatttggctttgcatccattttaattttttgaaataattattgatttacaggacgattcaaagatagagagcttccatgtgcccttcacccagtccccccaatggtttcctcttatgtgacacaatatgaaaatcaggaagttcacaatggtgcagtgtgtgttaatggttcgctctcattttatcacgtgtttatttgcgtaactacctacacaatcaggacatagacctatccatcataacaaacatctccctcatgcttattcttactagtcataccgccacctcccactgtccctaacctggcaagcaccaatttgtcctccatatctataattttgtcatttcaacaatgctatataaatggaatgatacaagatgtgaccttttgagaagggctttttcactaagcacaatgcccttgagatctatccaagtttttgcatctatcaatagttcttttttccttacttttttttttgctgagtagtagtccatagtatgaatatatcacagtttgtttaaccacttacctatagtaggacattttggttgtttccaagtttgggctgttacaaatgaaactgctctgaacaattgtgtacaggttcttgtgtagacacagttttcatttctctgggataaatgctcagaagtatggttactgggctgtatggtaagtatatgcttagtttttaaagaaactgccagacttttccagagtggctatttcattttacatttccatcaacaatgtaagagtgatctggtttctccacatcctcaccagtatctggtgccactatgttgtaaccatttttaactgaaaaaaacaacaaacaaacaaaaaccagttaaaaagttatctgcacaaaaaggttaaatggggcaggactcttactatggagtataagttcttttctaataagaatacttacttgtcacggactttgaggatttaagcattagaaaccatttttactatgtcgtgatttttgcaaatacaggcataaatgagaaacagaatttgctcaatagagaagtgaattctacttaatagaatgcagataatagtgaccattctttgtgcctttttaaattttttggaggactaggactgaatacatggaaacactataagatatagttttatatacctctgtgcctatgtatgatatagtccattagaaggagcactggacttgaaattagaaagctgttttctagacttcactgtttaatagcttaagaaaagttggataaaaaactcaacctatctgtccctcaaattcttcttagtacagtaaagatggcatcaccatcatggaaatgtttgagatataaataatacctctcttacttaacaaaattttactaagtgcctaccatgtgccagtcaccgtactaggcatcacagattctgtgatgaataagacattattgctgatttcaaggaagtggttgcagtacaaatatgaagagtgaagtgacctaacatttatttgcacatgtgccacatactttatgtaagtcgtgtcctgttatcctctcaagaactctaggagatagatggagttcttaccatttaacaaatagggaagcaaaagagattcaaggtattgttcaaggtcagagtaatagccaagaattaaacctagatctctttggcatcaaaacccagtatttttaccaccgtaatatgtggttcatgtgaaaacaccttgtaaataactaaatactgaatgcaaatactagtaatcataaaatttatcattaatatttctgttctattagcaatacaaaaacttgaaaacttaaagtttatttttctctaagctattagagttttgtttagaaaggtcagttcaagttccgcagtgttgccaattccattattatcaacaggacatatggtctgtttataatgaagacatgaaggcattgcaagatctttgtattagttttctagtgctgcataacaactacaaatatagcagcttaaaacactacccattttttagctcaaagttttgtaggtggcaagcctggtcatggcatgactgggttctctgatcaaagtcttaaaagactaaaatcaaggtgttgcccagggcatactttatttggagtttggggtcttgttctaggctcacataattgtgacagaattcaattccttgtgattgtaagactggggtccctgtttctttgctaagtatctgccaaggaattgtagcagctcctagagattgccctcatttcttgccccatggccccttgcatatttaaagccagcaaaggagaatcttcctcttgttgaatccctctcacatattgagtttattttgccaggaagaacccagacccttttaagagaccacttggttaggtcagctctctctccctcaaaaataatctctctttcttaaagtcagctgatttgggatcttaattacatcagcaaactcccttttgctgggtgatataatcatgaaaatgaaatccaatacattcacagtcctagtctcccacacacaaggggagggaattataaaagatgcatggctagggcaggggttttgggaccaccttagaattctgggtttgattaaataaataatgggactgtagcctagcaagtcgacacatcagaaaagccatcacaatcctgaaacatcaagtaaaaatttggtttgcattttaggattgtaaatgttactgtggtgtatgcgtgtgtgtgtgtgtgtaacctggtgttccctatgtttgtgggagtttgaaggagacactttgttgataggaatggtgtcttcacttttttaggttgtctcatttttgtacagtgataagacaaatgaggtcctgggttttaataacacttcagcttgaaagcaaaaattaaacacttattcattgactacacccttgtaatatcacttctttgcctttccactagcaagaagttcattttcgtggaagccattctgcctgtccaggaattggaggagagtgatagacacagttgtcagccatagcttgggtagaataaggatgtgaatgtccttggcttatctttattaatcttgtgatggaaaaatatctgacattgttcttagtccattttaagcttaatttatgttcttagtggcatagaaattcagagctgaaagaagtatcatgtctcactctccccttgaggaacaagagttaacgtcatctgacagtactaccataacaaatcaatagcttaataggcatataagtggctttatataaaatgttggtttttttcccccagcatctcagttggttcttaaatatctaattccatgatcctcaaacttttcccactgtaacaaattagagagaggaggaacatgttcatcagtgactgtagttcaaatacagcagaaatgtgcagcagtgatttcaaactgagagaatcctggatgcccttctccattgtgtgccccccccccccacccgccccacatatcagggaacaatttaaaatcctggcacaataatgagaagggagagtgacaaactgataagttccagttaagaatcacttagacaggccaggtgtggcagcccacgcctgtaataccagcactttgggaggctgaggcaggtggatcacctgaggtcaggaatttgagaccagcctggccaacatgataaaaccctgtctctaccaaaattacaaaatttagctgggcatggtggcgcctgcctataatcccagctacttgggaggctgagacaggagaattgcttgaacccgggaggtggaggttgcagtgagctgagaccacaccactgcactccagcctgggcaacagagtgagactccatctcaaaaataaaaaaaaaagaatcacttagacagtattttttgttctgtaatcttcctctctgtcattgaaatgtctcatatttcttgtttcacatagaatctgggagtcatcaagtagtcatctagtctatctcatcatctaatttacgaattatttctatcacaccccaagtagctggcgattcagattttttactgaaagcttgacatgatgagacattattacttcccagaggatcttggtctgttatcaggcaatttcagttcttccttaaatcaagctaaaacttgctactattctcactcactgggagggctgtagtatttctctcaagtttatacctctctgtgatgaaaatctctgttcttttagccattgctcatggtttcgggatccccattctagtcatcttcctttggagcaattccctgttagatcttcaaatgtgatgagtaaaactgagctcaggactgcgtttgttggtcacacccttctcccttccaaatgactgctgctgtaactttctgacaggtcctgcttccctgtaatccattctccatgtggctgccaaagaagatttgaaataagtcaggttatttttcttccctatttaaagccttccagccgcttgtcattgctcttagaataaaatccaaactctcccaacaagcttttaagactctacatggtcttgccccagcaaactcttcccatttgatctcatcactgaattccagtcacaatggccatcctttggtttctcaaagttgccgttcctttggcatctcttcctccctgtcttcacaatgtaggctccttcccatcctaggcttagtttcgttcctctccctagaggcctttccttactgcctcagcttctcccactctgcacactcagggtcctctgcattgttcactgctggccttcaagagcctagaggagttcctccccatggtgggctttcaataagtgtttgtggaataagtgaaaaatgagtggtcacaccaggatcgaatgcacctcttacttttgttaatatattcaaattaatattaatatattaatattctattaatatatcctaaaattttgttgtgtataggggcagggaccaatgaaagaacatttcattgtcagctcatcttgactatgagatcagatattggctatattttgaaggtagagtcagcaggatttgctgtttaactagagtgggatgtgggagaaatcagggagtcaggtataactccaaggttttcagcctcaagcaactgaagaaatggggtcaccacgactgaaatgaggaaaatgagagggggagtttagtgggggatggggtgatgaaaatatcaggattcattgtggaacatgaacagttaagattaactgaagagcctgaattcactgttcctcagtttctacaactataaggaggggttaataatttctcacatcataattgtgaggatttgaggagttgaggtacacaattaaaaacaaaagcacaggggaagtaaggaaaaatacatcaattaagcatggctattagaccatacagccttaccaaatccattggggattagatacaaaaatccaagcatgatctccaaaattttttcctggtagggttttaggctatactgaagtgacttttctttggcataagaagatattcagttatacagttggaaataaaggtattgatttggagtatccaaaaacatctctcagtagagatccacaaccaaagaagcataaaaaaaagtcttccattcactcgcaaaactgtcttatgaccattgcaaccctcaacagcaaacatatggagtctctactacatatagaggaaaatgctagattctgaaaagctaatatctataaaacagagtttatgatgttgttatattctggggattgatgtaagcattttaccaggacttattatagcattataagcttaacacaagaaatatgtggctatcattatggagtgaaaggctggaaaattcctcacctgtgacctgagagatagtgcgtggtagaatttgaaagaagtgttgatttcagaggaatgctagtgcttgcttagggcagtattcagaagaactcttccaatcacacagccccttgtacaggcaaactccggaatactttcagtacatttacttggctttattgttagagcagaaacttcacagctaaaattatggtcttaggggtttataagatgcaattaaacttaatttttaaaagttctcacaaatcttatttgaagtcttaatatcttaatttcctttatataggatatgtggatatatttttatgataaaagtaacatgtgaccaacttaataacagcttcgttaatatcctaaagccactcatcttactttacatataaacagtccttctgaagcctaattatgcatggataattgcagagtgagtttgggaaaagaccaaccgggcacatctctctccattttaaccacaatgagaagagagtacctagtggtgcatcttcctcgcttaggttccctgagtctgtctttacaggaagacttcattgttacttgaaggtacattcttggaactttacacacccagccactcaacacatgaatacatacttattttagttaactgagtacttagtgagtgctaagctttgttttaagccctttgcatgtagtttattaactttgttaatagttacaacagtccttcaagatacatgtactgtgttatactagtgtaacagttgaggacaccaaagaacaaagaggtgttgacacgtggtcacggttccatagagtgttaagttagagttgggttcaaaccctggcagtgtggccacagagccttgttctcagccactgcactgcactacctcctcccgtgaaacataagaaaaatgtgagaaatgcttaagtaagtgtagtttttattcataaataaaatttacataagtacattatgtgtaatttgttttatgtatatgtgtgtatgtataaataaatacatgtaaaaataaggccacagttttaatttttttccatctctataataaagcatgtattatagaccattagcagaatttaaagtgttatagtaaatattaattgtgacttttgttttcttcttccccagtttcatggttttctccaaatgaaaatattcttattgttattttcccaatttttgctatactcctgttctggggacagtttggtattaaaagtaagtattatttctacttttcatttatgtttcagtgatgatatagttatttctacgagacattgtcagcgaaatatttaaagttgtactaggaaaagtgctattatgataaatatgagtatgtaatttgaatactactagtctccttgaagtatatgttgtcgcccacattttgctgcagttcacttttaattcctaagaaggttgttttcacttggtgtttttttaatctcttaagaatgaatagtaggaatattagtaccaacaccttaaactcatgtcacattttaatattcacagaacatctacacacacattatgttattaggtaaacaggtggtgacagcctgcattagttttaaggtaggacgttatactttggagcatttagattcccctctttttattttcccagtttgattttctctgtgtacacgtgttcacccttggaaaagtccagtcggaactatgttttgtcatcctctgcgtgcagttctgcagcctctaaagaagcagccaccagagagttaggttctttgatcttgctttcctataatagtaacgtaaccagacttctgaaggcagatcttgatgctgcattagatttagcttcaacaacacagaattgtcattactaggcaaataggtaatatgcattacggttaatgtttaatcaaccatattttcatattttggtaaagaaaatttacaaaattaatgaagttctgaggtgacagtctaaacttttaagctttttaaatacaagatttattccttctttccttgtagcatcctgaagaccagtaaacatttatataagcaaggaataaatactgcttatttaatttattctgcaacctttaaacacacaaaagctagtaaactattgcagtggattgccctgttgtatattttatgaattttacttttactcagcagtttaagctgtctatatctatggtggtgtataaacatggaagggagatgactgattgattatatgttttaagcgcttttctcagtgtatggcattctggaaatgcttagtgatttcagaaatgttctcaacttttgtctgaaaggaaaaaagggggaagaaaggggtggcagtggcaactgtcaagacattttataacttttactttcaagatagtgtctagacttcttttggaaattttcttataatctcttagttttttatgtcaagaaaaggactggtgtagcattaagacctattctggcatcaatgatattagggaaagcttttaaccatattggcagagccagactttaagggctaagtcatatgacttgggcagggaacagcctttttcaaagatcatgaaattatttcacatagtgcgattttttgagtttggcttgatggatgtttgctgatccagacgttatcattggtgacattatttttagtgaaatgaaccaggagtgagtttgttcactgttggctgtatttttacaaaatgagctttacaatattttttctgacttaaaaaagtcatacatatcactttagaacacctcgtacaaagtagggagttatttaaaaaaaaaaaaaaaaaactcatctgtagtccccaaacctagaaataaactattggtatattctggtgaatttcctgtttccctttctgtctttttctattcatttttgttatcttttttttaaaaaaaggtaaccataaatggactcgtacagcttatatgcttgtacgttctgatgttcccccatgtcttcagaaatcttgttatagctgcctgtgttctacttttgtggatgcactataatttacttaactctcatcttgatggattttaaggatgtttccaatttttttgatactatgaaaataactctgctaaagatatgaagcctggaattggcagagatattttaagactctagatacatactggcaaaatgttttccagaaacgtttgtatcaactactattgcagatgagaatacctgtctttcacctcagttataattctgatattataacgctaaaatctctgcaaatttgatgggtgaaaaagcatcttattgtaattgtaatttttgtgttagtaaggtggaatgttcttttttatattttttgtactggtcatattttaaggaacgtgcttataaacctaaagaaatattttggtgggaattttttgttttggctcatcttgaaacaggtagatgtgtgtgtatgtgcatggaagaggtatgtctatacatgattcacccagcctgcgctcacatttaaaggtgttgatgataatagtagctaaccatttgtggagctcttgctctgcttgacaggttctgtgcaaagtactctatatctgaaatgacatttatttctcccagaaactctatgggcatagacactgttgttattcccgttttgtagataagaaacaggcacagatagattaggcaatttgcccgcaatcacccagccgtttcctgatagtactgggatttgaactagtactgtttaccactgcactgtactgcctccccgttttgcatttattttgaggattttatttccatgaagggtgaacctatatctaagcacataatactgagtagctaaaacttattaggagagcagaatgttgacctgatttgtttacttattctgcaaatacctagtgacagggtgcctactgattgctggacaccaagctacatgcctgaaatgtgggtgtgatgtgcatattgccctggttttgtagcacccacatttaagccagggagacacgaatgtatgtgcatcccatgccttgtcacatctttgagatagtcatggatccaaatctaacttttctgttagtgcctctgttgatggcctgagcattccaccaaatagaaatagaaagcacctctattctccacctgcttagatgtatattttttagaatccagtatggtaaatcttctaaagcattcataataactcagacccatgacttttattttatagatttctagttctgcttaacttttcctgattaccatttatggtctgtacttggctacaggggatttctctcagtggtttttccagctctgcatgtgagtctttgtggtccaagacaaacctggacctttaccaggctaaactttcagtaaagacagcaggttatgccctcatttgctcactctgaggagaaagagtttctttataccagagctgtatcttgaaagatgtctcaggatgccattggtcctactgaggaagaagcctgagaagactcttaaactcccagagcccagccagcacttggtgagccctggaccacgtttcaaagataaaggcctcttacagggaaatgttcctaaataccttcacctttagcttagctttaacttaggaacttttaagcagaatctctatgcttagcaaacagctcagagattgctagaatcaaacaccaaggcttaactgatagtattgaatttcagacgctttgtttgtgttctgaaaactacaccaactcacagtttgccactctactgacaattaagttcctggctgattttcaggattcttctttctcactctgatatcattttaagtgctgtccacctagtcctccagttcctgcaggattaaggtccaactgtatgtaaagaactggctaacattttgaaattctttgagataggcctatctgttctttctttgcctttgtaactttgttttatacaggcaatactggcttcgcacaaaaccgcaagaccataaacatgaccatgtaagctgaaattctgcaaaacaatcttcatgatgaatgggaaaaactattattatattgttccatgacctttaaaattttttttgttgttaaaaccttaaaaactctgttattatcaataacaacggcattgggaaatgaaaaatagtaaagctagtatttagtatgtggtaaattaaatcattagaaacatcgagaatgaaagtgtgttatcaagagtagtttgaacaacacttgtgtgttcttcttgcataactttggatacagagcaagcatcttctctatggcttggtgaattgtcatacccctttctaagtttggatcggtttctaccgttttatcctttgcactctcaatgtcatgaaagaattcctttaatgtttttttgaatgtttaaagtttattttattgccagtcacttcctctgggacatctttattctttttgttacatcctcctcactttcctcatttatatcagtaagtccaccttcgctgagttcttctggcttcatctctagagtttcttgcattgcagtaatgtcgacattcgcacaatcagctatttcttctcttactccatttatggtctattcaaatttcactcctagcatttgtcagtttttatttctttactctttcatctttcttgcctagtttcctcttttgatttatccattataaaatgtcacatgggtttatcactgggaaacaaggaggtaacaaaactccatactttgctgtctgtgcatgacctaaatatcagatgtacagtgactagtcaccaacagtcttcaaaagaagtgacatggttgatcactgatcatgatgggacatctgctatttacatagttatttttgaactgaagaaatagcagtgaagttgtactttatgcagttactcagttaatatattgtggtaattgaaatttggactgtttatgagggatttatttgattaaaccatggtaactggaattcatatcagaatagtgcaaagtgaggactgctgtacttgattccaaatttaaaactgtattctaggcatctttaatttttttttttcaactttcccctccctgctcaaaattactccacagagattttggttacggctcagatgtctttgagtagatgttcctgtagaaatcattgttagaaaattcaaggaaaaaagttctcattacactaccttgagatcctaccaagtccttgagttttgacttgaggacagcttaataatgaaagtaattcagccatgaaagcatatttaaaataagtaatggaacaggaaatttcttcatagattagaaaaattattctgaaaaagtgaagacatagcctatcttggactaggaaatttccatccaagaaagtaaaatatacaataaatattatcacaaagaatcattctgtgacctggttgatggcccctggtaataagactttgttattaagtttactgtgaattctttttttttttttttttttttttttttttttttgagacggagtctcgctctgttgcccaggctggagtgcagtggcgtgatctcggctcactgcaacctccgcctcccaggttcaagcgattcttctgtctcagcctcccgagtagctacaggctactggcgtgcaccaccatgcctgggtaatttttgtatttttagtagtgatggggtttcaccatattggccaggctggtctcgaactcctgacctcgtgatccgtctgtctcggcctcccaaagtgctgagattacaggcattagccaccatgcatggccccttacagtgaattctgttcaataatcttgaactcccagtgctttcccttggtcctgtccatataatgatcacattctgttattaaataatgtgcttatgtgctgattttattgtaggaggaattgaacaaatctatagctcagcattttactcttttgtagaaatcccttgaccctttccaaatccagacatcacaaccttctccatatactcaaaaattgtgaaatccacaaggcagggaaagttgataatgaaaaaaaggaagtttggtctgttggacatcctcttctttcatcttttatgttcaaagtacttgatgcacaaagcctggactcttttatctctgtcctatagactgatgtctagtcagtattcattgtgacaaaattgtttcattagagcaagttggatagtgtatggagattattataaaatgatttaacttgtcttctgtcaagtaattttataattggtattaatgttcatttttttccagtaccagtttcagaagctttcatttgtgtatgcatgtgtgtgttaaataagtgtattagatacttgaaaaatagtaatttaaatttaaacaatttaaaaaatgaaattgtaattttatgtgtaaaggtgtctgatgtgctttattctgcactgaaaacaatattcatttacagctcaacagacaccaaatatattctaaaattactttcctagagttatcagaaaacagcactgtaataatgaaatcaaacccatctttctttatgatttattcttagtctgatacgcatcagcctggtaagccttcctgtctctctgcttacttaccaatcactccaaatgtcatgtctttgggcaggcattaaattcttgggttttgggttttgttggatggactgcagtctgtgtgagcctatatgggtgtgtcaaatccagtctttggggtgtcatggaaacttagcatgatagacttgattttatccccaagttgacttggtaatttcattagatttcatcagtcacaacctgcattttatcttgtatgtgctgtctattggtcacaaaatcagaaaaccttcttgtccattcataacattagctgttttttcaggtggctagagggacatgtcattgcttcatctgcatgaatttgaaagattaaatgcataaaggaattttcttagagtagagtaggccttcaccatctctttaactgggaaagaagttttgggagtaacatactcatcactcacacccccctcccctcaaacacacacactcacccattagaatgtaagggccttgaggcaggccttgtgtttgcttttcgtgttcactgtcagcatttagaatagtgatagtcatatagatggatagtgctcaatcaattattgtcaaataaagtaatctacttgttccttgatttagactagcaaaaggggctggtacattgtaggtacacagtaaatatttgtagaacagatgaatgaaccaacccaacagatttcttagaagtagcctgcttttggttactaattattttatagaacatataaaagaaaattttagaatacctaatttgttcacaaaaatgttatattgtctcccctgatacttggtattcctgtgtcaggctgagcactaaggttatacattttttgttaagcatgtaattactgttatttctcatgtactttattttgtgtattgatttcagtagttgtagctgagggtaatccttaagagcatgcagattttaaaaattacatagattgccttgccaagccgtctgaaagtcatactagaaaatttgataagagcctgctgttaacaacacattgagttatttttatcttaatgctaagtggagaattacttgaatttattttttttgctgcatttaatgttttgatacattcaataaataaggataaatccacttactgaaggacaagaaaagatcaataagagatccttgaggttattgggttctatggcatctattatatcaaattccttgccctttcactatactgagaaatttgacgactgggctagcaacaagatacctaccccacattagctgtggtacctgctgaaatgtgaccatgtgagcaaagaaaatgagccaaaagaaactctttctgaagattcactgaagaaaacctggagatactctttcattagtagaccccacagagacaggtggagtgtccgttatccagaatgcctgggaccagaagtgttttggatttcagattttttcacacttgggatcctcaacatgtgtatttgttggaaatggctacttaatttaaggaaaagtttaaggtggcctggaaaataagtagtagttgaactaatcaacaggaaccaaaactactttcaatatatagtgtacttatactcaaagagagacggaccattttatggtggaaccctgtctatggtagtattttgtgatgttttatttttgttgctattgtttgcactgttttctcctataacagctcttctaagccttaagaggataaattttatatgagattcaaactcttatttttgttaagaaggatgtaagtcaccaggcatggtggctcacacctgtaatcccagcactttgggaggccgtggcaggtggatcatgaggtaaggagctcgagatcagcctggccaacaatgtgaaaccccgtctctactaaaaaaaaaaaaaaaagaattcaaaaattagccaggcctggtggcgcatgcctgtagtcccagctactcgggaggctgaggcaggagaatcattgaacctgggaggcagagtttgcagtgagccgagattgtgccactgcactccagcctgggtaacggagtgagagactctgtctccaaaaaaaaaaaaaaagaaaagaaaaagaaaaatatagaagaagaacataagtcaacctttttccatgaaattatactgtactctaaggcaaattcttgttgcttgatcttagtagcttttttatgcattgacttaaacctggaagagtttcttatgaatgtttgattgactgtgaggtgcatttgaaacagcttctactttataaccctttgcagaactttcaagctctatttagataacaatatatatgggattaaaatggaaaatgtgacatatgtctagagaaagttcctttttctgtgtatgtgccttttgtgacttatacactcagtttcattctagccagcatttgagcctagaaaatagggaagttatgaaaactctgtgccttataagaaggcaaggctatgactgagtattagacacataagtccagggctgggcggaagtaatgaatcaaataaaaacttggggaacttgccataggatgttttctgattaatatgggtttctcttctctgattaatgtggatttcagttactcaactgtgtaactgaaatccacattaataaattggttatttattgcttaggacttatctccccataaagagactaaaattgaggttataaattatgggtagatagaaattccagaaatatttaggtacctgtcatataccttaaacatagatttttatcacaatcatttaggggcttatttattgcttttactttatttaatgtttgtcactgtagaagaaaaaaaactaaatgctaaatataacatttaaaatattttccccttcatgacagccaccaattgattactgtcatggagactatctctatgtggatgacacacagcagcatttcaatcacacgctgtttctttccgctactcagtttgcttttaggttgccttaaacaacttccttggtgaaaaaatcaacttcaatattaaccaaaatttaaaagattcatatatagtaaaagaactaatattcgcagattgacccattccattctttcagagaaggtatgagatacttgacagtggagctagcagcagaaacaataacatgtatgatgaatcccattgagtctttgcagtttatttttattaaaatattttaattgaacaatttaagctttttttcttcatcaggatttcacaagggtgtaatctgcccagttttattctgtcatttttttcaaaacaaaataactagatttccagattgtttactaattttttataaggtaggacaaaattctctttttctcaatattgttaattaacccattatttcccattagtattatgtacatcagtgttgtgttgtcacttggagtggttttgctcatcgggggcattgacaatgtctgaaaacagtttgattaacataactggcatctagttactagcatctagtatgtagaggccagggatgctcctcaacattctatattatgcagagcagtgtcccccctccccccaaaaacttatctagctcaatttattagtaatacatcaaccgagaaagactgatgtatagaacctcatttgttagtgtaggaaaataaggtgtcacctataaattcaccatccatatttatactgccgtgacgttatccatttgcttatgaaagagatgtgaggtgacttgatgatattaaggagttgttcctcataagttattacattatagtacttctgtcagtttgtctctgtaccttacaagttattaaaatggcttcacttgtgattgagttcatataattctttgtttttcttttttaagcacttaaatatagatccggtggtatggatgagaaaacaattgctttacttgttgctggactagtgatcactgtcattgtcattgttggagccattcttttcgtcccaggtaagatgtgcagttcctaggcaggaacgcaggaggtagatgagtgcattccaaggtgaggagggctgcattagtttcctagggctgttggaacagatgaccacaaactgggtggcttaaacaatagcaattccctttccatgctggaggccataagtctgaaatcaggacgtcatcagggccacactccctctcaaggctgtatggaagaatccatttcttggctcttctagcttctggaatttgctgaagaatgacagtgtttatcattccttagtttgcggcagcataactccaccctctgcctctctggtcacattgcctcctccttcctctatgtctttgcctctgtgtttttgttaaatctccctcagcctctctcttatgagcacatttgtcattggaattagagcccactcatataatccagggtaagctcctcctttcagatccttaacttcatcatatcttttgccatataatgcagtattcactcttttgctgtattaggtaatattcacaggttttagggattaggaggtagaaataattttagggccatcattcaactcactacagaggcaaacttcattggccaagacatgaaagtagaatgaatgtgggaaccaagtctggaaattggcagttgcatttggaggatgggaatgtgagtgggaaatgaggatatgtagcacatgtaaagaaaagcaaaggaaggctgggactcaacctaataactatagcacaccatcgtttgtggagaagaatgcagtgcaggtgtaatatttaggaacatgggcttggcagctctattggtatctgaagttagtgaagacagcaggaagaaggaggtgaattcagaagacaacttgaaggaacaattgatttgactttggggcatactgaatacagcagatgaagggaacagtaaaagatgaatacacagtttctcactggaattctgtaaactagttaagaaggaggcagagccaattcaaggtttcagcatgttgaaacttaaatgttgagatttagatgtcttaagtttggtcttaatcaaaaaatagtcactaattttgtgtgaggttttcagggaaacgtatatttttaaaattttcacagttgtcaagcacagaatttagattagtacatttaaaaagtatgtaggaaaatattttaaatgtttttatttattgacaggtgaatattcattaaagaatgctactggccttggtttaattgtgacttctacagggatattaatattacttcactactatgtgtttagtacaggtgagttttcattgttacagtatgattttgtctacctttttcatttacaaaacagcagttttggtgaaaatgctcatataaattttttacaactcaataagagtaggtttattaaaagatttttcatgctattcttagtgacattttcccattcatattaattttaaggttattctaggttagctgttttgtaaaaagtgactttcatatgtatttgatgccaatcagcataattttaaattatgccatataacttcttaatgattattttcatattctaatttcagttttttgaagttataagtggatgttaaacgcagtctttctcttcttttgctaagcttctgcttctgcattaagaagactggttctataaaatggaattatgaatcacaaatagtagaaacatatttgttttaattattagcaaaccttaatactgtagttttaagagatggtatggaaatccaaactataatcagtatcattttcactgcattttgaaagtagatcactaccatatttagttattactattaaagagtcacatttaacacttggtcttagagcctatctctggctcgttgtacattggaaaggtattagtttgttataaaatgacatctagagatagagagcatgatgtttacagtcaggtatttgcataatggttccctcagcaagagcaattccatgtgcatgtggagaagacattcataatagccattcatattgtaaaatgcacaagtgtggtaaaagcagcattgttctaagatttagggtaaaaacttccagtccagcttagctgactgtgaaaattaaaggctgccgaatgtgtgaatttggagctgactacttgtgtggaagggttagatcactgagttaacatctaccgtcaaacaatgaactccagagtcagtctttggtcttaggagatccctgattataccaaatgcaagtggaagttattgctttttaaatactcttgacatgcttctgttacatccttttccttcctccagcgattggattaacctccttcgtcattgccatattggttattcaggtgatagcctatatcctcgctgtggttggactgagtctctgtattgcgggtaagagtcatctttctgtagacctaatttgggttacttttggacagagctcttcttcctttttctttttctttctctctttttaaaaatatatagacttgatttttttttttagtgcagtttttggttcatggcaaaattgagtggaaagtacattcacatggatatttttatgtggacataagttttcaattccatttgggcaaataccaaggagtgcaattacgggactgtaaagagtatgttgaaataaactgccaaactctctgtatcaaaatagctgtaccattttgtattatcaccaccaataaataagagtttttgttcattcatatacttgacagcatttagcattctgagcttttctttttaaacttccaatttaccccagtggtaatagtgctttcctcctcgctacaaagatattagctgtatatatggcttggtggctgattgttctagcacccaaactgatatgcctgtatttgtggaagagcttttaaaataactgggctcaaattggttggagccttagacttgaaacaccagttcccatttcttgatatgataaggtatgtgttatgcaaaggagggctttgttcttctaataattttgagtcattttactggttaagttaataaacatatatggataatttttgttttttgatcgttagaataactctcttaaaacttgggattattactgttttttagtaagttatttcatatgcttttctaatacagaattttatttgtttttacagcgtgtataccaatgcatggccctcttctgatttcaggtttgagtatcttagctctagcacaattacttggactagtttatatgaaatttgtgggtaagtcaatcttattttcattaacctatgccaataatttcagatatatcacttagaaaatgctttttagtttgttcttccagtttaggaccaaaaatgagaaaatacaattggagtgattcgaggataattaaagagggtagaagacatataggatttttagttggttcttccagtttaagaccagaagtcagaaaatacaattggaatgatttgagggtaattaaagagggtagaagacatataggattaatgaaaatttggtttccaaagtagtttaaaggaaaatggctttatctattagaatgtgtacctttttatactaagtaaaaggggagagatctttgaggatccattttaagtaatagaataggatttttaattgttccagtgtttctgtgatagagctgtcctgcacagacctgtttgtttgtcacttgctctttttcttgcagacatagacaccccagacaggaattaaaattcacaatctatcaattttgttcatttaaagagcagtgacctctaatgcatgactttaaaacagttctagttaaaaccaatataatgaaaacattgagtttcaaaatttaggcttttactccttttaaaatcaattattagtaagtatggaatttacttcattgtttctaacttgtatatttaatctgccaattttcaagtaacatttctgcataaattcttattttttattgagatatatgtacacagagagatattttcaattgtgcctgaaactaatgttatcttacctaagctcaagatgttcccaataatgtaatttatattagtttccgttttttaaaaaaattatatttttatgaaataaaacatactcttaaccacctatcaaaataatcaaaagttataaattaatggagtaaaaaaatagtgtttctgctttgctttaggtaaactttgctgtatgtgtttctaaaacttaatacgaaacttgaattgttatagtcaaataatttctcatatgactcacataatagtttcaaaaaacttttacctttatttctgaactttggttcttgatgattgttaattgaattcaattctgtcatatattctgtgtctttctttaattaatgcttattagataaataattaaaatacttaactaaaatctgcgtatccttagcatatgagttcataagtcttagttgttgctcaatgaaattttctaattttataccacataatgccataaaatacaatggagacatctaaagcagaatggaattcatgtggtagctacagtgaacatcttgaatgttggtgcatattctatttttgttacatcttccaatcaccatgtgtctggttctggaagatgacactcctggttttgttgctccccacaaatgcctgagaatagtgtgtgatttgcagtatccatacaactctggtgaagtagtatgagatacctttggctgacgggcagcacgctcttattttttcctcactatctgggttgtcctcccttttactcccatgccaccccatgccttccatatctagcatagaatgatcttcagtacagttgccagcaggtctggtgacaatgtctcaagtggaactaagcattgtctatctgccacctccttaacttcactctcctgccttctccatcacttacgttcctccaagcctgtgaaaccactgtactgtacctgtcactgttctgacattaaaattaaaatgacttaaatcttgacaagtaccccaaattattttttctttgtcataggttagcatataagtatactatatgctaaaatttatgctatatgtttaaaatttagtgcaattttattgatagtgtcctaattttattgatagtatcctaaattaactttttaaatcaacttgtctgatgccagggttcagagggacacctacagtcagttgaaaggcaagaagagacaaggtacaggaaagttgctctttagataacatggtagactaggaggaacattaatatggtttgcttatataatctgactgtgtaaatctgaatctatgtaacattaaggttgcaaatttgagcgtttatattaggaagttaaaattttaagtgtctccaaaataatttttactcattgcacgtgttctgttttagaaaagcctaatgattgtgttttgatttaaatgcaataaaatcctcaaatagttaaaaatccaagctttctcttcaagaagaagttaatgtcgctatgagatttttaacttttataatttttattatttccaactttaaatttgtagccttaatttgctattttaaagagtaggcctttcactttctacaactttctgtgaaagtgactttcactttctattttttaactttttaaactgtgttgtattttttttcttttattggaagcattttaattttataagatgagaaaaaggactgggcacaataacttaatgtgaaagcatagaaaagattacaagaacctaaccaaactcactaaagttgggcttgttgtttgtagagaacgtttatataattataaggatcaatactttctcatttttaaagccattaccagttagttcaatataagggcatatagtgttttgatacaaatcaatctggtagcagtaagtaccatatttaccacaacatcccagatattttagaatgatgcagatgcagaatatatacgtagaatttatatctatgtatagatacaaattcagatattttcttgttcaatttaaggagaggtaaatttggtatcaatagaaaaaatgtttctgaaaaatttaaaccctggaaatgtatttatggcatggagtcagatgtttcagggagagaagaacaaatcaagaagcattgcaagtatgctcatatggaatgcttaaggcttgtggttaaaaaatatatatatatggctgtcaatgtcttaggctcatggtagcagcagaaatcgtaataattcttttgtcacatgggttatatccatattggagagaattaactcaggtgaaattaacttgtacactgtttggttttataatatttagagggatcacaactgactgatgtccctttgaagtaccattcttcataaatctttttttttcagaatgggccagccaactgtgacatcccttggatcggagatttagaactagaaagtattctttctacattattagggaagaaaaggagttacttggcggttagcaatattctattttgttttgttttgtttttagagacagggtctcattatgttgaccaggctggcctcgagctcctgggctcaagcaatgctcccacctcagcctcccaagtagctgggactacaggcatgtgccactacacctggcagtgtttattctgataaatacatttatgagctcaaaaatgtaactctaaaaccttatctctgaacttccatattaccatcagaaatttagatagttgtttagttctctttttctttgtagaacatagatataaggcatggtttcattgaagtcagttgtatatacatgtaactatcctgatgttcccaaataaagctctgtatttctgcttagtttattggggaggctgctaaatgtagtgcatcccaacccattttaccctgttctactttaaaaagaggttggcttcttgtttggatacaaggaccaagtcactcccccaggttcctccacagtaagggaggcctatttaaagccgcccatggcactaacagaaactggactcctatgagctcagatacataactgggcctcacaggggtgggacagtatgtagtctaggaattggaaggatccattccatatcaaagaactgaagcatcgtgttgccctctcagcagcaagagtaaggtgatgcccctgtcagttatagttcctgagttcctctgtctttgattctttgcctattagccagctagctcaccctcttgtttatgccactgttttttatcctattcatgccttctcacagacaacttttcttacctacagctttggactcatccttgtctcctttctgtttctttttcactttcccttcccatcaccaactttctgggtttttttctgtttcttcttagagtccagtggcagggagaaacttgtcagtccagtctgttgccatttttcctgtttgagaaagactcaccagcttttggctggctcacagattggctttccttgggtcaggacccacccttttccctgccagctttggaagcttgacagaattcgagtgtgcagtggtggtaaataaatagtaaggaacacagagcagtcctggaggcgtgcctccatctgctgatgagaaaatccagtgctgtcatccagcccaggtcccagcggaatgggcctctctgttcagtaggatccccctcctgctgagtggttcatggcatgtttctgttcaacgcttttccatctgtaggattcttattctgtatttatttgtttttttgggtttttttattttttgagatggagtctcgctctgtcgcccaggctggagtgcagtggcacgaccccagctcgctgcagcctctgcctcccaggacgagggagatcctcccacctcagccttccacgtagctgggactacaggcatgcaccacaggcatgcaccaccacgccagctaatttttgtatttttggtagagacagggttgcatcatgttgcccaggctggtcttgaatgcctgagctcaagcaatctatttgccttggcctcccaaagtgctgggattacaggcatgagccaccacggccagccttctcatttgttttttttataaggaagctatctcttcttccctccccaactagggtattctttttccctttcgtcactttgctcatgtactgtattccttcaacttcattaatgaatccatttggaagcagtgaaaaaggcaactcagaaagctaagaagaaatagatagaggaatactcagagctatctgagtattttctttagtttgttagctctttggagctttgaaactggaaagacccagggagtgatgtggagaaagagactgagcttgtaagacacaggagcagtgagctaagggagatggagtagtggggacaaattctggcacattctgtctacactctgggtagatagaggagggaggatggagcacccatggtgggggtatgttggtgacagcattttcccaccagccagtgtaacaagtggctgatttgggggaaagatggcataaacaaatgagagaatgtgtttactatttgatgtagatgggttatttgcttcatttttcaaatcagtgtatataatcaagaatattcagcatgtttgaatagactgtcagagctggaactctttcattaacatctctggcacctttagttttagccctgaacattttatcttaaaattaaacattaccaaatgccttagtttatttcatttattaaatttatattcttatttgttatttatatcagcttccaatcagaagactatacaacctcctagggtaagttaaagtttattaaatgaattgtgaatgatcatttgagggattagactgaggaacttggtaattgagatattttgctatctgttttgtctcacgtcaaattaagagaatgttgaagtcattgcatgacctttgcatgaatgggtccagttctattttaaaacctgtgtttggtcattttagtgtcaatgggatggaataaatgatttcttaagattgtactgacttctcacacccaaaactggaaagtaggaataatggctatattatctctgcaatcagaaggaagctgattccaatatatcacctcacctgttggattcattggatgtgcatacacagaatgacaatttcaggcttaaaaatgaggagaaatctatactaagttgacatcactgataattataatctataaaataaatgtaaatattgctgaaaacatctgttcggagttatgattcgatcctctcccatacaaatgttttataaacattttttcccttaaaactgtgcttaaggtttcattgtaccttagataccttattaagccatctgaggaaattgcaagaaaggagtaattttaggagggcataaatgaagagaaaagcatatttataaacatagacttatcacagtgacaggcccaagaggtatgttgtggacataagctctggaaaggattatattgtacttagcttcctataacggagtgatgatagatgtatctggaatgccaaagagagtcttctgtctgtggccggaggtagaggtcacatatgcttctaagtctgacaagcctcattgtgcctaaagggcaggtggggcgggtatgtgtgtctaccacaggggtatattctagaaagattggtgccatagctatgttggtcacaagaggccagcaacttaactggacctgtgaatcctaacacattttctttcccagttactgagttcaatttgcgatacttaaagatgattctgttttgcttccacctcttcactgtgttatttattctgttgttgctatttatgcttgcactttcatatttttagaagttagaatttcttgagctgagagtggtgaagtgggaaattctgtttagaaaaataatattaagagaataatacagttattattaaactattaacccgacttggcaagctttgctttaacatttacaggcttatttcgttgttttgttttttcttttttttttgactgtaggatttactgcttactacgtaatttaaatattagcatatataagttttactataaaatgacatgacataaattatatttttatgtaaatatattttaaatatttttcagaaagctgtagaggaaccccttaatggtatgtggttatttcactcttaatcctttaccagatcataatttgatctggcccgcaaaacagttagaatgccctgtctatgccttaggaagaatctaggtttttttcctctttttttctttccttggcatctctactcttgattattcatcaagaattatgggctgggtgcggtggctcacgcttgcgatcccggcaccttgggaggccaaggcgggcagatcacgaggtcaagagattgagaccatcctggccaacatgttgaaaccctgtctctactgaaaatacaaaaattacctgggcatggtggtgtgtatctgcagtcccagctactcgggaggctgaggcaggagaattgcttgaacccggaaggcagatgttgcagtgagttgagatcatgccaccgcactccagcctggtgacagagtgagactctgtctcaaaaaaaataaaaaagaattatgaatattacttttataatattctcaccactgggaaaaatgcactattctgtgtctaagtagctgcttaccttaaccaagtgatatttgggcaaggggatcgttgccttttgctactggttgagacgaagcatggcaccccctagtagagaaggatcccaattacttccaatttgtgatgtacacattttagaaagatacaggctattgccacagagatagaccaaaacatctcattttctttctttgtaaacctagatctgatttcccaactaagtctgtttcctttgtgaatgctgtgggtatgatccacagaaaggctacataatgaaatgatagctttacaattaatttggctgtagagttgtagactagttagcatatcattgcatatttgtttatttagaaatgatttccaattgtggaacctcactaactgcctgcttggcttgttactaatcctagcattcaaagaatcaaaaggaatgatgaatgatggtaagtataaaatgcacttaataattatagatcagttaaaacatggacattggaaaacaaaaaagcttcttgaaaatgtggctcttttttagtaaaagggacactgtcagatgataaaggttcacatttcttgatgtatacaacttaaatctactttgctaaaaattgcaaaactactactgtaaaaactgtagggtgtcacgaatcagactccagtcatatggctcccagcaaagagaaattaccactttttgtaaaatgtttttcagattccgtgtctggtgactgtaactttaagatgccttttataaggcacataaataatctggcacaaatctttatcattttgacagagtttcttttatgcttgtgttggtgattttgttgcatttaacccatggggacttaacatctctgcttttccaatcaggagcttggttctaaccttttgggagatgattaaagagagaggttgagagagagagagagagagagagagagagagagagagagagagagagagatgtttcgtcctcagcttttgcttccattttttttttaagaactctgggctaataacttctaatctttatagaatatttcaaagaaatatatttgttcttaaagatacataggtttgagatattgagtgctacaagcatttattttggttttaccttaacatattatgattcctcagttttgttggcatttagtaattatgtttatgtttttatcttatcaaaaaatgtcttcttacctttgatatttataatcactcctcggtcatgtaaatagtttgctttatattttactgttttaaagtctgtgaccttacctgccctcttctgtagcaaagtgcagcatttaactcaggaagctatattccccccaagtgtcattaatatttgcataagattaaaaacactccagtcggccgggcgcggtggctcacgcctgtaatcccagcactttgggaggccgaggcgggcggatcacaaggtcaggagatcgagaccatcctggctaacacggtgaaaccccgtctctactaaaactacaaaaaattagccgggcatggtggcgggcacctgtggtcccagctacttgggaggctgaggcaggagaatggcgtgaacccgggaggcagagcttgcagtgagccaagatcgtgccactacactccagcctgggtgacagagcaagactccgtctcaagaaaaaaaaaaaaaaaaaaaaaaaaaaaacactccagtcatgcattggtgaacaaagtttaaaacaacgtgtattcagcatggagtcacagaatgatcctacttttgtatgtttgtgtcacagcctttaaaagcatgtcttgttatataagccattaccctcctaaaaaagactatagttcacaggaataagttaaaagacataacaaaacataaaatgactagtaccaggaattgtgaccatggtttgttctggtaactgtggcatggcatggtttgttccaggaattgtggcatggtttgttcatgtttacattctgatgtcctattttttttttttaatttctatgtcctttccttttccttggtggtgtcattgttctgtagctgtatgaagaaactaaacttttctccattttcaggaaagcaatctaagaatcttgagtgcctcttcctttgttaatttctcttaagatgtgacttttttaaactactgcatcaggaaatattgtaaaacagttttgccttgaatatttgtgatgaaatctacgatgatcttcaagattctcttaattttgctaatattcagctgatcagaatttgtttttaaaatgtctggctggtgggtacttcccactgacaactgcttattgcttacagtatgtctgccttgtcaatgaatgaggttcagggtgcttcctagggatcagagtcagtaccatttttctctttcatctacagctgatcagatgtttattttacttacattaaatgaatgatggagatccaaagtgaatattatagaatattattctaggatcaacatcttttgctttgaaaaatcaacatctcttggcttttcctcagccaacccagcaaacagagattatcagactctgttgattttttactttcatttggcattggcctttttcttactgaaattaaaaaggctaatgatttgcctggtttctgtctctgacctttgcaggtctattttcttaatttttagatactatatatctgaaactttttttaatgtgtcaactttttaatggatagaaaatagacacgaatagtgattatgtgttcatttttcaattttccagaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 30 - DNA, 4657 bp, Homo sapiens
  source 1..4657: mol_type = genomic DNA
atagtagcaggaccactatagagagaacactcatgtagcaggtcatggaacagtgctagagccacagttcaggagtgagagggtggtggggattaaggggagaagagggcctgagggatgagagggacggagggaagggctggaggagcaggaggtgaggaaaaggagcagaggaaagaattccaaagcagcagaactcttaggtttaaacacattgttttatagattttaatacatccatctacagagcttcgctgggtgttctttgcagttggcctttaatatcttatgtgggtctgcctagaaactaattgttttttatgttaatcaggtttaaaaaatactaagtattcctaaaaaatatacactccactcacatgtggatacttcctaaaaacaggcagtgcgtgagcactagtgaggggcattgtgactgcactgaacacttacaactgtgaggtgaataaagtttgtgctggctcctggttgcaacatatagtaacatagtgtggtactttgtcttgaggagatgtcctggactcacacggaaacttagggctacggaatgaaggtaaatttaaaataaaacaagcgggagtcacagatacactgtctgggaaagtgaaacttaagagctttgtgagtcgtgttgtaatgcttttagatgcatttatataccaacaggccaaagtcacattttttaccgattagattcctgatcattcaggggttaccaaggttatgctacccactatagttaataaacaaaaagcaaactggtctctattctatctcatgcactcaggcacaacttttccagatttaagggggaaaaaaaaccctgtctttacacctacaatcccagggcgagctcactctctggcaacaagctccctggggtgatttttcttctagaagagtacaggaggacaggcaaggagtgggaggcagggagtccagttcagggacagggattccgggatgaaaagtgaagggagagggccagggaccttgccgagggtttctccctggtttctcagacagctcctgggccaagactcagggagacactgagacagaacgcttggcacaagagtagcggggtcagggcgaagtcccagggcctcaagcgtggctctcagggtctcaggccccacaggcggtgtatgggttggggaggccccgcgttggggattctctcctccttctcctaacctgtgtcgggtccttcttcctggatactcaccgggcggccccagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggtgagtgcggggtcaggagggaaacagcccctgcgcggaggagggaggggccggcccggcgggggcgcaggactcggcagccgcgccgggaggagggtcgggcgggtctcaacccctcctcgcccccaggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagtgagtaactccggcccagggagcagatcacgacccccacctccatgccccacggacggcccgggtactcccgagtctccgggtctgggatccaccccgaggccgcgggacccgcccagaccctctacctgggagaaccccaaggcgcctttaccaaaatccccgcgggtgggtccgggcgagggcgaggctcggtgggcggggctgaccgagggggtggggccaggttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcgggtaccaggggcagtggggcgcctccctgatctcctgtagacctctcagcctggcctagcacaaggagaggaggaaaatgggaccaacactagaatatcgccctccctctggtcctgagggagaggaatcctcctgggtttccagatcctgtaccagagagtgattctgagggtccgtcctgctctctgggacaattaagggatgaagtctctgagggagtggaggggaagacaatccctggaagactgatcaggggttccctttgaccccacagcagccttggcaccaggacttttcccctcaggccttgttctctgcctcacactcaatgtgtgtgggggtctgactccagctcctctgagtcccttggcctccactcaggtcagaaccggaggtccctgctcccccgctcagagactagaactttccaaggaataggagattatcccaggtgcccgtgtccaggctggtgtctgggttctgtgctcccttccccaccccaggtatctggttcattcttaggatggtcacatccaggtgctgctggagtgtcccatgagagatgcaaagtgcttgaattttctgactcttcctttcagacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggagtaaggagggagatggaggcatcatgtctgttagggaaagcaggagcctctctgaagacctttaacagggtcggtggtgagggctgggggtcagagaccctcaccttcacctcctttcccagagcagtcttccctgcccaccatccccatcatgggtatcgttgctggcctggttgtccttgcagctgtagtcactggagctgcggtcgctgctgtgctgtggagaaagaagagctcaggtaaggaaggggtgacaagtggggtctgagttttcttgtcccactgggggtttcaagccccaggtagaagtgtgccctgcctggttactgggaagcaccatccacactcatgggcctacccagcctgggccctgtgtgccagcaccttctcttttgtaaagcacctgtgacaatgaaggacagatttattaccttgatgattgtagtgatggggacctgatcccagtaatcacaggtcaggagaaggtccctggctaaggacagaccttaggagggcagttggtcgaggacccacatctgctttccttgtttttcctgatcccgccctgggtctgcagtcacacatttctggaaacttctcgagggtccaagactaggaggttcctctaggacctcatggccctgccacctttctggcctctcacaggacattttcttcccacagattgaaaaggagggagctactctcaggctgcaagtaagtatgaaggaggctgatccctgagatccttgggatcttgtgtttgggagcccatgggggagctcacccaccccacaattcctcctctggccacatctcctgtggtctctgaccaggtgctgtttttgttctactctaggcagtgacagtgcccagggctctaatgtgtctctcacggcttgtaaatgtgacaccccggggggcctgatgtgtgtgggttgttgaggggaacaggggacatagctgtgctatgaggtttctttgacttcaatgtattgagcatgtgatgggctgtttaaagtgtcacccctcactgtgactgatatgaatttgttcatgaatatttttctgtagtgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaactttccacagta

SEQ ID NO: 31 - DNA, 23 bp, synthetic construct
  misc_feature 1..23: note = Synthetic
  source 1..23: mol_type = other DNA
tccagcattggaacttctgatct

SEQ ID NO: 32 - skipped

SEQ ID NO: 33 - DNA, 23 bp, synthetic construct
  misc_feature 1..23: note = Synthetic
  source 1..23: mol_type = other DNA
ccaccctgttgtgataaccacta

SEQ ID NO: 34 - skipped

SEQ ID NO: 35 - DNA, 23 bp, synthetic construct
  misc_feature 1..23: note = Synthetic
  source 1..23: mol_type = other DNA
gccacccactgtccttttataat

SEQ ID NO: 36 - skipped

SEQ ID NO: 37 - DNA, 22 bp, synthetic construct
  misc_feature 1..22: note = Synthetic
  source 1..22: mol_type = other DNA
ttggatttgtaaggcactttat

SEQ ID NO: 38 - skipped

SEQ ID NO: 39 - DNA, 20 bp, synthetic construct
  misc_feature 1..20: note = Synthetic
  source 1..20: mol_type = other DNA
ggctcatcgacgcctgtgac

SEQ ID NO: 40 - skipped

SEQ ID NO: 41 - DNA, 22 bp, synthetic construct
  misc_feature 1..22: note = Synthetic
  source 1..22: mol_type = other DNA
aatagcagacctcagactgcca

SEQ ID NO: 42-43 - skipped (2 entries)

SEQ ID NO: 44 - DNA, 24 bp, synthetic construct
  misc_feature 1..24: note = Synthetic
  source 1..24: mol_type = other DNA
cagtgttggaacgggacagtattt

SEQ ID NO: 45-46 - skipped (2 entries)

SEQ ID NO: 47 - DNA, 20 bp, synthetic construct
  misc_feature 1..20: note = Synthetic
  source 1..20: mol_type = other DNA
ccaaactaaacttgctgctt

SEQ ID NO: 48 - skipped

SEQ ID NO: 49 - protein, 312 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNE

SEQ ID NO: 50 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 51 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 52 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 53 - protein, 245 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 54 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 55 - protein, 174 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 56 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 57 - DNA, 19 bp, synthetic construct
  misc_feature 1..19: note = Synthetic
  source 1..19: mol_type = other DNA
tgtgagcaagacaaagtac

SEQ ID NO: 58-59 - skipped (2 entries)

SEQ ID NO: 60 - DNA, 23 bp, synthetic construct
  misc_feature 1..23: note = Synthetic
  source 1..23: mol_type = other DNA
acttaaaaggcccaagcactgaa

SEQ ID NO: 61 - skipped

SEQ ID NO: 62 - DNA, 25 bp, synthetic construct
  misc_feature 1..25: note = Synthetic
  source 1..25: mol_type = other DNA
tattttgttacttggtacaccagca

SEQ ID NO: 63 - skipped

SEQ ID NO: 64 - DNA, 22 bp, synthetic construct
  misc_feature 1..22: note = Synthetic
  source 1..22: mol_type = other DNA
cgccaaactaaacttgctgctt

SEQ ID NO: 65 - DNA, 10 bp, synthetic construct
  misc_feature 1..10: note = Synthetic
  source 1..10: mol_type = other DNA
acttgctgct

SEQ ID NO: 66 - DNA, 22 bp, synthetic construct
  misc_feature 1..22: note = Synthetic
  source 1..22: mol_type = other DNA
atccctaatttgagggtcagtt

SEQ ID NO: 67 - DNA, 13 bp, synthetic construct
  misc_feature 1..13: note = Synthetic
  source 1..13: mol_type = other DNA
tttgagggtcagt

SEQ ID NO: 68 - DNA, 25 bp, synthetic construct
  misc_feature 1..25: note = Synthetic
  source 1..25: mol_type = other DNA
ggtgttggatttgtaaggcacttta

SEQ ID NO: 69 - skipped

SEQ ID NO: 70 - DNA, 23 bp, synthetic construct
  misc_feature 1..23: note = Synthetic
  source 1..23: mol_type = other DNA
aaggaatgggcccgtgggatgca

SEQ ID NO: 71 - skipped

SEQ ID NO: 72 - DNA, 23 bp, synthetic construct
  misc_feature 1..23: note = Synthetic
  source 1..23: mol_type = other DNA
atttcttttgaagatatattgta

SEQ ID NO: 73 - skipped

SEQ ID NO: 74 - DNA, 391 bp, synthetic construct
  misc_feature 1..391: note = Synthetic
  source 1..391: mol_type = other DNA
attgaaaaggagggagctactctcaggctgcaatgtgaaacagctgccctgtgtgggactgagtggcaagatttgttcatgccttccctttgtgacttcaagaaccctgacttctctttctgcagagaccagcccacccctgtgcccaccatgaccctcttcctcatgctgaactgcattccttccccaatcacctttcctgttccagaaaaggggctgggatgtctccgtctctgtctcaaatttgtggtgcactgagctataacttacttctgtattaaaattagaatctgagtataaatttactttttcaaattatttccaagagagattgatgggttaattaaaggagaagattcctgaaatttgagagacaaaataaatggaagac

SEQ ID NO: 75 - DNA, 14 bp, synthetic construct
  misc_feature 1..14: note = Synthetic
  source 1..14: mol_type = other DNA
atttgttcatgcct

SEQ ID NO: 76 - DNA, 1563 bp, Homo sapiens
  source 1..1563: mol_type = genomic DNA
ggcaagttggacgcccgcaagatccgcgagattctcattaaggccaagaagggcggaaagatcgccgtgtaacaattggcagagctcagaattcaagcgatcgccacaaagagggaagtgaacagtgctatctgaacctgtggtcttgggagccagggtgacctgatatgacatctaaagaagcttctggactctgaacaagaattcggtggcctgcagagcttgccatttgcacttttcaaatgcctttggatgacccagcactttaatctgaaacctgcaacaagactagccaacacctggccatgaaacttgccccttcactgatctggactcacctctggagcctatggctttaagcaagcactactgcactttacagaattaccccactggatcctggacccacagaattccttcaggatccttcttgctgccagactgaaagcaaaaggaattatttcccctcaagttttctaagtgatttccaaaagcagaggtgtgtggaaatttccagtaacagaaacagatgggttgccaatagagttattttttatctatagcttcctctgggtactagaagaggctattgagactatgagctcacagacagggcttcgcacaaactcaaatcataattgacatgttttatggattactggaatcttgatagcataatgaagttgttctaattaacagagagcatttaaatatacactaagtgcacaaattgtggagtaaagtcatcaagctctgtttttgaggtctaagtcacaaagcatttgttttaacctgtaatggcaccatgtttaatggtggttttttttttgaactacatctttcctttaaaaattattggtttctttttatttgtttttaccttagaaatcaattatatacagtcaaaaatatttgatatgctcatacgttgtatctgcagcaatttcagataagtagctaaaatggccaaagccccaaactaagcctccttttctggccctcaatatgactttaaatttgacttttcagtgcctcagtttgcacatctgtaatacagcaatgctaagtagtcaaggcctttgataattggcactatggaaatcctgcaagatcccactacatatgtgtggagcagaagggtaactcggctacagtaacagcttaattttgttaaatttgttctttatactggagccatgaagctcagagcattagctgacccttgaactattcaaatgggcacattagctagtataacagacttacataggtgggcctaaagcaagctccttaactgagcaaaatttggggcttatgagaatgaaagggtgtgaaattgactaacagacaaatcatacatctcagtttctcaattctcatgtaaatcagagaatgcctttaaagaataaaactcaattgttattcttcaacgttctttatatattctacttttgggtaacgcgtaagcggccgcggcatctagattcgaagaaaatgaccgaccaagcgacgcccaacctgccatcacgagatttcgattccaccgccgccttctatgaaagg

SEQ ID NO: 77 - DNA, 1033 bp, Homo sapiens
  source 1..1033: mol_type = genomic DNA
gacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 78 - DNA, 2692 bp, Homo sapiens
  source 1..2692: mol_type = genomic DNA
tccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 79 - DNA, 2692 bp, Homo sapiens; identical to SEQ ID NO: 78
  source 1..2692: mol_type = genomic DNA

SEQ ID NO: 80 - DNA, 4197 bp, Homo sapiens
  source 1..4197: mol_type = genomic DNA
ctgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 81 - DNA, 4193 bp, Homo sapiens
  source 1..4193: mol_type = genomic DNA
agtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 82 - DNA, 4197 bp, Homo sapiens; identical to SEQ ID NO: 80
  source 1..4197: mol_type = genomic DNA

SEQ ID NO: 83 - DNA, 282 bp, Homo sapiens
  source 1..282: mol_type = genomic DNA
aaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaa

SEQ ID NO: 84 - DNA, 282 bp, Homo sapiens; identical to SEQ ID NO: 83
  source 1..282: mol_type = genomic DNA

SEQ ID NO: 85 - DNA, 282 bp, Homo sapiens; identical to SEQ ID NO: 83
  source 1..282: mol_type = genomic DNA

SEQ ID NO: 86 - DNA, 282 bp, Homo sapiens; identical to SEQ ID NO: 83
  source 1..282: mol_type = genomic DNA

SEQ ID NO: 87 - DNA, 2692 bp, Homo sapiens; identical to SEQ ID NO: 78
  source 1..2692: mol_type = genomic DNA

SEQ ID NO: 88 - DNA, 4193 bp, Homo sapiens; identical to SEQ ID NO: 81
  source 1..4193: mol_type = genomic DNA

SEQ ID NO: 89 - DNA, 748 bp, Homo sapiens
  source 1..748: mol_type = genomic DNA
aaaggagggagctactctcaggctgcaagtaagtatgaaggaggctgatccctgagatccttgggatcttgtgtttgggagcccatgggggagctcacccaccccacaattcctcctctggccacatctcctgtggtctctgaccaggtgctgtttttgttctactctaggcagtgacagtgcccagggctctaatgtgtctctcacggcttgtaaatgtgacaccccggggggcctgatgtgtgtgggttgttgaggggaacaggggacatagctgtgctatgaggtttctttgacttcaatgtattgagcatgtgatgggctgtttaaagtgtcacccctcactgtgactgatatgaatttgttcatgaatatttttctgtagtgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaactttccacagta

SEQ ID NO: 90 - DNA, 23 bp, Homo sapiens
  source 1..23: mol_type = genomic DNA
aagaggctattgagactatgagc

SEQ ID NO: 91 - DNA, 26 bp, Homo sapiens
  source 1..26: mol_type = genomic DNA
aagcactactgcactttacagaatta

SEQ ID NO: 92 - DNA, 23 bp, Homo sapiens
  source 1..23: mol_type = genomic DNA
tggatcctggacccacagaattc

SEQ ID NO: 93 - DNA, 25 bp, Homo sapiens
  source 1..25: mol_type = genomic DNA
gagagcatttaaatatacactaagt

SEQ ID NO: 94 - DNA, 21 bp, Homo sapiens
  source 1..21: mol_type = genomic DNA
gaaattgactaacagacaaat

SEQ ID NO: 95 - DNA, 24 bp, Homo sapiens
  source 1..24: mol_type = genomic DNA
gttctaattaacagagagcattta

SEQ ID NO: 96 - DNA, 22 bp, Homo sapiens
  source 1..22: mol_type = genomic DNA
ggtctaagtcacaaagcatttg

SEQ ID NO: 97 - DNA, 23 bp, Homo sapiens
  source 1..23: mol_type = genomic DNA
atttattacctctgatacctcaa